(19) United States
(12) Patent Application Publication
(10) Pub. No.: US 2025/0195484 A1
(43) Pub. Date: Jun. 19, 2025

(54) CEREBLON LIGANDS AND USES THEREOF

(71) Applicants: REGENTS OF THE UNIVERSITY OF MICHIGAN, Ann Arbor, MI (US); Oncopia Therapeutics, Inc. d/b/a SK Life Science Labs, King Of Prussia, PA (US)

(72) Inventors: Haibin Zhou, Ann Arbor, MI (US); Dimin Wu, Ann Arbor, MI (US); Changwei Wang, Ann Arbor, MI (US); Mi Wang, Ann Arbor, MI (US); Shaomeng Wang, Superior Township, MI (US); Rohan Rej, Ann Arbor, MI (US); Zhixiang Chen, Ann Arbor, MI (US); Ranjan Kumar Acharyya, Ann Arbor, MI (US); Longchuan Bai, Ann Arbor, MI (US); Paul Kirchhoff, Saline, MI (US); Guozhang Xu, New York, NY (US); Xuqing Zhang, New York, NY (US); Zhenwu Li, New York, NY (US); Biao Hu, Ann Arbor, MI (US); Jianfeng Lu, Ann Arbor, MI (US)

(21) Appl. No.: 18/849,796

(22) PCT Filed: Mar. 24, 2023
(86) PCT No.: PCT/US23/16289

§ 371 Date: Sep. 23, 2024

Related U.S. Application Data
(60) Provisional application No. 63/446,105, filed on Feb. 16, 2023.
(60) Provisional application No. 63/446,112, filed on Feb. 16, 2023.
(60) Provisional application No. 63/427,277, filed on Nov. 22, 2022.
(60) Provisional application No. 63/412,194, filed on Sep. 30, 2022.
(60) Provisional application No. 63/408,744, filed on Sep. 21, 2022.
(60) Provisional application No. 63/408,758, filed on Sep. 21, 2022.
(60) Provisional application No. 63/408,601, filed on Sep. 21, 2022.
(60) Provisional application No. 63/408,633, filed on Sep. 21, 2022.
(60) Provisional application No. 63/388,297, filed on Jul. 12, 2022.
(60) Provisional application No. 63/388,299, filed on Jul. 12, 2022.
(60) Provisional application No. 63/388,300, filed on Jul. 12, 2022.
(60) Provisional application No. 63/388,302, filed on Jul. 12, 2022.
(60) Provisional application No. 63/331,558, filed on Apr. 15, 2022.
(60) Provisional application No. 63/323,792, filed on Mar. 25, 2022.

(51) Int. Cl. A61K 31/438; A61K 31/55; C07D 491/20

(52) U.S. Cl. CPC A61K 31/438; A61K 31/55; C07D 491/20

(57) ABSTRACT

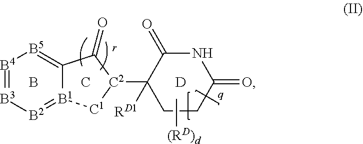

Described herein are compounds or conjugates of Formula II and pharmaceutically acceptable salts, solvates, or stereoisomers thereof, as well as their uses (e.g., as cereblon-binding agents or bifunctional degraders for degrading certain proteins).

### RELATED APPLICATIONS

This application claims the benefit of and priority to U.S. Provisional Application No. 63/412,194, filed Sep. 30, 2022; U.S. Provisional Application No. 63/323,792, filed Mar. 25, 2022; U.S. Provisional Application No. 63/446,105, filed Feb. 16, 2023; U.S. Provisional Application No. 63/331,558, filed Apr. 15, 2022; U.S. Provisional Application No. 63/446,112, filed Feb. 16, 2023; U.S. Provisional Application No. 63/388,300, filed Jul. 12, 2022; U.S. Provisional Application No. 63/408,744, filed Sep. 21, 2022; U.S. Provisional Application No. 63/427,277, filed Nov. 22, 2022; U.S. Provisional Application No. 63/388,302, filed Jul. 12, 2022; U.S. Provisional Application No. 63/408,758, filed Sep. 21, 2022; U.S. Provisional Application No. 63/388,297, filed Jul. 12, 2022; U.S. Provisional Application No. 63/408,601, filed Sep. 21, 2022; U.S. Provisional Application No. 63/388,299, filed Jul. 12, 2022; and U.S. Provisional Application No. 63/408,633, filed Sep. 21, 2022; the contents of which are incorporated herein by reference in their entireties.

### BACKGROUND

Cereblon (CRBN), a component of the DDB1-CUL4α-Rocl ubiquitin ligase complex, is a molecular target of immunomodulatory agents such as thalidomide, lenalidomide, and pomalidomide. Inhibition of CRBN ubiquitination by these agents may allow CRBN to accumulate, leading to the increased cullin-4 RING E3 ligase-mediated degradation of target proteins.

The discovery process of CRBN type E3 ligase ligand is related to the study of thalidomide's mechanism of action. In 2010, while studying the toxicity of thalidomide, scientists discovered that cereblon is a binding protein of thalidomide (Science 2010, 327, 1345). Cerebellar protein is part of the E3 ubiquitin ligase protein complex, which acts as a substrate receptor to select ubiquitinated proteins. The study shows that thalidomide-cerebellar protein binding in vivo may be the cause of thalidomide teratogenicity. Subsequent studies found that the compound and related structures can be used as anti-inflammatory agents, anti-angiogenic agents and anti-cancer agents. Lenalidomide and pomalidomide obtained by further modification of the structure of thalidomide have greatly improved their safety and significantly reduced their teratogenic effects. Lenalidomide has been approved by the FDA in 2006 for marketing. Two groundbreaking papers published in Science in 2014 pointed out that lenalidomide works by degrading two special B cell transcription factors, Ikaros family zinc finger structural proteins 1 and 3 (IKZF1 and IKZF3), which further reveals the structure of thalidomide may be combined with the E3 ubiquitin ligase protein complex of the cerebellar protein to further play a role in degrading the target protein (Science, 2014, 343, 301; Science, 2014, 343, 305).

On this basis, CRBN ligands are widely used in protein degradation, and a series of proteolysis targeting chimera (PROTAC) molecules based on CRBN ligands have been developed. Due to the influence of CRBN ligand itself on the target point, it may additionally degrade zinc finger domain protein. Therefore, the design and synthesis of new and highly selective CRBN ligands is also particularly important in the synthesis of PROTAC molecules.

### SUMMARY

In certain aspects, the present disclosure provides compounds or conjugates of Formula II:

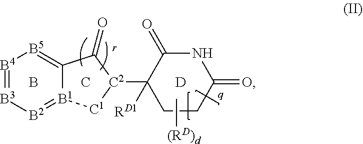

wherein each of the variables in Formula II is described, embodied, and exemplified herein.

In certain aspects, the present disclosure provides pharmaceutical compositions comprising a compound or a conjugate disclosed herein, and a pharmaceutically acceptable excipient.

In certain aspects, provided herein are methods of binding cereblon E3 ubiquitin ligase protein complex in a subject or biological sample comprising administering a compound or a conjugate described herein to the subject or contacting the biological sample with a compound or a conjugate described herein.

In certain aspects, provided herein are uses of a compound or a conjugate described herein in the manufacture of a medicament for binding cereblon E3 ubiquitin ligase protein complex in a subject or biological sample.

In certain aspects, provided herein are compounds or conjugates described herein for use in binding cereblon E3 ubiquitin ligase protein complex in a subject or biological sample.

In certain aspects, provided herein are methods of degrading a protein in a subject or biological sample comprising administering a compound or a conjugate described herein to the subject or contacting the biological sample with a compound described herein.

In certain aspects, provided herein are uses of a compound or a conjugate described herein in the manufacture of a medicament for degrading a protein in a subject or biological sample.

In certain aspects, provided herein are compounds or conjugates described herein for use in degrading a protein in a subject or biological sample.

In certain aspects, provided herein are methods of treating or preventing a disease or disorder a subject in need thereof, comprising administering to the subject a compound or a conjugate described herein.

In certain aspects, provided herein are uses of a compound or a conjugate described herein in the manufacture of a medicament for treating or preventing a disease or disorder in a subject in need thereof.

In certain aspects, provided herein are compounds or oncjugates described herein for use in treating or preventing a disease or disorder in a subject in need thereof.

### DETAILED DESCRIPTION

The present disclosure relates to compounds that show cereblon-binding activity, bifunctional degraders comprising a cereblon-binding moeity, and pharmaceutical compositions comprising such compounds or bifunctional degraders. The present disclosure further relates to methods of degrading a protein in a subject or biological sample comprising administering a compound described herein to the subject or contacting the biological sample with a compound described herein. The present disclosure also relates to methods of treating or preventing a disease or disorder a subject in need thereof, comprising administering to the subject a compound described herein.

### Compounds of the Present Disclosure

### Cereblon Ligands

In certain aspects, the present disclosure provides compounds of Formula II:

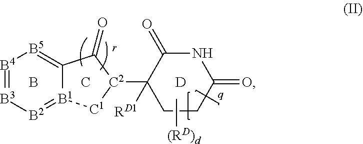

and pharmaceutically acceptable salts, solvates, or stereoisomers thereof, wherein:
B² is N or CR^{B2};
B³ is N or CR^{B3};
B⁴ is N or CR^{B4};
B⁵ is N or CR^{B5};
R^{B2}, R^{B3}, R^{B4}, and R^{B5} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{C}S(═O)R^{a}, —NR^{C}S(═O)₂OR^{b}, —NR^{C}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
wherein one of R^{B2} and R^{B3}, R^{B3} and R^{B4}, or R^{B4} and R^{B5}, together with the carbon atoms to which they are bonded, form Ring A, wherein Ring A is optionally substituted 7- to 16-membered spiro heterocycle;
denotes an optional covalent bond between B¹ and C¹;
i) when the bond between B¹ and C¹ is present:
   r is 1;
   B¹ is C;
   C¹ is —C(R^{C1})₂—, —C(═O)—, —(C═O)—N(R^{C1}′)—*, or —N═C(R^{C1}′)—*;
   each R^{C1} is independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}; or
   two R^{C1}, together with the carbon atom to which they are attached, form C₃₋₆ carbocycle or 3- to 6-membered heterocycle, wherein the carbocycle or heterocycle is optionally substituted with one or more R^{u};
   R^{C1}′ is H or C₁₋₆ alkyl optionally substituted with one or more R, and * denotes attachment to Ring B; and
   C² is N;
ii) when the bond between B¹ and C¹ is absent:
   r is 0 or 1;
   B¹ is N or CR^{B1};
   R^{B1} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
   C¹ is absent; or
   C¹ is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
   C₂ is N or O;
   wherein i) when C² is N, then C¹ is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}; ii) when C₂ is O, then C¹ is absent;
R^{D1} is hydrogen, deuterium, or C₁₋₆ alkyl optionally substituted with one or more R^{u};
q is an integer from 0 to 2,
each RD is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; and
d is an integer selected from 0 to 5,
wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{C}S(═O)R^{a}, —NR^{C}S(═O)₂OR^{b}, —NR^{b}s(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₃₋₆ carbocyclyl, 3- to 6-membered heterocyclyl, C₆ aryl, and 5- or 6-membered heteroaryl; or
two R^{u}, together with the one or more intervening atoms, form C₆₋₁₀ aryl, 5- to 10-membered heteroaryl, C₃₋₁₂ carbocyclyl, or 3- to 12-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl or 5- to 10-membered heteroaryl, wherein the heterocyclyl or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₃₋₆ carbocyclyl, and 3- to 6-membered heterocyclyl;
wherein each of R^{a}, Rb, R^{c}, and R^{d} is independently and optionally substituted with one or more R^{z};
each R^{z} is independently oxo, halogen, —CN, —NO₂, —OH, —NH₂, C₁₋₆ alkyl, C₁₋₆ alkoxy, C₁₋₆ alkylamino, C₃₋₆ carbocyclyl, 3- to 6-membered heterocyclyl, C₆ aryl, or 5- or 6-membered heteroaryl.

In certain embodiments, the compound or conjugate of Formula II is a compound or conjugate of Formula II-1

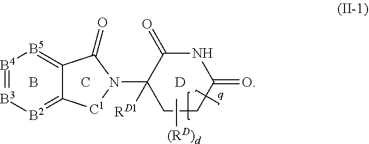

In certain embodiments, the compound or conjugate of Formula II is a compound or conjugate of Formula II-2

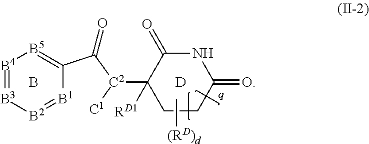

In certain embodiments, B² is N or CR^{B2}. In certain embodiments, B² is N. In certain embodiments, B² is CR^{B2}.

In certain embodiments, B³ is N or CR^{B3}. In certain embodiments, B³ is N. In certain embodiments, B³ is CR^{B3}.

In certain embodiments, B⁴ is N or CR^{B4}. In certain embodiments, B² is N. In certain embodiments, B⁴ is CR^{B4}.

In certain embodiments, B⁵ is N or CR^{B5}. In certain embodiments, B² is N. In certain embodiments, B⁵ is CR^{B5}.

In certain embodiments, one of B², B³, B⁴, and B⁵ is N. In certain embodiments, two of B², B³, B⁴, and B⁵ are N.

In certain embodiments, R^{B2}, R^{B3}, R^{B4}, and R^{B5} are independently hydrogen, halogen (e.g., —F, —Cl, —Br, or —I), —CN, —NO₂, —OH, —NH₂, C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, S-butylpentylamino, t-butylpentylamino, n-butylhexylamino, i-butylhexylamino, S-butylhexylamino, t-butylhexylamino, or pentylhexylamino), C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)), C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)), C₃₋₁₂ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₇), cycloheptenyl(C₇), cycloheptadienyl(C₇), cycloheptatrienyl(C₇), cyclooctyl(C₈), cyclooctenyl(C₅), bicyclo[2.2.1]heptanyl(C₇), bicyclo[2.2.2]octanyl(C₈), cyclononyl(C₉), cyclononenyl(C₉), cyclodecyl(C₁₀), cyclodecenyl(C₁₀), octahydro-1H-indenyl(C₉), decahydronaphthalenyl(C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}Rd, —NR^{C}S(═O)₂R^{a}, —NR^{C}S(═O)R^{a}, —NR^{C}S(═O)₂OR^{b}, —NR^{C}S(═O)₂NR^{c}R^{d}, —NR^{b}C(═O)NR^{c}Rd, —NR^{b}C(═O)R^{a}, —NR^{o}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, R^{B2}, R^{B3}, R^{B4}, and R^{B5} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃-12 carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, R^{B2}, R^{B3}, R^{B4}, and R^{B5} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, R^{B2}, R^{B3}, R^{B4}, and R^{B5} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, R^{B2}, R^{B3}, R^{B4}, and R^{B5} are independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, R^{B4} and R^{B5} are both hydrogen. In certain embodiments, R^{B2} and R^{B5} are both hydrogen.

In certain embodiments, R^{B2} and R^{B3}, R^{B3} and R^{B4}, or R^{B4} and R^{B5}, together with the carbon atoms to which they are bonded, form Ring A, wherein Ring A is optionally substituted 7- to 16-membered spiro heterocycle.

In certain embodiments, only one of R^{B2} and R^{B3}, R^{B3} and R^{B4}, or R^{B4} and R^{B5}, together with the carbon atoms to which they are bonded, form Ring A.

In certain embodiments, R^{B2} and R^{B3}, together with the carbon atoms to which they are bonded, form Ring A.

In certain embodiments, R^{B3} and R^{B4}, together with the carbon atoms to which they are bonded, form Ring A.

In certain embodiments, Ring A is optionally substituted 7- to 16-membered spiro heterocycle (e.g., heterocyclyl comprising two 4- to 8-membered spiro rings and 1-5 heteroatoms selected from N, O, and S).

In certain embodiments, Ring A is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{C}S(═O)R^{a}, —NR^{C}S(═O)₂OR^{b}, —NR^{C}S(═O)₂NR^{c}R^{d}, —NR^{b}C(═O)NR^{c}R^{d}, —NR^{o}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, Ring A is optionally substituted with one or more R^{u}, R^{A1}R^{A1}′, R^{A2}, or R^{A2}′.

In certain embodiments, R^{u} is R^{A1}. In certain embodiments, R^{u} is R^{A1}′. In certain embodiments, R^{u} is R^{A2}. In certain embodiments, R^{u} is R^{A2}′.

In certain embodiments,
Ring A is:

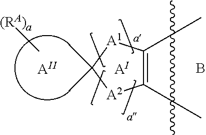

or
Ring A attached to -L-T is

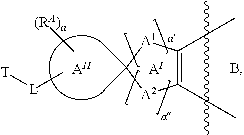

wherein:

** denotes attachment to C;
Ring A^{II} is C₃₋₈ carbocycle or 3- to 8-membered heterocycle;
each A¹ is independently —C(R^{A1})₂—, —NR^{A1}′—, —O—, —S—, —S(═O)—, or —S(═O)₂—;
each A² is independently —C(R^{A2})₂—, —NR^{A2}′—, —O—, —S—, —S(═O)—, or —S(═O)₂—;
each occurrence of RAI and R^{A2} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{C}S(═O)R^{a}, —NR^{C}S(═O)₂OR^{b}, —NR^{C}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
two geminal R^{A1} or two geminal R^{A2} together form oxo; or
two geminal R^{A1} or two geminal R^{A2}, together with the carbon atom to which they are attached, form C₃₋₆ carbocycle or 3- to 6-membered heterocycle, wherein the carbocycle or heterocycle is optionally substituted with one or more R^{u};
each occurrence of R^{A1}′ and R^{A2}′ is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
a′ and a″ are independently an integer selected from 0-3, wherein one of a′ and a″ is 0, and, and a′ and a″ are not both 0;
each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{C}S(═O)R^{a}, —NR^{C}S(═O)₂OR^{b}, —NR^{C}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
a is an integer selected from 0 to 8, as valency permits.

In certain embodiments, Ring A¹ is heterocycle. In certain embodiments, Ring A¹ is not carbocycle.

In certain embodiments,
Ring A is:

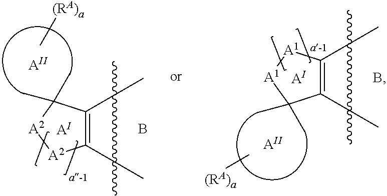

or
Ring A attached to -L-T is

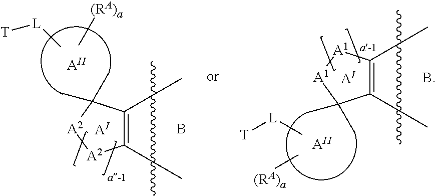

In certain embodiments, Ring A¹ is C₃₋₈ carbocycle (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₇), cycloheptenyl(C₇), cycloheptadienyl(C₇), cycloheptatrienyl(C₇), cyclooctyl(C₈), cyclooctenyl(C₈), bicyclo[2.2.1]heptanyl(C₇), or bicyclo[2.2.2]octanyl(C₈)) or 3- to 8-membered heterocyclyl (e.g., heterocyclyl comprising one or two 3- to 8-membered rings and 1-5 heteroatoms selected from N, O, and S).

In certain embodiments, each A¹ is independently —C(R^{A1})₂—, —NR^{A1}′—, —O—, —S—, —S(═O)—, or —S(═O)₂—. In certain embodiments, each A¹ is independently —C(R^{A1})₂—, —NR^{A1}′—, or —O—.

In certain embodiments, each A² is independently —C(R^{A2})₂—, —NR^{A2}′—, —O—, —S—, —S(═O)—, or —S(═O)₂—. In certain embodiments, each A² is independently —C(R^{A2})₂—, —NR^{A2}′—, or —O—.

In certain embodiments, each occurrence of R^{A1} and R^{A2} is independently hydrogen, halogen (e.g., —F, —Cl, —Br, or —I), —CN, —NO₂, —OH, —NH₂, C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, S-butylhexylamino, t-butylhexylamino, or pentylhexylamino), C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)), C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)), C₃₋₁₂ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₇), cycloheptenyl(C₇), cycloheptadienyl(C₇), cycloheptatrienyl(C₇), cyclooctyl(C₈), cyclooctenyl(C₅), bicyclo[2.2.1]heptanyl(C₇), bicyclo[2.2.2]octanyl(C₈), cyclononyl(C₉), cyclononenyl(C₉), cyclodecyl(C₁₀), cyclodecenyl(C₁₀), octahydro-1H-indenyl(C₉), decahydronaphthalenyl(C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{c}S(═O)R^{a}, —NR^{C}S(═O)₂OR^{b}, —NR^{C}S(═O)₂NR^{c}R^{d}, —NR^{b}C(═O)NR^{c}R^{d}, —NR^{o}C(═O)R^{a}, —NR^{o}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, each occurrence of RAI and R^{A2} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each occurrence of RAI and R^{A2} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each occurrence of R^{A1} and R^{A2} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each occurrence of RAI and R^{A2} is independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, each occurrence of RAI and R^{A2} is hydrogen.

In certain embodiments, two geminal RAI or two geminal R^{A2} together form oxo.

In certain embodiments, two geminal R^{A1} or two geminal R^{A2}, together with the carbon atom to which they are attached, form C₃₋₆ carbocycle (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), or cyclohexadienyl(C₆)) or 3- to 6-membered heterocyclyl (e.g., heterocyclyl comprising one 3- to 6-membered ring and 1-3 heteroatoms selected from N, O, and S), wherein the carbocycle or heterocycle is optionally substituted with one or more R^{u}.

In certain embodiments, each occurrence of R^{A1}′ and R^{A2}′ is independently hydrogen, C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)), C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)), C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)), C₃₋₁₂ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₇), cycloheptenyl(C₇), cycloheptadienyl(C₇), cycloheptatrienyl(C₇), cyclooctyl(C₈), cyclooctenyl(C₅), bicyclo[2.2.1]heptanyl(C₇), bicyclo[2.2.2]octanyl(C₈), cyclononyl(C₉), cyclononenyl(C₉), cyclodecyl(C₁₀), cyclodecenyl(C₁₀), octahydro-1H-indenyl(C₉), decahydronaphthalenyl(C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, each occurrence of R^{A1}′ and R^{A2}′ is independently hydrogen, C₁₋₆ alkyl, C₂₋₆ alkenyl, C₂₋₆ alkynyl, C₃₋₆ carbocyclyl, 3- to 6-membered heterocyclyl, C₆ aryl, 5- to 6-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, each occurrence of R^{A1}′ and R^{A2}′ is independently hydrogen, C₁₋₆ alkyl, C₂₋₆ alkenyl, C₂₋₆ alkynyl, C₃₋₆ carbocyclyl, or 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, or heterocyclyl is optionally substituted with one or more R^{u}.

In certain embodiments, each occurrence of R^{A1}′ and R^{A2}′ is independently hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}.

In certain embodiments, each occurrence of R^{A1}′ and R^{A2}′ is independently hydrogen or C₁₋₆ alkyl. In certain embodiments, each occurrence of R^{A1}′ and R^{A2}′ is hydrogen.

In certain embodiments, a′ is 0. In certain embodiments, a′ is 1. In certain embodiments, a′ is 2. In certain embodiments, a′ is 3.

In certain embodiments, a″ is 0. In certain embodiments, a″ is 1. In certain embodiments, a″ is 2. In certain embodiments, a″ is 3.

In certain embodiments, one of a′ and a″ is 0. In certain embodiments, a′ and a″ are not both 0.

In certain embodiments, Ring A¹ is heterocyclyl.

In certain embodiments, each R^{A} is independently oxo, halogen (e.g., —F, —Cl, —Br, or —I), —CN, —NO₂, —OH, —NH₂, C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, S-butylhexylamino, t-butylhexylamino, or pentylhexylamino), C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)), C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)), C₃₋₁₂ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₇), cycloheptenyl(C₇), cycloheptadienyl(C₇), cycloheptatrienyl(C₇), cyclooctyl(C₈), cyclooctenyl(C₅), bicyclo[2.2.1]heptanyl(C₇), bicyclo[2.2.2]octanyl(C₈), cyclononyl(C₉), cyclononenyl(C₉), cyclodecyl(C₁₀), cyclodecenyl(C₁₀), octahydro-1H-indenyl(C₉), decahydronaphthalenyl(C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{c}S(═O)R^{a}, —NR^{C}S(═O)₂OR^{b}, —NR^{C}S(═O)₂NR^{c}R^{d}, —NR^{b}C(═O)NR^{c}R^{d}, —NR^{o}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each R^{A} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, a is 0. In certain embodiments, a is 1. In certain embodiments, a is 2. In certain embodiments, a is 3. In certain embodiments, a is 4, as valency permits. In certain embodiments, a is 5, as valency permits. In certain embodiments, a is 6, as valency permits. In certain embodiments, a is 7, as valency permits. In certain embodiments, a is 0. In certain embodiments, a is 8, as valency permits.

In certain embodiments, RA may be present on either Ring A¹ or Ring A″.

In certain embodiments,
Ring A is

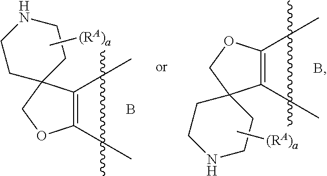

or
Ring A attached to -L-T is

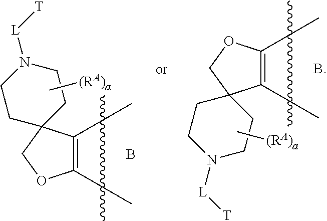

In certain embodiments, the compound or conjugate of Formula II is
1) a compound of Formula II-1-a-i, II-1-a-ii, II-1-a-iii, II-1-a-iv, II-1-a-v, II-1-a-vi, II-1-a-vii, II-1-a-viii, II-1-a-ix, II-1-a-x, II-1-a-xi, or II-1-a-xii:

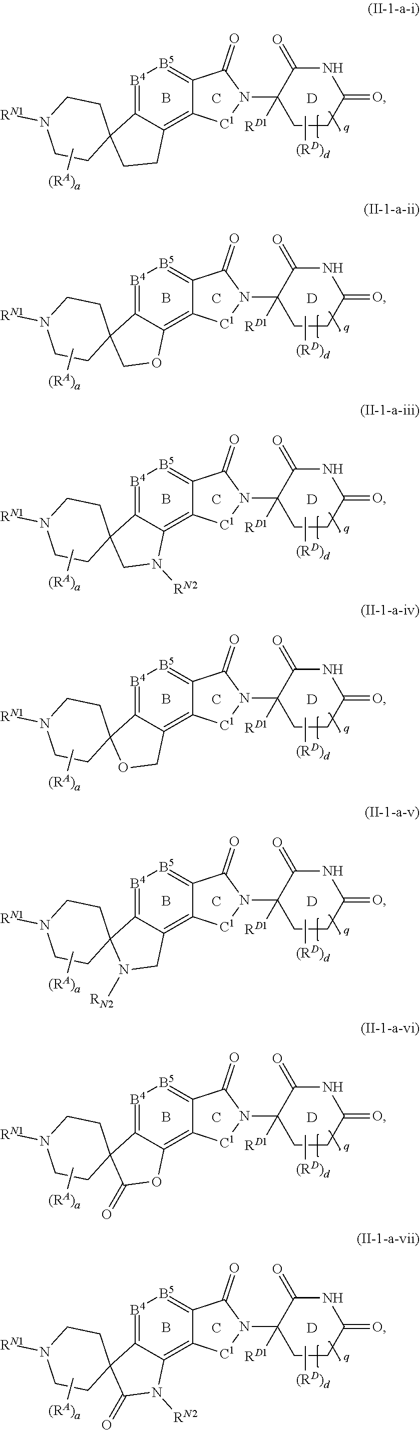

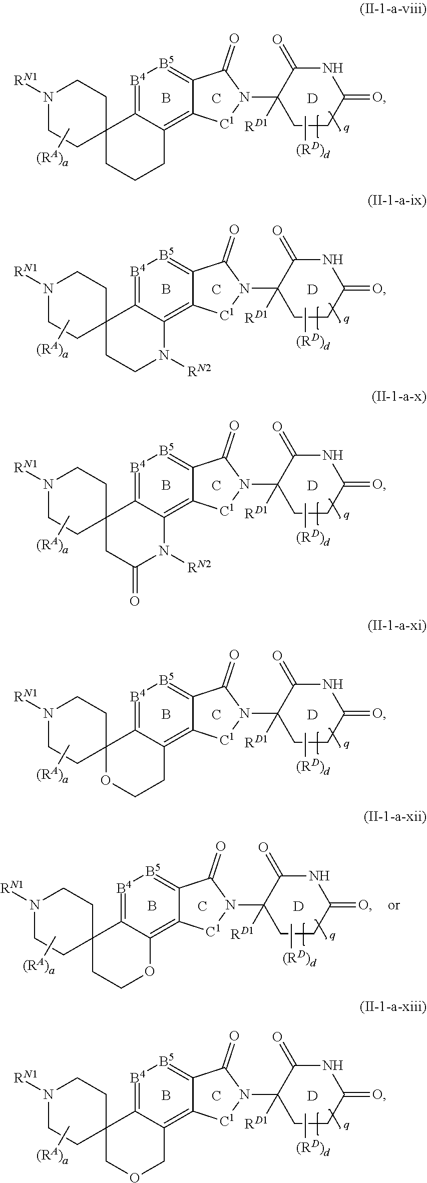

or a pharmaceutically acceptable salt, solvate, or stereoisomer thereof, or
2) a conjugate of Formula II′-1-a-i, II′-1-a-ii, II′-1-a-iii, II′-1-a-iv, II′-1-a-v, II′-1-a-vi, II′-1-a-vii, II′-1-a-viii, II′-1-a-ix, II′-1-a-x, II′-1-a-xi, or II′-1-a-xii

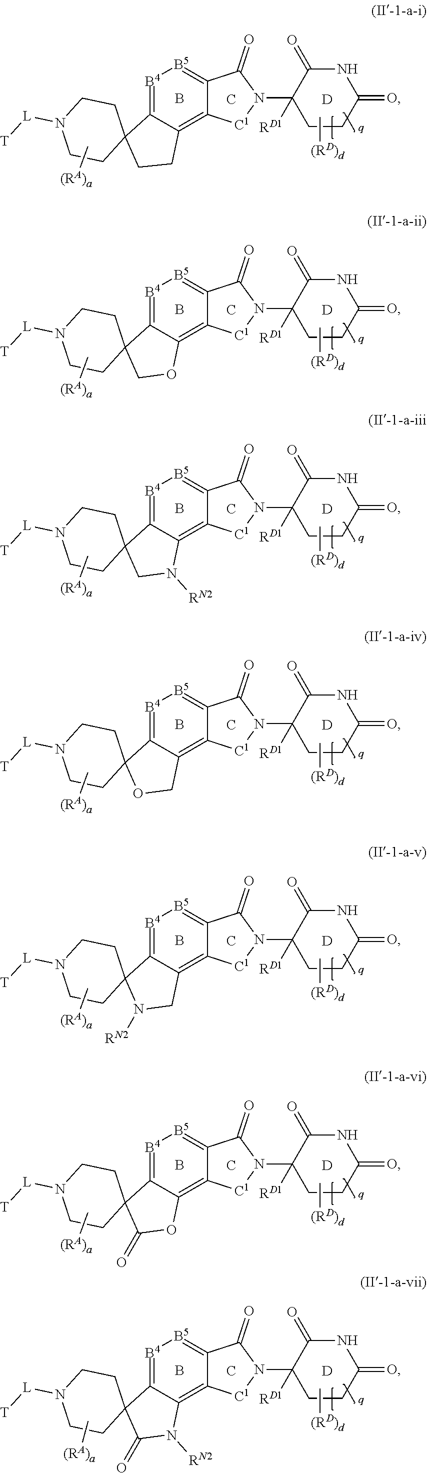

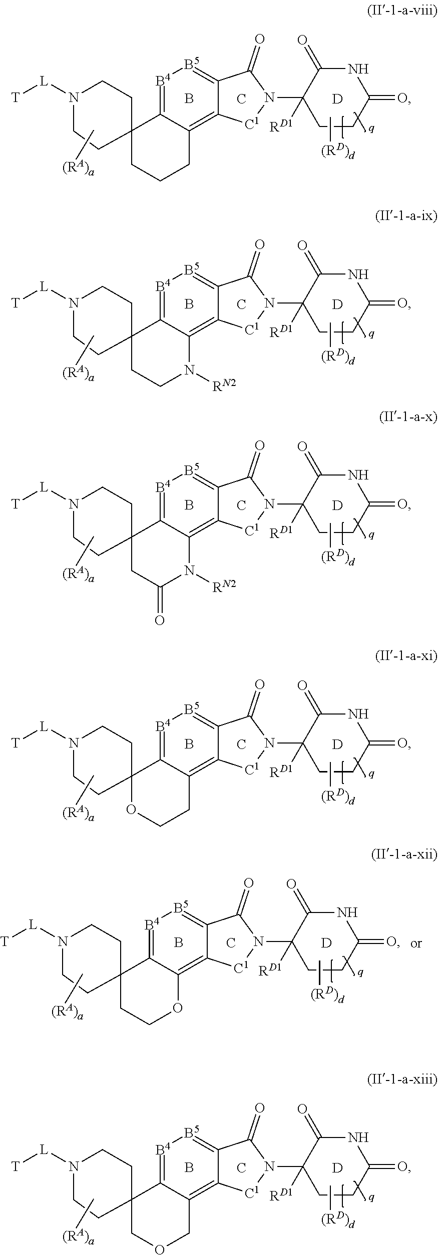

or a pharmaceutically acceptable salt, solvate, or stereoisomer thereof,

wherein:
R^{N1} and R^{N2} are independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}; or
R^{N1} and R^{N2} are independently an amino-protecting group.

In certain embodiments, the compound or conjugate of Formula II is
1) a compound of Formula II-2-a-i, II-2-a-ii, II-2-a-iii, II-2-a-iv, II-2-a-v, II-2-a-vi, II-2-a-vii, II-2-a-viii, II-2-a-ix, II-2-a-x, II-2-a-xi, II-2-a-xii, or II-2-a-xiii:

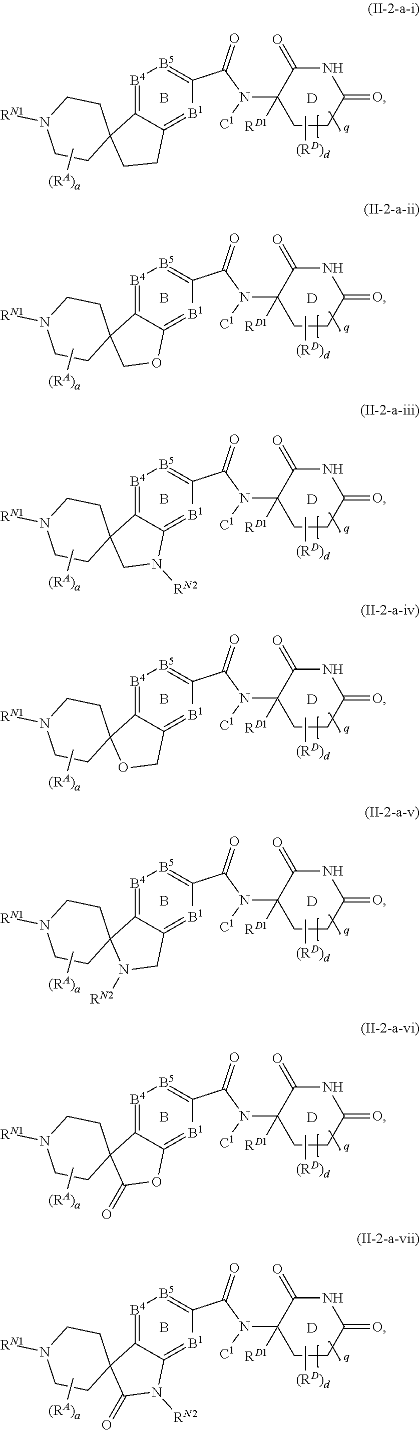

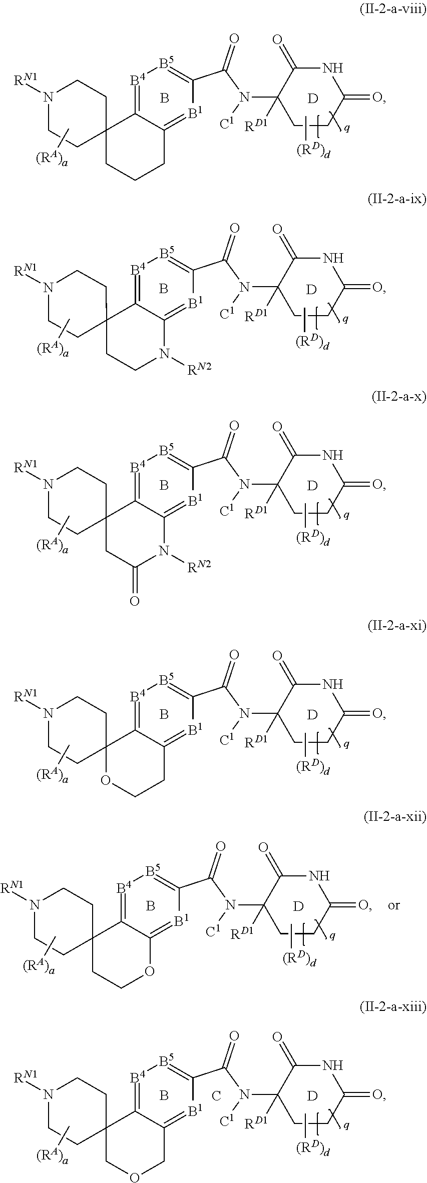

or a pharmaceutically acceptable salt, solvate, or stereoisomer thereof, or
1) a conjugate of Formula II′-2-a-i, II′-2-a-ii, II′-2-a-iii, II′-2-a-iv, II′-2-a-v, II′-2-a-vi, II′-2-a-vii, II′-2-a-viii, II′-2-a-ix, II′-2-a-x, II′-2-a-xi, II′-2-a-xii, or II′-2-a-xiii:

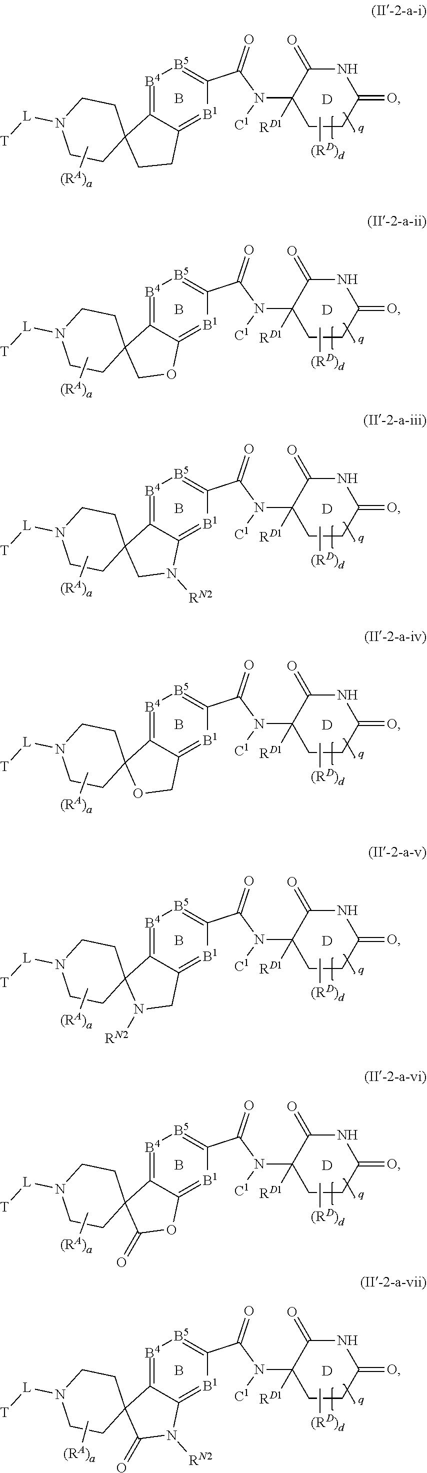

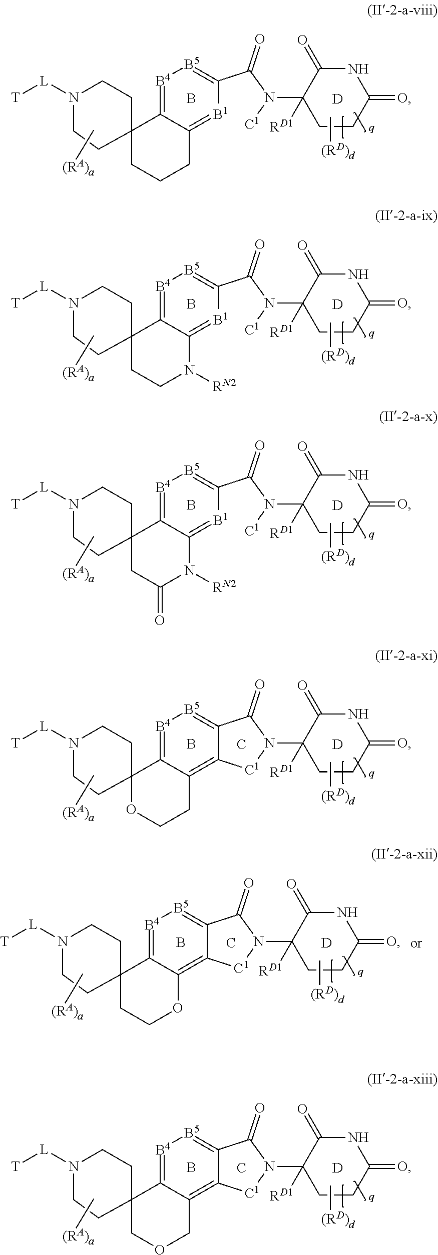

or a pharmaceutically acceptable salt, solvate, or stereoisomer thereof,

wherein:
R^{N1} and R^{N2} are independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}; or
R^{N1} and R^{N2} are independently an amino-protecting group.

In certain embodiments, the compound or conjugate of Formula II is
1) a compound of Formula II-1-b-i, II-1-b-ii, II-1-b-iii, II-1-b-iv, II-1-b-v, II-1-b-vi, II-1-b-vii, II-1-b-viii, II-1-b-ix, II-1-b-x, II-1-b-xi, II-1-b-xii, or II-1-b-xiii:

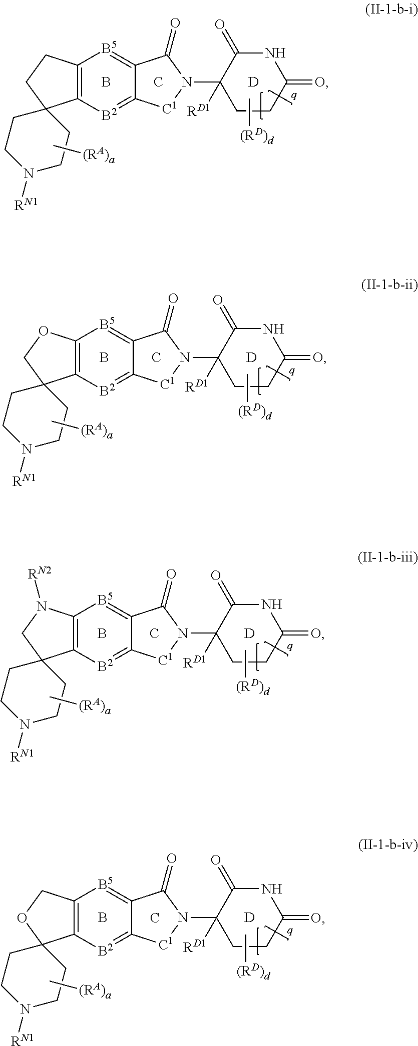

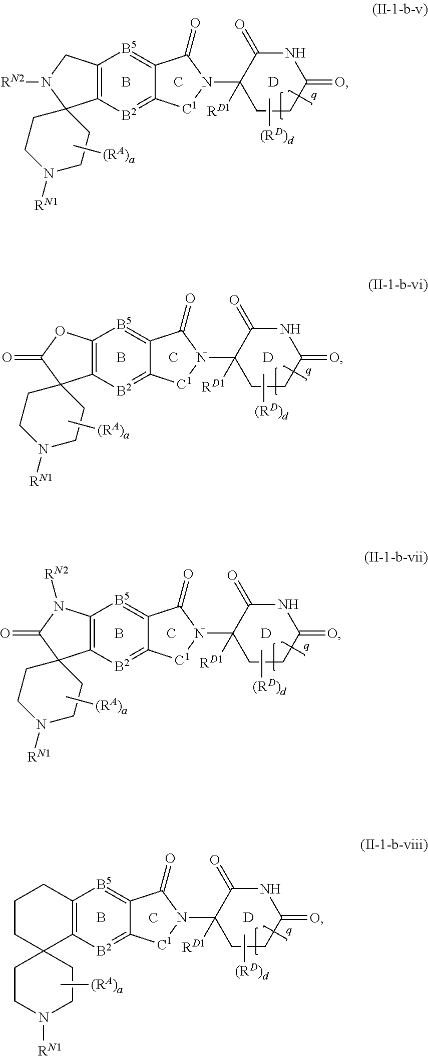

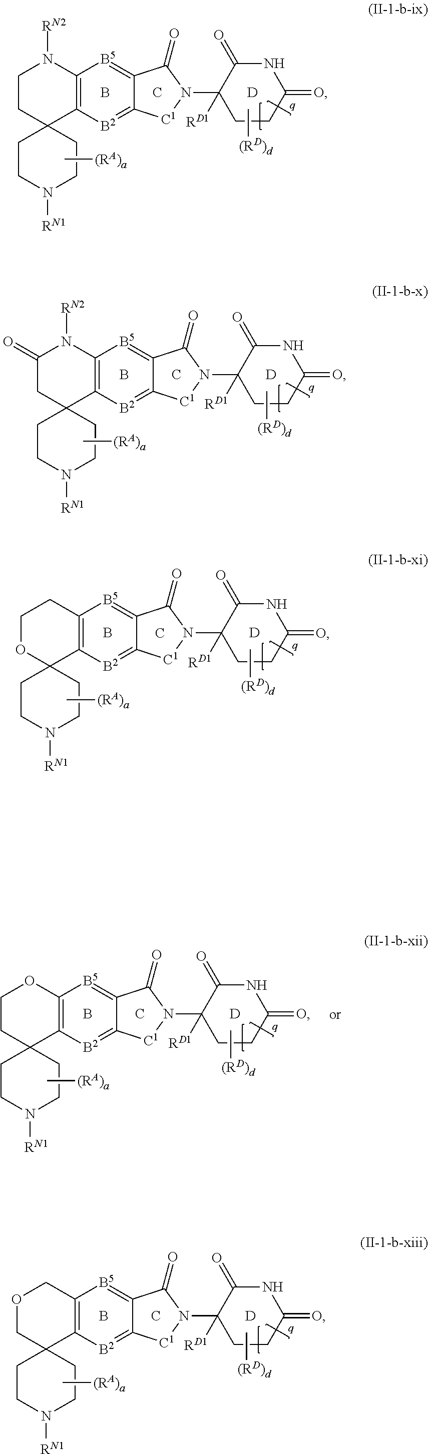

or a pharmaceutically acceptable salt, solvate, or stereoisomer thereof, or
2) a conjugate of Formula II′-1-b-i, II′-1-b-ii, II′-1-b-iii, II′-1-b-iv, II′-1-b-v, II′-1-b-vi, II′-1-b-vii, II′-1-b-viii, II′-1-b-ix, II′-1-b-x, II′-1-b-xi, II′-1-b-xii, or II′-1-b-xiii:

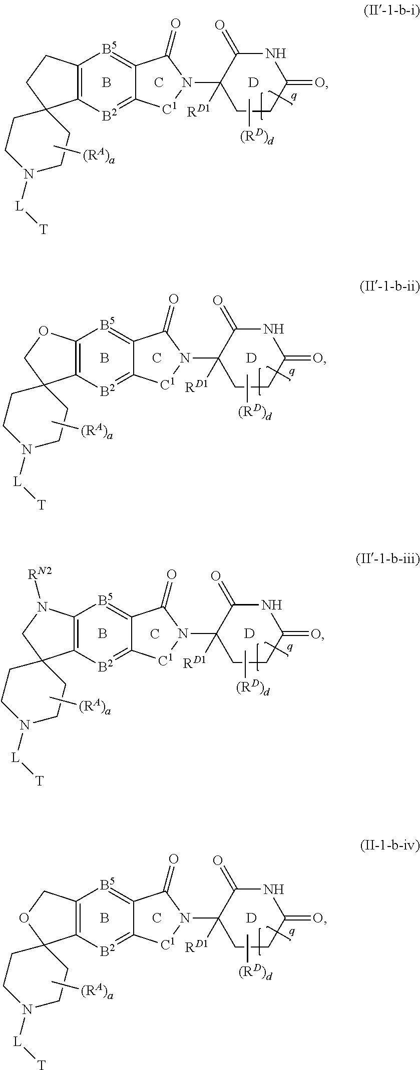

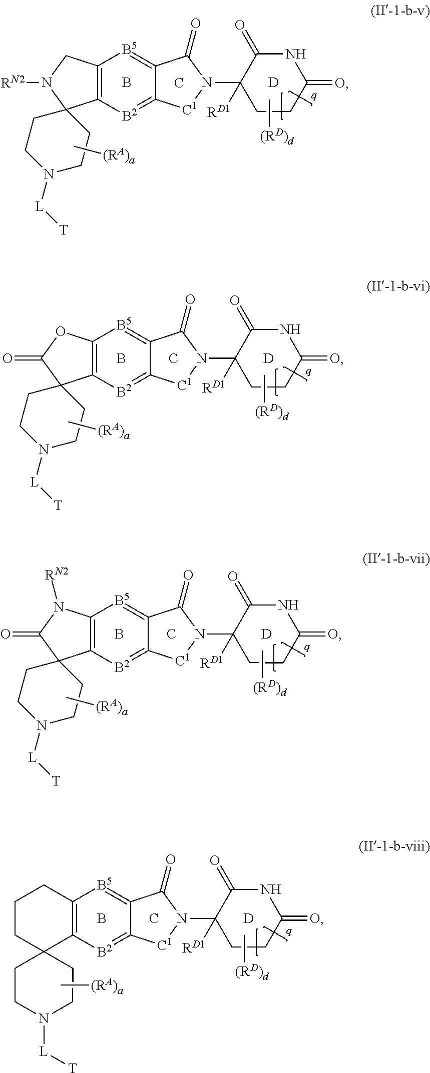

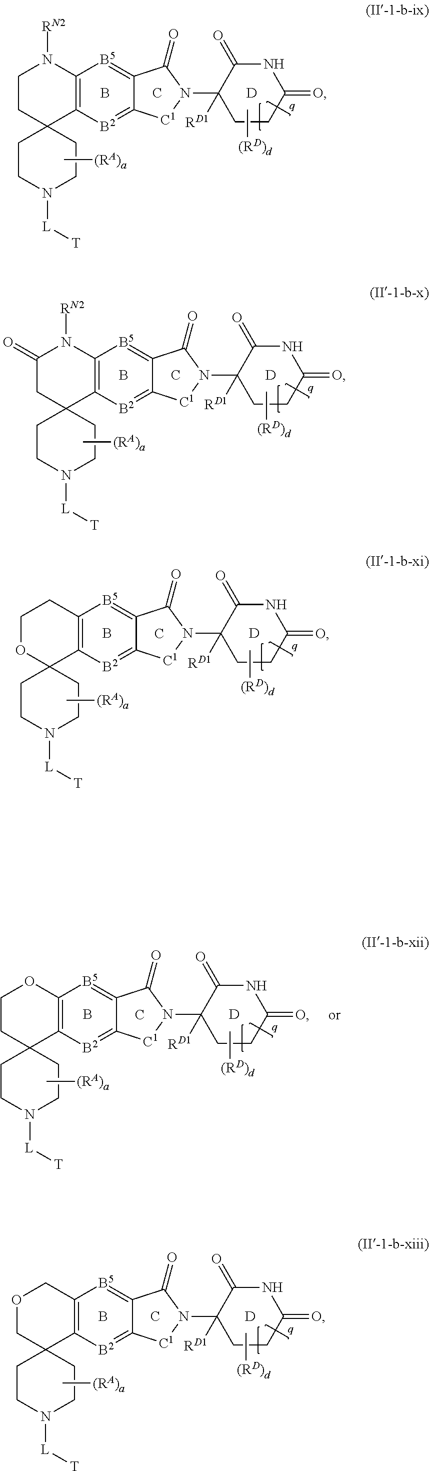

or a pharmaceutically acceptable salt, solvate, or stereoisomer thereof,

wherein:
R^{N1} and R^{N2} are independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}; or
R^{N1} and R^{N2} are independently an amino-protecting group.

In certain embodiments, the compound or conjugate of Formula II is
1) a compound of Formula II-2-b-i, II-2-b-ii, II-2-b-iii, II-2-b-iv, II-2-b-v, II-2-b-vi, II-2-b-vii, II-2-b-viii, II-2-b-ix, II-2-b-x, II-2-b-xi, II-2-b-xii, or II-2-b-xiii:

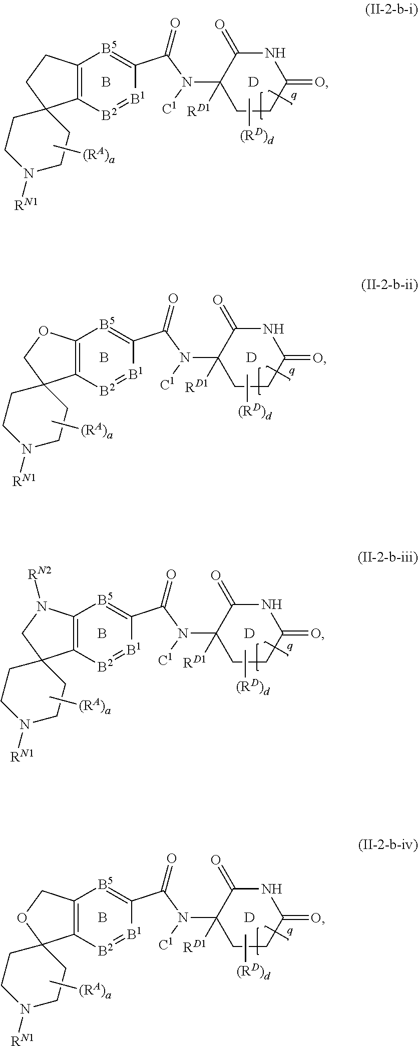

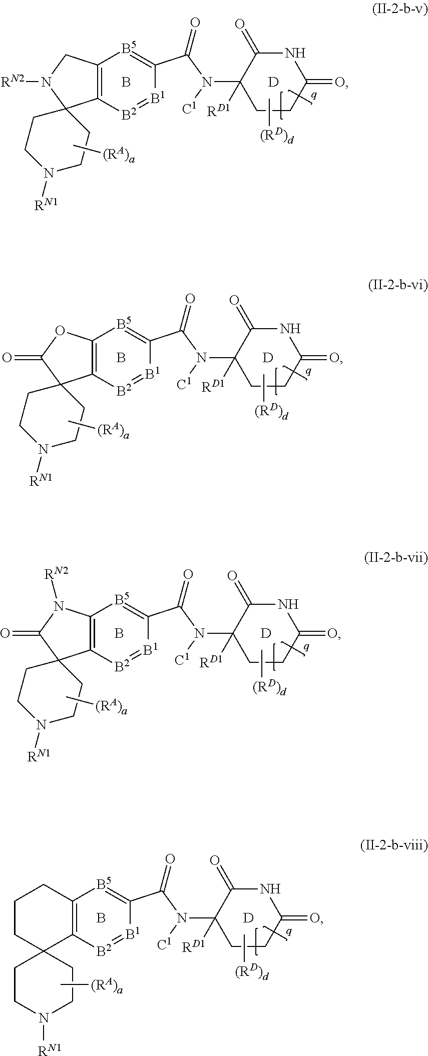

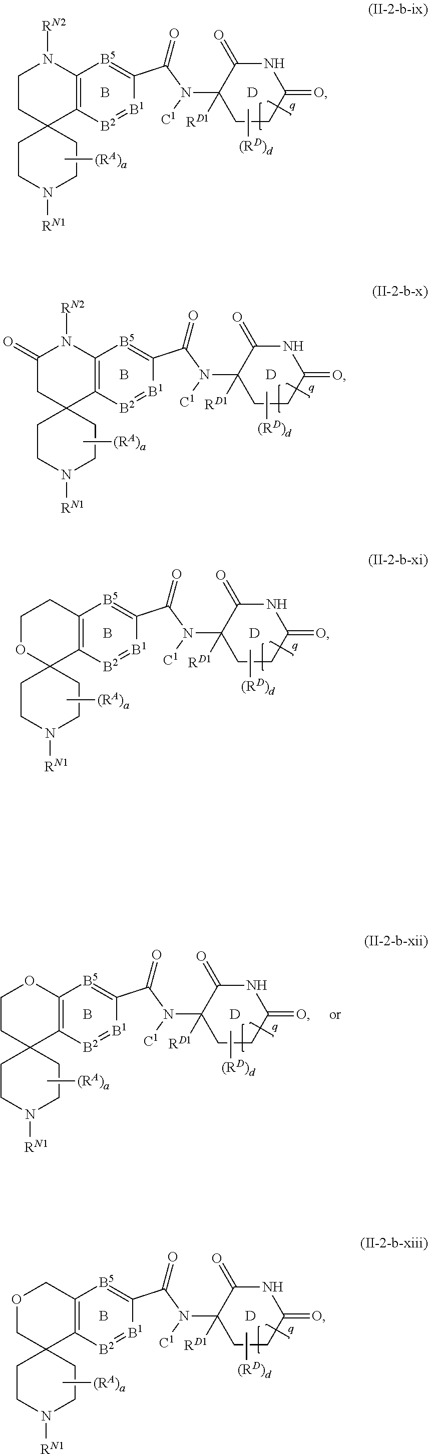

or a pharmaceutically acceptable salt, solvate, or stereoisomer thereof, or
2) a conjugate of Formula II′-2-b-i, II′-2-b-ii, II′-2-b-iii, II′-2-b-iv, II′-2-b-v, II′-2-b-vi, II′-2-b-vii, II′-2-b-viii, II′-2-b-ix, II′-2-b-x, II′-2-b-xi, II′-2-b-xii, or II′-2-b-xiii:

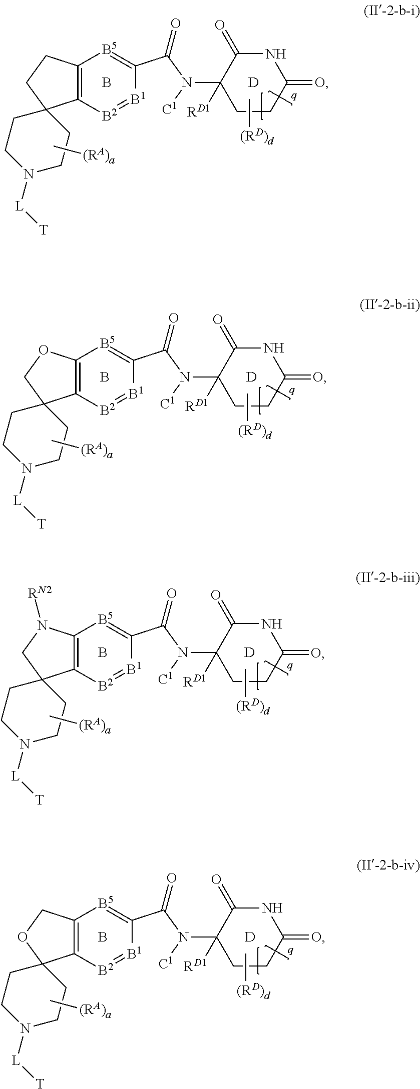

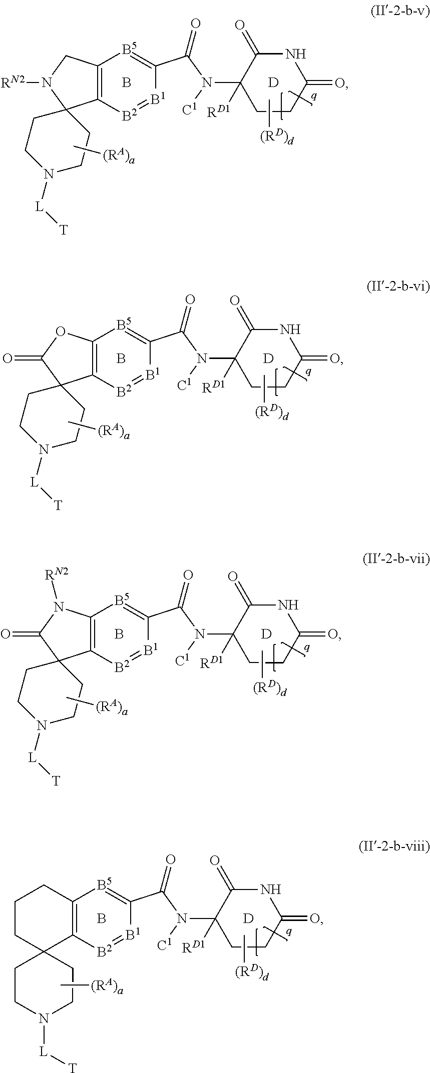

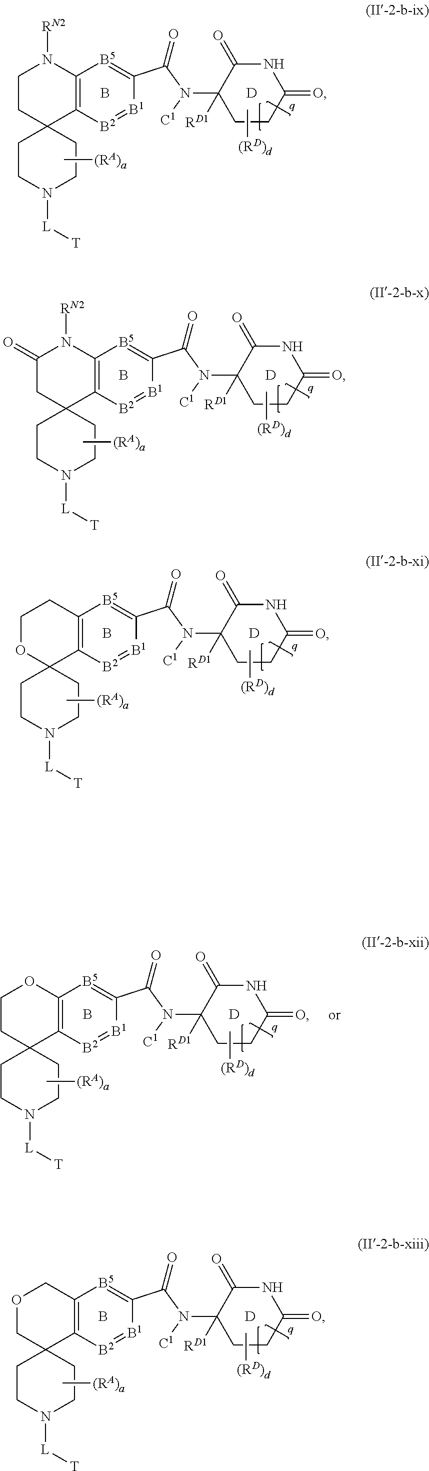

or a pharmaceutically acceptable salt, solvate, or stereoisomer thereof,

wherein:
R^{N1} and R^{N2} are independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}; or
R^{N1} and R^{N2} are independently an amino-protecting group.

In certain embodiments, R^{N1} and R^{N2} are independently hydrogen, C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)), C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)), C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)), C₃₋₁₂ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₇), cycloheptenyl(C₇), cycloheptadienyl(C₇), cycloheptatrienyl(C₇), cyclooctyl(C₈), cyclooctenyl(C₈), bicyclo[2.2.1]heptanyl(C₇), bicyclo[2.2.2]octanyl(C₈), cyclononyl(C₉), cyclononenyl(C₉), cyclodecyl(C₁₀), cyclodecenyl(C₁₀), octahydro-1H-indenyl(C₉), decahydronaphthalenyl(C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, R^{N1} and R^{N2} are independently hydrogen, C₁₋₆ alkyl, C₂₋₆ alkenyl, C₂₋₆ alkynyl, C₃₋₆ carbocyclyl, 3- to 6-membered heterocyclyl, C₆ aryl, 5- to 6-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, R^{N1} and R^{N2} are independently hydrogen, C₁₋₆ alkyl, C₂₋₆ alkenyl, C₂₋₆ alkynyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, or heterocyclyl is optionally substituted with one or more R^{u}.

In certain embodiments, R^{N1} and R^{N2} are independently hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}.

In certain embodiments, R^{N1} and R^{N2} are independently an amino-protecting group.

In certain embodiments, the bond between B¹ and C₁ is present. In certain embodiments, the bond between B¹ and C¹ is absent.

In certain embodiments, B¹ is N, C, or CR^{B1}. In certain embodiments, B¹ is N. In certain embodiments, B¹ is C. In certain embodiments, B¹ is CR^{B1}.

In certain embodiments, R^{B1} is hydrogen, halogen (e.g., —F, —Cl, —Br, or —I), —CN, —NO₂, —OH, —NH₂, C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)), C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)), C₃₋₁₂ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₇), cycloheptenyl(C₇), cycloheptadienyl(C₇), cycloheptatrienyl(C₇), cyclooctyl(C₈), cyclooctenyl(C₈), bicyclo[2.2.1]heptanyl(C₇), bicyclo[2.2.2]octanyl(C₈), cyclononyl(C₉), cyclononenyl(C₉), cyclodecyl(C₁₀), cyclodecenyl(C₁₀), octahydro-1H-indenyl(C₉), decahydronaphthalenyl(C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments, R^{B1} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, R^{B1} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, R^{B1} is hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}.

In certain embodiments, R^{B1} is hydrogen or halogen.

In certain embodiments, C¹ is absent. In certain embodiments, C¹ is hydrogen, C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)), C₃₋₆ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), or cyclohexadienyl(C₆)), 3- to 6-membered heterocyclyl (e.g., heterocyclyl comprising one 3- to 6-membered ring and 1-3 heteroatoms selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}.

In certain embodiments, C¹ is —C(R^{C1})₂—, —C(═O)—, —(C═O)—N(R^{C1}′)—*, or —N═C(R^{C1}′)—*

In certain embodiments, R^{C1}′ is H or C₁₋₆ alkyl optionally substituted with one or more R^{u}, and * denotes attachment to Ring B.

In certain embodiments, each R^{C1} is independently hydrogen, halogen (e.g., —F, —Cl, —Br, or —I), —CN, —NO₂, —OH, —NH₂, C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, S-butylhexylamino, t-butylhexylamino, or pentylhexylamino), C₃₋₆ carbocyclyl (e.g., cyclopropyl (C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), or cyclohexadienyl(C₆)) or 3- to 6-membered heterocyclyl (e.g., heterocyclyl comprising one 3- to 6-membered ring and 1-3 heteroatoms selected from N, O, and S), wherein the alkyl, alkoxy, alkylamino, carbocyclyl, or heterocyclyl is optionally substituted with one or more R^{u}.

In certain embodiments, two R^{C1}, together with the carbon atom to which they are attached, form C₃₋₆ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), or cyclohexadienyl(C₆)) or 3- to 6-membered heterocyclyl (e.g., heterocyclyl comprising one 3- to 6-membered ring and 1-3 heteroatoms selected from N, O, and S), wherein the carbocyclyl or heterocyclyl is optionally substituted with one or more R^{u}.

In certain embodiments, C² is N or O. In certain embodiments, C² is N. In certain embodiments, C² is O.

In certain embodiments, when C² is N, C¹ is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}.

In certain embodiments, when C₂ is O, C¹ is absent.

In certain embodiments, r is 0. In certain embodiments, r is 1.

In certain embodiments, R^{D1} is hydrogen, deuterium, or C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)) optionally substituted with one or more R^{u}.

In certain embodiments, q is 0. In certain embodiments, q is 1. In certain embodiments, q is 2.

In certain embodiments, each RD is independently halogen (e.g., —F, —Cl, —Br, or —I), —CN, —NO₂, —OH, —NH₂, C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-1-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)), C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)), C₃₋₁₂ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₇), cycloheptenyl(C₇), cycloheptadienyl(C₇), cycloheptatrienyl(C₇), cyclooctyl(C₈), cyclooctenyl(C₅), bicyclo[2.2.1]heptanyl(C₇), bicyclo[2.2.2]octanyl(C₈), cyclononyl(C₉), cyclononenyl(C₉), cyclodecyl(C₁₀), cyclodecenyl(C₁₀), octahydro-1H-indenyl(C₉), decahydronaphthalenyl(C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkoxy, alkylamino, alkenyl, alkynyl, carbocyclyl, heterocyclyl, aryl, or heteroaryl is optionally substituted with one or more R^{u}.

In certain embodiments, each RD is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more R^{u}.

In certain embodiments, each RD is independently halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl, or heterocyclyl is optionally substituted with one or more R^{u}.

In certain embodiments, each RD is independently halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl, is optionally substituted with one or more R^{u}.

In certain embodiments, d is 0. In certain embodiments, d is 1. In certain embodiments, d is 2. In certain embodiments, d is 3. In certain embodiments, d is 4. In certain embodiments, d is 5.

In certain embodiments, each R^{a} is independently C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)), C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆), C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)), C₃₋₁₂ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₇), cycloheptenyl(C₇), cycloheptadienyl(C₇), cycloheptatrienyl(C₇), cyclooctyl(C₈), cyclooctenyl(C₈), bicyclo[2.2.1]heptanyl(C₇), bicyclo[2.2.2]octanyl(C₈), cyclononyl(C₉), cyclononenyl(C₉), cyclodecyl(C₁₀), cyclodecenyl(C₁₀), octahydro-1H-indenyl(C₉), decahydronaphthalenyl(C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkenyl, alkynyl, carbocyclyl, heterocyclyl, aryl, or heteroaryl is optionally substituted with one or more R^{u}.

In certain embodiments, each R^{a} is independently C₁₋₆ alkyl, C₂₋₆ alkenyl, C₂₋₆ alkynyl, C₃₋₆ carbocyclyl, 3- to 6-membered heterocyclyl, C₆ aryl, or 5- to 6-membered heteroaryl.

In certain embodiments, each R^{a} is independently C₁₋₆ alkyl, C₂₋₆ alkenyl, C₂₋₆ alkynyl, C₃₋₆ carbocyclyl, or 3- to 6-membered heterocyclyl.

In certain embodiments, each R^{a} is independently C₁₋₆ alkyl, C₃₋₆ carbocyclyl, or 3- to 6-membered heterocyclyl, wherein the alkyl, carbocyclyl, or heterocyclyl is optionally substituted with one or more R^{u}.

In certain embodiments, each R^{b} is independently hydrogen, C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)), C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆), C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)), C₃₋₁₂ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₇), cycloheptenyl(C₇), cycloheptadienyl(C₇), cycloheptatrienyl(C₇), cyclooctyl(C₈), cyclooctenyl(C₈), bicyclo[2.2.1]heptanyl(C₇), bicyclo[2.2.2]octanyl(C₈), cyclononyl(C₉), cyclononenyl(C₉), cyclodecyl(C₁₀), cyclodecenyl(C₁₀), octahydro-1H-indenyl(C₉), decahydronaphthalenyl(C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkenyl, alkynyl, carbocyclyl, heterocyclyl, aryl, or heteroaryl is optionally substituted with one or more R^{u}.

In certain embodiments, each R^{b} is independently hydrogen, C₁₋₆ alkyl, C₂₋₆ alkenyl, C₂-6 alkynyl, C₃₋₆ carbocyclyl, 3- to 6-membered heterocyclyl, C₆ aryl, or 5- to 6-membered heteroaryl.

In certain embodiments, each R^{b} is independently hydrogen, C₁₋₆ alkyl, C₂₋₆ alkenyl, C₂-6 alkynyl, C₃₋₆ carbocyclyl, or 3- to 6-membered heterocyclyl.

In certain embodiments, each R^{b} is independently hydrogen, C₁₋₆ alkyl, C₃₋₆ carbocyclyl, or 3- to 6-membered heterocyclyl, or C₂₋₆ alkynyl, wherein the alkyl, carbocyclyl, or heterocyclyl is optionally substituted with one or more R^{u}.

In certain embodiments, each R^{c} and each R^{d} is independently hydrogen, C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)), C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆), C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)), C₃₋₁₂ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₇), cycloheptenyl(C₇), cycloheptadienyl(C₇), cycloheptatrienyl(C₇), cyclooctyl(C₈), cyclooctenyl(C₈), bicyclo[2.2.1]heptanyl(C₇), bicyclo[2.2.2]octanyl(C₈), cyclononyl(C₉), cyclononenyl(C₉), cyclodecyl(C₁₀), cyclodecenyl(C₁₀), octahydro-1H-indenyl(C₉), decahydronaphthalenyl(C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), or 5- to 10-membered heteroaryl (e.g., heteroaryl comprising one or two 5- or 6-membered rings and 1-5 heteroatoms selected from N, O, and S), wherein the alkyl, alkenyl, alkynyl, carbocyclyl, heterocyclyl, aryl, or heteroaryl is optionally substituted with one or more R^{u}.

In certain embodiments, each Re and each R^{d} is independently hydrogen, C₁₋₆ alkyl, C₃-6 carbocyclyl, or 3- to 6-membered heterocyclyl, wherein the alkyl, carbocyclyl, or heterocyclyl is optionally substituted with one or more R^{u}.

In certain embodiments, R^{c} and R^{d}, together with the nitrogen atom to which they are attached, form 3- to 12-membered heterocyclyl (e.g., heterocyclyl comprising one or two 3- to 8-membered rings and 1-5 heteroatoms selected from N, O, and S), wherein the heterocyclyl is optionally substituted with one or more R^{u}.

In certain embodiments, R^{a}, Rb, R^{c}, and R^{d} is independently and optionally substituted with one or more R^{z}.

In certain embodiments, R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

In certain embodiments, each R^{u} is independently oxo, halogen, —CN, —NO₂, —OH, —NH₂, C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)), C₁₋₆ alkoxy (e.g., methoxy (C₁), ethoxy (C₂), propoxy (C₃), i-propoxy (C₃), n-butoxy (C₄), i-butoxy (C₄), s-butoxy (C₄), t-butoxy (C₄), pentoxy (C₅), or hexoxy (C₆)), C₁₋₆ alkylamino (e.g., dimethylamino, diethylamino, di-n-propylamino, di-i-propylamino, di-n-butylamino, di-i-butylamino, di-s-butylamino, di-t-butylamino, dipentylamino, dihexylamino, methylethylamino, methyl-n-propylamino, methyl-i-propylamino, methyl-n-butylamino, methyl-1-butylamino, methyl-s-butylamino, methyl-t-butylamino, methylpentylamino, methylhexylamino, ethyl-n-propylamino, ethyl-1-propylamino, ethyl-n-butylamino, ethyl-s-butylamino, ethyl-1-butylamino, ethyl-t-butylamino, ethylpentylamino, ethylhexylamino, propyl-n-butylamino, propyl-1-butylamino, propyl-s-butylamino, propyl-t-butylamino, propylpentylylamino, propylhexylamino, n-butylpentylamino, i-butylpentylamino, s-butylpentylamino, t-butylpentylamino, n-butylhexylamino, i-butylhexylamino, s-butylhexylamino, t-butylhexylamino, or pentylhexylamino), C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)), C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)), C₃₋₁₂ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₇), cycloheptenyl(C₇), cycloheptadienyl(C₇), cycloheptatrienyl(C₇), cyclooctyl(C₈), cyclooctenyl(C₈), bicyclo[2.2.1]heptanyl(C₇), bicyclo[2.2.2]octanyl(C₈), cyclononyl(C₉), cyclononenyl(C₉), cyclodecyl(C₁₀), cyclodecenyl(C₁₀), octahydro-1H-indenyl(C₉), decahydronaphthalenyl(C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{C}S(═O)R^{a}, —NR^{C}S(═O)₂OR^{b}, —NR^{C}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, 3- to 6-membered heterocyclyl, C₆ aryl, or 5- to 6-membered heteroaryl, wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl, wherein the alkyl, alkoxy, alkylamino, alkenyl, alkynyl, carbocyclyl or heterocyclyl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, each R^{u} is independently oxo, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl or heterocyclyl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, two R^{u}, together with the carbon atom(s) to which they are attached, form C₃₋₆ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), or cyclohexadienyl(C₆)) or 3- to 6-membered heterocyclyl (e.g., heterocyclyl comprising one 3- to 6-membered ring and 1-3 heteroatoms selected from N, O, and S).

In certain embodiments, two geminal R^{u}, together with the carbon atom to which they are attached, form C₃₋₆ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), or cyclohexadienyl(C₆)) or 3- to 6-membered heterocyclyl (e.g., heterocyclyl comprising one 3- to 6-membered ring and 1-3 heteroatoms selected from N, O, and S).

In certain aspects, the present disclosure provides compounds of Formula I:

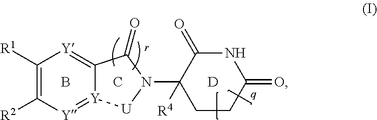

and pharmaceutically acceptable salts, solvates, or stereoisomers thereof,

wherein:
R¹ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{C}S(═O)R^{a}, —NR^{C}S(═O)₂OR^{b}, —NR^{C}S(═O)₂NR^{c}R^{d}, —NR^{b}C(═O)NR^{c}R^{d}, —NR^{o}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; or
R¹ and R², together with the intervening carbon atoms, form optionally substituted 7- to 16-membered spiro heterocycle;
Y″ is N or CR³;
R³ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{C}S(═O)R^{a}, —NR^{C}S(═O)₂OR^{b}, —NR^{C}S(═O)₂NR^{c}R^{d}, —NR^{b}C(═O)NR^{c}R^{d}, —NR^{o}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; or
R² and R³, together with the intervening carbon atoms, form optionally substituted 7- to 16-membered spiro heterocycle;
provided that either R¹ and R², or R^{z} and R³ form optionally substituted 7- to 16-membered spiro heterocycle;
Y′ is N or CRY;
R^{Y}′ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, 5- to 10-membered heteroaryl, carbocyclyl, or heterocyclyl is optionally substituted with one or more R^{u};
---denotes an optional covalent bond between Y and U;
when the bond between Y and U is absent:
r is 0 or 1;
Y is N or CRY;
R^{Y} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u};
U is hydrogen or C₁₋₆ alkyl optionally substituted with one or more R^{u};
when the bond between Y and U is present:
r is 1;
Y is C;
U is —CH₂—, —C(═O)—, —(C═O)—N(R^{U})—*, —N═C(R^{U})—*;
R^{U} is H or C₁₋₆ alkyl optionally substituted with one or more R^{u}, and * denotes attachment to Ring B;
R⁴ is hydrogen, deuterium, C₁₋₆ haloalkyl, or C₁₋₆ alkyl; and
q is an integer from 0 to 2,

wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{C}S(═O)R^{a}, —NR^{C}S(═O)₂OR^{b}, —NR^{C}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl; or two R^{u}, together with the one or more intervening atoms, form C₆₋₁₀ aryl, 5- to 10-membered heteroaryl, C₃₋₁₂ carbocyclyl or 3- to 12-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each Re and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl,
wherein each occurrence of R^{a}, Rb, R^{c}, and R^{d} is independently and optionally substituted with one or more R²; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

In certain embodiments, the compound of Formula I is a compound of Formula I-1

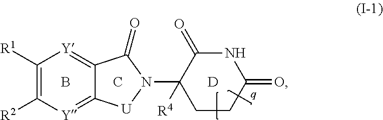

or a pharmaceutically acceptable salt, solvate, or stereoisomer thereof.

In certain embodiments, U is —CH₂- or —C(═O)—.

In certain embodiments, the compound of Formula I is a compound of Formula I-2

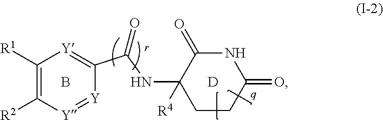

or a pharmaceutically acceptable salt, solvate, or stereoisomer thereof.

In certain embodiments, Y is N.

In certain embodiments, Y is CRY.

In certain embodiments, R^{Y} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, R^{Y} is hydrogen, halogen, C₁₋₆ alkoxy.

In certain embodiments, R¹ and R², together with the intervening carbon atoms, form optionally substituted 7- to 16-membered spiro heterocycle.

In certain embodiments, the 7- to 16-membered spiro heterocycle is optionally substituted with one or more R^{u}. In certain embodiments, the 7- to 16-membered spiro heterocycle is optionally substituted with one or more Rⁱ. In certain embodiments, the 7- to 16-membered spiro heterocycle is optionally substituted with one or more R^{X1}. In certain embodiments, the 7- to 16-membered spiro heterocycle is optionally substituted with one or more R^{Z1}. In certain embodiments, the 7- to 16-membered spiro heterocycle is optionally substituted with one or more R^{X2}. In certain embodiments, the 7- to 16-membered spiro heterocycle is optionally substituted with one or more R^{Z2}.

In certain embodiments, R^{u} is Rⁱ. In certain embodiments, R^{u} is R^{X1}. In certain embodiments, R^{u} is R^{X2}. In certain embodiments, R^{u} is R^{Z1}. In certain embodiments, R^{u} is R^{Z2}. In certain embodiments, Rⁱ is R^{X1}. In certain embodiments, Rⁱ is R^{X2}. In certain embodiments, Rⁱ is R^{Z1}. In certain embodiments, Rⁱ is R^{Z2}.

In certain embodiments, the 7- to 16-membered spiro heterocycle is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{a}, —NR^{C}S(═O)₂R^{a}, —NR^{C}S(═O)R^{a}, —NR^{C}S(═O)₂OR^{b}, NR^{C}S(═O)₂NR^{c}R^{d}, —NR^{o}C(═O)NR^{c}R^{a}, —NR^{o}C(═O)R^{a}, —NR^{o}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, the 7- to 16-membered spiro heterocycle is

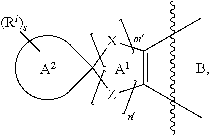

wherein:
Ring A² is C₃₋₁₂ carbocycle or 3- to 12-membered heterocycle;
each X is independently —C(R^{X1})₂—, —NR^{X2}—, —O—, —S—, —S(═O)—, or —S(═O)₂—;
each Z is independently —C(R^{Z1})₂—, —NR^{z2}—, —O—, —S—, —S(═O)—, or —S(═O)₂—;
each occurrence of R^{X1} and R^{Z1} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{C}S(═O)R^{a}, —NR^{C}S(═O)₂OR^{b}, —NR^{C}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
two geminal R^{X1} or two geminal R^{Z1} together form oxo; or
two R^{X1} or two R^{Z1}, together with the intervening carbon atom(s), form C₃₋₁₂ carbocyclyl or 3- to 12-membered heterocyclyl, wherein the carbocyclyl or heterocyclyl is optionally substituted with one or more R^{u};
each occurrence of R^{X2} and R⁷² is independently hydrogen or C₁₋₆ alkyl optionally substituted with one or more R^{u};
m′ and n′ are independently an integer selected from 0 to 3;
provided that either m′ or n′ is 0;
each Rⁱ independently is oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{C}S(═O)R^{a}, —NR^{C}S(═O)₂OR^{b}, —NR^{C}S(═O)₂NR^{c}R^{d}, —NR^{b}C(═O)NR^{c}R^{d}, —NR^{o}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
s is an integer selected from 0 to 10.

In certain embodiments, the 7- to 16-membered spiro heterocycle is

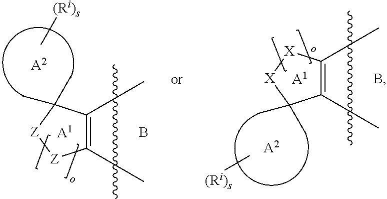

wherein o is an integer selected from 0 to 2.

In certain embodiments, Ring A¹ is 4- to 6-membered heterocycle.

In certain embodiments, X is —C(R^{X1})₂—, —NR^{X2}—, or —O—, and Z is —C(R^{Z1})₂—, —NR^{Z2}—, or —O—.

In certain embodiments, the compound of Formula I-1 is a compound of Formula I-1-a-i, I-1-a-ii, I-1-a-iii, I-1-a-iv, I-1-a-v, I-1-a-vi, I-1-a-vii, I-1-a-viii, I-1-a-ix, I-1-a-x, I-1-a-xi, I-1-a-xii, or I-1-a-xiii:

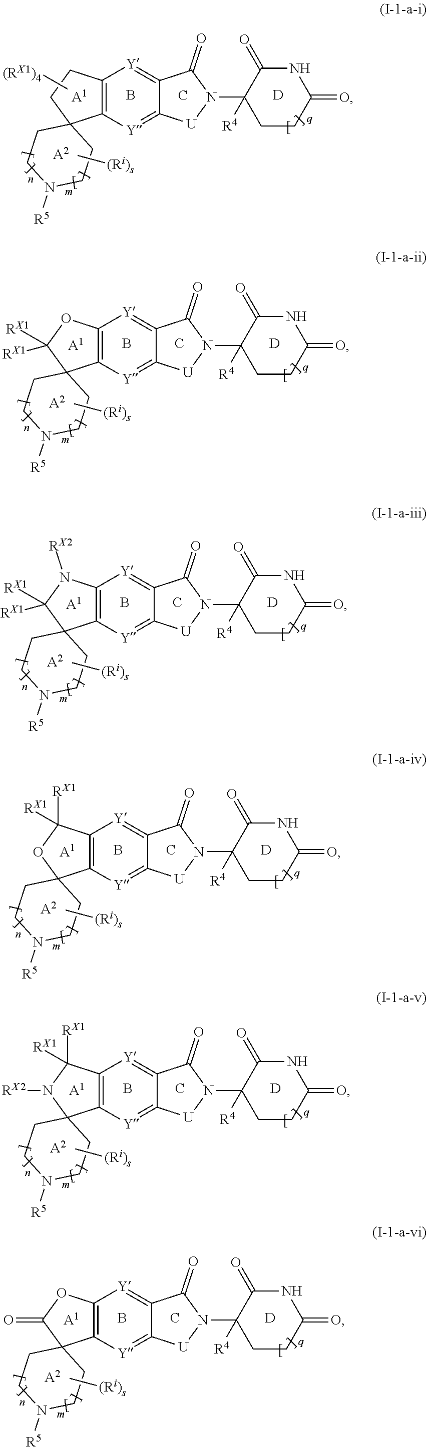

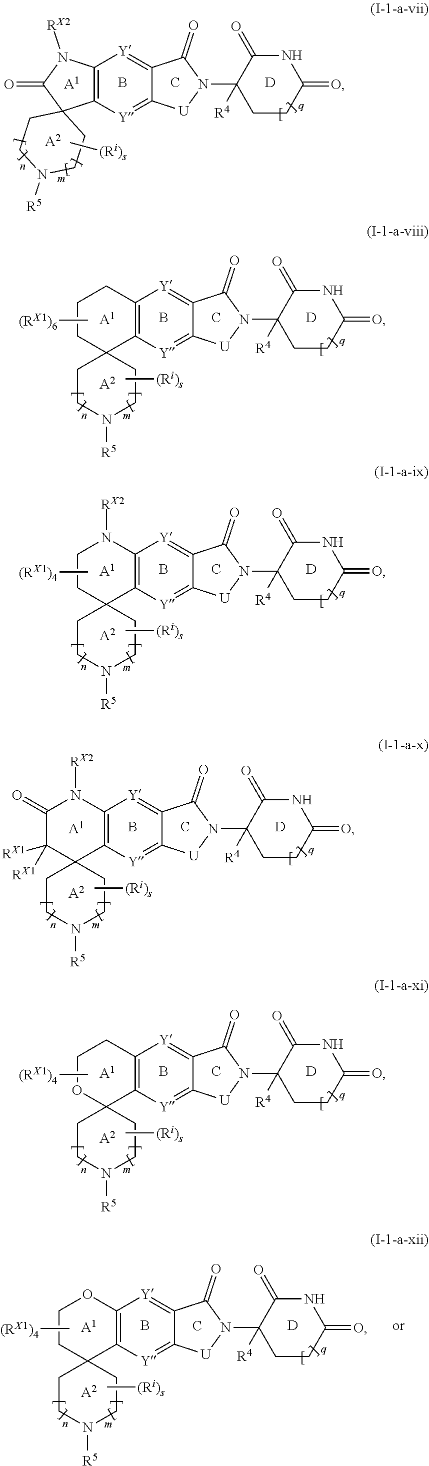

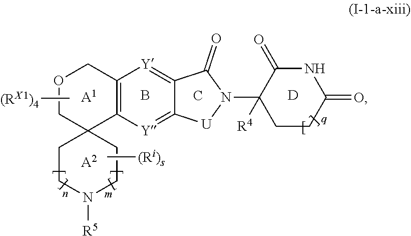

or a pharmaceutically acceptable salt, solvate, or stereoisomer thereof, wherein
R⁵ is hydrogen, C₁₋₆ alkyl, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}; or
R⁵ is an amino-protecting group; and
m and n are independently an integer selected from 0 to 2.

In certain embodiments, the compound of Formula I-2 is a compound of Formula I-2-a-i, I-2-a-ii, I-2-a-iii, I-2-a-iv, I-2-a-v, I-2-a-vi, I-2-a-vii, I-2-a-viii, I-2-a-ix, I-2-a-x, I-2-a-xi, I-2-a-xii, or I-2-a-xiii:

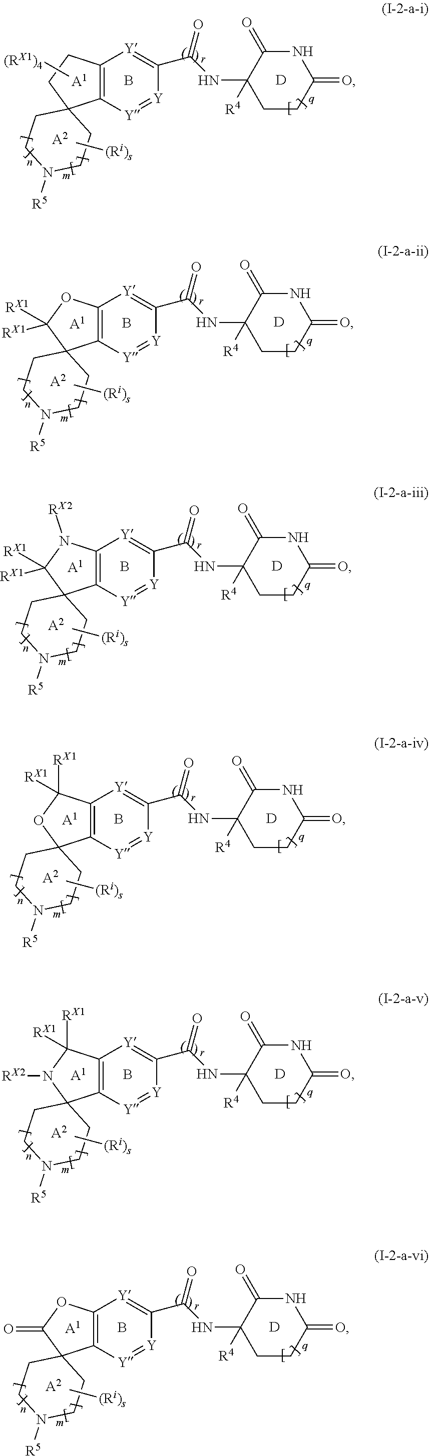

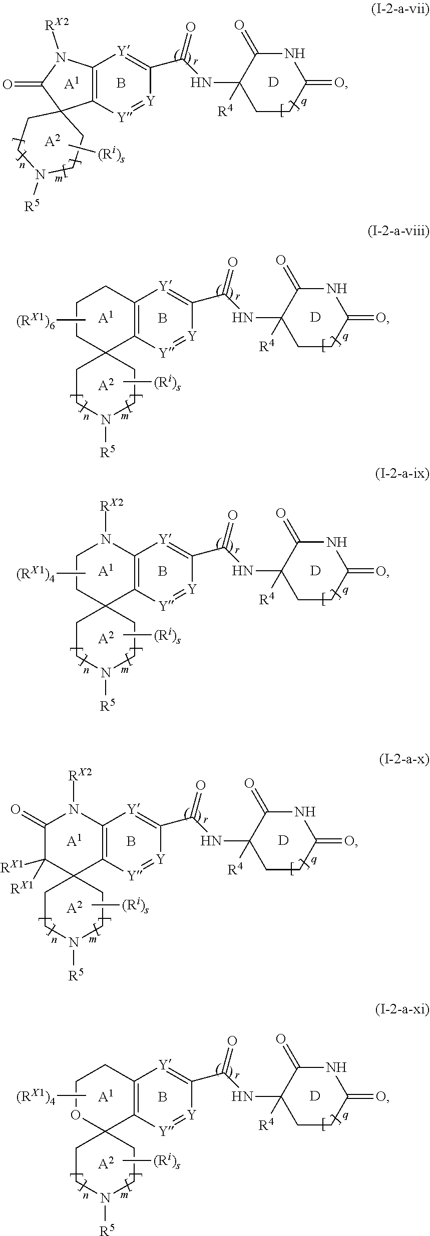

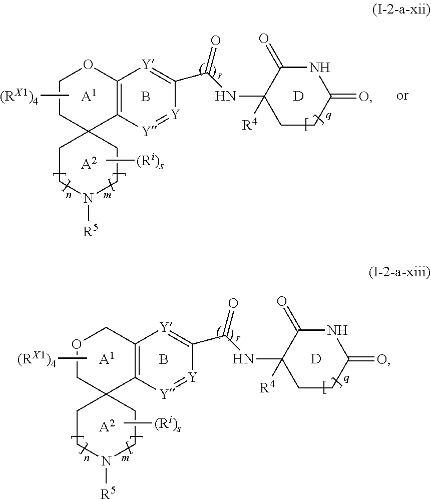

or a pharmaceutically acceptable salt, solvate, or stereoisomer thereof, wherein
R⁵ is hydrogen, C₁₋₆ alkyl, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}; or
R⁵ is an amino-protecting group; and
m and n are independently an integer selected from 0 to 2.

In certain embodiments, each R⁵ is independently hydrogen, C₁₋₆ alkyl, C₃₋₆ carbocyclyl, 3- to 6-membered heterocyclyl, —S(═O)₂R^{a}, or —C(═O)R^{a}, wherein the alkyl, carbocyclyl, or heterocyclyl is optionally substituted with one or more R^{u}.

In certain embodiments, each R⁵ is independently hydrogen or C₁₋₆ alkyl optionally substituted with one or more R^{u}.

In certain embodiments, Y″ is N.

In certain embodiments, Y″ is CR³.

In certain embodiments, R³ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, R³ is hydrogen.

In certain embodiments, R² and R³, together with the intervening carbon atoms, form optionally substituted 7- to 16-membered spiro heterocycle.

In certain embodiments, the 7- to 16-membered spiro heterocycle is optionally substituted with one or more R^{u}. In certain embodiments, the 7- to 16-membered spiro heterocycle is optionally substituted with one or more Rⁱ. In certain embodiments, the 7- to 16-membered spiro heterocycle is optionally substituted with one or more R^{X1}. In certain embodiments, the 7- to 16-membered spiro heterocycle is optionally substituted with one or more R^{Z1}. In certain embodiments, the 7- to 16-membered spiro heterocycle is optionally substituted with one or more R^{X2}. In certain embodiments, the 7- to 16-membered spiro heterocycle is optionally substituted with one or more R^{Z2}.

In certain embodiments, R^{u} is Rⁱ. In certain embodiments, R^{u} is R^{X1}. In certain embodiments, R^{u} is R^{X2}. In certain embodiments, R^{u} is R^{Z1}. In certain embodiments, R^{u} is R^{Z2}. In certain embodiments, Rⁱ is R^{X1}. In certain embodiments, Rⁱ is R^{X2}. In certain embodiments, Rⁱ is R^{Z1}. In certain embodiments, Rⁱ is R^{Z2}.

In certain embodiments, the 7- to 16-membered spiro heterocycle is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{a}, —NR^{C}S(═O)₂R^{a}, —NR^{C}S(═O)R^{a}, —NR^{C}S(═O)₂OR^{b}, —NR^{C}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, the 7- to 16-membered spiro heterocycle is

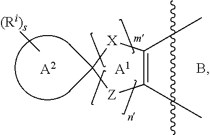

wherein:
Ring A² is C₃₋₁₂ carbocycle or 3- to 12-membered heterocycle;
each X is independently —C(R^{X1})₂—, —NR^{X2}—, —O—, —S—, —S(═O)—, or —S(═O)₂—;
each Z is independently —C(R^{Z1})₂—, —NR^{z2}—, —O—, —S—, —S(═O)—, or —S(═O)₂—;
each occurrence of R^{X1} and R^{Z1} is independently hydrogen, halogen, —CN, —NO₂, —OH, —NH₂, C₁₋₆ alkyl, C₁₋₆ alkoxy, C₁₋₆ alkylamino. C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{C}S(═O)R^{a}, —NR^{C}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
two geminal R^{X1} or two geminal R^{Z1} together form oxo; or
two R^{X1} or two R^{Z1}, together with the intervening carbon atom(s), form C₃₋₁₂ carbocyclyl or 3- to 12-membered heterocyclyl, wherein the carbocyclyl or heterocyclyl is optionally substituted with one or more R^{u};
each occurrence of R^{X2} and R⁷² is independently hydrogen or C₁₋₆ alkyl optionally substituted with one or more R^{u};
m′ and n′ are independently an integer selected from 0 to 3;
provided that either m′ or n′ is 0;
each Rⁱ independently is oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{C}S(═O)R^{a}, —NR^{C}S(═O)₂OR^{b}, —NR^{C}S(═O)₂NR^{c}R^{d}, —NR^{o}C(═O)NR^{c}R^{d}, —NR^{o}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
s is an integer selected from 0 to 10.

In certain embodiments, the 7- to 16-membered spiro heterocycle is

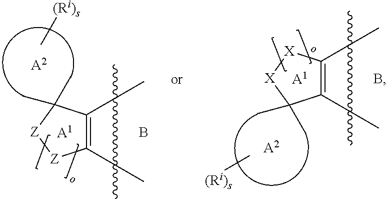

wherein o is an integer selected from 0 to 2.

In certain embodiments, Ring A¹ is 4- to 6-membered heterocycle.

In certain embodiments, X is —C(R^{X1})₂—, —NR^{X2}—, or —O—, and Z is —C(R^{Z1})₂—, —NR^{Z2}, or

In certain embodiments, the compound of Formula I-1 is a compound of Formula I-1-b-i, I-1-b-ii, I-1-b-iii, I-1-b-iv, I-1-b-v, I-1-b-vi, I-1-b-vii, I-1-b-viii, I-1-b-ix, I-1-b-x, I-1-b-xi, I-1-b-xii, or I-1-b-xiii:

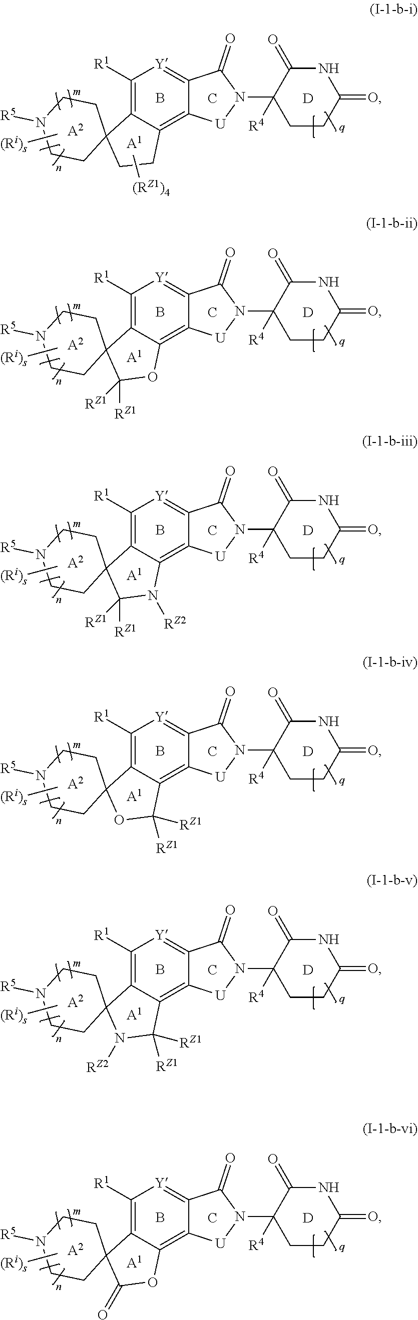

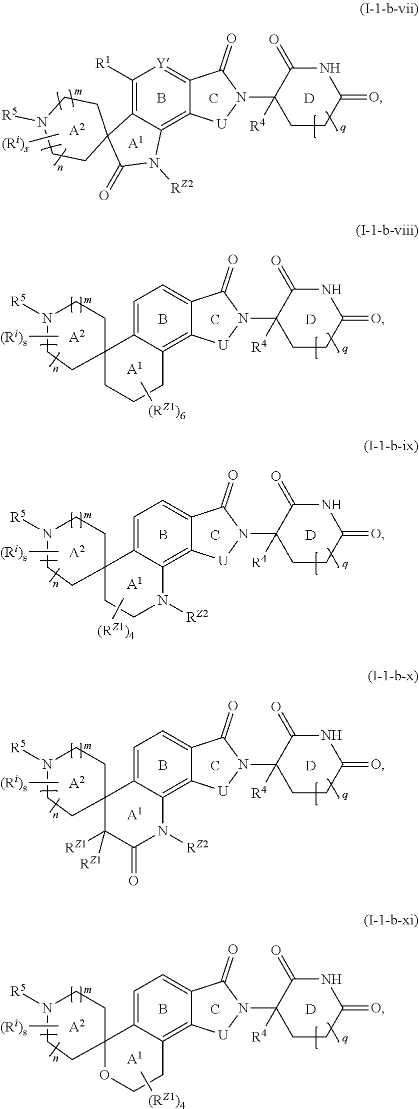

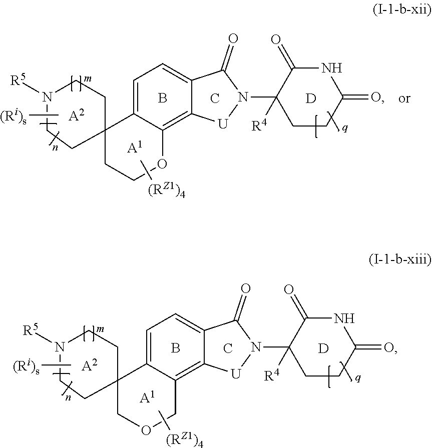

or a pharmaceutically acceptable salt, solvate, or stereoisomer thereof, wherein
R⁵ is hydrogen, C₁₋₆ alkyl, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}; or
R⁵ is an amino-protecting group; and
m and n are independently an integer selected from 0 to 2.

In certain embodiments, the compound of Formula I-2 is a compound of Formula I-2-b-i, I-2-b-ii, I-2-b-iii, I-2-b-iv, I-2-b-v, I-2-b-vi, I-2-b-vii, I-2-b-viii, I-2-b-ix, I-2-b-x, I-2-b-xi, I-2-b-xii, or I-2-b-xiii:

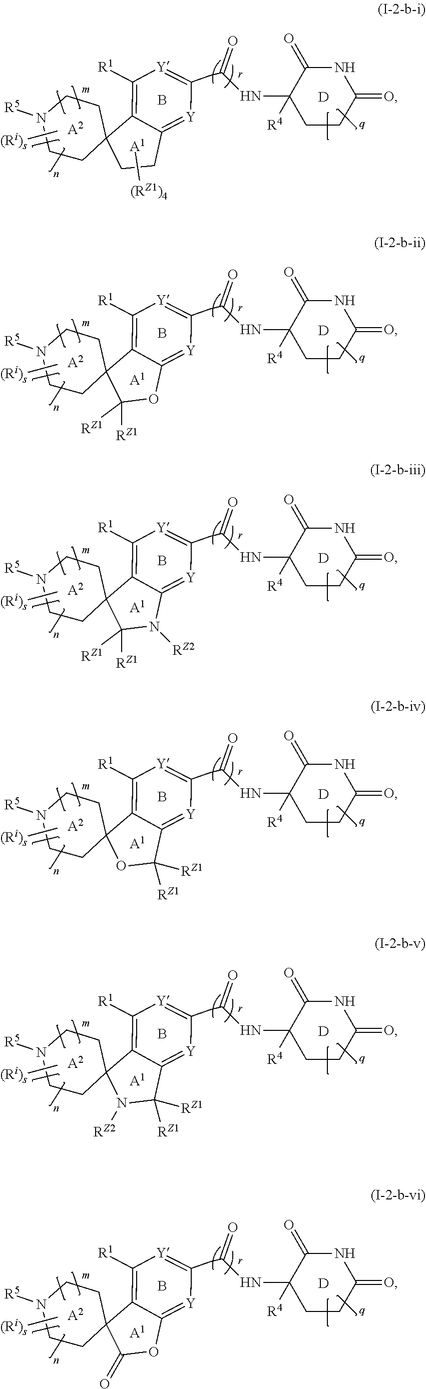

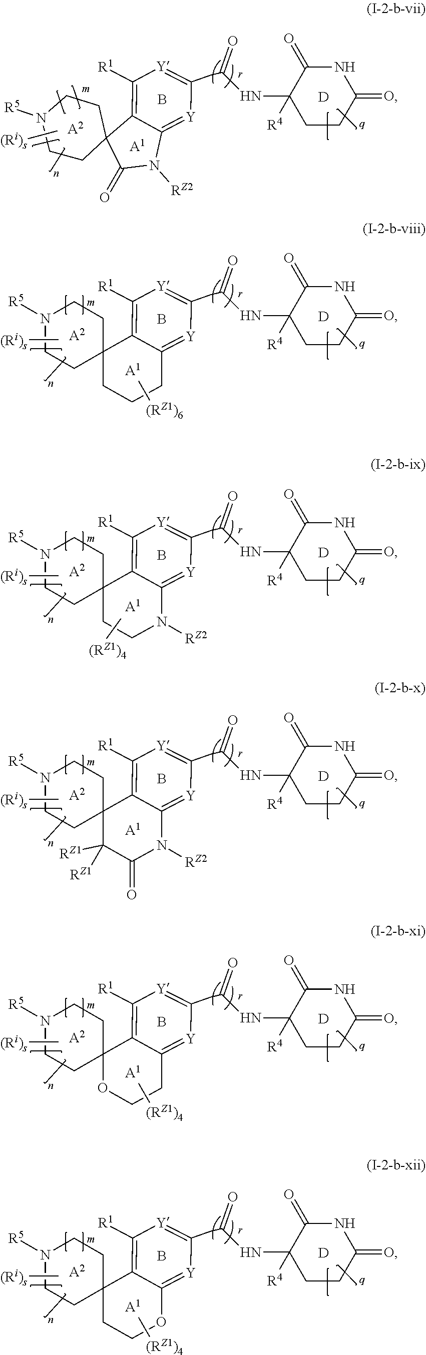

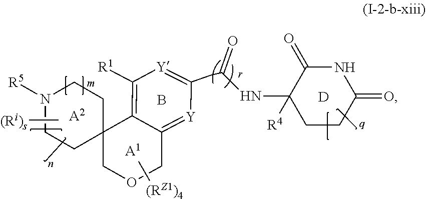

or a pharmaceutically acceptable salt, solvate, or stereoisomer thereof, wherein
R⁵ is hydrogen, C₁₋₆ alkyl, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}; or
R⁵ is an amino-protecting group; and
m and n are independently an integer selected from 0 to 2.

In certain embodiments, each R⁵ is independently hydrogen, C₁₋₆ alkyl, C₃₋₆ carbocyclyl, 3- to 6-membered heterocyclyl, —S(═O)₂R^{a}, or —C(═O)R^{a}, wherein the alkyl, carbocyclyl, or heterocyclyl is optionally substituted with one or more R^{u}.

In certain embodiments, each R⁵ is independently hydrogen or C₁₋₆ alkyl.

In certain embodiments, R¹ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, R¹ is hydrogen.

In certain embodiments, Y′ is N.

In certain embodiments, Y′ is CRY′.

In certain embodiments, R^{Y}′ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, R^{Y}′ is hydrogen.

In certain embodiments, each Rⁱ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, s is an integer selected from 0 to 8, as valency permits. In certain embodiments, s is an integer selected from 0 to 7, as valency permits. In certain embodiments, s is an integer selected from 0 to 6, as valency permits. In certain embodiments, s is an integer selected from 0 to 5, as valency permits. In certain embodiments, s is an integer selected from 0 to 4, as valency permits. In certain embodiments, s is an integer selected from 0 to 3, as valency permits. In certain embodiments, s is an integer selected from 0 to 2, as valency permits. In certain embodiments, s is 0 or 1, as valency permits.

In certain embodiments, s is 0. In certain embodiments, s is 1. In certain embodiments, s is 2. In certain embodiments, s is 3. In certain embodiments, s is 4. In certain embodiments, s is 5. In certain embodiments, s is 6. In certain embodiments, s is 7. In certain embodiments, s is 8.

In certain embodiments, R⁴ is hydrogen. In certain embodiments, R⁴ is deuterium. In certain embodiments, R⁴ is C₁₋₆ haloalkyl. In certain embodiments, R⁴ is C₁₋₆ alkyl.

In certain embodiments, q is 0. In certain embodiments, q is 1. In certain embodiments, q is 2. In certain embodiments, q is 0 or 1. In certain embodiments, q is 0 or 2. In certain embodiments, q is 1 or 2.

### Bifunctional Degraders

In certain aspects, the present disclosure provides conjugates comprising a compound disclosed herein being connected to a ligand for a protein (e.g., via a linker).

In certain aspects, the present disclosure provides conjugates of Formula II:

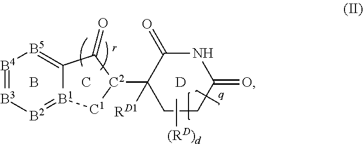

and pharmaceutically acceptable salts, solvates, or stereoisomers thereof, wherein: B² is N or CR^{B2};
B³ is N or CR^{B3};
B⁴ is N or CR^{B4};
B⁵ is N or CR^{B5};
R^{B2}, R^{B3}, R^{B4}, and R^{B5} are independently hydrogen, halogen, —CN, —NO₂, —OH, —NH₂, C₁₋₆ alkyl,
C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{C}S(═O)R^{a}, —NR^{C}S(═O)₂OR^{b}, —NR^{C}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
wherein one of R^{B2} and R^{B3}, R^{B3} and R^{B4}, or R^{B4} and R^{B5}, together with the carbon atoms to which they are bonded, form Ring A attached to L-T, wherein Ring A is optionally substituted 7- to 16-membered spiro heterocycle;
- - - denotes an optional covalent bond between B¹ and C₁;
i) when the bond between B¹ and C¹ is present:
   r is 1;
   B¹ is C;
   C¹ is —C(R^{C1})₂—, —C(═O)—, —(C═O)—N(R^{C1}′)—*, or —N═C(R^{C1}′)—*;
   each R^{C1} is independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}; or
   two R^{C1}, together with the carbon atom to which they are attached, form C₃₋₆ carbocycle or 3- to 6-membered heterocycle, wherein the carbocycle or heterocycle is optionally substituted with one or more R^{u};
   R^{C1}′ is H or C₁₋₆ alkyl optionally substituted with one or more R^{u}, and * denotes attachment to Ring B; and
   C² is N;
ii) when the bond between B¹ and C¹ is absent:
   r is 0 or 1;
   B¹ is N or CR^{B1};
   R^{B1} is hydrogen, halogen, —CN, —NO₂, —OH, —NH₂, C₁₋₆ alkyl, C₁₋₆ alkoxy, C₁₋₆ alkylamino,
   C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
   C¹ is absent; or
   C¹ is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
   C₂ is Nor O;
   wherein i) when C² is N, then C¹ is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}; ii) when C₂ is O, then C¹ is absent;
R^{D1} is hydrogen, deuterium, or C₁₋₆ alkyl optionally substituted with one or more R^{u};
q is an integer from 0 to 2,
each RD is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
d is an integer selected from 0 to 5;
L is a linker; and
T is a ligand for a protein,

wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{C}S(═O)R^{a}, —NR^{C}S(═O)₂OR^{b}, —NR^{b}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₃₋₆ carbocyclyl, 3- to 6-membered heterocyclyl, C₆ aryl, and 5- or 6-membered heteroaryl; or
two R^{u}, together with the one or more intervening atoms, form C₆₋₁₀ aryl, 5- to 10-membered heteroaryl, C₃₋₁₂ carbocyclyl, or 3- to 12-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl or 5- to 10-membered heteroaryl, wherein the heterocyclyl or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₃₋₆ carbocyclyl, and 3- to 6-membered heterocyclyl;
wherein each of R^{a}, Rb, R^{c}, and R^{d} is independently and optionally substituted with one or more R^{z};
each R^{z} is independently oxo, halogen, —CN, —NO₂, —OH, —NH₂, C₁₋₆ alkyl, C₁₋₆ alkoxy, C₁₋₆ alkylamino, C₃₋₆ carbocyclyl, 3- to 6-membered heterocyclyl, C₆ aryl, or 5- or 6-membered heteroaryl,
wherein each of the variables in Formula II is described herein.

L, a linker, is a divalent chemical moiety that connects the ligand of a protein with the cereblon ligand disclosed herein. L configures the ligand and the cereblon ligand such that the construct functions as a bifunctional degrader which binds the cereblon ligand and selectively degrades the target protein.

In certain embodiments, L is a linker comprising C₁₋₆ alkylene, C₂₋₆ alkenylene, C₂₋₆ alkynylene, C₃₋₁₂ carbocyclylene, 3- to 12-membered heterocyclylene, C₆₋₁₀ arylene, 5- to 10-membered heteroarylene, —C(═O)—, —C(═O)N(R^{L}′)—, —C(═O)O—, —N(R^{L}′)—, —O—, —S—, or —S(═O)₂—, wherein the alkylene, alkenylene, carbocyclylene, heterocyclylene, arylene, or heteroarylene is optionally substituted by one or more R^{u}.

In certain embodiments, L is of Formula II-2

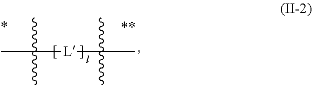

wherein:
* denotes attachment to T and ** denotes attachment to C;
each L′ is independently C₁₋₆ alkylene, C₂₋₆ alkenylene, C₂₋₆ alkynylene, C₃₋₁₂ carbocyclylene, 3- to 12-membered heterocyclylene, C₆₋₁₀ arylene, 5- to 10-membered heteroarylene, —C(═O)—, —C(═O)N(R^{L}′)—, —C(═O)O—, —N(R^{L}′)—, —O—, —S—, or —S(═O)₂—, wherein the alkylene, alkenylene, carbocyclylene, heterocyclylene, arylene, or heteroarylene is optionally substituted with one or more R^{u};
each occurrence of R^{L}′ is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}; and
i is an integer selected from 0 to 6.

In certain embodiments, each L′ is independently C₁₋₆ alkylene (e.g., methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), and hexylene (—CH₂CH₂CH₂CH₂CH₂CH₂—)), C₂₋₆ alkenylene (e.g., ethenylene (C₂), 1-propenylene (C₃), 2-propenylene (C₃), 1-butenylene (C₄), 2-butenylene (C₄), butadienylene (C₄), pentenylene (C₅), pentadienylene (C₅), or hexenylene (C₆)), C₂₋₆ alkynylene (e.g., ethynylene (C₂), 1-propynylene (C₃), 2-propynylene (C₃), 1-butynylene (C₄), 2-butynylene (C₄), pentynylene (C₅), or hexynylene (C₆)), C₃₋₁₂ carbocyclylene (e.g., cyclopropylene (C₃), cyclopropenylene (C₃), cyclobutylene (C₄), cyclobutenylene (C₄), cyclopentylene (C₅), cyclopentenylene (C₅), cyclohexylene (C₆), cyclohexenylene (C₆), cyclohexadienylene (C₆), cycloheptylene (C₇), cycloheptenylene (C₇), cycloheptadienylene (C₇), cycloheptatrienylene (C₇), cyclooctylene (C₈), cyclooctenylene (C₈), bicyclo[2.2.1]heptanylene (C₇), bicyclo[2.2.2]octanylene (C₈), cyclononylene (C₉), cyclononenylene (C₉), cyclodecylene (C₁₀), cyclodecenylene (C₁₀), octahydro-1H-indenylene (C₉), decahydronaphthalenylene (C₁₀), or spiro[4.5]decanylene (C₁₀)), 3- to 12-membered heterocyclylene (e.g., heterocyclylene comprising one or two 3- to 8-membered rings and 1-5 heteroatoms selected from N, O, and S), C₆₋₁₀ arylene (e.g., phenylene or naphthylene), 5- to 10-membered heteroarylene (e.g., heteroarylene comprising one or two 5- or 6-membered rings and 1-5 heteroatoms selected from N, O, and S), —C(═O)—, —C(═O)N(R^{L2})—, —C(═O)O—, —N(R^{L2})—, —O—, —S—, or —S(═O)₂—, wherein the alkylene, alkenylene, carbocyclylene, heterocyclylene, arylene, or heteroarylene is optionally substituted with one or more R^{u}.

In certain embodiments, each L′ is independently C₁₋₆ alkylene, C₃₋₁₂ carbocyclylene, 3- to 12-membered heterocyclylene, —C(═O)—, —C(═O)N(R^{L}′)—, —C(═O)O—, —N(R^{L}′)—, —O—, —S—, or —S(═O)₂—, wherein the alkylene, alkenylene, carbocyclylene, heterocyclylene, arylene, or heteroarylene is optionally substituted with one or more R^{u}.

In certain embodiments, each occurrence of R^{L}′ is independently hydrogen, C₁₋₆ alkyl (e.g., methyl(C₁), ethyl(C₂), n-propyl(C₃), i-propyl(C₃), n-butyl(C₄), i-butyl(C₄), s-butyl(C₄), t-butyl(C₄), pentyl(C₅), or hexyl(C₆)), C₂₋₆ alkenyl (e.g., ethenyl(C₂), 1-propenyl(C₃), 2-propenyl(C₃), 1-butenyl(C₄), 2-butenyl(C₄), butadienyl(C₄), pentenyl(C₅), pentadienyl(C₅), or hexenyl(C₆)), C₂₋₆ alkynyl (e.g., ethynyl(C₂), 1-propynyl(C₃), 2-propynyl(C₃), 1-butynyl(C₄), 2-butynyl(C₄), pentynyl(C₅), or hexynyl(C₆)), C₃₋₁₂ carbocyclyl (e.g., cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), cycloheptyl(C₇), cycloheptenyl(C₇), cycloheptadienyl(C₇), cycloheptatrienyl(C₇), cyclooctyl(C₈), cyclooctenyl(C₅), bicyclo[2.2.1]heptanyl(C₇), bicyclo[2.2.2]octanyl(C₈), cyclononyl(C₉), cyclononenyl(C₉), cyclodecyl(C₁₀), cyclodecenyl(C₁₀), octahydro-1H-indenyl(C₉), decahydronaphthalenyl(C₁₀), or spiro[4.5]decanyl(C₁₀)), 3- to 12-membered heterocyclyl (e.g., heterocyclyl comprising one or two 3- to 8-membered rings and 1-5 heteroatoms selected from N, O, and S), C₆₋₁₀ aryl (e.g., phenyl or naphthyl), 5- to 10-membered heteroaryl (e.g., heteroaryl comprising one or two 5- or 6-membered rings and 1-5 heteroatoms selected from N, O, and S), —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}.

In certain embodiments, each occurrence of R^{L}′ is independently hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}.

In certain embodiments, 1 is 0. In certain embodiments, t is 1. In certain embodiments, 1 is 2. In certain embodiments, 1 is 3. In certain embodiments, 1 is 4. In certain embodiments, 1 is 5. In certain embodiments, 1 is 6.

T, a ligand of a protein, is a chemical entity that competitively or non-competitively binds a protein.

In certain embodiments, the protein is B7.1 and B7, TINFRIm, TNFR2, NADPH oxidase, BclIBax and other partners in the apotosis pathway, C₅a receptor, HMG-COA reductase, PDE V phosphodiesterase type, PDE IV phosphodiesterase type 4, PDE I, PDEII, PDEIII, squalene cyclase inhibitor, CXCR1, CXCR2, nitric oxide (NO) synthase, cyclo-oxygenase 1, cyclo-oxygenase 2, 5HT receptors, dopamine receptors, G Proteins, i.e., Gq, histamine receptors, 5-lipoxygenase, tryptase serine protease, thymidylate synthase, purine nucleoside phosphorylase, GAPDH trypanosomal, glycogen phosphorylase, Carbonic anhydrase, chemokine receptors, JAW STAT, RXR and similar, HIV 1 protease, HIV 1 integrase, influenza, neuramimidase, hepatitis B reverse transcriptase, sodium channel, multi drug resistance (MDR), protein P-glycoprotein (and MRP), tyrosine kinases, CD23, CD124, tyrosine kinase p56 lck, CD4, CD5, IL-2 receptor, IL-1 receptor, TNF-alphaR, ICAM1, Cat+ channels, VC AM, VLA-4 integrin, selectins, CD40/CD40L, newokinins and receptors, inosine monophosphate dehydrogenase, p38 MAP Kinase, RaslRafIMEWERK pathway, interleukin-1 converting enzyme, caspase, HCV, NS3 protease, HCV NS3 RNA helicase, glycinamide ribonucleotide formyl transferase, rhinovirus 3C protease, herpes simplex virus-1 (HSV-I), protease, cytomegalovirus (CMV) protease, poly(ADP-ribose) polymerase, cyclin dependent kinases, vascular endothelial growth factor, oxytocin receptor, microsomal transfer protein inhibitor, bile acid transport inhibitor, 5 alpha reductase inhibitors, angiotensin 11, glycine receptor, noradrenaline reuptake receptor, endothelin receptors, neuropeptide Y and receptor, estrogen receptors, androgen receptors (AR), adenosine receptors, adenosine kinase and AMP deaminase, purinergic receptors (P2Y1, P2Y2, P2Y4, P2Y6, P2X1-7), farnesyl transferases, geranylgeranyl transferase, TrkA a receptor for NGF, beta-amyloid, tyrosine kinase Flk-IIKDR, vitronectin receptor, integrin receptor, Her-21 neu, telomerase inhibition, cytosolic phospholipaseA2 and EGF receptor tyrosine kinase. Additional protein targets include, for example, ecdysone 20-monooxygenase, ion channel of the GABA gated chloride channel, acetylcholinesterase, voltage-sensitive sodium channel protein, calcium release channel, and chloride channels. Still further target proteins include Acetyl-CoA carboxylase, adenylosuccinate synthetase, protoporphyrinogen oxidase, and enolpyruvylshikimate-phosphate synthase.

In certain embodiments, the protein is an androgen receptor (AR), an estrogen receptor (ER), signal transducer and activator of transcription 3 (STAT3), signal transducer and activator of transcription 5 (STAT5), CREB-binding protein/EP300 (E1A) binding protein (CBP/p300), SWI/SNF Related, Matrix Associated, Actin Dependent Regulator Of Chromatin, Subfamily A, Member 2/4 (SMARCA2/4), Ikaros Zinc Finger (IKZF)1, IKZF2, or IKZF3, Kirsten rat sarcoma viral oncogene homolog G12D (KRAS G12D), Src homology region 2-containing protein tyrosine phosphatase 2 (SHP2), or bromodomain-containing protein 4 (BRD4).

In certain embodiments, T is a small molecule.

In certain embodiments, T is an antibody.

In certain embodiments, T is a peptide. In certain embodiments, the peptide has about 5 amino acids. In certain embodiments, the peptide has about 10 amino acids. In certain embodiments, the peptide has about 15 amino acids. In certain embodiments, the peptide has about 20 amino acids. In certain embodiments, the peptide has about 25 amino acids. In certain embodiments, the peptide has about 30 amino acids. In certain embodiments, the peptide has about 35 amino acids. In certain embodiments, the peptide has about 40 amino acids. In certain embodiments, the peptide has about 45 amino acids. In certain embodiments, the peptide has about 50 amino acids.

In certain embodiments, T is a ligand for an estrogen receptor. In certain embodiments, T is a ligand for SMARCA2/4 protein. In certain embodiments, T is a ligand for STAT3 protein. In certain embodiments, T is a ligand for CBP/p300 protein. In certain embodiments, T is a ligand for Ikaros Zinc Finger (IKZF)1, IKZF2, or IKZF3. In certain embodiments, T is ligand for an androgen receptor. In certain embodiments, T is a ligand for BRD9 protein.

In certain embodiments, T is an estrogen receptor inhibitor. In certain embodiments, T is a SMARCA2/4 protein inhibitor. In certain embodiments, T is a STAT3 protein inhibitor. In certain embodiments, T is a CBP/p300 protein inhibitor. In certain embodiments, T is a Ikaros Zinc Finger (IKZF)1, IKZF2, or IKZF3 degrader. In certain embodiments, T is an androgen receptor inhibitor. In certain embodiments, T is a BRD9 protein inhibitor.

In certain aspects, the present disclosure provides conjugates of Formula I′:

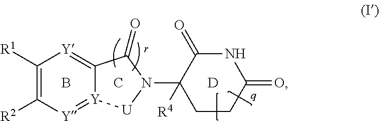

and pharmaceutically acceptable salts, solvates, or stereoisomers thereof,

wherein:
R¹ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{C}S(═O)R^{a}, —NR^{C}S(═O)₂OR^{b}, —NR^{C}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
R¹ and R², together with the intervening carbon atoms, form optionally substituted 7- to 16-membered spiro heterocycle attached to -L-T;
Y″ is N or CR³;
R³ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{C}S(═O)R^{a}, —NR^{C}S(═O)₂OR^{b}, —NR^{C}S(═O)₂NR^{c}R^{d}, —NR^{o}C(═O)NR^{c}R^{d}, —NR^{o}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
R² and R³, together with the intervening carbon atoms, form optionally substituted 7- to 16-membered spiro heterocycle attached to -L-T;
provided that either R¹ and R², or R^{z} and R³ form optionally substituted 7- to 16-membered spiro heterocycle attached to -L-T;
Y′ is N or CRY;
R^{Y} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u};
---denotes an optional covalent bond between Y and U;
when the bond between Y and U is absent:
r is 0 or 1;
Y is N or CRY;
R^{Y} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u};
U is hydrogen or C₁₋₆ alkyl optionally substituted with one or more R^{u};
when the bond between Y and U is present:
r is 1;
Y is C;
U is —CH₂—, —C(═O)—, —(C═O)—N(R^{U})—*, —N═C(R^{U})—*;
R^{U} is H or C₁₋₆ alkyl optionally substituted with one or more R^{u}, and * denotes attachment to Ring B;
R⁴ is hydrogen, deuterium, C₁₋₆ haloalkyl, or C₁₋₆ alkyl; and
q is an integer from 0 to 2,
L is a linker; and
T is a ligand for a protein;

wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{C}S(═O)R^{a}, —NR^{C}S(═O)₂OR^{b}, —NR^{C}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl; or two R^{u}, together with the one or more intervening atoms, form C₆₋₁₀ aryl, 5- to 10-membered heteroaryl, C₃₋₁₂ carbocyclyl or 3- to 12-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; or
R^{c} and R^{d}, together with the nitrogen atom to which they are attached, form 3- to 12-membered heterocyclyl,
wherein each occurrence of R^{a}, Rb, R^{c}, and R^{d} is independently and optionally substituted with one or more R²; and
each R^{z} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, or 3- to 6-membered heterocyclyl.

In certain embodiments, the conjugate of Formula I′ is a conjugate of Formula I′-1

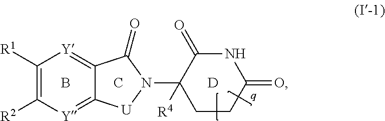

or a pharmaceutically acceptable salt, solvate, or stereoisomer thereof.

In certain embodiments, U is —CH₂- or —C(═O)—.

In certain embodiments, the conjugate of Formula I′ is a conjugate of Formula I′-2

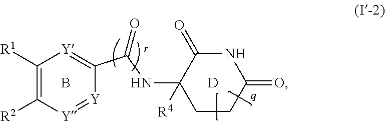

or a pharmaceutically acceptable salt, solvate, or stereoisomer thereof.

In certain embodiments, Y is N.

In certain embodiments, Y is CRY.

In certain embodiments, R^{Y} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, R^{Y} is hydrogen, halogen, C₁₋₆ alkoxy.

In certain embodiments, R¹ and R², together with the intervening carbon atoms, form optionally substituted 7- to 16-membered spiro heterocycle attached to -L-T.

In certain embodiments, the 7- to 16-membered spiro heterocycle is optionally substituted with one or more R^{u}. In certain embodiments, the 7- to 16-membered spiro heterocycle is optionally substituted with one or more Rⁱ. In certain embodiments, the 7- to 16-membered spiro heterocycle is optionally substituted with one or more R^{X1}. In certain embodiments, the 7- to 16-membered spiro heterocycle is optionally substituted with one or more R^{Z1}. In certain embodiments, the 7- to 16-membered spiro heterocycle is optionally substituted with one or more R^{X2}. In certain embodiments, the 7- to 16-membered spiro heterocycle is optionally substituted with one or more R^{Z2}.

In certain embodiments, R^{u} is Rⁱ. In certain embodiments, R^{u} is R^{X1}. In certain embodiments, R^{u} is R^{X2}. In certain embodiments, R^{u} is R^{Z1}. In certain embodiments, R^{u} is R^{Z2}. In certain embodiments, Rⁱ is R^{X1}. In certain embodiments, Rⁱ is R^{X2}. In certain embodiments, Rⁱ is R^{Z1}. In certain embodiments, Rⁱ is R^{Z2}.

In certain embodiments, the 7- to 16-membered spiro heterocycle is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{C}S(═O)R^{a}, —NR^{C}S(═O)₂OR^{b}, —NR^{C}S(═O)₂NR^{c}R^{a}, —NR^{o}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, the 7- to 16-membered spiro heterocycle is

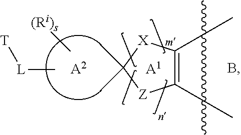

wherein:
Ring A² is C₃₋₁₂ carbocycle or 3- to 12-membered heterocycle;
each X is independently —C(R^{X1})₂—, —NR^{X2}—, —O—, —S—, —S(═O)—, or —S(═O)₂—;
each Z is independently —C(R^{Z1})₂—, —NR^{z2}—, —O—, —S—, —S(═O)—, or —S(═O)₂—;
each occurrence of R^{X1} and R^{Z1} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{C}S(═O)R^{a}, —NR^{C}S(═O)₂OR^{b}, —NR^{C}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
two geminal R^{X1} or two geminal R^{Z1} together form oxo; or
two R^{X1} or two R^{Z1}, together with the intervening carbon atom(s), form C₃₋₁₂ carbocyclyl or 3- to 12-membered heterocyclyl, wherein the carbocyclyl or heterocyclyl is optionally substituted with one or more R^{u};
each occurrence of R^{X2} and R^{z2} is independently hydrogen or C₁₋₆ alkyl optionally substituted with one or more R^{u};
m′ and n′ are independently an integer selected from 0 to 3;
provided that either m′ or n′ is 0;
each Rⁱ independently is oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{C}S(═O)R^{a}, —NR^{C}S(═O)₂OR^{b}, —NR^{C}S(═O)₂NR^{c}R^{d}, —NR^{o}C(═O)NR^{c}R^{a}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
s is an integer selected from 0 to 10.

In certain embodiments, the 7- to 16-membered spiro heterocycle is

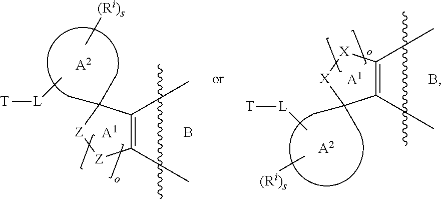

wherein o is an integer selected from 0 to 2.

In certain embodiments, Ring A¹ is 4- to 6-membered heterocycle.

In certain embodiments, X is —C(R^{X1})₂—, —NR^{X2}—, or —O—, and Z is —C(R^{Z1})₂—, —NR^{z2}, or O—.

In certain embodiments, the conjugate of Formula I′-1 is a conjugate of Formula I′-1-a-i, I′-1-a-ii, I′-1-a-iii, I′-1-a-iv, I′-1-a-v, I′-1-a-vi, I′-1-a-vii, I′-1-a-viii, I′-1-a-ix, I′-1-a-x, I′-1-a-xi, I′-1-a-xii, or I′-1-a-xiii:

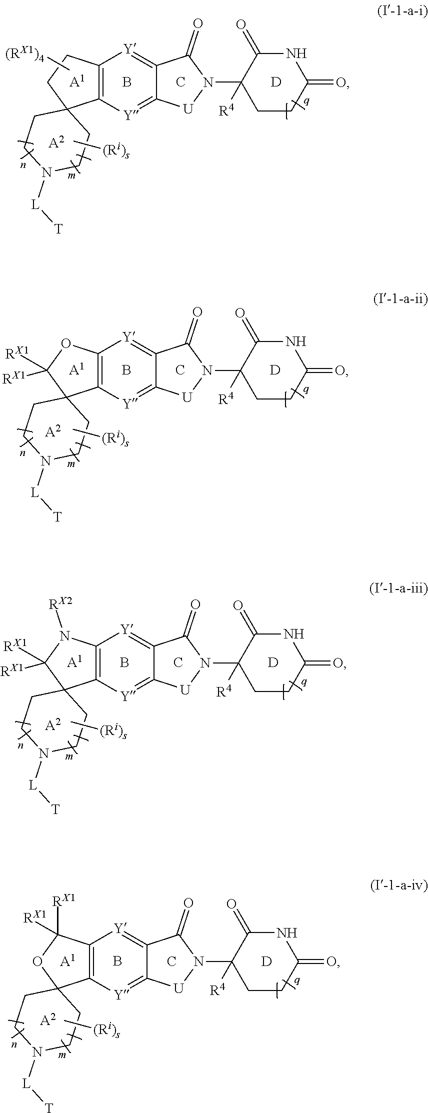

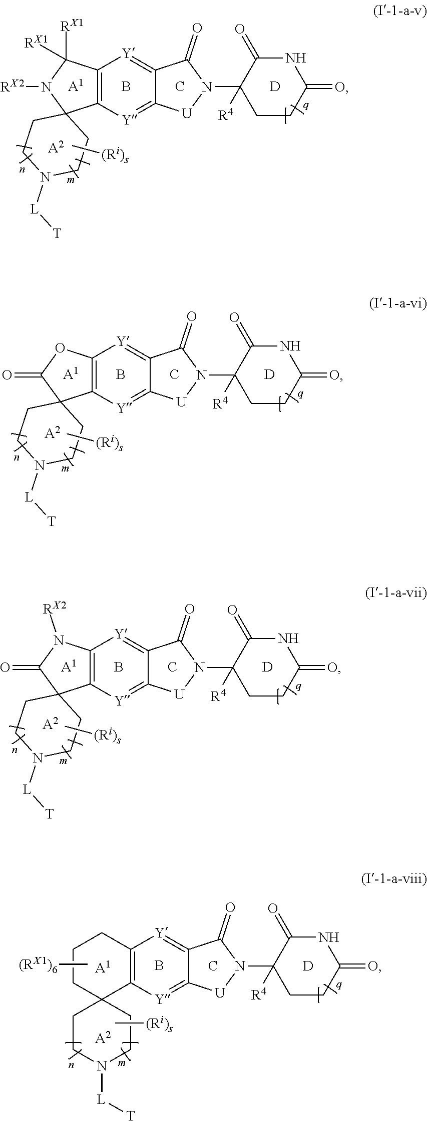

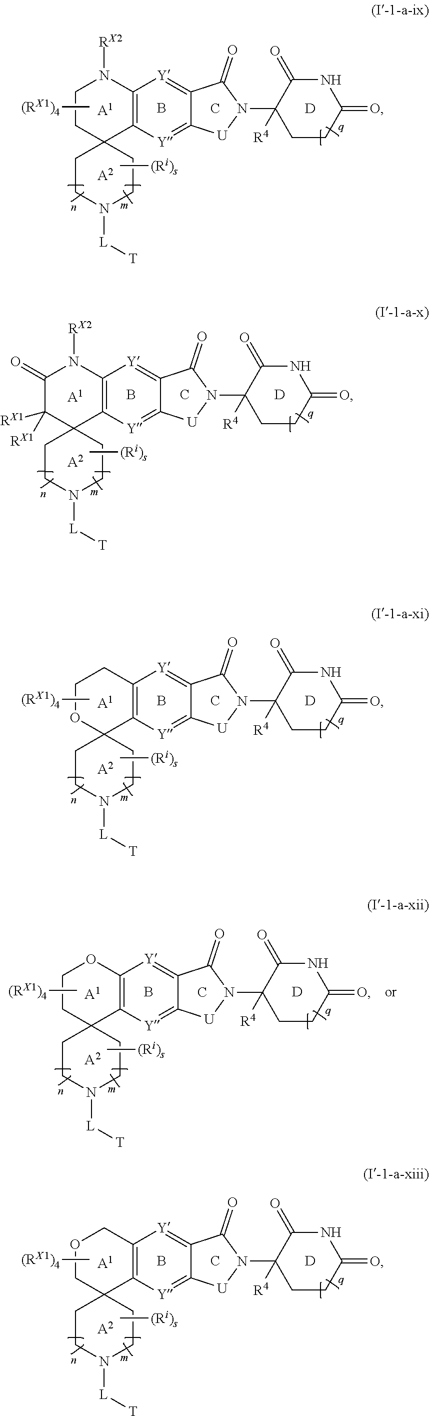

or a pharmaceutically acceptable salt, solvate, or stereoisomer thereof, wherein
m and n are independently an integer selected from 0 to 2.

In certain embodiments, the conjugate of Formula I′-2 is a conjugate of Formula I′-2-a-i, I′-2-a-ii, I′-2-a-iii, I′-2-a-iv, I′-2-a-v, I′-2-a-vi, I′-2-a-vii, I′-2-a-viii, I′-2-a-ix, I′-2-a-x, I′-2-a-xi, I′-2-a-xii, or I′-2-a-xiii:

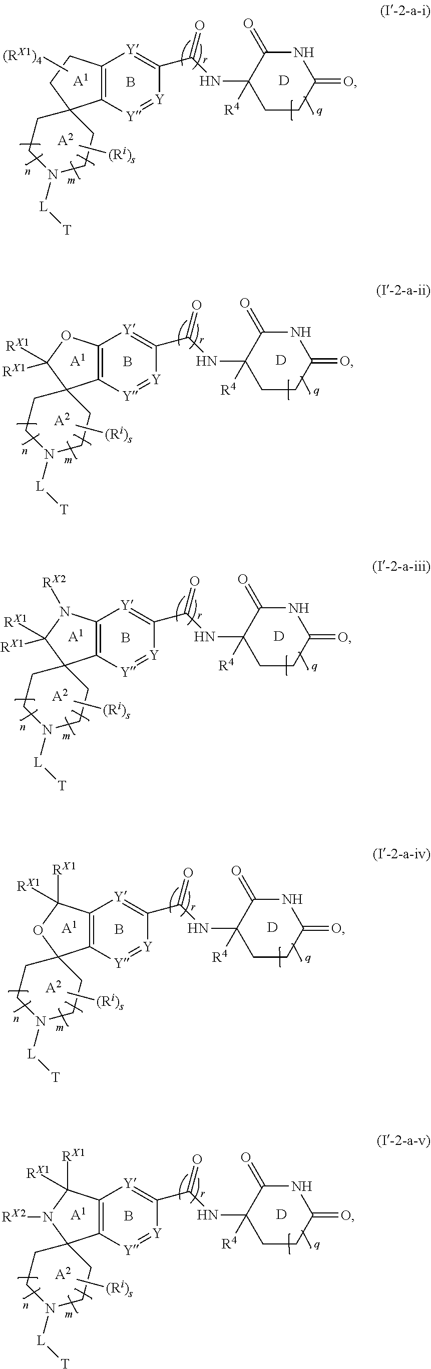

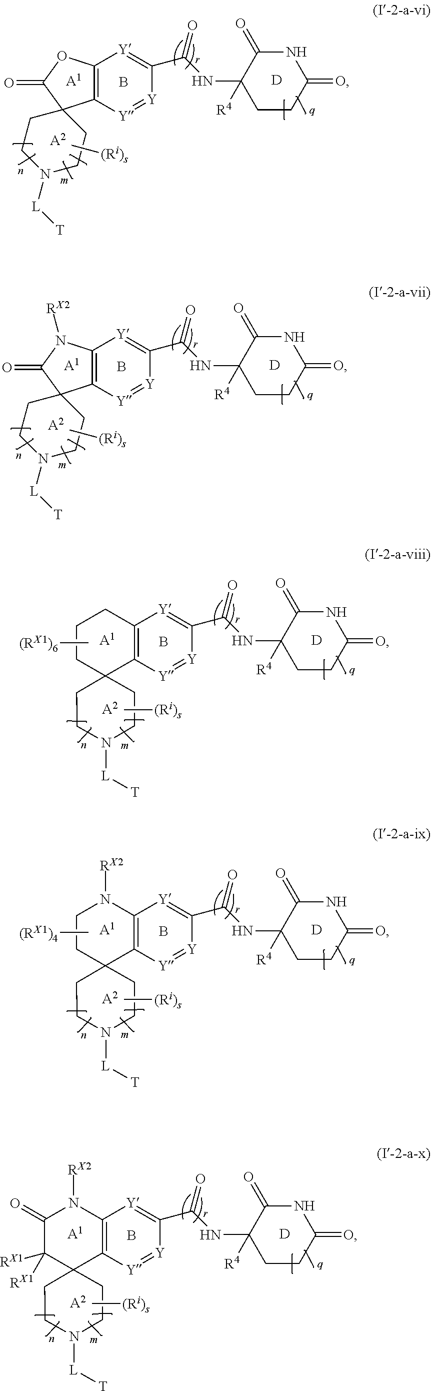

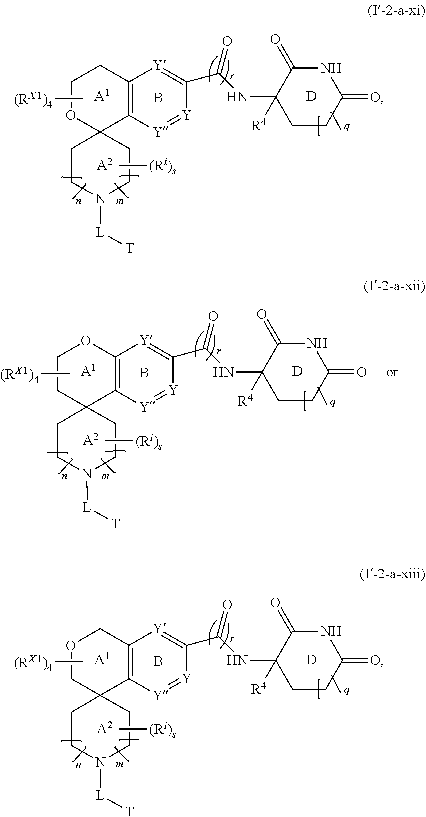

or a pharmaceutically acceptable salt, solvate, or stereoisomer thereof, wherein
m and n are independently an integer selected from 0 to 2.

In certain embodiments, Y″ is N.

In certain embodiments, Y″ is CR³.

In certain embodiments, R³ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, R³ is hydrogen.

In certain embodiments, R² and R³, together with the intervening carbon atoms, form optionally substituted 7- to 16-membered spiro heterocycle attached to -L-T.

In certain embodiments, the 7- to 16-membered spiro heterocycle is optionally substituted with one or more R^{u}. In certain embodiments, the 7- to 16-membered spiro heterocycle is optionally substituted with one or more Rⁱ. In certain embodiments, the 7- to 16-membered spiro heterocycle is optionally substituted with one or more R^{X1}. In certain embodiments, the 7- to 16-membered spiro heterocycle is optionally substituted with one or more R^{Z1}. In certain embodiments, the 7- to 16-membered spiro heterocycle is optionally substituted with one or more R^{X2}. In certain embodiments, the 7- to 16-membered spiro heterocycle is optionally substituted with one or more R^{Z2}.

In certain embodiments, R^{u} is Rⁱ. In certain embodiments, R^{u} is R^{X1}. In certain embodiments, R^{u} is R^{X2}. In certain embodiments, R^{u} is R^{Z1}. In certain embodiments, R^{u} is R^{Z2}. In certain embodiments, Rⁱ is R^{X1}. In certain embodiments, Rⁱ is R^{X2}. In certain embodiments, Rⁱ is R^{Z1}. In certain embodiments, Rⁱ is R^{Z2}.

In certain embodiments, the 7- to 16-membered spiro heterocycle is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{C}S(═O)R^{a}, —NR^{C}S(═O)₂OR^{b}, —NR^{C}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₆ carbocyclyl, and 3- to 6-membered heterocyclyl.

In certain embodiments, the 7- to 16-membered spiro heterocycle is

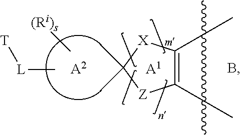

wherein:
Ring A² is C₃₋₁₂ carbocycle or 3- to 12-membered heterocycle;
each X is independently —C(R^{X1})₂—, —NR^{X2}—, —O—, —S—, —S(═O)—, or —S(═O)₂—;
each Z is independently —C(R²¹)₂-, —NR^{z2}—, —O—, —S—, —S(═O)—, or —S(═O)₂—;
each occurrence of R^{X1} and R^{Z1} is independently hydrogen, halogen, —CN, —NO₂, —OH, —NH₂, membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{C}S(═O)R^{a}, —NR^{C}S(═O)₂OR^{b}, —NR^{c}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
two geminal R^{X1} or two geminal R^{Z1} together form oxo; or
two R^{X1} or two R^{Z1}, together with the intervening carbon atom(s), form C₃₋₁₂ carbocyclyl or 3- to 12-membered heterocyclyl, wherein the carbocyclyl or heterocyclyl is optionally substituted with one or more R^{u};
each occurrence of R^{X2} and R⁷² is independently hydrogen or C₁₋₆ alkyl optionally substituted with one or more R^{u};
m′ and n′ are independently an integer selected from 0 to 3;
provided that either m′ or n′ is 0;
each Rⁱ independently is oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{C}S(═O)R^{a}, —NR^{C}S(═O)₂OR^{b}, —NR^{C}S(═O)₂NR^{c}R^{d}, —NR^{o}C(═O)NR^{c}R^{d}, —NR^{o}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
s is an integer selected from 0 to 10.

In certain embodiments, the 7- to 16-membered spiro heterocycle is

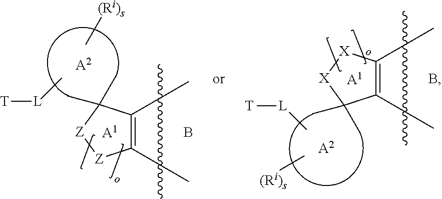

wherein o is an integer selected from 0 to 2.

In certain embodiments, Ring A¹ is 4- to 6-membered heterocycle.

In certain embodiments, X is —C(R^{X1})₂—, —NR^{X2}—, or —O—, and Z is —C(R^{Z1})₂—, —NR^{z2}—, or —O—,

In certain embodiments, the conjugate of Formula I′-1 is a conjugate of Formula I′-1-b-i, I′-1-b-ii, I′-1-b-iii, I′-1-b-iv, I′-1-b-v, I′-1-b-vi, I′-1-b-vii, I′-1-b-viii, I′-1-b-ix, I′-1-b-x, I′-1-b-xi, I′-1-b-xii, or I′-1-b-xiii:

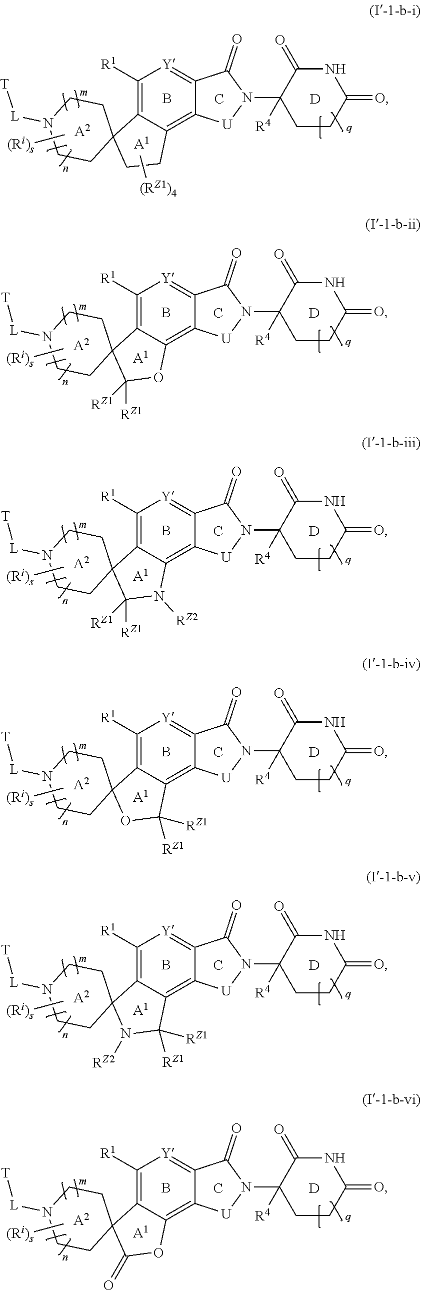

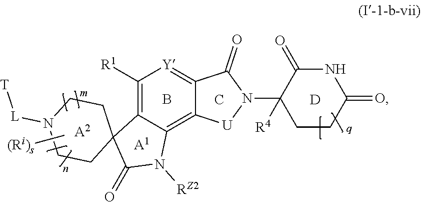

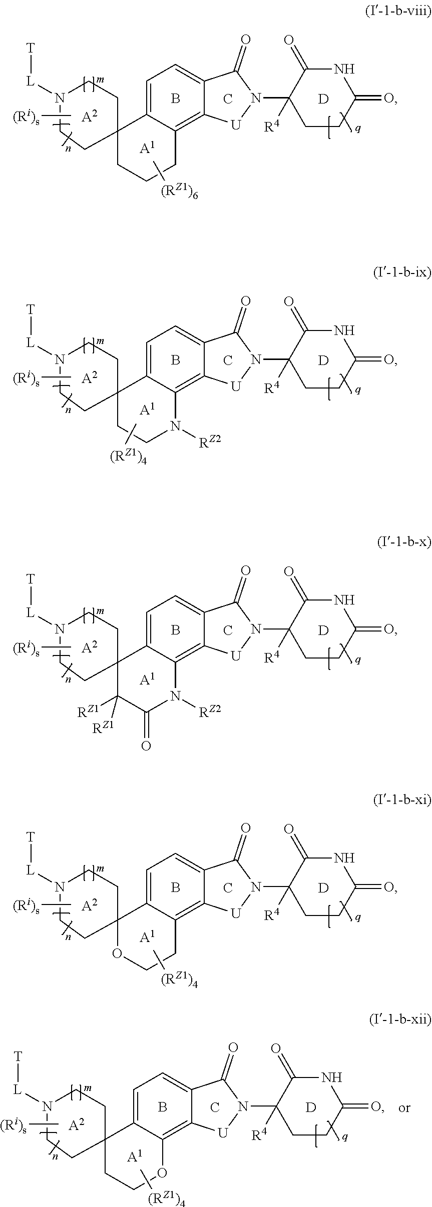

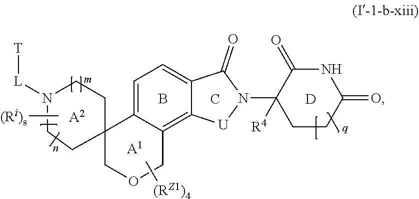

or a pharmaceutically acceptable salt, solvate, or stereoisomer thereof, wherein
m and n are independently an integer selected from 0 to 2.

In certain embodiments, the conjugate of Formula I′-2 is a conjugate of Formula I′-2-b-i, I′-2-b-ii, I′-2-b-iii, I′-2-b-iv, I′-2-b-v, I′-2-b-vi, I′-2-b-vii, I′-2-b-viii, I′-2-b-ix, I′-2-b-x, I′-2-b-xi, I′-2-b-xii, or I′-2-b-xiii:

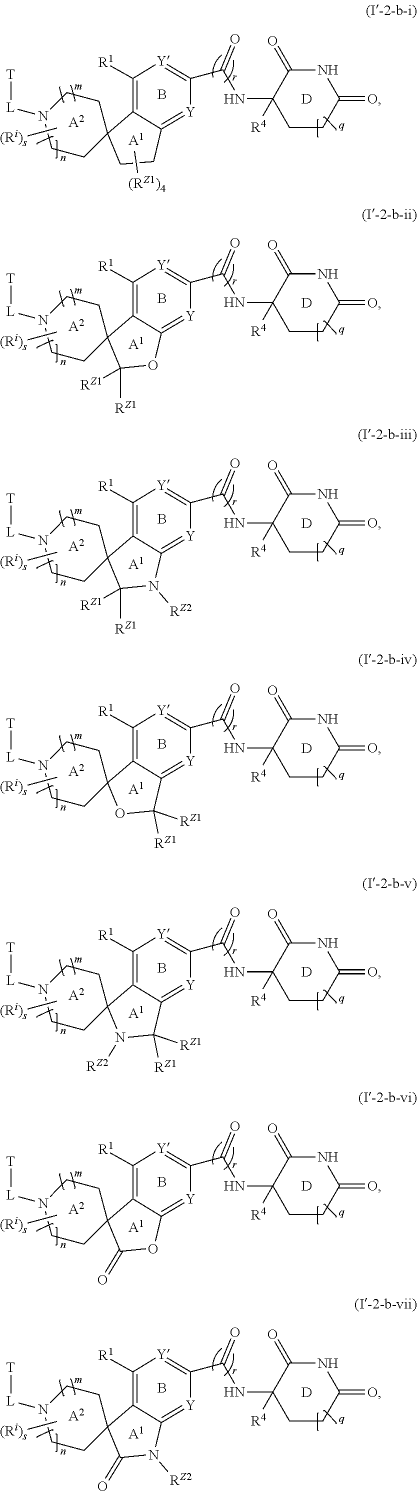

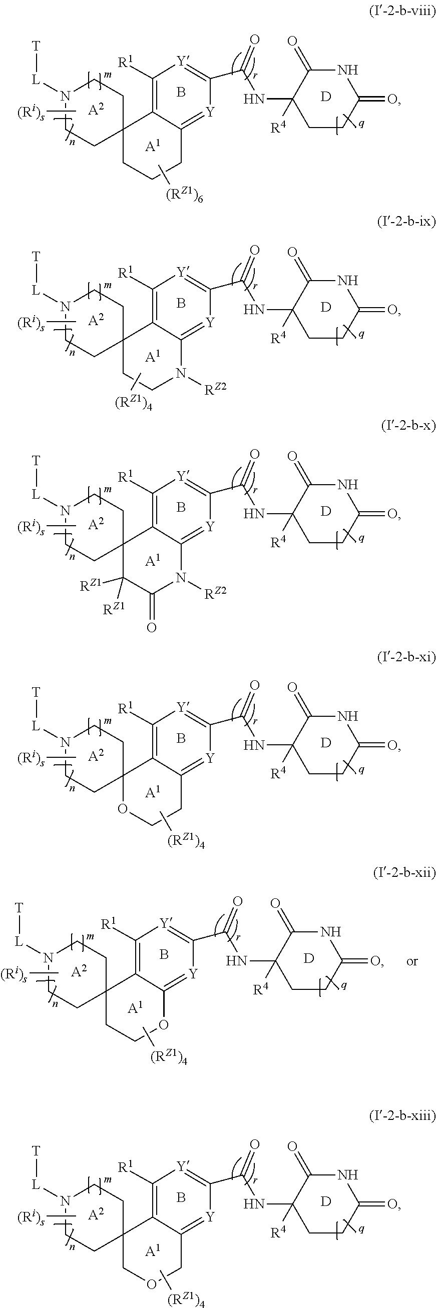

or a pharmaceutically acceptable salt, solvate, or stereoisomer thereof, wherein
m and n are independently an integer selected from 0 to 2.

In certain embodiments, R¹ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, R¹ is hydrogen.

In certain embodiments, Y′ is N.

In certain embodiments, Y′ is CRY′.

In certain embodiments, R^{Y}′ is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, R^{Y}′ is hydrogen.

In certain embodiments, each Rⁱ is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkoxy, alkylamino, alkenyl, alkynyl, aryl, heteroaryl, carbocyclyl, or heterocyclyl is optionally substituted with one or more R^{u}.

In certain embodiments, s is an integer selected from 0 to 8, as valency permits. In certain embodiments, s is an integer selected from 0 to 7, as valency permits. In certain embodiments, s is an integer selected from 0 to 6, as valency permits. In certain embodiments, s is an integer selected from 0 to 5, as valency permits. In certain embodiments, s is an integer selected from 0 to 4, as valency permits. In certain embodiments, s is an integer selected from 0 to 3, as valency permits. In certain embodiments, s is an integer selected from 0 to 2, as valency permits. In certain embodiments, s is 0 or 1, as valency permits.

In certain embodiments, s is 0. In certain embodiments, s is 1. In certain embodiments, s is 2. In certain embodiments, s is 3. In certain embodiments, s is 4. In certain embodiments, s is 5. In certain embodiments, s is 6. In certain embodiments, s is 7. In certain embodiments, s is 8.

In certain embodiments, R⁴ is hydrogen. In certain embodiments, R⁴ is deuterium. In certain embodiments, R⁴ is C₁₋₆ haloalkyl. In certain embodiments, R⁴ is C₁₋₆ alkyl.

In certain embodiments, q is 0. In certain embodiments, q is 1. In certain embodiments, q is 2. In certain embodiments, q is 0 or 1. In certain embodiments, q is 0 or 2. In certain embodiments, q is 1 or 2.

L, the linker, is a chemical moiety that connects the ligand of a protein with the cereblon ligand disclosed herein. L configures the ligand and the cereblon ligand such that the construct functions as a bifunctional degrader which binds the cereblon ligand and selectively degrades the target protein.

In certain embodiments, L is a linker comprising 6- to 10-membered heteroarylene, C₆-10 arylene, C₃₋₁₂ membered carbocyclylene, or 3- to 12-membered heterocyclylene, wherein the arylene, heteroarylene, carbocyclylene, or heterocyclylene is optionally substituted by one or more R^{u}, and is directly attached to T.

In certain embodiments, L is of formula

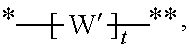

wherein:
* denotes attachment to T and ** denotes attachment to C;
each occurrence of —W′— is independently C₁₋₃ alkylene, C² alkenylene, C² alkynylene, C₃₋₁₂ carbocyclylene, 3- to 12-membered heterocyclylene, C₆₋₁₀ arylene, 5- to 10-membered heteroarylene, —C(═O)—, —N(R^{L})—, —O—, —S—, or —S(═O)₂—, wherein the alkylene, alkenylene, carbocyclylene, heterocyclylene, arylene, or heteroarylene is optionally substituted with one or more R^{u};
each occurrence of R^{L} is independently hydrogen, C₁₋₆ alkyl, C₂₋₆ alkenyl, C₂₋₆ alkynyl, C₆₋₁₀ aryl, 5- to 10-membered heteroaryl, C₃₋₁₂ carbocyclyl, or 3- to 12-membered heterocyclyl, wherein the alkyl, alkenyl, alkynyl, carbocyclyl, heterocyclyl, aryl, or heteroaryl is optionally substituted with one or more R^{u}; and
t is an integer selected from 1 to 15.

In certain embodiments, L is of formula

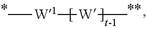

wherein:
W′1 is 6- to 10-membered heteroarylene, C₆₋₁₀ arylene, C₃₋₁₂ membered carbocyclylene, or 3- to 12-membered heterocyclylene, wherein the arylene, heteroarylene, carbocyclylene, or heterocyclylene is optionally substituted by one or more R^{u}; and
each —W′— is independently C₁₋₃ alkylene, —C(═O)—, —N(R^{L})—, —O—, C₃₋₁₂ carbocyclylene, or 3- to 12-membered heterocyclylene, wherein the alkylene, carbocyclylene, or heterocyclylene is optionally substituted with one or more R^{u}.

In certain embodiments, L is of Formula:

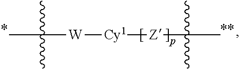

wherein:
W is absent; or
W is C₁₋₃ alkylene, —O—, —NR^{W}—, or —(C═O)—, wherein the alkylene is optionally substituted by one or more R^{u};
Cy¹ is absent; or
Cy¹ is 6-membered heteroarylene, C₆ arylene, C₃₋₁₂ membered carbocyclylene, or 3- to 12-membered heterocyclylene, wherein the arylene, heteroarylene, carbocyclylene, or heterocyclylene is optionally substituted by one or more R^{u};
Z′ is absent; or
each Z′ is independently C₁₋₃ alkylene, —O—, —NR^{W}—, —(C═O)—, C₃₋₁₂ membered carbocyclylene, or 3- to 12-membered heterocyclylene, wherein the alkylene, carbocyclylene, or heterocyclylene is optionally substituted by one or more R^{u};
R^{W} is hydrogen or C₁₋₆ alkyl optionally substituted with one or more R^{u}; and
p is an integer selected from 0 to 8.

T, a ligand for a protein, is a chemical entity that competitively or non-competitively binds a protein.

In certain embodiments, the protein is B7.1 and B7, TINFRIm, TNFR2, NADPH oxidase, BclIBax and other partners in the apotosis pathway, C₅a receptor, HMG-COA reductase, PDE V phosphodiesterase type, PDE IV phosphodiesterase type 4, PDE I, PDEII, PDEIII, squalene cyclase inhibitor, CXCR1, CXCR2, nitric oxide (NO) synthase, cyclo-oxygenase 1, cyclo-oxygenase 2, 5HT receptors, dopamine receptors, G Proteins, i.e., Gq, histamine receptors, 5-lipoxygenase, tryptase serine protease, thymidylate synthase, purine nucleoside phosphorylase, GAPDH trypanosomal, glycogen phosphorylase, Carbonic anhydrase, chemokine receptors, JAW STAT, RXR and similar, HIV 1 protease, HIV 1 integrase, influenza, neuramimidase, hepatitis B reverse transcriptase, sodium channel, multi drug resistance (MDR), protein P-glycoprotein (and MRP), tyrosine kinases, CD23, CD124, tyrosine kinase p56 lck, CD4, CD5, IL-2 receptor, IL-I receptor, TNF-alphaR, ICAM1, Cat+ channels, VC AM, VLA-4 integrin, selectins, CD40/CD40L, newokinins and receptors, inosine monophosphate dehydrogenase, p38 MAP Kinase, RaslRafIMEWERK pathway, interleukin-1 converting enzyme, caspase, HCV, NS3 protease, HCV NS3 RNA helicase, glycinamide ribonucleotide formyl transferase, rhinovirus 3C protease, herpes simplex virus-1 (HSV-I), protease, cytomegalovirus (CMV) protease, poly(ADP-ribose) polymerase, cyclin dependent kinases, vascular endothelial growth factor, oxytocin receptor, microsomal transfer protein inhibitor, bile acid transport inhibitor, 5 alpha reductase inhibitors, angiotensin 11, glycine receptor, noradrenaline reuptake receptor, endothelin receptors, neuropeptide Y and receptor, estrogen receptors, androgen receptors (AR), adenosine receptors, adenosine kinase and AMP deaminase, purinergic receptors (P2Y1, P2Y2, P2Y4, P2Y6, P2X1-7), farnesyl transferases, geranylgeranyl transferase, TrkA a receptor for NGF, beta-amyloid, tyrosine kinase Flk-IIKDR, vitronectin receptor, integrin receptor, Her-21 neu, telomerase inhibition, cytosolic phospholipaseA2 and EGF receptor tyrosine kinase. Additional protein targets include, for example, ecdysone 20-monooxygenase, ion channel of the GABA gated chloride channel, acetylcholinesterase, voltage-sensitive sodium channel protein, calcium release channel, and chloride channels. Still further target proteins include Acetyl-CoA carboxylase, adenylosuccinate synthetase, protoporphyrinogen oxidase, and enolpyruvylshikimate-phosphate synthase.

In certain embodiments, the protein is an androgen receptor (AR), an estrogen receptor (ER), signal transducer and activator of transcription 3 (STAT3), signal transducer and activator of transcription 5 (STAT5), CREB-binding protein/EP300 (E1A) binding protein (CBP/p300), SWI/SNF Related, Matrix Associated, Actin Dependent Regulator Of Chromatin, Subfamily A, Member 2/4 (SMARCA2/4), Kirsten rat sarcoma viral oncogene homolog G12D (KRAS G12D), Src homology region 2-containing protein tyrosine phosphatase 2 (SHP2), or bromodomain-containing protein 4 (BRD4).

In certain embodiments, T is a small molecule.

In certain embodiments, T is a peptide. In certain embodiments, the peptide has about 5 amino acids. In certain embodiments, the peptide has about 10 amino acids. In certain embodiments, the peptide has about 15 amino acids. In certain embodiments, the peptide has about 20 amino acids. In certain embodiments, the peptide has about 25 amino acids. In certain embodiments, the peptide has about 30 amino acids. In certain embodiments, the peptide has about 35 amino acids. In certain embodiments, the peptide has about 40 amino acids. In certain embodiments, the peptide has about 45 amino acids. In certain embodiments, the peptide has about 50 amino acids.

In certain embodiments, T is an antibody.

In certain embodiments, T is a ligand for an estrogen receptor. In certain embodiments, T is ligand for an androgen receptor. In certain embodiments, T is ligand for a STAT1/3 protein.

In certain embodiments, T is an estrogen receptor inhibitor. In certain embodiments, T is an androgen receptor inhibitor. In certain embodiments, T is a STAT1/3 protein inhibitor.

In certain embodiments, the compound is selected from the compounds in Tables 1 and 2 and pharmaceutically acceptable salts thereof.

TABLE 1

| Compound | | |
|---|---|---|
| No. | Structure | Chemical Name |
| A1 | 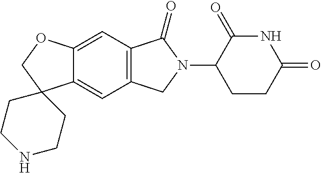 [SMILES: O=C1CCC(N2Cc3cc4c(cc3C2=O)OCC42CCNCC2)C(=O)N1] | 3-(7-oxo-5,7-dihydro-2H,6H- spiro[furo[2,3-f]isoindole-3,4′- piperidin]-6-yl)piperidine-2,6-dione |
| A2 | 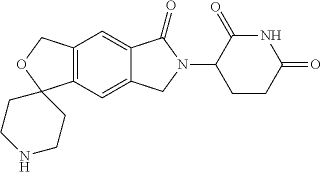 [SMILES: O=C1CCC(N2Cc3cc4c(cc3C2=O)COC42CCNCC2)C(=O)N1] | 3-(5-oxo-5,7-dihydrospiro[furo[3,4- f]isoindole-1,4′-piperidin]-6(3H)- yl)piperidine-2,6-dione |
| A3 | 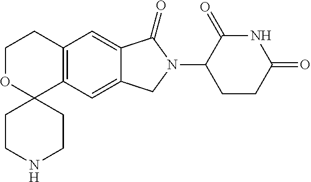 [SMILES: O=C1CCC(N2Cc3cc4c(cc3C2=O)CCOC42CCNCC2)C(=O)N1] | 3-(1′-oxo-1′,3′,7′,8′-tetrahydro-2′H- spiro[piperidine-4,5′-pyrano[3,4- f]isoindol]-2′-yl)piperidine-2,6-dione |
| A4 | 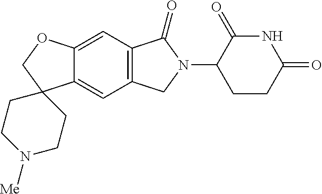 [SMILES: CN1CCC2(CC1)COc1cc3c(cc12)CN(C1CCC(=O)NC1=O)C3=O] | 3-(1′-methyl-7-oxo-5,7-dihydro-2H,6H- spiro[furo[2,3-f]isoindole-3,4′- piperidin]-6-yl)piperidine-2,6-dione |
| A5 | 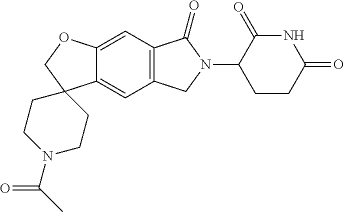 [SMILES: CC(=O)N1CCC2(CC1)COc1cc3c(cc12)CN(C1CCC(=O)NC1=O)C3=O] | 3-(1′-acetyl-7-oxo-5,7-dihydro-2H,6H- spiro[furo[2,3-f]isoindole-3,4′- piperidin]-6-yl)piperidine-2,6-dione |
| A6 | 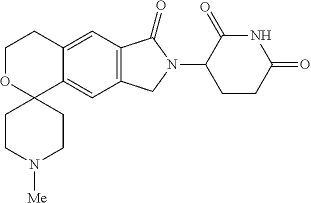 [SMILES: CN1CCC2(CC1)OCCc1cc3c(cc12)CN(C1CCC(=O)NC1=O)C3=O] | 3-(1-methyl-1′-oxo-1′,3′,7′,8′-tetrahydro- 2′H-spiro[piperidine-4,5′-pyrano[3,4- f]isoindol]-2′-yl)piperidine-2,6-dione |
| A7 | 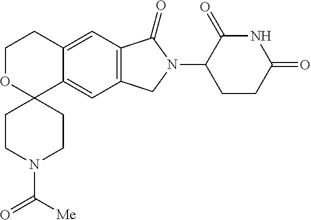 [SMILES: CC(=O)N1CCC2(CC1)OCCc1cc3c(cc12)CN(C1CCC(=O)NC1=O)C3=O] | 3-(1-acetyl-1′-oxo-1′,3′,7′,8′-tetrahydro- 2′H-spiro[piperidine-4,5′-pyrano[3,4- f]isoindol]-2′-yl)piperidine-2,6-dione |
| A8 | 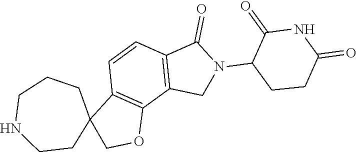 [SMILES: O=C1CCC(N2Cc3c(ccc4c3OCC43CCCNCC3)C2=O)C(=O)N1] | 3-(6′-oxo-6′,8′-dihydro-2′H,7′H- spiro[azepane-4,3′-furo[2,3-e]isoindol]- 7′-yl)piperidine-2,6-dione |
| A9 | 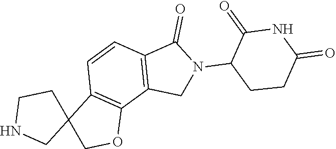 [SMILES: O=C1CCC(N2Cc3c(ccc4c3OCC43CCNC3)C2=O)C(=O)N1] | 3-(6-oxo-6,8-dihydro-2H,7H- spiro[furo[2,3-e]isoindole-3,3′- pyrrolidin]-7-yl)piperidine-2,6-dione |
| A14 | 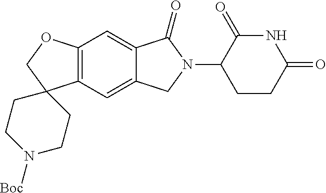 [SMILES: CC(C)(C)OC(=O)N1CCC2(CC1)COc1cc3c(cc12)CN(C1CCC(=O)NC1=O)C3=O] | tert-butyl 6-(2,6-dioxopiperidin-3-yl)-7- oxo-6,7-dihydro-2H,5H-spiro[furo[2,3- f]isoindole-3,4′-piperidine]-1′- carboxylate |
| A15 | 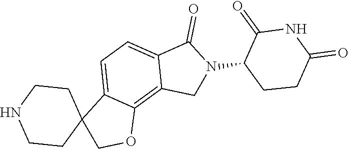 [SMILES: O=C1CC[C@H](N2Cc3c(ccc4c3OCC43CCNCC3)C2=O)C(=O)N1] | (S)-3-(6-oxo-6,8-dihydro-2H,7H- spiro[furo[2,3-e]isoindole-3,4′- piperidin]-7-yl)piperidine-2,6-dione |
| A16 | 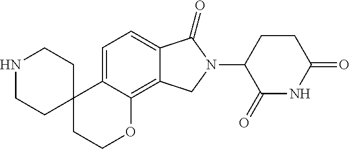 [SMILES: O=C1CCC(N2Cc3c(ccc4c3OCCC43CCNCC3)C2=O)C(=O)N1] | 3-(7′-oxo-2′,3′,7′,9′-tetrahydro-8′H- spiro[piperidine-4,4′-pyrano[2,3- e]isoindol]-8′-yl)piperidine-2,6-dione |
| A17 | 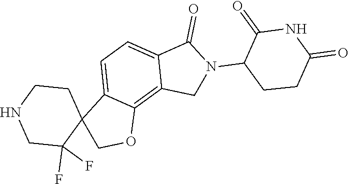 [SMILES: O=C1CCC(N2Cc3c(ccc4c3OCC43CCNCC3(F)F)C2=O)C(=O)N1] | 3-(3′,3′-difluoro-6-oxo-6,8-dihydro- 2H,7H-spiro[furo[2,3-e]isoindole-3,4′- piperidin]-7-yl)piperidine-2,6-dione |
| A18 | 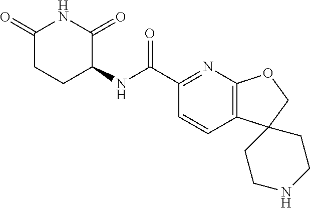 [SMILES: O=C1CC[C@H](NC(=O)c2ccc3c(n2)OCC32CCNCC2)C(=O)N1] | (S)-N-(2,6-dioxopiperidin-3-yl)-2H- spiro[furo[2,3-b]pyridine-3,4′- piperidine]-6-carboxamide |
| A19 | 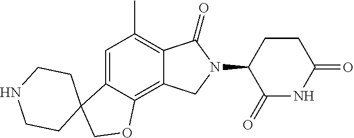 [SMILES: Cc1cc2c(c3c1C(=O)N([C@H]1CCC(=O)NC1=O)C3)OCC21CCNCC1] | (S)-3-(5-methyl-6-oxo-6,8-dihydro- 2H,7H-spiro[furo[2,3-e]isoindole-3,4′- piperidin]-7-yl)piperidine-2,6-dione |
| A20 | 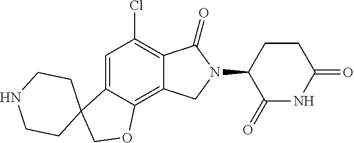 [SMILES: O=C1CC[C@H](N2Cc3c4c(cc(Cl)c3C2=O)C2(CCNCC2)CO4)C(=O)N1] | (S)-3-(5-chloro-6-oxo-6,8-dihydro- 2H,7H-spiro[furo[2,3-e]isoindole-3,4′- piperidin]-7-yl)piperidine-2,6-dione |
| A21 | 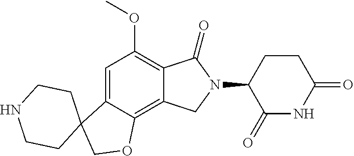 [SMILES: COc1cc2c(c3c1C(=O)N([C@H]1CCC(=O)NC1=O)C3)OCC21CCNCC1] | (S)-3-(5-methoxy-6-oxo-6,8-dihydro- 2H,7H-spiro[furo[2,3-e]isoindole-3,4′- piperidin]-7-yl)piperidine-2,6-dione |
| A22 | 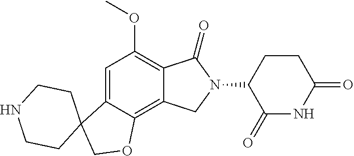 | (R)-3-(5-methoxy-6-oxo-6,8-dihydro- 2H,7H-spiro[furo[2,3-e]isoindole-3,4′- piperidin]-7-yl)piperidine-2,6-dione |

TABLE 2

| Compound | | |
|---|---|---|
| No. | Structure | Chemical Name |
| B0 | 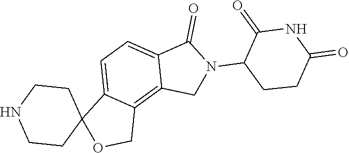 [SMILES: O=C1CCC(N2Cc3c(ccc4c3COC43CCNCC3)C2=O)C(=O)N1] | 3-(6-oxo-6,8-dihydrospiro[furo[3,4- e]isoindole-3,4′-piperidin]-7(1H)- yl)piperidine-2,6-dion |
| B1 | 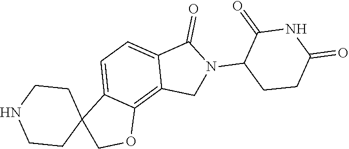 [SMILES: O=C1CCC(N2Cc3c(ccc4c3OCC43CCNCC3)C2=O)C(=O)N1] | 3-(6-oxo-6,8-dihydro-2H,7H- spiro[furo[2,3-e]isoindole-3,4′- piperidin]-7-yl)piperidine-2,6-dione |
| B2 | 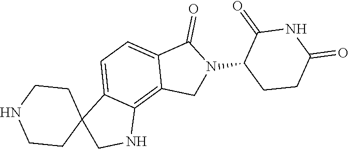 [SMILES: O=C1CC[C@H](N2Cc3c(ccc4c3NCC43CCNCC3)C2=O)C(=O)N1] | (S)-3-(6′-oxo-1′,2′,6′,8′-tetrahydro-7′H- spiro[piperidine-4,3′-pyrrolo[3,4- g]indol]-7′-yl)piperidine-2,6-dione |
| B3 | 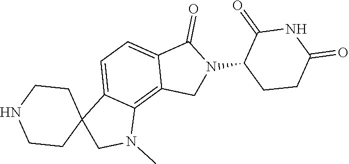 [SMILES: CN1CC2(CCNCC2)c2ccc3c(c21)CN([C@H]1CCC(=O)NC1=O)C3=O] | (S)-3-(1′-methyl-6′-oxo-1′,2′,6′,8′- tetrahydro-7′H-spiro[piperidine-4,3′- pyrrolo[3,4-g]indol]-7′-yl)piperidine- 2,6-dione |
| B4 | 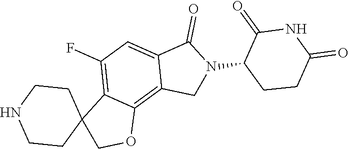 [SMILES: O=C1CC[C@H](N2Cc3c(cc(F)c4c3OCC43CCNCC3)C2=O)C(=O)N1] | (S)-3-(4-fluoro-6-oxo-6,8-dihydro- 2H,7H-spiro[furo[2,3-e]isoindole-3,4′- piperidin]-7-yl)piperidine-2,6-dione |
| B5 | 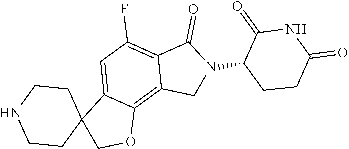 [SMILES: O=C1CC[C@H](N2Cc3c4c(cc(F)c3C2=O)C2(CCNCC2)CO4)C(=O)N1] | (S)-3-(5-fluoro-6-oxo-6,8-dihydro- 2H,7H-spiro[furo[2,3-e]isoindole-3,4′- piperidin]-7-yl)piperidine-2,6-dione |

The compounds of the present disclosure may possess advantageous characteristics, as compared to known compounds, such as known cereblon-binding agents or known degraders comprising such cereblon-binding agents. For example, the compounds of the present disclosure may display more potent cereblon-binding activity or more potent degradation activity against certain proteins, more favorable pharmacokinetic properties (e.g., as measured by Cₘₐₓ, Tₘₐₓ, and/or AUC), and/or less interaction with other cellular targets (e.g., hepatic cellular transporter such as OATP1B¹) and accordingly improved safety (e.g., drug-drug interaction). These beneficial properties of the compounds of the present disclosure can be measured according to methods commonly available in the art, such as methods exemplified herein.

Due to the existence of double bonds, the compounds of the present disclosure may be in cis or trans, or Z or E, configuration. It is understood that although one configuration may be depicted in the structure of the compounds or formulae of the present disclosure, the present disclosure also encompasses the other configuration. For example, the compounds or formulae of the present disclosure may be depicted in cis or trans, or Z or E, configuration.

In one embodiment, a compound of the present disclosure (e.g., a compound of any of the formulae or any individual compounds disclosed herein) is a pharmaceutically acceptable salt. In another embodiment, a compound of the present disclosure (e.g., a compound of any of the formulae or any individual compounds disclosed herein) is a solvate. In another embodiment, a compound of the present disclosure (e.g., a compound of any of the formulae or any individual compounds disclosed herein) is a hydrate.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### Forms of Compounds Disclosed Herein

### Pharmaceutically Acceptable Salts

In certain embodiments, the compounds disclosed herein exist as their pharmaceutically acceptable salts. In certain embodiments, the methods disclosed herein include methods of treating diseases by administering such pharmaceutically acceptable salts. In certain embodiments, the methods disclosed herein include methods of treating diseases by administering such pharmaceutically acceptable salts as pharmaceutical compositions.

In certain embodiments, the compounds described herein possess acidic or basic groups and therefor react with any of a number of inorganic or organic bases, and inorganic and organic acids, to form a pharmaceutically acceptable salt. In certain embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In certain embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺ (C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In certain embodiments, water or oil-soluble or dispersible products are obtained by such quaternization.

### Solvates

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates are within the scope of the invention.

It will also be appreciated by those skilled in organic chemistry that many organic compounds can exist in more than one crystalline form. For example, crystalline form may vary from solvate to solvate. Thus, all crystalline forms or the pharmaceutically acceptable solvates thereof are contemplated and are within the scope of the present invention.

In certain embodiments, the compounds described herein exist as solvates. The present disclosure provides for methods of treating diseases by administering such solvates. The present disclosure further provides for methods of treating diseases by administering such solvates as pharmaceutical compositions.

Solvates contain either stoichiometric or non-stoichiometric amounts of a solvent, such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Isomers/Stereoisomers

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

In certain embodiments, the compounds described herein exist as geometric isomers. In certain embodiments, the compounds described herein possess one or more double bonds. The compounds disclosed herein include all cis, trans, syn, anti, entgegen (E), and zusammen (Z) isomers as well as the corresponding mixtures thereof. All geometric forms of the compounds disclosed herein are contemplated and are within the scope of the invention.

In certain embodiments, the compounds disclosed herein possess one or more chiral centers and each center exists in the R configuration or S configuration. The compounds disclosed herein include all diastereomeric, enantiomeric, and epimeric forms as well as the corresponding mixtures thereof. All diastereomeric, enantiomeric, and epimeric forms of the compounds disclosed herein are contemplated and are within the scope of the invention.

In additional embodiments of the compounds and methods provided herein, mixtures of enantiomers and/or diastereoisomers, resulting from a single preparative step, combination, or interconversion are useful for the applications described herein. In certain embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In certain embodiments, dissociable complexes are preferred. In certain embodiments, the diastereomers have distinct physical properties (e.g., melting points, boiling points, solubilities, reactivity, etc.) and are separated by taking advantage of these dissimilarities. In certain embodiments, the diastereomers are separated by chiral chromatography, or preferably, by separation/resolution techniques based upon differences in solubility. In certain embodiments, the optically pure enantiomer is then recovered, along with the resolving agent.

### Tautomers

In certain embodiments, compounds described herein exist as tautomers. The compounds described herein include all possible tautomers within the formulas described herein.

Tautomers are compounds that are interconvertible by migration of a hydrogen atom, accompanied by a switch of a single bond and an adjacent double bond. In bonding arrangements where tautomerization is possible, a chemical equilibrium of the tautomers will exist. All tautomeric forms of the compounds disclosed herein are contemplated and are within the scope of the invention. The exact ratio of the tautomers depends on several factors, including temperature, solvent, and pH.

### Pharmaceutical Compositions

In certain embodiments, the compound or conjugate described herein is administered as a pure chemical. In certain embodiments, the compound or conjugate described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21st Ed. Mack Pub. Co., Easton, PA (2005)).

Accordingly, the present disclosure provides pharmaceutical compositions comprising a compound or a conjugate described herein, or a pharmaceutically acceptable salt, solvate, or stereoisomer thereof, and a pharmaceutically acceptable excipient.

In certain embodiments, the compound or conjugate provided herein is substantially pure, in that it contains less than about 5%, less than about 1%, or less than about 0.1% of other organic small molecules, such as unreacted intermediates or synthesis by-products that are created, for example, in one or more of the steps of a synthesis method.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

In certain embodiments, the pharmaceutical composition is formulated for oral, topical (including buccal and sublingual), rectal, vaginal, transdermal, parenteral, intrapulmonary, intradermal, intrathecal and epidural and intranasal administration. Parenteral administration includes intramuscular, intravenous, intraarterial, intraperitoneal, or subcutaneous administration. In certain embodiments, the pharmaceutical composition is formulated for intravenous injection, oral administration, inhalation, nasal administration, topical administration, or ophthalmic administration. In certain embodiments, the pharmaceutical composition is formulated for oral administration. In certain embodiments, the pharmaceutical composition is formulated for intravenous injection. In certain embodiments, the pharmaceutical composition is formulated as a tablet, a pill, a capsule, a liquid, an inhalant, a nasal spray solution, a suppository, a suspension, a gel, a colloid, a dispersion, a suspension, a solution, an emulsion, an ointment, a lotion, an eye drop, or an ear drop. In certain embodiments, the pharmaceutical composition is formulated as a tablet.

### Preparation and Characterization of the Compounds

The compounds or conjugates of the present disclosure can be prepared in a number of ways well known to those skilled in the art of organic synthesis. By way of example, the compounds or conjugates of the present disclosure can be synthesized using the methods described below, together with synthetic methods known in the art of synthetic organic chemistry, or variations thereon as appreciated by those skilled in the art. The compounds or conjugates of the present disclosure (i.e., a compound or a conjugate of the present application (e.g., a compound or a conjugate of any of the formulae or any individual compounds disclosed herein)) can be synthesized by following the general synthetic scheme below as well as the steps outlined in the examples, schemes, procedures, and/or synthesis described herein (e.g., Examples).

### General Synthetic Scheme

The compounds or conjugates of the present disclosure can generally be prepared by first preparing pools of intermediates, including a pool of cereblon ligands, a pool of linkers, and a pool of inhibitors, as detailed in the Example section, then followed by subsequent reactions to connect a linker to an inhibitor and a cereblon ligand via metal-catalyzed coupling reactions and reductive amination. Large pool of compounds or conjugates can be prepared by selecting different combinations of cereblon ligands, linkers, and inhibitors from each pool. General synthetic routes for preparing inhibitor-linker conjugate via metal-catalyzed coupling reactions, which is further coupled to cerebon ligand via reductive amination, are summarize below.

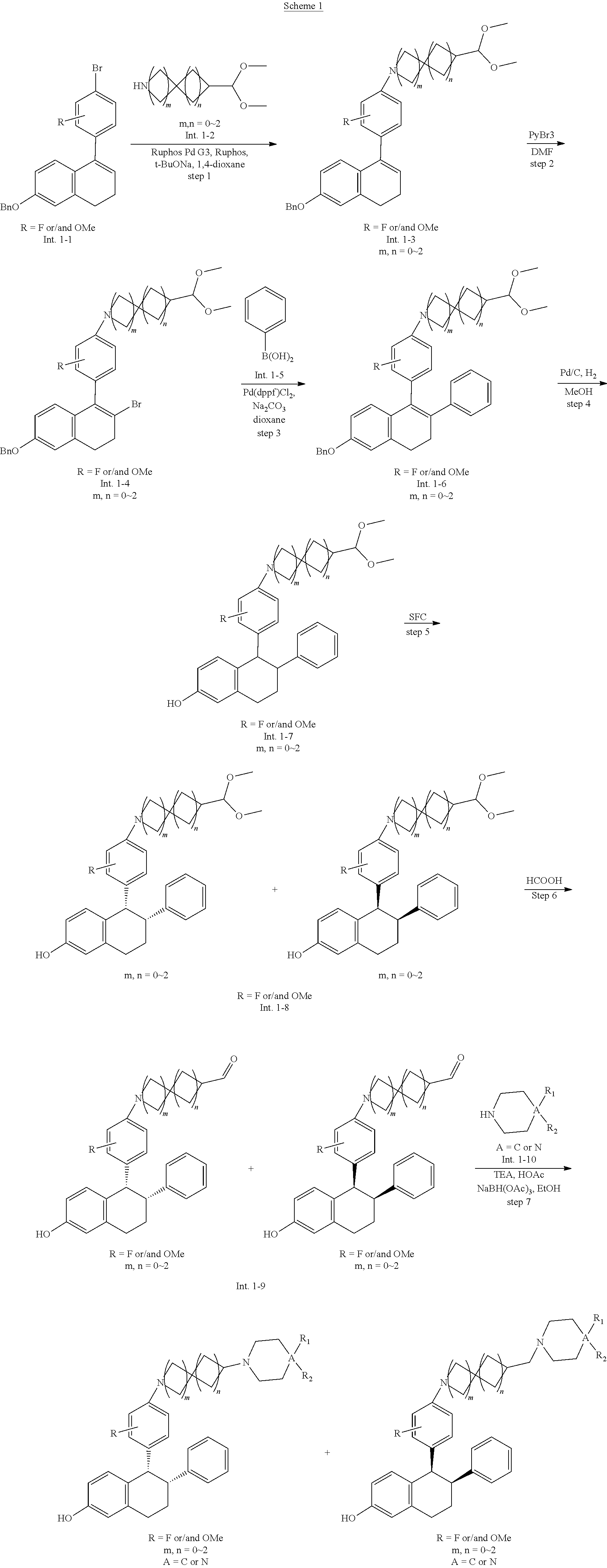

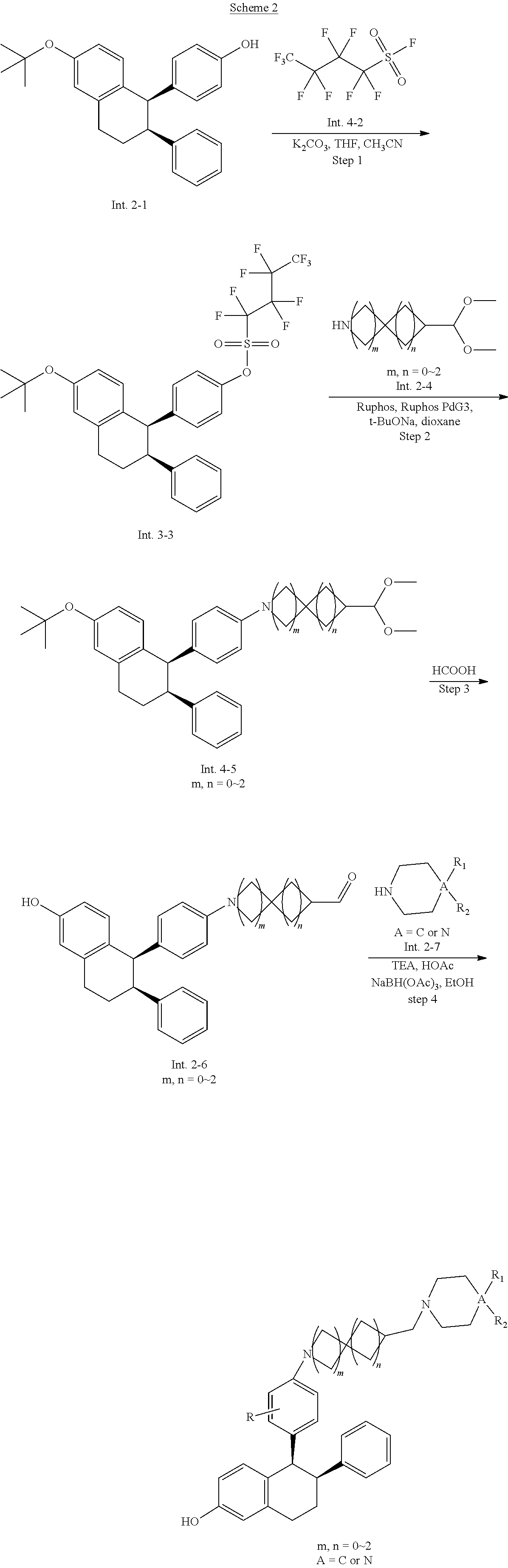

Those skilled in the art will recognize if a stereocenter exists in the compounds of the present disclosure (e.g., a compound of any of the formulae or any individual compounds disclosed herein). Accordingly, the present disclosure includes both possible stereoisomers (unless specified in the synthesis) and includes not only racemic compound but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH, Inc. (Toronto, Canada), Bionet (Cornwall, U.K.), Chem Service Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes. Specific and analogous reactants are optionally identified through the indices of known chemicals prepared by the Chemical Abstract Service of the American Chemical Society, which are available in most public and university libraries, as well as through on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Analytical Methods, Materials, and Instrumentation

Unless otherwise noted, reagents and solvents were used as received from commercial suppliers. Proton nuclear magnetic resonance (NMR) spectra were obtained on either Bruker or Varian spectrometers at 400 MHz. Spectra are given in ppm (δ) and coupling constants, J, are reported in Hertz. Tetramethylsilane (TMS) was used as an internal standard. Liquid chromatography-mass spectrometry (LC/MS) were collected using a SHIMADZU LCMS-2020EV or Agilent 1260-6125B LCMS. Purity and low resolution mass spectral data were measured using Agilent 1260-6125B LCMS system (with Diode Array Detector, and Agilent G6125BA Mass spectrometer) or using Waters Acquity UPLC system (with Diode Array Detector, and Waters 3100 Mass Detector). The purity was characterized by UV wavelength 214 nm, 220 nm, 254 nm and ESI. Column: poroshell 120 EC-C18 2.7 μm 4.6×100 mm; Flow rate 0.8 mL/min; Solvent A (100/0.1 water/formic acid), Solvent B (100 acetonitrile); gradient: hold 5% B to 0.3 min, 5-95% B from 0.3 to 2 min, hold 95% B to 4.8 min, 95-5% B from 4.8 to 5.4 min, then hold 5% B to 6.5 min. Or, column: Acquity UPLC BEH C18 1.7 μm 2.1×50 mm; Flow rate 0.5 mL/min; Solvent A (0.1% formic acid water), Solvent B (acetonitrile); gradient: hold 5% B for 0.2 min, 5-95% B from 0.2 to 2.0 min, hold 95% B to 3.1 min, then 5% B at 3.5 min.

### Biological Assays

The biological activities of the compounds of the present disclosure can be assessed with methods and assays known in the art.

The CRBN-DDB1 binding potency of the present disclosure was determined using HTRF assay technology (Perkin Elmer). Compounds are serially diluted and are transferred multi-well plate. The reaction was conducted with addition of His tagged (e.g., CRBN+DDB-DLS7+CXU4) followed by addition of 60 nM fluorescent probe (e.g., Cy⁵-labeled Thalidomide), and MAb Anti-6HIS Tb cryptate Gold in the assay buffer. After one hour incubation at room temperature, the HTRF signals were read on Envision reader (Perkin Elemer).

ERa degradative activity of compounds can be assessed in MCF-7 and T47D Cells. MCF-7 and T47D cell are seeded and are subsequently treated with the compounds at certain concentrations (e.g., 0.02 to 300 nM). DMSO can be used as vehicle control. Cells are fixed and are blocked with Intercept (PBS) Blocking Buffer (e.g., Li—COR, Odyssey Blocking Buffer), and are stained with ER (e.g., 1:500, Cell signaling) primary antibody for overnight at cold room. Secondary Antibody (e.g., IRDye 800CW Goat anti-Rabbit IgG) and CellTag 700 Stain are added in Intercept (PBS) Blocking Buffer. Finally, cell plate is placed in incubator to dry. Image and signal were captured on Odyssey® DLx Imaging System.

In vitro assay can be accomplished by MCF-7 and T47D Cell Titer Glo (CTG) assay. MCF-7 and T47D cell (From HDB) are cultured in multi-well white plate with phenol red-free RPMI1640+10% CS-FBS+1% P/S medium (e.g., at 1,000 cells/well). On day 0: Cells were treated with compound at certain concentrations (e.g., 0.5 to 10000 nM) (DMSO and Staurosporine as control). On day 0 and day 6: add Cell Titer Glo reagent and read on En Vision after 30 min incubation for data generation.

In-cell western blot analysis. Cells are seeded in multi-well plates (e.g., at 40,000 or 10,000 cells/well). Diluted compounds at certain concentration are added (final 0.5% DMSO) and cells are incubated for certain period of time (e.g., 16 hours). Formaldehyde (e.g., PBS: FA-9:1) is added and followed by washing with PBS. The cells are blocked with Licor blocking buffer (Li-Cor). The relative ER percentage in treated cells were obtained by comparing the values of treated wells to those in untreated and DMSO-treated wells as 100%.

Western Blot Analysis. Cells that are treated with the compounds are lysed in Radioimmunoprecipitation Assay Protein Lysis and Extraction Buffer (e.g., 25 mmol/L Tris.HCl, pH 7.6, 150 mmol/L NaCl, 1% Nonidet P-40, 1% sodium deoxycholate, and 0.1% sodium dodecyl sulfate) containing proteinase inhibitor cocktail. Equal amounts of total protein are electrophoresed through 10% SDS-polyacrylamide gels after determination of protein concentration by BCA assay. The separated protein bands were transferred onto PVDF membranes and blotted against different antibodies. The blots are scanned, and the band intensities were quantified (e.g., by using GelQuant.NET software provided by biochemlabsolutions.com). The relative mean intensity of target proteins is expressed after normalization to the intensity of glyceraldehyde-3-phosphate dehydrogenase bands.

Cell Growth Assay. Cells were seeded at certain concentration (e.g., at 1500/well) in multi-well plates overnight. Cells are subsequently treated with the compounds. A certain period of time (e.g., 4 days) after the compound treatment, 10% WST-8 reagent was added to the culture medium and incubate under certain condiction (e.g., in a CO₂ incubator at 37° C. for 2.5 hours). The absorbance is measured on each sample using a microplate reader at certain wavelength (e.g., 450 nm). The relative absorbance is calculated against the vehicle control from three individually repeats.

In vivo pharmacodynamic and efficacy studies. To develop breast cancer cell line xenografts, mice is given 17β-Estradiol in drinking water for certain period of time. Certain number (e.g., five million) of cells in 50% Matrigel are injected subcutaneously into SCID mice to induce tumor formation. When tumors reach certain size (e.g., 100-400 mm³), mice are treated with vehicle control (e.g., 5% DMSO, 10% solutol, 85% Water) or the compound, and sacrificed at indicated time points. Tumor tissue is harvested for analysis. Tumor sizes and animal weights were measured 2-3 times per week. Tumor volume (mm³)=(lengthxwidth2)/2. Tumor growth inhibition is calculated using TGI (%)=(Vc−Vt)/(Vc−Vo)×100, where Vc, Vt are the median of control and treated groups at the end of the study and Vo at the start.

### Methods of Use

In certain aspects, provided herein are methods of binding cereblon E3 ubiquitin ligase protein complex in a subject or biological sample comprising administering a compound described herein to the subject or contacting the biological sample with a compound described herein.

In certain aspects, provided herein are uses of a compound described herein in the manufacture of a medicament for binding cereblon E3 ubiquitin ligase protein complex in a subject or biological sample.

In certain aspects, provided herein are compounds described herein for use in binding cereblon E3 ubiquitin ligase protein complex in a subject or biological sample.

In certain aspects, provided herein are methods of degrading a protein in a subject or biological sample comprising administering a compound or a conjugate described herein to the subject or contacting the biological sample with a compound described herein.

In certain aspects, provided herein are uses of a compound or a conjugate described herein in the manufacture of a medicament for degrading a protein in a subject or biological sample.

In certain aspects, provided herein are compounds or conjugates described herein for use in degrading a protein in a subject or biological sample.

In certain embodiments, the protein is an estrogen receptor, STAT3 protein, SMARCA2/4 protein, CBP/p300 protein, an androgen receptor, or a BRD9 protein.

In certain aspects, provided herein are methods of treating or preventing a disease or disorder a subject in need thereof, comprising administering to the subject a compound or a conjugate described herein.

In certain aspects, provided herein are uses of a compound or a conjugate described herein in the manufacture of a medicament for treating or preventing a disease or disorder in a subject in need thereof.

In certain aspects, provided herein are compounds or conjugates described herein for use in treating or preventing a disease or disorder in a subject in need thereof.

In certain embodiments, the disease or disorder is an estrogen receptor-mediated disease or disorder, STAT3-mediated disease or disorder, SMARCA2/4-mediated disease or disorder, CBP/p300-mediated disease or disorder, an androgen receptor-mediated disease or disorder, or a BRD9-mediated disease or disorder.

In certain embodiments, the subject is a mammal.

In certain embodiments, the subject is a human.

### Definitions

As used in the specification and appended claims, unless specified to the contrary, the following terms have the meaning indicated below.

### Chemical Definitions

Definitions of specific functional groups and chemical terms are described in more detail below. The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5th Edition, John Wiley &amp; Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPFC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. F. Eliel, Ed., Univ. of Notre Dame Press, Notre Dame, IN 1972).

The invention additionally encompasses compounds described herein as individual isomers substantially free of other isomers, and alternatively, as mixtures of various isomers.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₄, C₄₋₆, C₄₋₅, and C₅₋₆ alkyl.

The following terms are intended to have the meanings presented therewith below and are useful in understanding the description and intended scope of the present invention. When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e., at least one) of the grammatical objects of the article. By way of example “an analogue” means one analogue or more than one analogue.

“Alkyl” as used herein, refers to a radical of a straight-chain or branched saturated hydrocarbon group having from 1 to 20 carbon atoms (“C₁₋₂₀ alkyl”). In certain embodiments, an alkyl group has 1 to 12 carbon atoms (“C₁₋₁₂ alkyl”). In certain embodiments, an alkyl group has 1 to 10 carbon atoms (“C₁₋₁₀ alkyl”). In certain embodiments, an alkyl group has 1 to 9 carbon atoms (“C₁₋₉ alkyl”). In certain embodiments, an alkyl group has 1 to 8 carbon atoms (“C₁₋₈ alkyl”). In certain embodiments, an alkyl group has 1 to 7 carbon atoms (“C₁₋₇ alkyl”). In certain embodiments, an alkyl group has 1 to 6 carbon atoms (“C₁₋₆ alkyl”, which is also referred to herein as “lower alkyl”). In certain embodiments, an alkyl group has 1 to 5 carbon atoms (“C₁₋₅ alkyl”). In certain embodiments, an alkyl group has 1 to 4 carbon atoms (“C₁₋₄ alkyl”). In certain embodiments, an alkyl group has 1 to 3 carbon atoms (“C₁₋₃ alkyl”). In certain embodiments, an alkyl group has 1 to 2 carbon atoms (“C₁₋₂ alkyl”). In certain embodiments, an alkyl group has 1 carbon atom (“C¹ alkyl”). Examples of C₁₋₆ alkyl groups include methyl(C₁), ethyl(C₂), n-propyl(C₃), isopropyl(C₃), n-butyl(C₄), tert-butyl(C₄), sec-butyl(C₄), isobutyl(C₄), n-pentyl(C₅), 3-pentanyl(C₅), amyl(C₅), neopentyl(C₅), 3-methyl-2-butanyl(C₅), tertiary amyl(C₅), and n-hexyl(C₆). Additional examples of alkyl groups include n-heptyl(C₇), n-octyl(C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁-10 alkyl. Common alkyl abbreviations include Me (—CH₃), Et (—CH₂CH₃), i-Pr(—CH(CH₃)₂), n-Pr(—CH₂CH₂CH₃), n-Bu (—CH₂CH₂CH₂CH₃), or i-Bu (—CH₂CH(CH₃)₂).

“Alkylene” as used herein, refers to an alkyl group wherein two hydrogens are removed to provide a divalent radical. When a range or number of carbons is provided for a particular “alkylene” group, it is understood that the range or number refers to the range or number of carbons in the linear carbon divalent chain. An “alkelene” group may be substituted or unsubstituted with one or more substituents as described herein. Exemplary unsubstituted divalent alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted divalent alkylene groups, e.g., substituted with one or more alkyl(methyl) groups, include but are not limited to, substituted methylene (—CH(CH₃)—, (—C(CH₃)₂—), substituted ethylene (—CH(CH₃) CH₂—, —CH₂CH(CH₃)—, —C(CH₃)₂CH₂—, —CH₂C(CH₃)₂—), substituted propylene (—CH(CH₃) CH₂CH₂—, —CH₂CH(CH₃) CH₂—, —CH₂CH₂CH(CH₃)—, —C(CH₃)₂CH₂CH₂—, —CH₂C(CH₃)₂CH₂—, —CH₂CH₂C(CH₃)₂—), and the like.

“Alkenyl” as used herein, refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In certain embodiments, an alkenyl group has 2 to 10 carbon atoms (“C₂₋₁₀ alkenyl”). In certain embodiments, an alkenyl group has 2 to 9 carbon atoms (“C₂-9 alkenyl”). In certain embodiments, an alkenyl group has 2 to 8 carbon atoms (“C₂₋₈ alkenyl”). In certain embodiments, an alkenyl group has 2 to 7 carbon atoms (“C₂₋₇ alkenyl”). In certain embodiments, an alkenyl group has 2 to 6 carbon atoms (“C₂₋₆ alkenyl”). In certain embodiments, an alkenyl group has 2 to 5 carbon atoms (“C₂₋₅ alkenyl”). In certain embodiments, an alkenyl group has 2 to 4 carbon atoms (“C₂₋₄ alkenyl”). In certain embodiments, an alkenyl group has 2 to 3 carbon atoms (“C₂₋₃ alkenyl”). In certain embodiments, an alkenyl group has 2 carbon atoms (“C² alkenyl”). The one or more carbon-carbon double bonds can be internal (such as in 2-butenyl) or terminal (such as in 1-butenyl). Examples of C₂₋₄ alkenyl groups include ethenyl(C₂), 1-propenyl(C₃), 2-propenyl(C₃), 1-butenyl(C₄), 2-butenyl(C₄), butadienyl(C₄), and the like. Examples of C₂₋₆ alkenyl groups include the aforementioned C₂₋₄ alkenyl groups as well as pentenyl(C₅), pentadienyl(C₅), hexenyl(C₆), and the like. Additional examples of alkenyl include heptenyl(C₇), octenyl(C₈), octatrienyl(C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkenylene” as used herein, refers to an alkenyl group wherein two hydrogens are removed to provide a divalent radical. When a range or number of carbons is provided for a particular “alkenylene” group, it is understood that the range or number refers to the range or number of carbons in the linear carbon divalent chain. An “alkenylene” group may be substituted or unsubstituted with one or more substituents as described herein. Exemplary unsubstituted divalent alkenylene groups include, but are not limited to, ethenylene (—CH═CH—) and propenylene (e.g., —CH═CHCH₂—, —CH₂—CH—CH—). Exemplary substituted divalent alkenylene groups, e.g., substituted with one or more alkyl(methyl) groups, include but are not limited to, substituted ethylene (—C(CH₃)═CH—, —CH═C(CH₃)—), substituted propylene (e.g., —C(CH₃)═CHCH₂—, —CH═C(CH₃) CH₂—, —CH═CHCH(CH₃)—, —CH═CHC(CH₃)₂—, —CH(CH₃)—CH═CH—, —C(CH₃)₂—CH═CH—, —CH₂—C(CH₃)═CH—, —CH₂—CH═C(CH₃)—), and the like.

“Alkynyl” as used herein, refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In certain embodiments, an alkynyl group has 2 to 10 carbon atoms (“C₂₋₁₀ alkynyl”). In certain embodiments, an alkynyl group has 2 to 9 carbon atoms (“C₂₋₉ alkynyl”). In certain embodiments, an alkynyl group has 2 to 8 carbon atoms (“C₂-8 alkynyl”). In certain embodiments, an alkynyl group has 2 to 7 carbon atoms (“C₂₋₇ alkynyl”). In certain embodiments, an alkynyl group has 2 to 6 carbon atoms (“C₂₋₆ alkynyl”). In certain embodiments, an alkynyl group has 2 to 5 carbon atoms (“C₂₋₅ alkynyl”). In certain embodiments, an alkynyl group has 2 to 4 carbon atoms (“C₂₋₄ alkynyl”). In certain embodiments, an alkynyl group has 2 to 3 carbon atoms (“C₂₋₃ alkynyl”). In certain embodiments, an alkynyl group has 2 carbon atoms (“C² alkynyl”). The one or more carbon-carbon triple bonds can be internal (such as in 2-butynyl) or terminal (such as in 1-butynyl). Examples of C₂₄ alkynyl groups include, without limitation, ethynyl(C₂), 1-propynyl(C₃), 2-propynyl(C₃), 1-butynyl(C₄), 2-butynyl(C₄), and the like. Examples of C₂₋₆ alkenyl groups include the aforementioned C₂₋₄ alkynyl groups as well as pentynyl(C₅), hexynyl(C₆), and the like. Additional examples of alkynyl include heptynyl(C₇), octynyl(C₈), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

“Alkynylene” as used herein, refers to a linear alkynyl group wherein two hydrogens are removed to provide a divalent radical. When a range or number of carbons is provided for a particular “alkynylene” group, it is understood that the range or number refers to the range or number of carbons in the linear carbon divalent chain. An “alkynylene” group may be substituted or unsubstituted with one or more substituents as described herein. Exemplary divalent alkynylene groups include, but are not limited to, substituted or unsubstituted ethynylene, substituted or unsubstituted propynylene, and the like.

The term “heteroalkyl,” as used herein, refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“heteroC₁₋₁₀ alkyl”). In certain embodiments, a heteroalkyl group is a saturated group having 1 to 9 carbon atoms and 1, 2, 3, or 4 heteroatoms (“heteroC₁₋₉ alkyl”). In certain embodiments, a heteroalkyl group is a saturated group having 1 to 8 carbon atoms and 1, 2, 3, or 4 heteroatoms (“heteroC₁₋₈ alkyl”). In certain embodiments, a heteroalkyl group is a saturated group having 1 to 7 carbon atoms and 1, 2, 3, or 4 heteroatoms (“heteroC₁₋₇ alkyl”). In certain embodiments, a heteroalkyl group is a group having 1 to 6 carbon atoms and 1, 2, or 3 heteroatoms (“heteroC₁₋₆ alkyl”). In certain embodiments, a heteroalkyl group is a saturated group having 1 to 5 carbon atoms and 1 or 2 heteroatoms (“heteroC₁₋₅ alkyl”). In certain embodiments, a heteroalkyl group is a saturated group having 1 to 4 carbon atoms and/or 2 heteroatoms (“heteroC₁₋₄ alkyl”). In certain embodiments, a heteroalkyl group is a saturated group having 1 to 3 carbon atoms and 1 heteroatom (“heteroC₁₋₃ alkyl”). In certain embodiments, a heteroalkyl group is a saturated group having 1 to 2 carbon atoms and 1 heteroatom (“heteroC₁₋₂ alkyl”). In certain embodiments, a heteroalkyl group is a saturated group having 1 carbon atom and 1 heteroatom (“heteroCl₁ alkyl”). In certain embodiments, a heteroalkyl group is a saturated group having 2 to 6 carbon atoms and 1 or 2 heteroatoms (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

The term “heteroalkenyl,” as used herein, refers to an alkenyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkenyl group refers to a group having from 2 to 10 carbon atoms, at least one double bond, and 1, 2, 3, or 4 heteroatoms (“heteroC₂₋₁₀ alkenyl”). In certain embodiments, a heteroalkenyl group has 2 to 9 carbon atoms at least one double bond, and 1, 2, 3, or 4 heteroatoms (“heteroC₂₋₉ alkenyl”). In certain embodiments, a heteroalkenyl group has 2 to 8 carbon atoms, at least one double bond, and 1, 2, 3, or 4 heteroatoms (“heteroC₂₋₈ alkenyl”). In certain embodiments, a heteroalkenyl group has 2 to 7 carbon atoms, at least one double bond, and 1, 2, 3, or 4 heteroatoms (“heteroC₂₋₇ alkenyl”). In certain embodiments, a heteroalkenyl group has 2 to 6 carbon atoms, at least one double bond, and 1, 2, or 3 heteroatoms (“heteroC₂₋₆ alkenyl”). In certain embodiments, a heteroalkenyl group has 2 to 5 carbon atoms, at least one double bond, and 1 or 2 heteroatoms (“heteroC₂₋₅ alkenyl”). In certain embodiments, a heteroalkenyl group has 2 to 4 carbon atoms, at least one double bond, and lor 2 heteroatoms (“heteroC₂₋₄ alkenyl”). In certain embodiments, a heteroalkenyl group has 2 to 3 carbon atoms, at least one double bond, and 1 heteroatom (“heteroC₂₋₃ alkenyl”). In certain embodiments, a heteroalkenyl group has 2 to 6 carbon atoms, at least one double bond, and 1 or 2 heteroatoms (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “heteroalkynyl,” as used herein, refers to an alkynyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms are inserted between a carbon atom and the parent molecule, i.e., between the point of attachment. In certain embodiments, a heteroalkynyl group refers to a group having from 2 to 10 carbon atoms, at least one triple bond, and 1, 2, 3, or 4 heteroatoms (“heteroC₂₋₁₀ alkynyl”). In certain embodiments, a heteroalkynyl group has 2 to 9 carbon atoms, at least one triple bond, and 1, 2, 3, or 4 heteroatoms (“heteroC₂₋₉ alkynyl”). In certain embodiments, a heteroalkynyl group has 2 to 8 carbon atoms, at least one triple bond, and 1, 2, 3, or 4 heteroatoms (“heteroC₂₋₈ alkynyl”). In certain embodiments, a heteroalkynyl group has 2 to 7 carbon atoms, at least one triple bond, and 1, 2, 3, or 4 heteroatoms (“heteroC₂₋₇ alkynyl”). In certain embodiments, a heteroalkynyl group has 2 to 6 carbon atoms, at least one triple bond, and 1, 2, or 3 heteroatoms (“heteroC₂₋₆ alkynyl”). In certain embodiments, a heteroalkynyl group has 2 to 5 carbon atoms, at least one triple bond, and 1 or 2 heteroatoms (“heteroC₂₋₅ alkynyl”). In certain embodiments, a heteroalkynyl group has 2 to 4 carbon atoms, at least one triple bond, and 1 or 2 heteroatoms (“heteroC₂₋₄ alkynyl”). In certain embodiments, a heteroalkynyl group has 2 to 3 carbon atoms, at least one triple bond, and 1 heteroatom (“heteroC₂₋₃ alkynyl”). In certain embodiments, a heteroalkynyl group has 2 to 6 carbon atoms, at least one triple bond, and 1 or 2 heteroatoms (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

Analogous to “alkylene,” “alkenylene,” and “alkynylene” as defined above, “heteroalkylene,” “heteroalkenylene,” and “heteroalkynylene,” as used herein, refer to a divalent radical of heteroalkyl, heteroalkenyl, and heteroalkynyl group respectively. When a range or number of carbons is provided for a particular “heteroalkylene,” “heteroalkenylene,” or “heteroalkynylene,” group, it is understood that the range or number refers to the range or number of carbons in the linear divalent chain. “Heteroalkylene,” “heteroalkenylene,” and “heteroalkynylene” groups may be substituted or unsubstituted with one or more substituents as described herein.

“Aryl” refers to a radical of a monocyclic or polycyclic (e.g., bicyclic or tricyclic)4n+2 aromatic ring system (e.g., having 6, 10, or 14× electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₋₄ aryl”; e.g., anthracyl).

Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particular aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆-14 aryl.

“Arylene” as used herein, refers to an aryl group wherein two hydrogens are removed to provide a divalent radical. When a range or number of carbons is provided for a particular “arylene” group, it is understood that the range or number refers to the range or number of carbons in the aryl group. An “arylene” group may be substituted or unsubstituted with one or more substituents as described herein.

“Heteroaryl” refers to a radical of a 5- to 14-membered monocyclic or polycyclic 4n+2 aromatic ring system (e.g., having 6, 10, or 14× electrons shared in a cyclic array) having ring carbon atoms and 1-8 ring heteroatoms provided in the aromatic ring system, wherein each heteroatom is independently selected from nitrogen, oxygen and sulfur (“5- to 14-membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings.

“Heteroaryl” also includes ring systems wherein the heteroaryl group, as defined above, is fused with one or more aryl groups wherein the point of attachment is either on the heteroaryl or the one or more aryl groups, and in such instances, the number of ring members designates the total number of ring members in the fused (aryl/heteroaryl) ring system. When substitution is indicated in such instances, unless otherwise specified, substitution can occur on either the heteroaryl or the one or more aryl groups. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In certain embodiments, a heteroaryl is a 5- to 10-membered aromatic ring system having ring carbon atoms and 1-4 ring heteroatoms provided in the aromatic ring system, wherein each heteroatom is independently selected from nitrogen, oxygen, and sulfur (“5- to 10-membered heteroaryl”). In certain embodiments, a heteroaryl is a 5- to 9-membered aromatic ring system having ring carbon atoms and 1-4 ring heteroatoms provided in the aromatic ring system, wherein each heteroatom is independently selected from nitrogen, oxygen, and sulfur (“5- to 9-membered heteroaryl”). In certain embodiments, a heteroaryl is a 5- to 8-membered aromatic ring system having ring carbon atoms and 1-4 ring heteroatoms provided in the aromatic ring system, wherein each heteroatom is independently selected from nitrogen, oxygen, and sulfur (“5- to 8-membered heteroaryl”). In certain embodiments, a heteroaryl group is a 5- to 6-membered aromatic ring system having ring carbon atoms and 1-4 ring heteroatoms provided in the aromatic ring system, wherein each heteroatom is independently selected from nitrogen, oxygen, and sulfur (“5- to 6-membered heteroaryl”). In certain embodiments, the 5- to 6-membered heteroaryl has 1-3 ring heteroatoms independently selected from nitrogen, oxygen, and sulfur. In certain embodiments, the 5- to 6-membered heteroaryl has 1-2 ring heteroatoms independently selected from nitrogen, oxygen, and sulfur. In certain embodiments, the 5- to 6-membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5- to 14-membered heteroaryl. In certain embodiments, the heteroaryl group is substituted 5- to 14-membered heteroaryl.

Exemplary 5-membered heteroaryl containing one heteroatom include, without limitation, pyrrolyl, furanyl and thiophenyl. Exemplary 5-membered heteroaryl containing two heteroatoms include, without limitation, imidazolyl, pyrazolyl, oxazolyl, isoxazolyl, thiazolyl, and isothiazolyl. Exemplary 5-membered heteroaryl containing three heteroatoms include, without limitation, triazolyl, oxadiazolyl, and thiadiazolyl. Exemplary 5-membered heteroaryl containing four heteroatoms include, without limitation, tetrazolyl. Exemplary 6-membered heteroaryl containing one heteroatom include, without limitation, pyridinyl. Exemplary 6-membered heteroaryl containing two heteroatoms include, without limitation, pyridazinyl, pyrimidinyl, and pyrazinyl. Exemplary 6-membered heteroaryl containing three or four heteroatoms include, without limitation, triazinyl and tetrazinyl, respectively. Exemplary 7-membered heteroaryl containing one heteroatom include, without limitation, azepinyl, oxepinyl, and thiepinyl. Exemplary 5,6-bicyclic heteroaryl include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl include, without limitation, naphthyridinyl, pteridinyl, quinolinyl, isoquinolinyl, cinnolinyl, quinoxalinyl, phthalazinyl, and quinazolinyl.

“Heteroarylene” as used herein, refers to a heteroaryl group wherein two hydrogens are removed to provide a divalent radical. When a range or number of ring members is provided for a particular “heteroarylene” group, it is understood that the range or number refers to the number of ring members in the heteroaryl group. A “heteroarylene” group may be substituted or unsubstituted with one or more substituents as described herein.

“Carbocyclyl” refers to a radical of a non-aromatic cyclic hydrocarbon group having from 3 to 12 ring carbon atoms (“C₃₋₁₂ carbocyclyl”) and zero heteroatoms in the nonaromatic ring system. In certain embodiments, a carbocyclyl group has 3 to 10 ring carbon atoms (“C₃₋₁₀ carbocyclyl”). In certain embodiments, a carbocyclyl group has 3 to 8 ring carbon atoms (“C₃₋₈ carbocyclyl”). In certain embodiments, a carbocyclyl group has 3 to 6 ring carbon atoms (“C₃₋₆ carbocyclyl”). In certain embodiments, a carbocyclyl group has 5 to 12 ring carbon atoms (“C₅₋₁₂ carbocyclyl”). In certain embodiments, a carbocyclyl group has 5 to 10 ring carbon atoms (“C₅₋₁₀ carbocyclyl”). In certain embodiments, a carbocyclyl group has 5 to 8 ring carbon atoms (“C₅₋₈ carbocyclyl”). In certain embodiments, a carbocyclyl group has 5 or 6 ring carbon atoms (“C₅₋₆ carbocyclyl”). Exemplary C₃₋₆ carbocyclyl include, without limitation, cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), and the like. Exemplary C₃₋₈ carbocyclyl include, without limitation, the aforementioned C₃₋₆ carbocyclyl groups as well as cycloheptyl(C₇), cycloheptenyl(C₇), cycloheptadienyl(C₇), cycloheptatrienyl(C₇), cyclooctyl(C₈), cyclooctenyl(C₈), bicyclo[2.2.1]heptanyl(C₇), bicyclo[2.2.2]octanyl(C₈), and the like. Exemplary C₃₋₁₀ carbocyclyl include, without limitation, the aforementioned C₃₋₈ carbocyclyl groups as well as cyclononyl(C₉), cyclononenyl(C₉), cyclodecyl(C₁₀), cyclodecenyl(C₁₀), octahydro-1H-indenyl(C₉), decahydronaphthalenyl(C₁₀), spiro[4.5]decanyl(C₁₀), and the like.

In certain embodiments, “carbocyclyl” is a monocyclic, saturated carbocyclyl group having from 3 to 12 ring carbon atoms (“C₃₋₁₂ carbocyclyl”). In certain embodiments, “carbocyclyl” is a monocyclic, saturated carbocyclyl group having from 3 to 10 ring carbon atoms (“C₃₋₁₀ carbocyclyl”). In certain embodiments, “carbocyclyl” is a monocyclic, saturated carbocyclyl group having from 3 to 8 ring carbon atoms (“C₃₋₈ carbocyclyl”). In certain embodiments, “carbocyclyl” is a monocyclic, saturated carbocyclyl group having from 3 to 6 ring carbon atoms (“C₃₋₆ carbocyclyl”). In certain embodiments, “carbocyclyl” is a monocyclic, saturated carbocyclyl group having from 5 to 12 ring carbon atoms (“C₅₋₁₂ carbocyclyl”). In certain embodiments, a carbocyclyl group has 5 to 10 ring carbon atoms (“C₅₋₁₀ carbocyclyl”). In certain embodiments, a carbocyclyl group has 5 to 8 ring carbon atoms (“C₅₋₈ carbocyclyl”). In certain embodiments, “carbocyclyl” is a monocyclic, saturated carbocyclyl group having 5 or 6 ring carbon atoms (“C₅₋₆ carbocyclyl”). Examples of C₅₋₆ carbocyclyl include cyclopentyl(C₅) and cyclohexyl(C₅). Examples of C₃₋₆ carbocyclyl include the aforementioned C₅₋₆ carbocyclyl groups as well as cyclopropyl(C₃) and cyclobutyl(C₄). Examples of C₃₋₈ carbocyclyl include the aforementioned C₃₋₆ carbocyclyl groups as well as cycloheptyl(C₇) and cyclooctyl(C₈). Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is unsubstituted C₃₋₁₂ carbocyclyl. In certain embodiments, the carbocyclyl group is substituted C₃₋₁₂ carbocyclyl.

As the foregoing examples illustrate, in certain embodiments, the carbocyclyl group is either monocyclic (“monocyclic carbocyclyl”) or polycyclic (“polycyclic carbocyclyl”) that contains a fused, bridged or spiro ring system and can be saturated or can be partially unsaturated. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₂ carbocyclyl. In certain embodiments, the carbocyclyl group is a substituted C₃₋₁₂ carbocyclyl.

“Fused carbocyclyl” or “fused carbocycle” refers to ring systems wherein the carbocyclyl group, as defined above, is fused with, i.e., share two common atoms (as such, share one common bond), one or more carbocyclyl groups, as defined above, wherein the point of attachment is on any of the fused rings. In such instances, the number of carbons designates the total number of carbons in the fused ring system. When substitution is indicated, unless otherwise specified, substitution can occur on any of the fused rings.

“Spiro carbocyclyl” or or “spiro carbocycle” refers to ring systems wherein the carbocyclyl group, as defined above, form spiro structure with, i.e., share one common atom with, one or more carbocyclyl groups, as defined above, wherein the point of attachment is on the carbocyclyl rings in which the spiro structure is embedded. In such instances, the number of carbons designates the total number of carbons of the carbocyclyl rings in which the spiro structure is embedded. When substitution is indicated, unless otherwise specified, substitution can occur on the carbocyclyl rings in which the spiro structure is embedded.

“Bridged carbocyclyl” or or “bridged carbocycle” refers to ring systems wherein the carbocyclyl group, as defined above, form bridged structure with, i.e., share more than two atoms (as such, share more than one bonds) with, one or more carbocyclyl groups, as defined above, wherein the point of attachment is on any of the carbocyclyl rings in which the bridged structure is embedded. In such instances, the number of carbons designates the total number of carbons of the carbocyclyl rings in which the bridged structure is embedded. When substitution is indicated, unless otherwise specified, substitution can occur on any of the carbocyclyl rings in which the bridged structure is embedded.

“Carbocyclylene” as used herein, refers to a carbocyclyl group wherein two hydrogens are removed to provide a divalent radical. The divalent radical may be present on different atoms or the same atom of the carbocyclylene group. When a range or number of carbons is provided for a particular “carbocyclyl” group, it is understood that the range or number refers to the range or number of carbons in the carbocyclyl group. A “carbocyclyl” group may be substituted or unsubstituted with one or more substituents as described herein.

“Heterocyclyl” refers to a radical of a 3- to 12-membered non-aromatic ring system having ring carbon atoms and 1 to 4 ring heteroatoms, wherein each heteroatom is independently selected from nitrogen, oxygen, sulfur, boron, phosphorus, and silicon (“3- to 12-membered heterocyclyl”). In heterocyclyl groups that contain one or more nitrogen atoms, the point of attachment can be a carbon or nitrogen atom, as valency permits. Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5 membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

In certain embodiments, a heterocyclyl group is a 5- to 12-membered non-aromatic ring system having ring carbon atoms and 1-4 ring heteroatoms, wherein each heteroatom is independently selected from nitrogen, oxygen, sulfur, boron, phosphorus, and silicon (“5- to 12-membered heterocyclyl”). In certain embodiments, a heterocyclyl group is a 5- to 10-membered non-aromatic ring system having ring carbon atoms and 1-4 ring heteroatoms, wherein each heteroatom is independently selected from nitrogen, oxygen, sulfur, boron, phosphorus, and silicon (“5- to 10-membered heterocyclyl”). In certain embodiments, a heterocyclyl group is a 5- to 8-membered non-aromatic ring system having ring carbon atoms and 1-4 ring heteroatoms, wherein each heteroatom is independently selected from nitrogen, oxygen, and sulfur (“5- to 8-membered heterocyclyl”). In certain embodiments, a heterocyclyl group is a 5- to 6-membered non-aromatic ring system having ring carbon atoms and 1-4 ring heteroatoms, wherein each heteroatom is independently selected from nitrogen, oxygen, and sulfur (“5- to 6-membered heterocyclyl”). In certain embodiments, the 5- to 6-membered heterocyclyl has 1-3 ring heteroatoms selected from nitrogen, oxygen, and sulfur. In certain embodiments, the 5- to 6-membered heterocyclyl has 1-2 ring heteroatoms selected from nitrogen, oxygen, and sulfur. In certain embodiments, the 5- to 6-membered heterocyclyl has one ring heteroatom selected from nitrogen, oxygen, and sulfur.

As the foregoing examples illustrate, in certain embodiments, a heterocyclyl group can either be monocyclic (“monocyclic heterocyclyl”) or polycyclic (“polycyclic heterocyclyl”) that contains a fused, bridged or spiro ring system, and can be saturated or can be partially unsaturated. Heterocyclyl polycyclic ring systems can include one or more heteroatoms in one or both rings. “Heterocyclyl” also includes ring systems wherein the heterocyclyl group, as defined above, is fused with one or more carbocyclyl groups wherein the point of attachment is either on the carbocyclyl or heterocyclyl ring, and in such instances, the number of ring members designates the total number of ring members in the entire ring system. When substitution is indicated in such instances, unless otherwise specified, substitution can occur on either the heterocyclyl or the one or more carbocyclyl groups. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3- to 12-membered heterocyclyl. In certain embodiments, the heterocyclyl group is substituted 3- to 12-membered heterocyclyl.

“Fused heterocyclyl” or “fused heterocycle” refers to ring systems wherein the heterocyclyl group, as defined above, is fused with, i.e., share two common atoms (as such, share one common bond) with, one or more heterocyclyl or carbocyclyl groups, as defined above, wherein the point of attachment is on any of the fused rings. In such instances, the number of ring members designates the total number of ring members in the fused ring system. When substitution is indicated, unless otherwise specified, substitution can occur on any of the fused rings.

“Spiro heterocyclyl” or “spiro heterocycle” refers to ring systems wherein the heterocyclyl group, as defined above, form spiro structure with, i.e., share one common atom with, one or more heterocyclyl or carbocyclyl groups, as defined above, wherein the point of attachment is on the heterocyclyl or carbocyclyl rings in which the spiro structure is embedded. In such instances, the number of ring members designates the total number of ring members of the heterocyclyl or carbocyclyl rings in which the spiro structure is embedded. When substitution is indicated, unless otherwise specified, substitution can occur on any of the heterocyclyl or carbocyclyl rings in which the spiro structure is embedded.

“Bridged heterocyclyl” or “bridged heterocycle” refers to ring systems wherein the heterocyclyl group, as defined above, form bridged structure with, i.e., share more than two atoms (as such, share more than one bonds) with, one or more heterocyclyl or carbocyclyl groups, as defined above, wherein the point of attachment is on the heterocyclyl or carbocyclyl rings in which the bridged structure is embedded. In such instances, the number of ring members designates the total number of ring members of the heterocyclyl or carbocyclyl rings in which the bridged structure is embedded. When substitution is indicated, unless otherwise specified, substitution can occur on any of the heterocyclyl or carbocyclyl rings in which the bridged structure is embedded.

“Heterocyclylene” as used herein, refers to a heterocyclyl group wherein two hydrogens are removed to provide a divalent radical. The divalent radical may be present on different atoms or the same atom of the heterocyclylene group. When a range or number of ring members is provided for a particular “heterocyclylene” group, it is understood that the range or number refers to the number of ring members in the heterocyclylene group. A “heterocyclylene” group may be substituted or unsubstituted with one or more substituents as described herein.

“Alkoxy” as used herein, refers to the group-OR, wherein R is alkyl as defined herein. C₁₋₆ alkoxy refers to the group-OR, wherein each R is C₁₋₆ alkyl, as defined herein. Exemplary C₁₋₆ alkyl is set forth above.

“Alkylamino” as used herein, refers to the group —NHR or —NR₂, wherein each R is independently alkyl, as defined herein. C₁₋₆ alkylamino refers to the group —NHR or —NR₂, wherein each R is independently C₁₋₆ alkyl, as defined herein. Exemplary C₁₋₆ alkyl is set forth above.

“Oxo” refers to ═O. When a group other than aryl and heteroaryl or an atom is substituted with an oxo, it is meant to indicate that two geminal radicals on that group or atom form a double bond with an oxygen radical. When a heteroaryl is substituted with an oxo, it is meant to indicate that a resonance structure/tautomer involving a heteroatom provides a carbon atom that is able to form two geminal radicals, which form a double bond with an oxygen radical.

“Halo” or “halogen” refers to fluoro (F), chloro(Cl), bromo (Br), and iodo (I). In certain embodiments, the halo group is either fluoro or chloro.

“Protecting group” as used herein is art-recognized and refers to a chemical moiety introduced into a molecule by chemical modification of a functional group (e.g., hydroxyl, amino, thio, and carboxylic acid) to obtain chemoselectivity in a subsequent chemical reaction, during which the unmodified functional group may not survive or may interfere with the chemical reaction. Common functional groups that need to be protected include but not limited to hydroxyl, amino, thiol, and carboxylic acid. Accordingly, the protecting groups are termed hydroxyl-protecting groups, amino-protecting groups, thiol-protecting groups, and carboxylic acid-protecting groups, respectively.

Common types of hydroxyl-protecting groups include but not limited to ethers (e.g., methoxymethyl (MOM), β-Methoxyethoxymethyl (MEM), tetrahydropyranyl (THP), p-methoxyphenyl (PMP), t-butyl, triphenylmethyl(Trityl), allyl, and benzyl ether (Bn)), silyl ethers (e.g., t-butyldiphenylsilyl (TBDPS), trimethylsilyl (TMS), triisopropylsilyl (TIPS), tri-iso-propylsilyloxymethyl (TOM), and t-butyldimethylsilyl (TBDMS)), and esters (e.g., pivalic acid ester (Piv) and benzoic acid ester (benzoate; Bz)).

Common types of amino-protecting groups include but not limited to carbamates (e.g., t-butyloxycarbonyl (Boc), 9-fluorenylmethyloxycarbonyl (Fmoc), p-methoxybenzyl carbonyl(Moz or MeOZ), 2,2,2-trichloroehtoxycarbonyl(Troc), and benzyl carbamate (Cbz)), esters (e.g., acetyl(Ac); benzoyl(Bz), trifluoroacetyl, and phthalimide), amines (e.g, benzyl (Bn), p-methoxybenzyl (PMB), p-methoxyphenyl (PMP), and triphenylmethyl(trityl)), and sulfonamides (e.g., tosyl(Ts), N-alkyl nitrobenzenesulfonamides (Nosyl), and 2-nitrophenylsulfenyl(Nps)).

Common types of thiol-protecting groups include but not limited to sulfide (e.g., p-methylbenzyl(Meb), t-butyl, acetamidomethyl(Acm), and triphenylmethyl(Trityl)).

Common types of carboxylic acid-protecting groups include but not limited to esters (e.g., methyl ester, triphenylmethyl(Trityl), i-butyl ester, benzyl ester (Bn), S-f-butyl ester, silyl esters, and orthoesters) and oxazoline.

These and other exemplary substituents are described in more detail in the Detailed Description, Examples, and claims. The invention is not intended to be limited in any manner by the above exemplary listing of substituents.

### Other Definitions

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, chlorobenzenesulfonic acid, 2-naphthalenesulfonic acid, 4-toluenesulfonic acid, camphorsulfonic acid, 4-methylbicyclo [2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium potassium, calcium, magnesium, ammonium, tetraalkylammonium, and the like; and when the compound contains a basic functionality, salts of nontoxic organic or inorganic acids, such as hydrochloride, hydrobromide, tartrate, mesylate, acetate, maleate, oxalate and the like.

The term “pharmaceutically acceptable cation” refers to an acceptable cationic counterion of an acidic functional group. Such cations are exemplified by sodium, potassium, calcium, magnesium, ammonium, tetraalkylammonium cations, and the like (see, e.g., Berge, et al., J. Pharm. Sci. 66 (1): 1-79 (January 77).

“Pharmaceutically acceptable vehicle” refers to a diluent, adjuvant, excipient or carrier with which a compound of the disclosure is administered.

“Pharmaceutically acceptable metabolically cleavable group” refers to a group which is cleaved in vivo to yield the parent molecule of the structural formula indicated herein. Examples of metabolically cleavable groups include —COR, —COOR, —CONR₂ and —CH₂OR radicals, where R is selected independently at each occurrence from alkyl, trialkylsilyl, carbocyclic aryl or carbocyclic aryl substituted with one or more of alkyl, halogen, hydroxy or alkoxy. Specific examples of representative metabolically cleavable groups include acetyl, methoxycarbonyl, benzoyl, methoxymethyl and trimethylsilyl groups.

The term “prodrug,” as used in this disclosure, means a compound which is convertible in vivo by metabolic means (e.g., by hydrolysis) to a disclosed compound.

Since prodrugs may enhance numerous desirable qualities of pharmaceuticals (e.g., solubility, bioavailability, manufacturing, etc.), the compounds of the present disclosure (e.g., a compound of any of the formulae or any individual compounds disclosed herein), or pharmaceutically acceptable salts, solvates, stereoisomers, or tautomers thereof can be delivered in prodrug form. Thus, the present disclosure is intended to cover prodrugs of a compound of the present disclosure (e.g., a compound of any of the formulae or any individual compounds disclosed herein), or a pharmaceutically acceptable salt, solvate, stereoisomer, or tautomer thereof, methods of delivering the same and compositions containing the same. “Prodrugs” are intended to include any covalently bonded carriers that release an active parent drug of the present disclosure in vivo when such prodrug is administered to a mammalian subject. Prodrugs are prepared by modifying functional groups present in the compound in such a way that the modifications are cleaved, either in routine manipulation or in vivo, to the parent compound. Prodrugs include compounds of the disclosure wherein a hydroxyl or amino, group is bonded to any group that, when the prodrug of the present disclosure is administered to a mammalian subject, it cleaves to form a free hydroxyl or free amino group, respectively. Examples of prodrugs include, but are not limited to, acetate, formate, and benzoate derivatives of alcohol and amine functional groups in the compounds of each of the formulae described herein or a pharmaceutically acceptable salt, solvate, stereoisomer, or tautomer thereof.

A “subject” to which administration is contemplated includes, but is not limited to, humans (i.e., a male or female of any age group, e.g., a pediatric subject (e.g, infant, child, adolescent) or an adult subject (e.g., young adult, middle aged adult or senior adult) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

An “effective amount” means the amount of a compound that, when administered to a subject for treating or preventing a disease, is sufficient to affect such treatment or prevention. The “effective amount” can vary depending on the compound, the disease and its severity, and the age, weight, etc., of the subject to be treated. A “therapeutically effective amount” refers to the effective amount for therapeutic treatment. A “prophylatically effective amount” refers to the effective amount for prophylactic treatment.

“Preventing”, “prevention” or “prophylactic treatment” refers to a reduction in risk of acquiring or developing a disease or disorder (i.e., causing at least one of the clinical symptoms of the disease not to develop in a subject not yet exposed to a disease-causing agent, or in a subject who is predisposed to the disease in advance of disease onset).

The term “prophylaxis” is related to “prevention,” and refers to a measure or procedure the purpose of which is to prevent, rather than to treat or cure a disease. Non limiting examples of prophylactic measures may include the administration of vaccines; the administration of low molecular weight heparin to hospital patients at risk for thrombosis due, for example, to immobilization, and the administration of an anti-malarial agent such as chloroquine, in advance of a visit to a geographical region where malaria is endemic or the risk of contracting malaria is high.

“Treating” or “treatment” or “therapeutic treatment” of any disease or disorder refers, in one embodiment, to ameliorating the disease or disorder (i.e., arresting the disease or reducing the manifestation, extent or severity of at least one of the clinical symptoms thereof). In another embodiment, “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

The term “about” when referring to a number or a numerical range means that the number or numerical range referred to is an approximation within experimental variability or within statistical experimental error, and thus the number or numerical range, in some instances, will vary between 1% and 15% of the stated number or numerical range. In certain embodiments, the number or numerical range vary by 1%, 2%, 3%, 4%, 5%, 6%, 7%, 8%, 9%, 10%, 11%, 12%, 13%, 14%, or 15% of the stated number or numerical range. In certain embodiments, the number or numerical range vary by 1%, 2%, 3%, 4%, or 5% of the stated number or numerical range. In certain embodiments, the number or numerical range vary by 1%, 2%, or 3% of the stated number or numerical range.

The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” may refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### I. Cereblon Ligands

### · Synthetic Routes and Procedures

### Compound A1:2. 3-(7-Oxo-5,7-Dihydro-2H,6H-Spiro[Furo [2,3-f]Isoindole-3,4′-Piperidin]-6-Yl) Piperidine-2,6-Dione

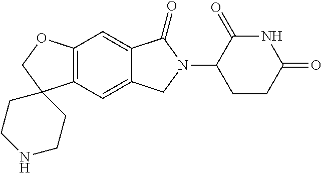 [SMILES: O=C1CCC(N2Cc3cc4c(cc3C2=O)OCC42CCNCC2)C(=O)N1]

### Step A: 4-Bromo-5-Hydroxy-2-Methylbenzoic Acid

To a solution of 5-hydroxy-2-methylbenzoic acid (5.0 g, 32.9 mmol, 1.0 eq) in a mixture of ethanol (20 mL) and acetic acid (10 mL) was added dropwise bromine (3.4 mL, 65.7 mmol, 2.0 equiv). The reaction mixture was stirred for 10 h at room temperature, quenched with aqueous sodium thiosulfate solution (50 mL), and concentrated. The aqueous layer was extracted with ethyl acetate (50 mL×3). The organic layer was dried over magnesium sulfate, filtered and concentrated under reduced pressure to get crude 4-bromo-5-hydroxy-2-methylbenzoic acid (7.6 g, yield 100%) as a white solid. The crude product was directly used in next step without further purification. LC-MS(ESI): mass calcd. for C₈H₇BrO₃, 229.96; m/z found, 231.2 [M+H]⁺.

### Step B: Methyl 4-Bromo-5-Hydroxy-2-Methylbenzoate

Con. H₂SO₄ (12 mL) was added to a suspension of 4-bromo-5-hydroxy-2-methylbenzoic acid (15 g, 65.72 mmol) in methanol (100 mL). The mixture was refluxed for 16 h. After evaporation, the residue was diluted with water (100 mL) and extracted with EA (100 mL×3). The organic layer was washed with H₂O (100 mL×2), saturated aqueous NaHCO₃ solution (100 mL×2) and brine (100 mL). The organic layer was dried over anhydrous Na₂SO₄, filtered and concentrated under reduced pressure. The residue purified by flash column chromatography on silica gel (PE/EA=4/1) to afford methyl 4-bromo-5-hydroxy-2-methylbenzoate (7.5 g, yield 47%) as a colorless solid. LC-MS(ESI): mass calcd. for C₉H₉BrO₃, 243.97; m/z found, 245.2 [M+H]⁺. ¹H NMR (400 MHZ, CDCl₃)δ 7.56 (s, 1H), 7.36 (s, 1H), 5.52 (s, 1H), 3.88 (s, 3H), 2.50 (s, 3H).

### Step C: 1-Benzyl-4-(Hydroxymethyl)Pyridin-1-Ium Bromide

To a solution of (pyridin-4-yl) methanol (8.9 g, 81.6 mmol, 1.0 eq) in CH₃CN (80 mL) was added a solution of (bromomethyl)benzene (11.705 mL, 97.9 mmol, 1.2 eq) in CH₃CN (40 mL). The reaction mixture was refluxed stirred at 90° C. for 3 h. After evaporation, the residue was slurried with methyl tert-butyl ether, filtered, and dried to afford 1-benzyl-4-(hydroxymethyl)pyridin-1-ium bromide (16.33 g, yield 100%) as a yellow solid. LC-MS(ESI): mass calcd. for C₁₃H₁₄NO, 200.11; m/z found, 200.3 [M]⁺

### Step D: (1-Benzyl-1,2,3,6-Tetrahydropyridin-4-Yl) Methanol

To a solution of 1-benzyl-4-(hydroxymethyl)pyridin-1-ium bromide (16.3 g, 81.4 mmol, 1.0 eq) in CH₃OH (150 mL) was added NaBH₄ (9.3 g, 244.2 mmol, 3.0 eq) in portions at −20° C. The mixture was stirred at −20° C. for 1 h. The reaction was quenched with brine (100 mL) and extracted with EtOAc (200 mL×3). The organic layer was washed with brine (100 mL×3), dried over anhydrous Na₂SO₄, filtered and concentrated under reduced pressure. The residue was purified by flash column chromatography on silica gel (CH₃OH in DCM, from 0% to 10%) to afford (1-benzyl-1,2,3,6-tetrahydropyridin-4-yl) methanol (15 g, yield 91%) as a red oil. LC-MS(ESI): mass calcd. for C₁₃H₁₇NO, 203.13; m/z found, 204.4 [M+H]⁺. ¹H NMR (400 MHZ, DMSO-d₆) § 7.24-7.18 (m, 4H), 7.16-7.12 (m, 1H), 5.43 (s, 1H), 4.61 (s, 1H), 3.71 (s, 2H), 3.42 (s, 2H), 2.76 (s, 2H), 2.39 (t, J=5.6 Hz, 2H), 1.91 (s, 2H).

### Step E: Methyl 5-[(1-Benzyl-1,2,3,6-Tetrahydropyridin-4-Yl) Methoxy]-4-Bromo-2-Methylbenzoate

To a solution of methyl 4-bromo-5-hydroxy-2-methylbenzoate (200 mg, 0.82 mmol, 1.0 eq), (1-benzyl-1,2,3,6-tetrahydropyridin-4-yl) methanol (166 mg, 0.82 mmol, 1.0 eq), and PPh₃ (321 mg, 1.22 mmol, 1.5 eq) in dry THF (10 mL) was added dropwise DIAD (0.25 mL, 1.22 mmol. 1.5 eq) at 0° C. under the N₂ atmosphere. The solution was stirred for 2 h. After evaporation, the residue was purified by flash column chromatography on silica gel (PE/EA=2/1 to 1/1) to afford methyl 5-[(1-benzyl-1,2,3,6-tetrahydropyridin-4-yl) methoxy]-4-bromo-2-methylbenzoate (300 mg, yield 85%) as a white solid. LC-MS(ESI): mass calcd. for C₂₂H₂₄BrNO₃, 429.09; m/z found, 431.30 [M+H]⁺.

### Step F: Methyl 1′-(Cyclohexylmethyl)-5-Methyl-2H-Spiro[1-Benzofuran-3,4′-Piperidine]-6-Carboxylate

Tributyl tin hydride (0.5 mL, 1.84 mmol, 4.0 equiv) was added to a solution of methyl 5-[(1-benzyl-1,2,3,6-tetrahydropyridin-4-yl) methoxy]-4-bromo-2-methylbenzoate (200 mg, 0.46 mmol, 1.0 eq) and AIBN (15 mg, 0.09 mmol, 0.2 eq) in toluene (10 mL). The solution was refluxed in a sealed tube for 6 h. After cooled down to room temperature, The solution was quenched with saturated potassium fluoride solution (40 mL) and stirred at room temperature for 0.5 h. The mixture was extracted with EA (40 mL×3). The organic layer was washed brine (40 mL), dried over anhydrous Na₂SO₄, filtered and concentrated under reduced pressure. The residue was purified by prep-TLC (EA/PE=1/1) to afford methyl 1′-(cyclohexylmethyl)-5-methyl-2H-spiro[1-benzofuran-3,4′-piperidine]-6-carboxylate (20 mg, yield 43%) as a yellow solid. LC-MS(ESI): mass calcd. for C₂₂H₂₅NO₃, 351.18; m/z found, 352.30 [M+H]⁺.

¹H NMR (400 MHZ, CDCl₃)δ 7.37-7.27 (m, 6H), 6.99 (s, 1H), 4.37 (s, 2H), 3.85 (s, 3H), 3.54 (s, 2H), 2.89 (d, J=10.2 Hz, 2H), 2.52 (s, 3H), 2.10-1.95 (m, 4H), 1.70 (d, J=11.4 Hz, 2H).

### Step G: Methyl 5-Methyl-2H-Spiro[Benzofuran-3,4′-Piperidine]-6-Carboxylate

A mixture of methyl 1′-benzyl-5-methyl-2H-spiro[1-benzofuran-3,4′-piperidine]-6-carboxylate (1.0 g, 2.845 mmol, 1.0 eq), acetic acid (1 mL, 5.7 mmol, 6.1 eq), and 10% Pd/C (200 mg) in MeOH (20 mL) was stirred at 50° C. under H₂ (1 atm) for 3 h. After filtration, the filtrate was concentrated to get methyl 5-methyl-2H-spiro[benzofuran-3,4′-piperidine]-6-carboxylate (970 mg, yield 100%) as a colorless oil, which was directly used in the next step without further purification. LC-MS(ESI): mass calcd. for C₁₅H₁₉NO₃, 261.14; m/z found, 262.40 (M+H)⁺.

### Step H: 1′-(Tert-Butyl)6-Methyl 5-Methyl-2H-Spiro[Benzofuran-3,4′-Piperidine]-1′,6-Dicarboxylate

To a stirred solution of methyl 5-methyl-2H-spiro[1-benzofuran-3,4′-piperidine]-6-carboxylate (970 mg, 3.7 mmol, 1.0 eq) and TEA (1 mL, 7.4 mmol, 2.0 eq) in DCM (10 mL) was added dropwise Boc₂O (0.8 mL, 3.7 mmol, 2.0 eq) at 0° C. The mixture was stirred at room temperature for 2 h. The reaction mixture was poured into water (10 mL) and extracted with DCM (30 mL×2). The organic phase was dried over anhydrous Na₂SO₄ and concentrated under reduced pressure to afford 1′-(tert-butyl)6-methyl 5-methyl-2H-spiro[benzofuran-3,4′-piperidine]-1′,6-dicarboxylate (1.28 g, yield 100%) as a white solid. LC-MS(ESI): mass calcd. for C₂₀H₂₇NO₅, 361.19; m/z found, 306.4 [M+H-56]+.

### Step I: 1′-(Tert-Butyl)6-Methyl 5-(Bromomethyl)-2H-Spiro[Benzofuran-3,4′-Piperidine]-1′,6-Dicarboxylate

A mixture of methyl 1′-benzyl-5-methyl-2H-spiro[1-benzofuran-3,4′-piperidine]-6-carboxylate (220 mg, 0.609 mmol, 1 eq), NBS(130 mg, 0.73 mmol, 1.2 eq), and BPO (60 mg, 0.243 mmol, 0.4 eq) in CCl₄ (10 mL) was refluxed for 4 h. After cooled to room temperature, the mixture was filtered, then the filtration was concentrated and to give 1′-tert-butyl 6-methyl 5-(bromomethyl)-2H-spiro[1-benzofuran-3,4′-piperidine]-1′,6-dicarboxylate (100 mg, yield 37%) as a light-yellow solid. LC-MS(ESI): mass calcd. for C₂₀H₂₆BrNO₅, 439.10; m/z found, 462.20, [M+Na]⁺.

### Step J: Tert-Butyl 6-(2,6-Dioxopiperidin-3-Yl)-7-Oxo-6,7-Dihydro-2H,5H-Spiro[Furo [2,3-Flisoindole-3,4′-Piperidine]-1′-Carboxylate

DIPEA (0.12 mL, 0.681 mmol, 3.0 eq) was added to 1′-tert-butyl 6-methyl 5-(bromomethyl)-2H-spiro[1-benzofuran-3,4′-piperidine]-1′,6-dicarboxylate (100 mg, 0.227 mmol, 1.0 eq) and 3-aminopiperidine-2,6-dione hydrochloride (56 mg, 0.341 mmol, 1.5 eq) in MeCN (5 mL) under nitrogen. The resulting suspension was stirred at 80° C. for 24 h. The reaction mixture was cooled to room temperature and filtered. The solid was washed with MeCN and purified by prep-TLC (100% EtOAc) to afford tert-butyl 6-(2,6-dioxopiperidin-3-yl)-7-oxo-6,7-dihydro-2H,5H-spiro[furo [2,3-f]isoindole-3,4′-piperidine]-1′-carboxylate (50 mg, yield 48%) as a white solid. LC-MS(ESI): mass calcd. for C₂₄H₂₉N₃O₆, 455.51; m/z found, 456.50, (M+H)⁺.

### Step K. 3-(7-Oxo-5,7-Dihydro-2H,6H-Spiro[Furo [2,3-f]Isoindole-3,4′-Piperidin]-6-Yl) Piperidine-2,6-Dione

To a solution of tert-butyl 6-(2,6-dioxopiperidin-3-yl)-7-oxo-2,5,6,7-tetrahydrospiro[furo [2,3-f]isoindole-3,4′-piperidine]-1′-carboxylate (50 mg, 0.11 mmol, 1.0 eq) in DCM (1 mL) was added HCl-dioxane solution (4 M, 1 mL, 4 mmol, 36 eq) and the mixture was stirred for 30 min. After evaporation, the residue was purified by prep-HPLC with YMC-TA C18 (5 μm, 20×250 mm), and mobile phase of 5-95% MeCN in water over 10 min, and then hold at 100% ACN for 2 min, at a flow rate of 25 mL/min to get 3-{7-oxo-2,5,6,7-tetrahydrospiro[furo [2,3-f]isoindole-3,4′-piperidine]-6-yl}piperidine-2,6-dione hydrochloride (30 mg, yield 70%) as a white solid. LC-MS(ESI): mass calcd. for C₁₉H₂₁N₃O₄, 355.19; m/z found, 356.20 [M+H]⁺.

¹H NMR (400 MHZ, DMSO-d₆) δ 10.98 (s, 1H), 8.78 (s, 2H), 7.36 (s, 1H), 7.06 (s, 1H), 5.11-5.06 (m, 1H), 4.58 (s, 2H), 4.38 (d, J=17.0 Hz, 1H), 4.25 (d, J=17.0 Hz, 1H), 3.30-3.27 (m, 2H), 3.04-2.92 (m, 2H), 2.93-2.84 (m, 1H), 2.62-2.56 (m, 1H), 2.44-2.29 (m, 1H), 2.09-1.97 (m, 3H), 1.90-1.79 (m, 2H).

### Compound A2:3-{5-Oxo-3,5,6,7-Tetrahydrospiro[Furo [3,4-f]Isoindole-1,4′-Piperidine]-6-Yl}Piperidine-2,6-Dione

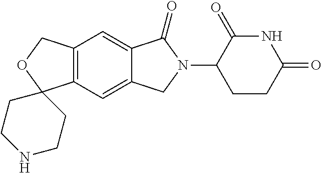 [SMILES: O=C1CCC(N2Cc3cc4c(cc3C2=O)COC42CCNCC2)C(=O)N1]

### Step A: 5-Bromo-6-Iodo-1,3-Dihydro-2-Benzofuran-1-One

To a solution of 5-bromo-1,3-dihydro-2-benzofuran-1-one (25 g, 117.35 mmol, 1 eq) in TFA (250 mL) and TfOH (25 mL) was added NIS(30.45 g, 176 mmol, 1.5 eq) at 0° C. in portions. The mixture was allowed to warm to room temperature and stirred overnight. The reaction mixture was poured into ice-water (500 mL) and yellow solid precipitated. The mixture was filtered and the filter cake was washed with aqueous Na₂S₂O₃ (300 mL×3). The filter cake was suspend in EA (250 mL) and stirred for 1 h. After filtration, the cake was dried to afford 5-bromo-6-iodo-1,3-dihydro-2-benzofuran-1-one (11 g, yield 28%) as a white solid. The filtrate was concentrated under reduced pressure to afford 5-bromo-4-iodo-1,3-dihydro-2-benzofuran-1-one (6 g, yield 15%) as a yellow solid. LC-MS(ESI): mass calcd. for C₈H₄BrIO₂, 337.84; m/z found, 338.85 [M+H]⁺.

5-bromo-6-iodo-1,3-dihydro-2-benzofuran-1-one: ¹H NMR (400 MHZ, DMSO-d₆) δ 8.31 (s, 1H), 8.11 (s, 1H), 5.33 (s, 2H).

5-bromo-4-iodo-1,3-dihydro-2-benzofuran-1-one: ¹H NMR (400 MHZ, DMSO-d₆) δ 7.92 (d, J=8.0 Hz, 1H), 7.78 (d, J=8.0 Hz, 1H), 5.20 (s, 2H).

### Step B: 5-Bromo-6-Ethenyl-1,3-Dihydro-2-Benzofuran-1-One

To a stirred solution of 5-bromo-6-iodo-1,3-dihydro-2-benzofuran-1-one (10 g, 29.51 mmol, 1.0 eq) and Potassium vinyltrifluoroborate (5.93 g, 44.26 mmol, 1.5 eq) in dioxane (250 mL) and H₂O (50 mL) was added Pd(dppf) Cl₂ (2.16 g, 2.95 mmol, 0.1 eq) and K₂CO₃ (12.23 g, 88.51 mmol, 3.0 eq). The reaction mixture was stirred under N₂ at 70° C. overnight. After cooled to room temperature, the mixture was filtered and extracted with EtOAc (150 mL×3). The combined organic layers were washed with brine (200 mL), dried over anhydrous Na₂SO₄ and concentrated under reduced pressure. The residue was purified by flash column chromatography on silica gel (PE/EtOAc=1/1) to give 5-bromo-6-ethenyl-1,3-dihydro-2-benzofuran-1-one (2.6 g, yield 37%) as a brown solid. LC-MS(ESI): mass calcd. for C₁₀H₇BrO₂, 237.96; m/z found, 238.97 [M+H]⁺.

### Step C: 6-Bromo-3-Oxo-1,3-Dihydro-2-Benzofuran-5-Carbaldehyde

To a stirred mixture of 5-bromo-6-ethenyl-1,3-dihydro-2-benzofuran-1-one (2.1 g, 8.78 mmol, 1.0 eq) in acetone (20 mL) and H₂O (10 mL) were added K₂OsO₄: 2H₂O (0.32 g, 0.88 mmol, 0.1 eq) and NMO (2.06 g, 17.57 mmol, 2.0 eq). The resulting mixture was stirred at room temperature for 1 h. NaIO₄ (4.51 g, 21.08 mmol, 2.5 eq) was added to above mixture and the resulting mixture was stirred at room temperature for 2 h. The reaction mixture was diluted with H₂O (50 mL) and extracted with EtOAc (50 mL×3). The combined organic layers were washed with brine (50 mL×3), dried over anhydrous Na₂SO₄ and concentrated. The residue was purified by flash column chromatography on silica gel (PE/EtOAc=1/1) to give 6-bromo-3-oxo-1,3-dihydro-2-benzofuran-5-carbaldehyde (1.1 g, yield 52%) as a white solid. LC-MS(ESI): mass calcd. for C₉H₅BrO₃, 239.94; m/z found, 240.95 [M+H]⁺. ¹H NMR (400 MHZ, DMSO-d₆) δ 10.28 (s, 1H), 8.19 (s, 1H), 8.16 (s, 1H), 5.49 (s, 2H).

### Step D: 5-Bromo-6-(Hydroxymethyl)-1,3-Dihydro-2-Benzofuran-1-One

To a stirred mixture of 6-bromo-3-oxo-1,3-dihydro-2-benzofuran-5-carbaldehyde (1 g, 4.15 mmol, 1.0 eq) in THF (15 mL) was added NaBH₄ (0.47 g, 12.45 mmol, 3.0 eq). The resulting mixture was stirred at room temperature for 1 h. The reaction mixture was quenched with ice-water (50 mL) and extracted with EtOAc (80 mL×3). The combined organic layers were washed with brine (100 mL), dried over anhydrous Na₂SO₄ and concentrated. The residue was purified by flash column chromatography on silica gel (PE/EtOAc=1/1) to give 5-bromo-6-(hydroxymethyl)-1,3-dihydro-2-benzofuran-1-one (700 mg, yield 69%) as a white solid. LC-MS(ESI): mass calcd. for C₉H₇BrO₃, 241.96; m/z found, 242.97 [M+H]⁺.

### Step E: Benzyl 4-[6-(Hydroxymethyl)-1-Oxo-1,3-Dihydro-2-Benzofuran-5-Yl]-1,2,3,6-Tetrahydropyridine-1-Carboxylate

To a stirred solution of 5-bromo-6-(hydroxymethyl)-1,3-dihydro-2-benzofuran-1-one (1.2 g, 4.94 mmol, 1.0 eq) and benzyl 4-(tetramethyl-1,3,2-dioxaborolan-2-yl)-1,2,3,6-tetrahydropyridine-1-carboxylate (1.69 g, 4.94 mmol, 1.0 eq) in dioxane (20 mL) and H₂O (2 mL) were added Pd(dppf) Cl₂ (0.36 g, 0.49 mmol, 0.1 eq) and K₂CO₃ (2.05 g, 14.81 mmol, 3.0 eq). The reaction mixture was stirred under nitrogen atmosphere at 90° C. for 2 h. After cooled to room temperature, the reaction mixture was filtered, and the cake was washed with EA (30 mL). The mixture was diluted with H₂O (40 mL) and extracted with EtOAc (100 mL×3). The combined organic layers were washed with brine (100 mL), dried over anhydrous Na₂SO₄ and concentrated. The residue was purified by flash column chromatography on silica gel (PE/EtOAc=1/1) to give benzyl 4-[6-(hydroxymethyl)-1-oxo-1,3-dihydro-2-benzofuran-5-yl]-1,2,3,6-tetrahydropyridine-1-carboxylate (1 g, yield 53%) as a white solid. LC-MS(ESI): mass calcd. for C₂₂H₂₁NO₅, 379.14; m/z found, 380.15 [M+H]⁺.

### Step F: Benzyl 3′-Bromo-5-Oxo-5,7-Dihydro-3H-Spiro[Benzo[1,2-c: 4,5-c′]Difuran-1,4′-Piperidine]-1′-Carboxylate

To a stirred mixture of benzyl 4-[6-(hydroxymethyl)-1-oxo-1,3-dihydro-2-benzofuran-5-yl]-1,2,3,6-tetrahydropyridine-1-carboxylate (1 g, 2.64 mmol, 1.0 eq) in MeCN (15 mL) was added NBS(0.7 g, 3.95 mmol, 1.5 eq). The resulting mixture was stirred at room temperature for 1 h. The reaction mixture was quenched with saturated aqueous Na₂S₂O₃ solution (30 mL) and extracted with EtOAc (50 mL×3). The combined organic layers were washed with H₂O (50 mL), dried over anhydrous Na₂SO₄ and concentrated. The residue was purified by flash column chromatography on silica gel (PE/EtOAc=1/1) to give benzyl 3′-bromo-5-oxo-5,7-dihydro-3H-spiro[benzo[1,2-c: 4,5-c′]difuran-1,4′-piperidine]-1′-carboxylate (945 mg, yield 78%) as a white solid. LC-MS(ESI): mass calcd. for C₂₂H₂₀BrNO₅, 457.05; m/z found, 458.06 [M+H]⁺.

### Step G: Benzyl 5-Oxo-5,7-Dihydro-3H-Spiro[Benzo[1,2-c: 4,5-c′]Difuran-1,4′-Piperidine]-1′-Carboxylate

To a stirred mixture of benzyl 3′-bromo-5-oxo-5,7-dihydro-3H-spiro[benzo[1,2-c: 4,5-c′]difuran-1,4′-piperidine]-1′-carboxylate (945 mg, 2.06 mmol, 1.0 eq) in toluene (20 mL) were added AIBN (0.610 mL, 4.12 mmol, 2.0 eq) and n-Bu₃SnH (3.0 g, 10.31 mmol, 5.0 eq). The resulting mixture was stirred at 85° c. overnight. After cooled to room temperature, the reaction mixture was quenched with aqueous KF solution (50 mL), stirred for 1 h, and extracted with EtOAc (100 mL×3). The combined organic layers were washed with brine (100 mL), dried over anhydrous Na₂SO₄ and concentrated. The residue was purified by flash column chromatography on silica gel (PE/EtOAc=2/1) to give benzyl 5-oxo-5,7-dihydro-3H-spiro[benzo[1,2-c: 4,5-c′]difuran-1,4′-piperidine]-1′-carboxylate (611 mg, yield 78%) as a white solid. LC-MS(ESI): mass calcd. for C₂₂H₂₁NO₅, 379.14; m/z found, 380.15 [M+H]⁺. ¹H NMR (400 MHZ, DMSO-d₆) δ 7.76 (s, 1H), 7.60 (s, 1H), 7.40-7.32 (m, 5H), 5.40 (s, 2H), 5.12 (s, 2H), 5.09 (s, 2H), 4.07-4.04 (m, 2H), 3.17 (s, 2H), 1.95-1.87 (m, 2H), 1.72-1.68 (m, 2H).

### Step H: 1′-[(Benzyloxy) Carbonyl]-6-(Hydroxymethyl)-3H-Spiro[2-Benzofuran-1,4′-Piperidine]-5-Carboxylic Acid

To a stirred mixture of benzyl 5-oxo-5,7-dihydro-3H-spiro[benzo[1,2-c: 4,5-c′]difuran-1,4′-piperidine]-1′-carboxylate (650 mg, 1.71 mmol, 1.0 eq) in THF (5 mL), MeOH (5 mL) and H₂O (5 mL) was added sodium hydroxide (342.64 mg, 8.57 mmol, 4.0 eq). The resulting mixture was stirred at room temperature for 2 h. The reaction mixture was adjusted to pH4-5 with diluted aqueous HCl solution (1 N) and extracted with EtOAc (50 mL×3). The combined organic layers were dried over anhydrous Na₂SO₄ and concentrated. The residue was purified by flash column chromatography on silica gel (PE/EtOAc=1/1) to give 1′-[(benzyloxy) carbonyl]-6-(hydroxymethyl)-3H-spiro[2-benzofuran-1,4′-piperidine]-5-carboxylic acid (610 mg, yield 89%) as a white solid. LC-MS: 398 (M+H)⁺. Revised as the following: LC-MS(ESI): mass calcd. for C₂₂H₂₃NO₆, 397.15; m/z found, 398.16 [M+H]⁺.

### Step I: 1′-[(Benzyloxy) Carbonyl]-6-Formyl-3H-Spiro[2-Benzofuran-1,4′-Piperidine]-5-Carboxylic Acid

To a stirred solution of 1′-[(benzyloxy) carbonyl]-6-(hydroxymethyl)-3H-spiro[2-benzofuran-1,4′-piperidine]-5-carboxylic acid (610 mg, 1.54 mmol, 1.0 eq) in DCM (15 mL) was add active MnO₂ (1334.36 mg, 15.35 mmol, 10 eq). The reaction mixture was stirred at room temperature for 2 h. The reaction mixture was filtered and the MnO₂ cake was washed with DCM (30 mL×3). The combined filtrates were concentrated to afford 1′-[(benzyloxy) carbonyl]-6-formyl-3H-spiro[2-benzofuran-1,4′-piperidine]-5-carboxylic acid (600 mg, yield 99%) as a white solid. LC-MS(ESI): mass calcd. for C₂₂H₂₁NO₆, 395.14; m/z found, 396.14 [M+H]⁺.

### Step J: 1′-[(Benzyloxy) Carbonyl]-6-{[(2,6-Dioxopiperidin-3-Yl)Amino]Methyl}-3H-Spiro[2-Benzofuran-1,4′-Piperidine]-5-Carboxylic Acid

To a stirred solution of 1′-[(benzyloxy) carbonyl]-6-formyl-3H-spiro[2-benzofuran-1,4′-piperidine]-5-carboxylic acid (600 mg, 1.52 mmol, 1.0 eq) and 3-Amino-2,6-piperidinedione hydrochloride (374.63 mg, 2.28 mmol, 1.5 eq) in MeOH (10 mL) was added NaOAc (186.64 mg, 2.28 mmol, 1.5 eq) and the reaction mixture was stirred at room temperature for 40 min. NaBH₃CN (286.79 mg, 4.55 mmol, 3.0 eq) was added to above mixture and the resulting reaction mixture was stirred at room temperature for 2 h. After evaporation, the residue was purified by Prep-TLC (DCM/MeOH=to obtain 1′-[(benzyloxy) carbonyl]-6-{[(2,6-dioxopiperidin-3-yl)amino]methyl}-3H-spiro[2-benzofuran-1,4′-piperidine]-5-carboxylic acid (550 mg, yield 71%) as a white solid. LC-MS(ESI): mass calcd. for C₂₇H₂₉N₃O₇, 507.20; m/z found, 508.21 [M+H]⁺.

### Step K: Benzyl 6-(2,6-Dioxopiperidin-3-Yl)-5-Oxo-3,5,6,7-Tetrahydrospiro[Furo [3,4-f]Isoindole-1,4′-Piperidine]-1′-Carboxylate

To a stirred solution of 1′-[(benzyloxy) carbonyl]-6-{[(2,6-dioxopiperidin-3-yl)amino]methyl}-3H-spiro[2-benzofuran-1,4′-piperidine]-5-carboxylic acid (550 mg, 1.08 mmol, 1.0 eq) in DMF (8 mL) were added HATU (494.15 mg, 1.30 mmol, 1.2 eq) and DIEA (0.72 mL, 4.34 mmol, 4.0 eq). The reaction mixture was stirred at room temperature for 3 h. After evaporation, the residue was purified by flash column chromatography on silica gel (DCM/MeOH=10/1) to obtain benzyl 6-(2,6-dioxopiperidin-3-yl)-5-oxo-3,5,6,7-tetrahydrospiro[furo [3,4-f]isoindole-1,4′-piperidine]-1′-carboxylate (250 mg, yield 47%) as a white solid. LC-MS(ESI): mass calcd. for C₂₇H₂₇N₃O₆, 489.19; m/z found, 490.20 [M+H]⁺.

### Step L: 3-{5-Oxo-3,5,6,7-Tetrahydrospiro[Furo [3,4-f]Isoindole-1,4′-Piperidine]-6-Yl}Piperidine-2,6-Dione

To a stirred solution of benzyl 6-(2,6-dioxopiperidin-3-yl)-5-oxo-3,5,6,7-tetrahydrospiro[furo [3,4-f]isoindole-1,4′-piperidine]-1′-carboxylate (200 mg, 0.41 mmol, 1.0 eq) in TFE (8 mL) was add 10% Pd/C (100 mg) and the reaction mixture was stirred under H₂ (1 atm) at 40° C. overnight. After evaporation, the filtrate was concentrated to afford 3-{5-oxo-3,5,6,7-tetrahydrospiro[furo [3,4-f]isoindole-1,4′-piperidine]-6-yl}piperidine-2,6-dione (100 mg, yield 69%) as a white solid. LC-MS(ESI): mass calcd. for C₁₉H₂₁N₃O₄, 355.15; m/z found, 356.16 [M+H]⁺.

### Compound A3:3-(1′-Oxo-1′,3′,7′,8′-Tetrahydro-2′H-Spiro[Piperidine-4,5′-Pyrano[3,4-f]Isoindol]-2′-Yl) Piperidine-2,6-Dione

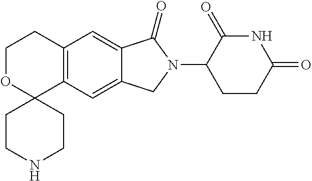 [SMILES: O=C1CCC(N2Cc3cc4c(cc3C2=O)CCOC42CCNCC2)C(=O)N1]

### Step A: 5-Bromo-6-Ethenyl-1,3-Dihydro-2-Benzofuran-1-One

To a solution of 5-bromo-6-iodo-1,3-dihydro-2-benzofuran-1-one (8.6 g, 25.37 mmol, 1.0 eq) and potassium vinyltrifluoroborate (5.10 g, 38.06 mmol, 1.5 eq) in dioxane (200 mL) and H₂O (40 mL) were added Pd(dppf) Cl₂ (1.86 g, 2.54 mmol, 0.1 eq) and K₂CO₃ (10.52 g, 76.12 mmol, 3.0 eq). The reaction mixture was stirred under N₂ at 70° C. overnight. After cooled to room temperature, the mixture was filtered, and the filtrate was extracted with EtOAc (150 mL×3). The combined organic layers were washed with brine (200 mL), dried over anhydrous Na₂SO₄ and concentrated under reduced pressure. The residue was purified by flash column chromatography on silica gel (PE/EtOAc=1/1) to give 5-bromo-6-ethenyl-1,3-dihydro-2-benzofuran-1-one (5.0 g, yield 82%) as a brown solid. LC-MS(ESI): mass calcd. for C₁₀H—BrO₂, 237.96; m/z found, 238.97 [M+H]⁺. ¹H NMR (400 MHZ, DMSO-d₆) δ 8.08 (s, 1H), 8.02 (s, 1H), 7.03 (dd, J=17.4, 11.0 Hz, 1H), 6.05 (d, J=17.4 Hz, 1H), 5.54 (d, J=11.0 Hz, 1H), 5.40 (s, 2H).

### Step B: 5-Bromo-6-(2-Hydroxyethyl)-1,3-Dihydro-2-Benzofuran-1-One

To a solution of 5-bromo-6-ethenyl-1,3-dihydro-2-benzofuran-1-one (5.0 g, 20.91 mmo1′1.0 eq) in THF (50 mL) at 0° C. was added 9-BBN (1 Nin THF) (25.2 mL, 25.2 mmol, 1.2 eq) and the reaction mixture was stirred at room temperature overnight. A solution of Sodium peroxyborate (3.42 g, 41.829 mmol, 2.0 eq) in water (100 mL) was added to above mixture and the reaction mixture was stirred at room temperature for 2 h. The reaction solution was quenched with diluted HCl solution (1 N, 100 mL), stirred for 1 h, and extracted with ethyl acetate (1500 mL×3). The organic layer was washed with brine (100 mL), dried over anhydrous sodium sulfate, filtered and concentrated under reduced pressure. The residue was purified by flash column chromatography on silica gel (PE/EA=1/2) to obtain 5-bromo-6-(2-hydroxyethyl)-1,3-dihydro-2-benzofuran-1-one (2.4 g, yield 44.6%) as a white solid. LC-MS(ESI): mass calcd. for C₁₀H₇BrO₂, 257.08; m/z found, 239.05 [M−OH]⁺. ¹H NMR (400 MHz, DMSO-d₆) δ 7.98 (s, 1H), 7.80 (s, 1H), 5.37 (s, 2H), 4.81 (t, J=5.2 Hz, 1H), 3.66 (dd, J=12.0, 6.6 Hz, 2H), 2.97 (t, J=6.6 Hz, 2H).

### Step C: Benzyl 4-[6-(2-Hydroxyethyl)-1-Oxo-1,3-Dihydro-2-Benzofuran-5-Yl]-1,2,3,6-Tetrahydropyridine-1-Carboxylate

To a solution of 5-bromo-6-(2-hydroxyethyl)-1,3-dihydro-2-benzofuran-1-one (2.6 g, 10.11 mmol, 1.0 eq) and benzyl 4-(tetramethyl-1,3,2-dioxaborolan-2-yl)-1,2,3,6-tetrahydropyridine-1-carboxylate (5.21 g, 15.17 mmol, 1.5 eq) in dioxane (50 mL) and H₂O (1 mL) were added Pd(dppf) Cl₂ (1.48 g, 2.02 mmol, 0.2 eq) and K₂CO₃ (4.19 g, 30.34 mmol, 3.0 eq). The reaction mixture was stirred under nitrogen atmosphere at 90° C. for 2 h. After cooled to room temperature, the reaction mixture was filtered, and the cake was washed with EA (30 mL). The filtrate was diluted with H₂O (100 mL) and extracted with EtOAc (200 mL×3). The combined organic layers were washed with brine (100 mL), dried over anhydrous Na₂SO₄, filtered, and concentrated under reduced pressure. The residue was purified by flash column chromatography on silica gel (PE/EtOAc=1/1) to give benzyl 4-[6-(2-hydroxyethyl)-1-oxo-1,3-dihydro-2-benzofuran-5-yl]-1,2,3,6-tetrahydropyridine-1-carboxylate (2.5 g, yield 62.8%) as a white solid. LC-MS(ESI): mass calcd. for C₂₃H₂₃NO₅, 393.44; m/z found, 394.15 [M+H]⁺.

### Step D: Benzyl 3′-Bromo-1-Oxo-1,3,7,8-Tetrahydrospiro[Furo [3,4-g]Isochromene-5,4′-Piperidine]-1′-Carboxylate

To a mixture of benzyl 4-[6-(2-hydroxyethyl)-1-oxo-1,3-dihydro-2-benzofuran-5-yl]-1,2,3,6-tetrahydropyridine-1-carboxylate (3.9 g, 9.91 mmol, 1.0 eq) in MeCN (30 mL) was added NBS(2.12 g, 11.89 mmol, 1.2 eq). The resulting mixture was stirred at room temperature for 1 h. The reaction mixture was quenched with saturated aqueous Na₂S₂O₃ solution (30 mL) and extracted with EtOAc (50 mL×3). The combined organic layers were washed with H₂O (50 mL), dried over anhydrous Na₂SO₄, filtered and concentrated under reduced pressure. The residue was purified by flash column chromatography on silica gel (PE/EtOAc=1/1) to give benzyl 3′-bromo-1-oxo-1,3,7,8-tetrahydrospiro[furo [3,4-glisochromene-5,4′-piperidine]-1′-carboxylate (3.5 g, 7.410 mmol, 74.75%) as a white solid. LC-MS(ESI): mass calcd. for C₂₃H₂₂BrNO₅, 472.34; m/z found, 472.06 [M+H]⁺. ¹H NMR (400 MHZ, DMSO-d₆) δ 7.75 (s, 1H), 7.69 (s, 1H), 7.39-7.33 (m, 5H), 5.44-5.32 (m, 2H), 5.18-5.04 (m, 2H), 4.79-4.76 (m, 1H), 4.25-4.14 (m, 2H), 4.07 (d, J=13.6 Hz, 1H), 3.85 (dt, J=11.4, 5.8 Hz, 1H), 3.32-3.18 (m, 2H), 3.05 (t, J=6.1 Hz, 2H), 2.78-2.65 (m, 1H), 1.61 (d, J=14.0 Hz, 1H).

### Step E: Benzyl 1-Oxo-1,3,7,8-Tetrahydrospiro[Furo [3,4-g]Isochromene-5,4′-Piperidine]-1′-Carboxylate

To a stirred mixture of benzyl 3′-bromo-1-oxo-1,3,7,8-tetrahydrospiro[furo [3,4-glisochromene-5,4′-piperidine]-1′-carboxylate (3.5 g, 7.41 mmol, 1.0 eq) in toluene (80 mL) were added AIBN (2.192 mL, 14.82 mmol, 2.0 eq) and n-Bu₃SnH (9.98 mL, 37.05 mmol, 5.0 eq). The resulting mixture was stirred under N₂ at 110° C. overnight. After cooled to room temperature, the reaction mixture was quenched with aqueous KF solution (100 mL), stirred for 2 h, and extracted with EtOAc (100 mL×3). The combined organic layers were washed with brine (100 mL), dried over anhydrous Na₂SO₄, filtered and concentrated under reduced pressure. The residue was purified by flash column chromatography on silica gel (PE/EtOAc=2/1) to give benzyl 1-oxo-1,3,7,8-tetrahydrospiro[furo [3,4-g]isochromene-5,4′-piperidine]-1′-carboxylate (2.3 g, yield 79%) as a white solid. LC-MS(ESI): mass calcd. for C₂₃H₂₃NO₅, 393.44; m/z found, 394.15 [M+H]⁺.

¹H NMR (400 MHZ, DMSO-d₆) δ 7.64 (s, 1H), 7.62 (s, 1H), 7.40-7.39 (m, 4H), 7.36-7.32 (m, 1H), 5.34 (s, 2H), 5.11 (s, 2H), 3.96 (d, J=14.2 Hz, 2H), 3.89 (t, J=5.6 Hz, 2H), 3.23-3.10 (m, 2H), 2.91 (t, J=5.4 Hz, 2H), 1.96-1.79 (m, 4H).

### Step F: 1′-((Benzyloxy) Carbonyl)-7-(Hydroxymethyl)Spiro[Isochromane-1,4′-Piperidine]-6-Carboxylic Acid

To a mixture of benzyl 1-oxo-1,3,7,8-tetrahydrospiro[furo [3,4-g]isochromene-5,4′-piperidine]-1′-carboxylate (2.3 g, 5.85 mmol, 1.0 eq) in THF (30 mL), MeOH (30 mL) and H₂O (15 mL) was added NaOH (1.17 g 29.23 mmol, 5.0 eq). The resulting mixture was stirred at room temperature for 2 h. The reaction mixture was adjusted to pH 4-5 with diluted aqueous HCl solution (1 N) and extracted with EtOAc (50 mL×3). The combined organic layers were dried over anhydrous Na₂SO₄, filtered and concentrated under reduced pressure. The residue was purified by flash column chromatography on silica gel (PE/EtOAc=1/1) to give 1′-((benzyloxy) carbonyl)-7-(hydroxymethyl)spiro[isochromane-1,4′-piperidine]-6-carboxylic acid (2.2 g, yield 91.5%) as a white solid. LC-MS(ESI): mass calcd. for C₂₃H₂₅NO₆, 411.45; m/z found, 412.16 [M+H]⁺.

### Step G: 1′-((Benzyloxy) Carbonyl)-7-Formylspiro[Isochromane-1,4′-Piperidine]-6-Carboxylic Acid

To a stirred solution of 1′-((benzyloxy) carbonyl)-7-(hydroxymethyl)spiro[isochromane-1,4′-piperidine]-6-carboxylic acid (240 mg, 0.58 mmol, 1.0 eq) in DCM (15 mL) was add active MnO₂ (506.62 mg, 5.83 mmol, 10 eq). The reaction mixture was stirred at room temperature for 2 h. The reaction mixture was filtered and the MnO₂ cake was washed with DCM (30 mL×3). The combined filtrates were concentrated to afford 1′-((benzyloxy) carbonyl)-7-formylspiro[isochromane-1,4′-piperidine]-6-carboxylic acid (160 mg, yield 67%) as a white solid. LC-MS(ESI): mass calcd. for C₂₃H₂₃NO₆, 409.44; m/z found, 410.14 [M+H]⁺.

### Step H: 1′-[(Benzyloxy) Carbonyl]-7-{[(2,6-Dioxopiperidin-3-Yl)Amino]Methyl}-3,4-Dihydrospiro[2-Benzopyran-1,4′-Piperidine]-6-Carboxylic Acid

To a stirred solution of 1′-[(benzyloxy) carbonyl]-7-formyl-3,4-dihydrospiro[2-benzopyran-1,4′-piperidine]-6-carboxylic acid (30 mg, 0.073 mmol, 1.0 eq), 3-aminopiperidine-2,6-dione hydrochloride (18.71 mg, 0.146 mmol, 2.0 eq) in MeOH (10 mL) was added NaOAc (8.98 mg, 0.110 mmol, 1.0 eq) and the reaction mixture was stirred at room temperature for 40 min. Sodium cyanoborohydride (4.59 mg, 0.073 mmol, 1.0 eq) was added to above mixture and the resulting reaction mixture was stirred at room temperature for 2 h. After evaporation, the residue was purified by Prep-TLC (DCM/MeOH=10/1) to obtain 1′-[(benzyloxy) carbonyl]-7-{[(2,6-dioxopiperidin-3-yl)amino]methyl}-3,4-dihydrospiro[2-benzopyran-1,4′-piperidine]-6-carboxylic acid (18 mg, yield 47.10%) as a white solid. LC-MS (ESI): mass calcd. for C₂₈H₃₁N₃O₇, 511.20; m/z found, 512.21 [M+H]⁺.

### Step I: Benzyl 2′—(2,6-Dioxopiperidin-3-Yl)-1′-Oxo-2′,3′,7′,8′-Tetrahydro-1′H-Spiro[Piperidine-4,5′-Pyrano[3,4-f]Isoindole]-1-Carboxylate

To a stirred solution of 1′-[(benzyloxy) carbonyl]-7-{[(2,6-dioxopiperidin-3-yl)amino]methyl}-3,4-dihydrospiro[2-benzopyran-1,4′-piperidine]-6-carboxylic acid (30 mg, 0.058 mmol, 1.0 eq) in DMF (3 mL) were added HATU (32.81 mg, 0.086 mmol, 1.0 eq) and DIPEA (0.019 mL, 0.115 mmol, 2.0 eq). The reaction mixture was stirred at room temperature for 3 h. After evaporation, the residue was purified by flash column chromatography on silica gel (DCM/MeOH=10/1) to obtain benzyl 2′—(2,6-dioxopiperidin-3-yl)-1′-oxo-2′,3′,7′,8′-tetrahydro-1′H-spiro[piperidine-4,5′-pyrano[3,4-f]isoindole]-1-carboxylate (8 mg, yield 27.62%) as a white solid. LC-MS(ESI): mass calcd. for C₂₈H₂₉N₃O₆, 503.19; m/z found, 504.20 [M+H]⁺. ¹H NMR (400 MHZ, DMSO-d₆) δ 10.99 (s, 1H), 7.53 (s, 1H), 7.50 (s, 1H), 7.40-7.39 (m, 4H), 7.36-7.32 (m, 1H), 5.14-5.06 (m, 3H), 4.38 (d, J=16.8 Hz, 1H), 4.26 (d, J=16.8 Hz, 1H), 3.96 (d, J=13.6 Hz, 2H), 3.88 (t, J=5.4 Hz, 2H), 3.16 (s, 2H), 2.89 (s, 3H), 2.60-2.56 (m, 1H), 2.48-2.42 (m, 1H), 2.00-1.84 (m, 5H).

### Step J: 3-{1′-Oxo-2′,3′,7′,8′-Tetrahydro-1′H-Spiro[Piperidine-4,5′-Pyrano[3,4-f]Isoindole]-2′-Yl}Piperidine-2,6-Dione

To a stirred solution of benzyl 2′—(2,6-dioxopiperidin-3-yl)-1′-oxo-2′,3′,7′,8′-tetrahydro-1′H-spiro[piperidine-4,5′-pyrano[3,4-f]isoindole]-1-carboxylate (300 mg, 0.596 mmol, 1.0 eq) in TFE (10 mL) was add 10% Pd/C (50 mg) and the reaction mixture was stirred under H₂ (1 atm) at 40° C. overnight. After filtration, the filtrate was concentrated to get 3-{1′-oxo-2′,3′,7′,8′-tetrahydro-1′H-spiro[piperidine-4,5′-pyrano[3,4-f]isoindole]-2′-yl}piperidine-2,6-dione (130 mg, yield 59.0%) as a white solid. LC-MS(ESI): mass calcd. for C₂₀H₂₃N₃O₄, 369.15; m/z found, 370.16 [M+H]⁺. ¹H NMR (400 MHZ, DMSO-d₆) δ 10.98 (s, 1H), 7.53 (s, 1H), 7.42 (s, 1H), 5.10-5.06 (m, 1H), 4.45 (d, J=16.8 Hz, 1H), 4.32 (d, J=16.8 Hz, 1H), 3.88 (s, 2H), 3.07-2.88 (m, 6H), 2.60-2.56 (m, 1H), 2.48-2.36 (m, 2H), 2.03-1.87 (m, 5H).

### Compound A4:3-(1′-Methyl-7-Oxo-5,7-Dihydro-2H,6H-Spiro[Furo [2,3-f]Isoindole-3,4′-Piperidin]-6-Yl) Piperidine-2,6-Dione

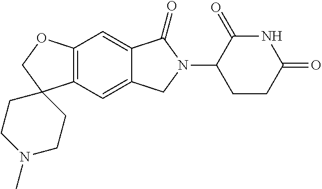 [SMILES: CN1CCC2(CC1)COc1cc3c(cc12)CN(C1CCC(=O)NC1=O)C3=O]

To a solution of 3-{7-oxo-2,5,6,7-tetrahydrospiro[furo [2,3-f]isoindole-3,4′-piperidine]-6-yl}piperidine-2,6-dione (50 mg, 0.141 mmol, 1.0 eq) in DMF (1 mL) were added formaldehyde (0.008 mL, 0.0423 mmol, 3.0 eq) and sodium cyanoborohydride (14 mg, 0.211 mmol, 1.5 eq). The reaction was stirred at room temperature for 3 h. The reaction mixture was diluted with saturated aqueous NaHCO₃ solution (1 mL) and extracted with DCM (1 mL×3). The organic layer was collected and concentrated to 1 mL of volume. The residue was diluted with EA (15 mL), stirred at room temperature for 2 h, and the solid precipitated. The solid was filtered and purified by prep-HPLC with YMC-TA C18 (5 μm, 20×250 mm), and mobile phase of 5-95% MeCN (0.1% HCOOH) in water over 10 min, and then hold at 100% ACN for 2 min, at a flow rate of 25 mL/min to give 3-{1′-methyl-7-oxo-2,5,6,7-tetrahydrospiro[furo [2,3-f]isoindole-3,4′-piperidine]-6-yl}piperidine-2,6-dione formate (10 mg, yield 19%) as a white solid. LC-MS(ESI): mass calcd. for C₂₀H₂₃N₃O₄, 369.17; m/z found, 370.20, (M+H)⁺. ¹H NMR (400 MHZ, DMSO-d₆) δ 10.97 (s, 1H), 8.14 (s, 1H), 7.43 (s, 1H), 7.01 (s, 1H), 5.09-5.05 (m, 1H), 4.47 (s, 2H), 4.35 (d, J=16.8 Hz, 1H), 4.22 (d, J=16.8 Hz, 1H), 2.92-2.87 (m, 3H), 2.64-2.55 (m, 1H), 2.42-2.37 (m, 1H), 2.29 (s, 3H), 2.15-2.11 (m, 1H), 2.03-1.92 (m, 4H), 1.74-1.70 (m, 2H).

### Compound A5:3-(1′-Acetyl-7-Oxo-5,7-Dihydro-2H,6H-Spiro[Furo [2,3-f]Isoindole-3,4′-Piperidin]-6-Yl) Piperidine-2,6-Dione

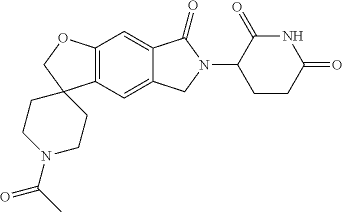 [SMILES: CC(=O)N1CCC2(CC1)COc1cc3c(cc12)CN(C1CCC(=O)NC1=O)C3=O]

### Step A: Methyl 1′-Acetyl-5-Methyl-2H-Spiro[1-Benzofuran-3,4′-Piperidine]-6-Carboxylate

To a solution of methyl 1′-benzyl-5-methyl-2H-spiro[1-benzofuran-3,4′-piperidine]-6-carboxylate (1.3 g, 3.7 mmol, 1.0 eq) in MeOH (15 mL) was added acetic anhydride (0.9 mL, 9.25 mmol, 2.5 eq) and 10% Pd/C (100 mg). The mixture was stirred under H₂ (1 atm) for 3 h. After filtration, the filtrate was concentrated to provide methyl 1′-acetyl-5-methyl-2H-spiro[1-benzofuran-3,4′-piperidine]-6-carboxylate (270 mg, yield 74%) as a white solid. LC-MS(ESI): mass calcd. for C₁₇H₂₁NO₄, 303.15; m/z found, 304.2 [M+H]⁺.

### Step B: Methyl 1′-Acetyl-5-(Bromomethyl)-2H-Spiro[1-Benzofuran-3,4′-Piperidine]-6-Carboxylate

A mixture of methyl 1′-benzyl-5-methyl-2H-spiro[1-benzofuran-3,4′-piperidine]-6-carboxylate (50 mg, 0.142 mmol, 1.0 eq), NBS(28 mg, 0.156 mmol, 1.1 eq), and BPO (7 mg, 0.03 mmol, 0.3 eq) in CCl₄ (2 mL) was refluxed for 4 h. After evaporation, the mixture was concentrated and purified by prep-TLC (EA/PE=1/4) to obtain methyl 1′-acetyl-5-(bromomethyl)-2H-spiro[1-benzofuran-3,4′-piperidine]-6-carboxylate (20 mg, yield 28%) as a yellow oil. LC-MS(ESI): mass calcd. for C₁₇H₂₀BrNO₄, 381.06; m/z found, 382.3 [M+H]⁺.

### Step C: 3-{1′-Acetyl-7-Oxo-2,5,6,7-Tetrahydrospiro[Furo [2,3-f]Isoindole-3,4′-Piperidine]-6-Yl}Piperidine-2,6-Dione

DIPEA (0.13 mL, 0.785 mmol, 3 eq) was added to a mixture of methyl 1′-acetyl-5-(bromomethyl)-2H-spiro[1-benzofuran-3,4′-piperidine]-6-carboxylate (100 mg, 0.262 mmol, 1.0 eq) and 3-aminopiperidine-2,6-dione hydrochloride (65 mg, 0.392 mmol, 1.5 eq) in MeCN (5 mL) under nitrogen. The resulting suspension was stirred at 80° C. for 24 h. The reaction mixture was cooled to room temperature and filtered. The cake was washed with MeCN and the product was purified by Prep-TLC (MeCN/DCM=1/1) to afford 3-{1′-acetyl-7-oxo-2,5,6,7-tetrahydrospiro[furo [2,3-f]isoindole-3,4′-piperidine]-6-yl}piperidine-2,6-dione (40 mg, yield 38%) as a white solid. LC-MS(ESI): mass calcd. for C₂₁H₂₃N₃O₅, 397.16; m/z found, 398.4 [M+H]⁺. ¹H NMR (400 MHZ, CDCl₃)δ 8.39 (s, 1H), 7.27 (s, 1H), 7.14 (s, 1H), 5.24-5.15 (m, 1H), 4.63 (d, J=13.2 Hz, 1H), 4.59-4.48 (m, 2H), 4.42-4.38 (m, 1H), 4.27 (d, J=15.6 Hz, 1H), 3.95-3.82 (m, 1H), 3.27-3.14 (m, 1H), 2.97-2.64 (m, 3H), 2.43-2.27 (m, 1H), 2.24-2.11 (m, 4H), 1.89-1.82 (m, 4H).

### Compound A6:3-(1-Methyl-1′-Oxo-1′,3′,7′,8′-Tetrahydro-2′H-Spiro[Piperidine-4,5′-Pyrano[3,4-f]Isoindol]-2′-Yl) Piperidine-2,6-Dione

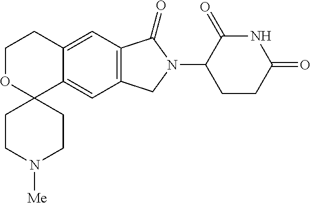 [SMILES: CN1CCC2(CC1)OCCc1cc3c(cc12)CN(C1CCC(=O)NC1=O)C3=O]

A solution of 3-{1′-oxo-2′,3′,7′,8′-tetrahydro-1′H-spiro[piperidine-4,5′-pyrano[3,4-f]isoindole]-2′-yl}piperidine-2,6-dione (50 mg, 0.135 mmol, 1.0 eq) and Formaldehyde (37% in H₂O) (0.5 mL, 10.0 eq) in DMF (2 mL) was stirred at room temperature for 40 min. NaBH(OAc)3 (57.10 mg, 0.271 mmol, 2.0 eq) was added to above mixture and the resulting reaction mixture was stirred at room temperature for 3 h. The residue was purified by Prep-HPLC with YMC-Actus Triart 18C (5 μm, 20×250 mm), and mobile phase of 5-99% ACN in water (0.1% FA) over 10 min and then hold at 100% ACN for 2 min, at a flow rate of 25 mL/min to obtain 3-{1-methyl-1′-oxo-2′,3′,7′,8′-tetrahydro-1′H-spiro[piperidine-4,5′-pyrano[3,4-f]isoindole]-2′-yl}piperidine-2,6-dione (2.0 mg, yield 4%) as a white solid.

LC-MS(ESI): mass calcd. for C₂₁H₂₅N₃O₄, 383.20; m/z found, 384.21 [M+H]⁺. ¹H NMR (400 MHZ, DMSO-d₆) δ 10.98 (s, 1H), 7.53 (s, 1H), 7.42 (s, 1H), 5.09-5.05 (m, 1H), 4.45 (d, J=16.8 Hz, 1H), 4.32 (d, J=16.8 Hz, 1H), 3.88 (s, 2H), 3.07 (t, J=12.2 Hz, 3H), 2.95 (d, J=6.8 Hz, 2H), 2.90 (s, 2H), 2.74 (s, 3H), 2.60-2.46 (m, 1H), 2.38-2.33 (m, 2H), 2.03-1.87 (m, 5H).

### Compound A7:3-(1-Acetyl-1′-Oxo-1′,3′,7′,8′-Tetrahydro-2′H-Spiro[Piperidine-4,5′-Pyrano[3,4-f]Isoindol]-2′-Yl) Piperidine-2,6-Dione

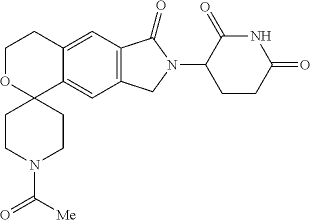 [SMILES: CC(=O)N1CCC2(CC1)OCCc1cc3c(cc12)CN(C1CCC(=O)NC1=O)C3=O]

To a stirred solution of benzyl 2′—(2,6-dioxopiperidin-3-yl)-1′-oxo-2′,3′,7′,8′-tetrahydro-1′H-spiro[piperidine-4,5′-pyrano[3,4-f]isoindole]-1-carboxylate (50 mg, 0.099 mmol, 1.0 eq) and Ac₂O (101.07 mg, 0.990 mmol, 10.0 eq) in TEA (10 mL) was add 10% Pd/C (30 mg) and the reaction mixture was stirred under H₂ (1 atm) at 40° C. overnight. After filtration, the filtrate was concentrated and the residue was purified by Prep-HPLC with YMC-Actus Triart 18C (5 μm, 20×250 mm), and mobile phase of 5-99% ACN in water (0.1% FA) over 10 min and then hold at 100% ACN for 2 min, at a flow rate of 25 mL/min to obtain 3-(1-acetyl-1′-oxo-1′,3′,7′,8′-tetrahydro-2′H-spiro[piperidine-4,5′-pyrano[3,4-f]isoindol]-2′-yl) piperidine-2,6-dione (1.5 mg, yield 3.67%) as a white solid.

LC-MS(ESI): mass calcd. for C₂₂H₂₅N₃O₅, 411.46; m/z found, 412.21 [M+H]⁺. ¹H NMR (400 MHZ, DMSO-d₆) § 10.97 (s, 1H), 7.54 (s, 1H), 7.50 (s, 1H), 5.11-5.07 (m, 1H), 4.37-4.26 (m, 3H), 3.89 (t, J=5.0 Hz, 2H), 3.72 (d, J=13.0 Hz, 1H), 3.34 (s, 1H), 2.91-2.83 (m, 4H), 2.59-2.56 (m, 1H), 2.48-2.42 (m, 1H), 2.05 (s, 3H), 1.99 (d, J=11.2 Hz, 2H), 1.91-1.77 (m, 3H).

### Compound A8:3-(6′-Oxo-6′,8′-Dihydro-2′H,7′H-Spiro[Azepane-4,3′-Furo [2,3-e]Isoindol]-7′-Yl) Piperidine-2,6-Dione

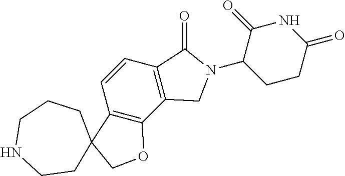 [SMILES: O=C1CCC(N2Cc3c(ccc4c3OCC43CCCNCC3)C2=O)C(=O)N1]

### Step A: Tert-Butyl-5-(((Trifluoromethyl) Sulfonyl)Oxy)-2,3,4,-Tetrahydro-1H-Azepine-1-Carboxylate

To a solution of tert-butyl 4-oxoazepane-1-carboxylate (5 g, 23.44 mmol, 1.0 eq) in THF (60 mL), was added dropwise LiHDMS solution (1 N in THF) (35.16 mL, 35.16 mmol, 1.5 eq) under nitrogen at −78° C. The mixture was stirred at this temperature for 20 min, then a solution of PhNTf₂ (12.56 g, 35.16 mmol, 1.5 eq) in THF (30 mL) was added dropwise to above mixture. The resulting mixture was warmed to 0° C. and stirred for 3 h. The reaction mixture was quenched with saturated aqueous NH₄Cl solution (50 mL) and extracted with EA (100 mL×3). The organic layer was washed with brine (100 mL), dried over anhydrous sodium sulfate, filtered and concentrated under reduced pressure. The crude product was purified by flash column chromatography on silica gel (ethyl acetate in petroleum ether, from 0% to 9%) to afford tert-butyl-4-(trifluoromethanesulfonyloxy)-2,3,6,7-tetrahydro-1H-azepine-1-carboxylate (4.5 g, yield 55%) as a light yellow oil. LC-MS(ESI): mass calcd. for C₁₂H₁₈F₃NO₅S, 345.18; m/z found, 346.2 [M+H]⁺.

### Step B: 1-(Tert-Butyl)4-Methyl 2,5,6,7-Tetrahydro-1H-Azepine-1,4-Dicarboxylate

To a mixture of tert-butyl-4-(trifluoromethanesulfonyloxy)-2,3,6,7-tetrahydro-1H-azepine-1-carboxylate (4.2 g, 12.16 mmol, 1.0 eq) and Pd(dppf) Cl₂ (445 mg, 0.608 mmol, 0.05 eq) in MeOH (100 mL) was added TEA (20 mL, 142.3 mmol, 12 eq). The mixture was stirred under CO (1 atm) at 70° C. for 16 h. After evaporation, the residue was diluted with water (30 mL) and extracted with EA (100 mL×3). The combined organic layers were washed with brine (100 mL), dried over anhydrous Na₂SO₄, filtered and concentrated under reduced pressure. The residue was purified by flash column chromatography on silica gel (ethyl acetate in petroleum ether, from 0% to 40%) to give 1-(tert-butyl)4-methyl 2,5,6,7-tetrahydro-1H-azepine-1,4-dicarboxylate (2.8 g, yield 89%) as a yellow solid. LC-MS(ESI): mass calcd. for C₁₃H₂₁NO₄, 255; m/z found, 256.1 [M+H]⁺. ¹H NMR (400 MHZ, DMSO-d₆) δ 6.92-6.91 (m, 1H), 4.02 (s, 2H), 3.66 (s, 3H), 3.46-3.41 (m, 2H), 2.46-2.41 (m, 2H), 1.74 (s, 2H), 1.39 (s, 9H).

### Step C: Tert-Butyl 5-(Hydroxymethyl)-2,3,4,7-Tetrahydro-1H-Azepine-1-Carboxylate

To a solution of 1-tert-butyl 4-methyl 2,3,6,7-tetrahydro-1H-azepine-1,4-dicarboxylate (2.8 g, 10.98 mmol, 1.0 eq) in THF (100 mL) was added dropwise DIBAL-H (1 N in THF) (16.5 mL, 16.5 mmol, 1.5 eq) under N₂. The mixture was stirred under N₂ at −70° C. for 5 h. The reaction mixture was diluted with THF (60 mL), slowly quenched by addition of Na₂SO₄: 10H₂O, and stirred for 30 min. After filtration, the filtrate was concentrated under reduced pressure to give tert-butyl 4-(hydroxymethyl)-2,3,6,7-tetrahydro-1H-azepine-1-carboxylate (2.3 g, yield 90%) as a colorless oil. LC-MS(ESI): mass calcd. for C₁₂H₂₁NO₃, 227; m/z found, 228.3 [M+H]⁺. ¹H NMR (400 MHZ, DMSO-d₆) δ 5.64 (d, J=16.0 Hz, 1H), 4.78 (t, J=4.0 Hz, 1H), 3.82 (s, 2H), 3.76 (s, 2H), 3.44 (t, J=5.8 Hz, 2H), 2.15-2.03 (m, 2H), 1.66 (s, 2H), 1.39 (s, 9H).

### Step D: Tert-Butyl-5-(((5-Bromo-1-Oxo-1,3-Dihydroisobenzofuran-4-Yl)Oxy)Methyl)-2,3,4,7-Tetrahydro-1H-Azepine-1-Carboxylate

To a solution of tert-butyl-(hydroxymethyl)-2,3,6,7-tetrahydro-1H-azepine-1-carboxylate (2.3 g, 10.13 mmol, 1.0 eq), 5-bromo-4-hydroxy-1,3-dihydro-2-benzofuran-1-one (2.45 g, 10.13 mmol, 1.0 eq) in THF (100 mL) was added PPh₃ (6.22 g, 15.20 mmol, 1.5 eq) and the mixture was stirred under N₂ at 0° C. for 10 min. Then DIAD (4.80 g, 15.20 mmol, 1.5 eq) was dropwise added to above mixture and the resulting solution was stirred under N₂ at 30° for 6 h. The reaction mixture was quenched with H₂O (20 mL) and extracted with EtOAc (20 mL×3). The organic layer was washed with brine (20 mL), dried over anhydrous Na₂SO₄, filtered and concentrated under reduced pressure. The crude product was purified by flash column chromatography on silica gel (ethyl acetate in petroleum ether, from 0% to 40%) to givethe tert-butyl-5-(((5-bromo-1-oxo-1,3-dihydroisobenzofuran-4-yl)oxy)methyl)-2,3,4,7-tetrahydro-1H-azepine-1-carboxylate (3.3 g, yield 75%) as a colorless oil. LC-MS(ESI): mass calcd. for C₁₂H₂₁NO₃, 437; m/z found, 438.2 [M+H]⁺.

### Step E: Tert-Butyl-6′-Oxo-6′,8′-Dihydro-2′H-Spiro[Azepane-4,3′-Benzo[2,1-b: 3,4-c′]Difuran]-1-Carboxylate

To a solution of tert-butyl-4-{[(5-bromo-1-oxo-1,3-dihydro-2-benzofuran-4-yl)oxy]methyl}-2,3,6,7-tetrahydro-1H-azepine-1-carboxylate (3.3 g, 7.58 mmol, 1.0 eq) in toluene (50 mL) were added tributylstannane (10.2 mL, 37.9 mmol, 5.0 eq) and AIBN (0.28 g, 1.52 mmol, 0.2 eq). The mixture was stirred under N₂ at 110° C. in a sealed tube for 5 h. After cooled to room temperature, the mixture was quenched with aqueous KF solution (100 mL), stirred for 1 h, and exacted with EA (100 mL×3). The organic layer was dried over anhydrous Na₂SO₄, filtered and concentrated under reduced pressure. The residue was purified by flash column chromatography on silica gel (ethyl acetate in petroleum ether, from 0% to 30%) to give tert-butyl 6′-oxo-6′,8′-dihydro-2′H-spiro[azepane-4,3′-benzo[2,1-b: 3,4-c′]difuran]-1-carboxylate (2.3 g, yield 74%) as a colorless oil. LC-MS(ESI): mass calcd. for C₂₀H₂₅NO₅, 359; m/z found, 360.2 [M+H]⁺. ¹H NMR (400 MHZ, DMSO-d₆) δ 7.44-7.39 (m, 2H), 5.35 (s, 2H), 4.54-4.46 (m, 2H), 3.51-3.33 (m, 4H), 1.99-1.67 (m, 6H), 1.43 (s, 9H).

### Step F: 1-(Tert-Butoxycarbonyl)-7′-(Hydroxymethyl)-2′H-Spiro[Azepane-4,3′-Benzofuran]-6′-Carboxylic Acid

To a solution of tert-butyl 6′-oxo-6′,8′-dihydro-2′H-spiro[azepane-4,3′-benzo[2,1-b: 3,4-c′]difuran]-1-carboxylate (1.5 g, 4.173 mmol, 1.0 eq) in THF (20 mL), MeOH (20 mL), and H₂O (10 mL) was added NaOH (834.6 mg, 20.867 mmol, 5.0 eq) and the mixture was stirred at 40° C. for 2 h. After evaporation, the residue was diluted with water (30 mL), acidified to pH 5-6 with aqueous HCl solution (1 N), and extracted with EA (100 mL×3). The combined organic layers were washed with brine (100 mL), dried over anhydrous Na₂SO₄, filtered and concentrated under reduced pressure to give crude 1-[(tert-butoxy) carbonyl]-7′-(hydroxymethyl)-2′Hspiro[azepane-4,3′-[1]benzofuran]-6′-carboxylic acid (1.4 g, yield 89%) as a yellow solid. The crude product was directly used in next step without further purification. LC-MS(ESI): mass calcd. for C₂₀H₂₇NO₆, 377.18; m/z found, 378.1 [M+H]⁺.

### Step G: 1-(Tert-Butoxycarbonyl)-7′-Formyl-2′H-Spiro[Azepane-4,3′-Benzofuran]-6′-Carboxylic Acid

To a solution of 1-(tert-butoxycarbonyl)-7′-(hydroxymethyl)-2′H-spiro[azepane-4,3′-benzofuran]-6′-carboxylic acid (1.4 g, 3.71 mmol, 1.0 eq) in DCM (50 mL) was added Manganese dioxide (6.45 g, 74.184 mmol, 20.0 eq). The mixture was stirred at room temperature for 1 h. After filtrated to remove MnO₂, the combined filtrates were concentrated under reduced pressure to give 1-[(tert-butoxy) carbonyl]-7′-formyl-2′H-spiro[azepane-4,3′-[1]benzofuran]-6′-carboxylic acid (1.0 g, yield 72%) as a yellow solid. LC-MS(ESI): mass calcd. for C₂₀H₂₅NO₆, 375.17; m/z found, 376.1 [M+H]⁺.

### Step H: Tert-Butyl 7′-(2,6-Dioxopiperidin-3-Yl)-6′-Oxo-7′,8′-Dihydro-2′H,6′H-Spiro[Azepane-4,3′-Furo [2,3-e]Isoindole]-1-Carboxylate

To a solution of 1-[(tert-butoxy) carbonyl]-7′-formyl-2′H-spiro[azepane-4,3′-[1]benzofuran]-6′-carboxylic acid (900 mg, 2.397 mmol, 1.0 eq) and 3-aminopiperidine-2,6-dione hydrochloride (460.75 mg, 3.596 mmol, 1.5 eq) in DMF (50 mL) was added AcOH (0.5 mL, 8.726 mmol, 3.64 eq) at room temperature. The reaction mixture was stirred at room temperature for 2 h. Sodium cyanoborohydride (451.94 mg, 7.192 mmol, 3.0 eq) was added to above mixture and the resulting reaction mixture was stirred at room temperature overnight. The reaction mixture was quenched with water (20 mL) and extracted with EtOAc (30 mL×3). The organic layer was washed with brine (30 mL), dried over anhydrous Na₂SO₄, filtered and concentrated under reduced pressure. The residue was purified by flash column chromatography on silica gel(methanol in dichloromethane, from 0% to 10%) to give tert-butyl 7′-(2,6-dioxopiperidin-3-yl)-6′-oxo-7′,8′-dihydro-2′H,6′H-spiro[azepane-4,3′-furo [2,3-e]isoindole]-1-carboxylate (130 mg, yield 12%) as a white solid. LC-MS(ESI): mass calcd. for C₂₅H₃₁N₃O₆, 469.22; m/z found, 468.1 (M−H).

### Step I: 3-(6′-Oxo-6′,8′-Dihydro-2′H,7′H-Spiro[Azepane-4,3′-Furo [2,3-e]Isoindol]-7′-Yl) Piperidine-2,6-Dione

To a solution of tert-butyl 7′-(2,6-dioxopiperidin-3-yl)-6′-oxo-2′,6′,7′,8′-tetrahydrospiro[azepane-4,3′-furo [2,3-e]isoindole]-1-carboxylate (110 mg, 0.234 mmol, 1.0 eq) in dioxane (3 mL) was added HCl-dioxane (4 N) (1.2 mL, 1.171 mmol, 5.0 eq) and the mixture was stirred at room temperature for 2 h. After evaporation, the residue was purified by Prep-HPLC with YMC-Actus Triart 18C (5 μm, 20×250 mm), and mobile phase of 5-99% ACN in water (0.1% HCl) over 10 min and then hold at 100% ACN for 2 min, at a flow rate of 25 mL/min to give 3-(6′-oxo-6′,8′-dihydro-2′H,7′H-spiro[azepane-4,3′-furo [2,3-e]isoindol]-7′-yl) piperidine-2,6-dione hydrochloride (16.5 mg, yield 19%) as a white solid. LC-MS(ESI): mass calcd. for C₂₀H₂₃N₃O₄, 369.17; m/z found, 370.2 [M+H]⁺. ¹H NMR (400 MHZ, DMSO-d₆) § 10.98 (d, J=6.8 Hz, 1H), 9.32-8.76 (m, 2H), 7.57-7.43 (m, 1H), 7.34-7.30 (m, 1H), 5.11-5.07 (m, 1H), 4.36-4.16 (m, 4H), 3.62-3.39 (m, 2H), 3.17-3.08 (m, 1H), 2.97-2.60 (m, 3H), 2.42-2.18 (m, 3H), 2.12-2.03-1.76 (m, 5H).

### Compound A9:3-(6-Oxo-6,8-Dihydro-2H,7H-Spiro[Furo [2,3-e]Isoindole-3,3′-Pyrrolidin]-7-Yl) Piperidine-2,6-Dione Hydrochloride

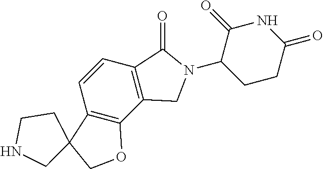 [SMILES: O=C1CCC(N2Cc3c(ccc4c3OCC43CCNC3)C2=O)C(=O)N1]

### Step A: Tert-Butyl Allyl(2-(Chloromethyl) Allyl) Carbamate

To a solution of tert-butyl N-(prop-2-en-1-yl) carbamate (18.5 g, 117.7 mmol, 1.0 eq) in DMF (250 mL) was added NaH (60% suspend in oil) (7.1 g, 176.5 mmol, 1.5 eq) under nitrogen at 0° C. The reaction mixture was stirred at 0° C. for 1 h, followed by a solution of 3-chloro-2-(chloromethyl) prop-1-ene (22.1 g, 176.5 mmol, 1.5 eq) in DMF (50 mL) was added dropwise. The reaction mixture was stirred at room temperature for 1 h. The reaction mixture was quenched with water (40 mL) and extracted with EtOAc (40 mL×3). The organic layer was washed with brine (40 mL×4), dried over anhydrous Na₂SO₄, filtered and concentrated under reduced pressure. The residue was purified by flash column chromatography on silica gel (PE/EA=30/1) to afford tert-butyl N-[2-(chloromethyl) prop-2-en-1-yl]-N-(prop-2-en-1-yl) carbamate (11 g, yield 38%) as a yellow oil. LC-MS(ESI): mass calcd. for C₁₄H₁₈ClN₃, 245.12; m/z found, 246.1 [M+H]⁺. ¹H NMR (400 MHZ, CDCl₃)δ 5.82-5.72 (m, 1H), 5.27 (s, 1H), 5.16-5.05 (m, 3H), 4.02 (s, 2H), 4.02 (s, 2H), 3.93 (s, 2H), 3.81 (s, 2H), 1.46 (s, 9H).

### Step B: Tert-Butyl 3-(Chloromethyl)-2,5-Dihydro-1H-Pyrrole-1-Carboxylate

To a solution of tert-butyl N-[2-(chloromethyl) prop-2-en-1-yl]-N-(prop-2-en-1-yl) carbamate (11 g, 44.8 mmol, 1.0 eq) in DCM (800 mL) was added Grubbs I catalyst (3.6 g, 4.5 mmol, 0.1 eq) at room temperature and the mixture was heated to 50° C. for 6 h. The reaction mixture was cooled to room temperature, filtered and the filtrate was concentrated under reduced pressure. The residue was purified by flash column chromatography on silica gel (PE/EA=20/1) to provide tert-butyl 3-(chloromethyl)-2,5-dihydro-1H-pyrrole-1-carboxylate (7.1 g, yield 69%) as a yellow oil. LC-MS(ESI): mass calcd. for C₁₀H₁₆ClNO₂, 217.09; m/z found, 218.1 [M+H]⁺. ¹H NMR (400 MHZ, CDCl₃)δ 5.80 (d, J=19.2 Hz, 1H), 4.23-4.10 (m, 6H), 1.48 (d, J=2.6 Hz, 9H).

### Step C: Tert-Butyl 3-(((5-Bromo-1-Oxo-1,3-Dihydroisobenzofuran-4-Yl)Oxy)Methyl)-2,5-Dihydro-1H-Pyrrole-1-Carboxylate

To a solution of 5-bromo-4-hydroxy-1,3-dihydro-2-benzofuran-1-one (6.5 g, 28.4 mmol, 1.0 eq) and tert-butyl 3-(chloromethyl)-2,5-dihydro-1H-pyrrole-1-carboxylate (6.8 g, 31.2 mmol, 1.1 eq) in DMF (120 mL) was added K₂CO₃ (11.8 g, 85 mmol, 3.0 eq) at room temperature. The mixture was stirred at 80° C. overnight. The reaction mixture was cooled to room temperature and filtered. The filtrate was quenched with water (40 mL) and extracted with EtOAc (40 mL×3). The organic layer was washed with brine (40 mL×4), dried over anhydrous Na₂SO₄, filtered and concentrated under reduced pressure. The residue was purified by flash column chromatography on silica gel (PE/EA=3/1) to afford tert-butyl3-{[(5-bromo-1-oxo-1,3-dihydro-2-benzofuran-4-yl)oxy]methyl}-2,5-dihydro-1H-pyrrole-1-carboxylate (9.1 g, yield 74%) as a yellow solid. LC-MS(ESI): mass calcd. for C₁₈H₂₀BrNO₅, 409.05; m/z found, 410.2 [M+H]⁺. ¹H NMR (400 MHZ, CDCl₃)δ 7.68 (dd, J=8.0, 3.8 Hz, 1H), 7.46 (dd, J=8.0, 3.2 Hz, 1H), 5.81 (d, J=17.6 Hz, 1H), 5.30 (d, J=1.6 Hz, 1H), 4.62 (d, J=3.2 Hz, 1H), 4.24-4.11 (m, 4H), 1.42 (d, J=2.0 Hz, 9H).

### Step D: Tert-Butyl 6-Oxo-6,8-Dihydro-2H-Spiro[Benzo[2,1-b: 3,4-c′]Difuran-3,3′-Pyrrolidine]-1′-Carboxylate

To a solution of tert-butyl 3-{[(5-bromo-1-oxo-1,3-dihydro-2-benzofuran-4-yl)oxy]methyl}-2,5-dihydro-1H-pyrrole-1-carboxylate (6.0 g, 14.6 mmol, 1.0 eq) and Tributyltin Hydride (17 g, 58.5 mmol, 4.0 eq) in toluene (180 mL) was added AIBN (0.48 g, 2.9 mmol, 0.2 eq) at room temperature under nitrogen. The reaction vessel was stirred at 105° C. in a sealed tube overnight. After cooled to room temperature, the reaction mixture was diluted with aqueous KF solution (200 mL), and stirred for 1 h, and extracted with EtOAc (100 mL×3). The organic layer was washed with brine (40 mL×2), dried over anhydrous Na₂SO₄, filtered and concentrated under reduced pressure. The residue was purified by flash column chromatography on silica gel (PE/EA=3/1) to afford tert-butyl 6-oxo-6,8-dihydro-2H-spiro[benzo[2,1-b: 3,4-c′]difuran-3,3′-pyrrolidine]-1′-carboxylate (2.4 g, yield 49%) as a white solid. LC-MS(ESI): mass calcd. for C₁₈H₂₁NO₅, 331.14; m/z found, 332.2 [M+H]⁺. ¹H NMR (400 MHZ, CDCl₃)δ 7.45 (d, J=7.8 Hz, 1H), 7.22 (d, J=7.8 Hz, 1H), 5.21 (s, 2H), 4.53-4.45 (m, 2H), 3.71-3.35 (m, 4H), 2.23-2.15 (m, 1H), 2.10-2.01 (m, 1H), 1.43-1.40 (m, 9H).

### Step E: 1′-(Tert-Butoxycarbonyl)-7-(Hydroxymethyl)-2H-Spiro[Benzofuran-3,3′-Pyrrolidine]-6-Carboxylic Acid

To a solution of tert-butyl 6-oxo-6,8-dihydro-2H-spiro[benzo[2,1-b: 3,4-c′]difuran-3,3′-pyrrolidine]-1′-carboxylate (2.8 g, 8.5 mmol, 1.0 eq) in THE/MeOH/H₂O (36 mL/36 mL/15 mL) was added NaOH (2 g, 50.7 mmol, 5.9 eq) and the mixture was stirred at 40° C. for 2 h. After evaporation, the resulting residue was diluted with water (30 mL), acidified to pH 5-6 with diluted aqueous HCl solution (1 N), and extracted with EtOAc (40 mL×3). The organic layer was dried over anhydrous Na₂SO₄, filtered and concentrated under reduced pressure to give 1′-[(tert-butoxy) carbonyl]-7-(hydroxymethyl)-2H-spiro[1-benzofuran-3,3′-pyrrolidine]-6-carboxylic acid (2.8 g, yield 85%) as a yellow solid. The crude product was directly used in the next step without further purification. LC-MS(ESI): mass calcd. for C₁₈H₂₃NO₆, 349.15; m/z found, 350.2 [M+H]⁺.

### Step F: 1′-(Tert-Butoxycarbonyl)-7-Formyl-2H-Spiro[Benzofuran-3,3′-Pyrrolidine]-6-Carboxylic Acid

To a solution of 1′-[(tert-butoxy) carbonyl]-7-(hydroxymethyl)-2H-spiro[1-benzofuran-3,3′-pyrrolidine]-6-carboxylic acid (2.8 g, 8.0 mmol, 1.0 eq) in DCM (50 mL) was added Manganese dioxide (11.8 g, 136.2 mmol, 17.0 eq) at room temperature under nitrogen. The reaction was heated to 25° C. for 2 h. The reaction mixture was filtered and the filtrate was concentrated under reduced pressure. The crude product was purified by flash column chromatography on silica gel (DCM/MeOH=10/1) to provide 1′-[(tert-butoxy) carbonyl]-7-formyl-2H-spiro[1-benzofuran-3,3′-pyrrolidine]-6-carboxylic acid (1.2 g, yield 39%) as a yellow solid. LC-MS(ESI): mass calcd. for C₁₈H₂₁NO₆, 347.14; m/z found, 348.2 [M+H]⁺.

### Step G: Tert-Butyl 7-(2,6-Dioxopiperidin-3-Yl)-6-Oxo-7,8-Dihydro-2H,6H-Spiro[Furo [2,3-e]Isoindole-3,3′-Pyrrolidine]-1′-Carboxylate

To a solution of 1′-[(tert-butoxy) carbonyl]-7-formyl-2H-spiro[1-benzofuran-3,3′-pyrrolidine]-6-carboxylic acid (1.2 g, 3.5 mmol, 1.0 eq) and 3-aminopiperidine-2,6-dione hydrochloride (0.7 g, 5.2 mmol, 1.5 eq) in DMF (25 mL) was added HOAc (1.2 mL) at room temperature and the reaction mixture was stirred at 25° C. for 1 h. NaBH(OAc)3 (2.2 g, 10.3 mmol, 3.0 eq) was added to above mixture and the reaction mixture was stirred at 30° C. overnight. The reaction mixture was quenched with water (100 mL) and concentrated under reduced pressure. The crude product was purified by flash column chromatography on silica gel (EA/PE=2/1) to provide tert-butyl 7-(2,6-dioxopiperidin-3-yl)-6-oxo-2,6,7,8-tetrahydrospiro[furo [2,3-e]isoindole-3,3′-pyrrolidine]-1′-carboxylate (300 mg, yield 19%) as a blue solid. LC-MS(ESI): mass calcd. for C₂₃H₂₇N₃O₆, 441.19; m/z found, 442.2 [M+H]⁺.

### Step H: 3-(6-Oxo-6,8-Dihydro-2H,7H-Spiro[Furo [2,3-e]Isoindole-3,3′-Pyrrolidin]-7-Yl) Piperidine-2,6-Dione

To a mixture of tert-butyl 7-(2,6-dioxopiperidin-3-yl)-6-oxo-2,6,7,8-tetrahydrospiro[furo [2,3-e]isoindole-3,3′-pyrrolidine]-1′-carboxylate (300 mg, 0.7 mmol, 1.0 eq) in DCM (10 mL) was added HCl-dioxane (4 N) (10 mL) and the mixture was stirred at room temperature for 2 h. The mixture was concentrated under reduced pressure to give 3-{6-oxo-2,6,7,8-tetrahydrospiro[furo [2,3-e]isoindole-3,3′-pyrrolidine]-7-yl}piperidine-2,6-dione hydrochloride (150 mg, yield 65%) as a green solid. LC-MS(ESI): mass calcd. for C₁₈H₁₉N₃O₄, 341.14; m/z found, 342.2 [M+H]⁺. ¹H NMR (400 MHZ, DMSO-d₆) δ 10.98 (s, 1H), 9.53 (s, 2H), 7.60 (d, J=7.8 Hz, 1H), 7.37 (d, J=7.8 Hz, 1H), 5.14-5.06 (m, 1H), 4.70 (dd, J=9.2, 6.8 Hz, 1H), 4.62 (dd, J=9.2, 6.0 Hz, 1H), 4.41 (dd, J=17.2, 3.8 Hz, 1H), 4.25 (dd, J=17.2, 4.4 Hz, 1H), 3.56-3.46 (m, 2H), 3.32 (s, 2H), 2.97-2.85 (m, 1H), 2.59 (d, J=17.8 Hz, 1H), 2.47-2.36 (m, 1H), 2.29-2.23 (m, 2H), 2.04-1.94 (m, 1H).

### Compound A14. Tert-Butyl 6-(2,6-Dioxopiperidin-3-Yl)-7-Oxo-6,7-Dihydro-2H,5H-Spiro[Furo [2,3-f]Isoindole-3,4′-Piperidine]-1′-Carboxylate

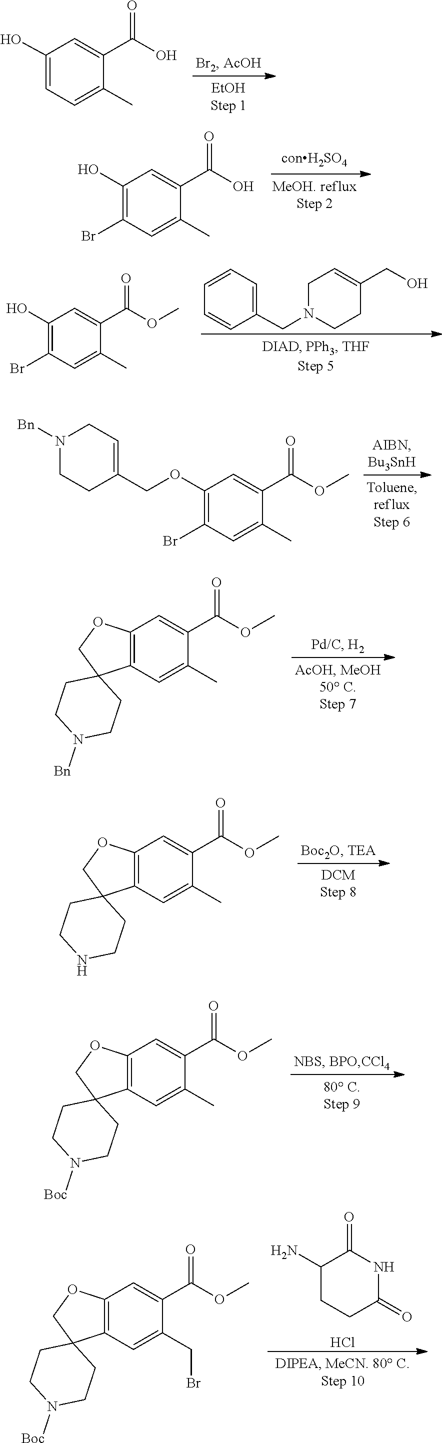

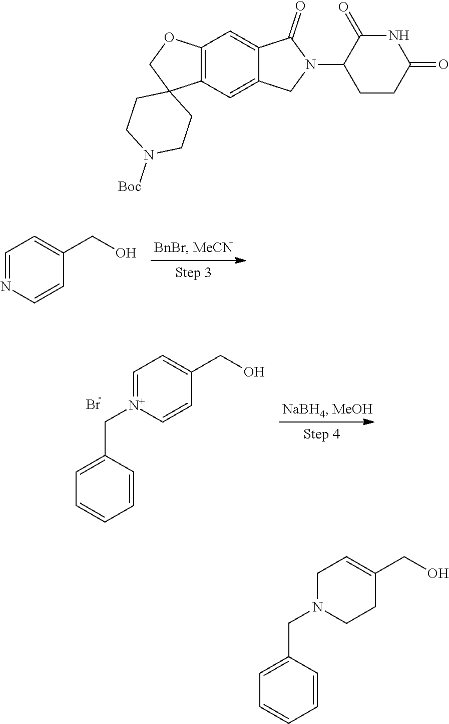 [SMILES: CC(C)(C)OC(=O)N1CCC2(CC1)COc1cc3c(cc12)CN(C1CCC(=O)NC1=O)C3=O ; OCc1ccncc1 ; OCc1cc[n+](Cc2ccccc2)cc1 ; Br ; OCC1=CCN(Cc2ccccc2)CC1]

### Step 1:4-Bromo-5-Hydroxy-2-Methylbenzoic Acid

To a solution of 5-hydroxy-2-methylbenzoic acid (5.0 g, 32.9 mmol, 1.0 eq) in a mixture of ethanol (20 mL) and acetic acid (10 mL) was added dropwise bromine (3.4 mL, 65.7 mmol, 2.0 eq.). The reaction mixture was stirred for 10 h at room temperature, quenched with aqueous sodium thiosulfate solution (50 mL), and concentrated. The aqueous layer was extracted with ethyl acetate (50 mL×3). The organic layer was dried over magnesium sulfate, filtered, and concentrated under reduced pressure to get crude 4-bromo-5-hydroxy-2-methylbenzoic acid (7.6 g, yield 100%) as a white solid. The crude product was directly used in next step without further purification. LC-MS(ESI): mass calcd. for C₈H₇BrO₃, 229.96; m/z found, 231.2 [M+H]⁺.

### Step 2: Methyl 4-Bromo-5-Hydroxy-2-Methylbenzoate

Con. H₂SO₄ (12 mL) was added to a suspension of 4-bromo-5-hydroxy-2-methylbenzoic acid (15 g, 65.72 mmol) in methanol (100 mL). The mixture was refluxed for 16 h. After evaporation, the residue was diluted with water (100 mL) and extracted with EA (100 mL×3). The organic layer was washed with H₂O (100 mL×2), saturated aqueous NaHCO₃ solution (100 mL×2) and brine (100 mL). The organic layer was dried over anhydrous Na₂SO₄, filtered and concentrated under reduced pressure. The residue purified by flash column chromatography on silica gel (PE/EA=4/1) to afford methyl 4-bromo-5-hydroxy-2-methylbenzoate (7.5 g, yield 47%) as a colorless solid. LC-MS(ESI): mass calcd. for C₉H₉BrO₃, 243.97; m/z found, 245.2 [M+H]⁺.

¹HNMR (400 MHZ, CDCl₃)δ 7.56 (s, 1H), 7.36 (s, 1H), 5.52 (s, 1H), 3.88 (s, 3H), 2.50 (s, 3H).

### Step 3:1-Benzyl-4-(Hydroxymethyl)Pyridin-1-Ium Bromide

To a solution of (pyridin-4-yl) methanol (8.9 g, 81.6 mmol, 1.0 eq) in CH₃CN (80 mL) was added a solution of (bromomethyl)benzene (11.705 mL, 97.9 mmol, 1.2 eq) in CH₃CN (40 mL). The reaction mixture was refluxed stirred at 90° C. for 3 h. After evaporation, the residue was washed with methyl tert-butyl ether, filtered, and dried to afford 1-benzyl-4-(hydroxymethyl)pyridin-1-ium bromide (16.33 g, yield 100%) as a yellow solid. LC-MS(ESI): mass calcd. for C₁₃H₁₄NO, 200.11; m/z found, 200.3 [M]⁺.

### Step 4: (1-Benzyl-1,2,3,6-Tetrahydropyridin-4-Yl) Methanol

To a solution of 1-benzyl-4-(hydroxymethyl)pyridin-1-ium bromide (16.3 g, 81.4 mmol, 1.0 eq) in CH₃OH (150 mL) was added NaBH₄ (9.3 g, 244.2 mmol, 3.0 eq) in portions at −20° C. The mixture was stirred at −20° C. for 1 h. The reaction was quenched with brine (100 mL) and extracted with EtOAc (200 mL×3). The organic layer was washed with brine (100 mL×3), dried over anhydrous Na₂SO₄, filtered and concentrated under reduced pressure. The residue was purified by flash column chromatography on silica gel (CH₃OH in DCM, from 0% to 10%) to afford (1-benzyl-1,2,3,6-tetrahydropyridin-4-yl) methanol (15 g, yield 91%) as a red oil. LC-MS(ESI): mass calcd. for C₁₃H₁₇NO, 203.13; m/z found, 204.4 [M+H]⁺.

¹H NMR (400 MHZ, DMSO-d₆) § 7.24-7.18 (m, 4H), 7.16-7.12 (m, 1H), 5.43 (s, 1H), 4.61 (s, 1H), 3.71 (s, 2H), 3.42 (s, 2H), 2.76 (s, 2H), 2.39 (t, J=5.6 Hz, 2H), 1.91 (s, 2H).

### Step 5: Methyl 5-[(1-Benzyl-1,2,3,6-Tetrahydropyridin-4-Yl) Methoxy]-4-Bromo-2-Methylbenzoate

To a solution of methyl 4-bromo-5-hydroxy-2-methylbenzoate (200 mg, 0.82 mmol, 1.0 eq), (1-benzyl-1,2,3,6-tetrahydropyridin-4-yl) methanol (166 mg, 0.82 mmol, 1.0 eq), and PPh₃ (321 mg, 1.22 mmol, 1.5 eq) in dry THF (10 mL) was added dropwise DIAD (0.25 mL, 1.22 mmol. 1.5 eq) at 0° C. under the N₂ atmosphere. The solution was stirred for 2 h. After evaporation, the residue was purified by flash column chromatography on silica gel (PE/EA=2/1 to 1/1) to afford methyl 5-[(1-benzyl-1,2,3,6-tetrahydropyridin-4-yl) methoxy]-4-bromo-2-methylbenzoate (300 mg, yield 85%) as a white solid. LC-MS(ESI): mass calcd. for C₂₂H₂₄BrNO₃, 429.09; m/z found, 431.30 [M+H]⁺.

### Step 6: Methyl 1′-(Cyclohexylmethyl)-5-Methyl-2H-Spiro[1-Benzofuran-3,4′-Piperidine]-6-Carboxylate

Tributyl tin hydride (0.5 mL, 1.84 mmol, 4.0 equiv) was added to a solution of methyl 5-[(1-benzyl-1,2,3,6-tetrahydropyridin-4-yl) methoxy]-4-bromo-2-methylbenzoate (200 mg, 0.46 mmol, 1.0 eq) and AIBN (15 mg, 0.09 mmol, 0.2 eq) in toluene (10 mL). The solution was refluxed in a sealed tube for 6 h. After cooled down to room temperature, The solution was quenched with saturated potassium fluoride solution (40 mL) and stirred at room temperature for 0.5 h. The mixture was extracted with EA (40 mL×3). The organic layer was washed brine (40 mL), dried over anhydrous Na₂SO₄, filtered and concentrated under reduced pressure. The residue was purified by prep-TLC (EA/PE=1/1) to afford methyl 1′-(cyclohexylmethyl)-5-methyl-2H-spiro[1-benzofuran-3,4′-piperidine]-6-carboxylate (20 mg, yield 43%) as a yellow solid. LC-MS(ESI): mass calcd. for C₂₂H₂₅NO₃, 351.18; m/z found, 352.30 [M+H]⁺.

¹H NMR (400 MHZ, CDCl₃)δ 7.37-7.27 (m, 6H), 6.99 (s, 1H), 4.37 (s, 2H), 3.85 (s, 3H), 3.54 (s, 2H), 2.89 (d, J=10.2 Hz, 2H), 2.52 (s, 3H), 2.10-1.95 (m, 4H), 1.70 (d, J=11.4 Hz, 2H).

### Step 7: Methyl 5-Methyl-2H-Spiro[Benzofuran-3,4′-Piperidine]-6-Carboxylate

A mixture of methyl 1′-benzyl-5-methyl-2H-spiro[1-benzofuran-3,4′-piperidine]-6-carboxylate (1.0 g, 2.845 mmol, 1.0 eq), acetic acid (1 mL, 5.7 mmol, 6.1 eq), and 10% Pd/C (200 mg) in MeOH (20 mL) was stirred at 50° C. under H₂ (1 atm) for 3 h. After filtration, the filtrate was concentrated to get methyl 5-methyl-2H-spiro[benzofuran-3,4′-piperidine]-6-carboxylate (970 mg, yield 100%) as a colorless oil, which was directly used in the next step without further purification. LC-MS(ESI): mass calcd. for C₁₅H₁₉NO₃, 261.14; m/z found, 262.40 (M+H)⁺.

### Step 8: 1′-(Tert-Butyl)6-Methyl 5-Methyl-2H-Spiro[Benzofuran-3,4′-Piperidine]-1′,6-Dicarboxylate

To a stirred solution of methyl 5-methyl-2H-spiro[1-benzofuran-3,4′-piperidine]-6-carboxylate (970 mg, 3.7 mmol, 1.0 eq) and TEA (1 mL, 7.4 mmol, 2.0 eq) in DCM (10 mL) was added dropwise Boc₂O (0.8 mL, 3.7 mmol, 2.0 eq) at 0° C. The mixture was stirred at room temperature for 2 h. The reaction mixture was poured into water (10 mL) and extracted with DCM (30 mL×2). The organic phase was dried over anhydrous Na₂SO₄ and concentrated under reduced pressure to afford 1′-(tert-butyl)6-methyl 5-methyl-2H-spiro[benzofuran-3,4′-piperidine]-1′,6-dicarboxylate (1.28 g, yield 100%) as a white solid. LC-MS(ESI): mass calcd. for C₂₀H₂₇NO₅, 361.19; m/z found, 306.4 [M+H-56]⁺.

### Step 9: 1′-(Tert-Butyl)6-Methyl 5-(Bromomethyl)-2H-Spiro[Benzofuran-3,4′-Piperidine]-1′,6-Dicarboxylate

A mixture of methyl 1′-benzyl-5-methyl-2H-spiro[1-benzofuran-3,4′-piperidine]-6-carboxylate (220 mg, 0.609 mmol, 1 eq), NBS(130 mg, 0.73 mmol, 1.2 eq), and BPO (60 mg, 0.243 mmol, 0.4 eq) in CCl₄ (10 mL) was refluxed for 4 h. After cooled to room temperature, the mixture was filtered, then the filtration was concentrated and to give 1′-tert-butyl 6-methyl 5-(bromomethyl)-2H-spiro[1-benzofuran-3,4′-piperidine]-1′,6-dicarboxylate (100 mg, yield 37%) as a light-yellow solid. LC-MS(ESI): mass calcd. for C₂₀H₂₆BrNO₅, 439.10; m/z found, 462.20, [M+Na]⁺.

### Step 10: Tert-Butyl 6-(2,6-Dioxopiperidin-3-Yl)-7-Oxo-6,7-Dihydro-2H,5H-Spiro[Furo [2,3-Flisoindole-3,4′-Piperidine]-1′-Carboxylate

DIPEA (0.12 mL, 0.681 mmol, 3.0 eq) was added to 1′-tert-butyl 6-methyl 5-(bromomethyl)-2H-spiro[1-benzofuran-3,4′-piperidine]-1′,6-dicarboxylate (100 mg, 0.227 mmol, 1.0 eq) and 3-aminopiperidine-2,6-dione hydrochloride (56 mg, 0.341 mmol, 1.5 eq) in MeCN (5 mL) under nitrogen. The resulting suspension was stirred at 80° C. for 24 h. The reaction mixture was cooled to room temperature and filtered. The solid was washed with MeCN and purified by prep-TLC (100% EtOAc) to afford tert-butyl 6-(2,6-dioxopiperidin-3-yl)-7-oxo-6,7-dihydro-2H,5H-spiro[furo [2,3-f]isoindole-3,4′-piperidine]-1′-carboxylate (50 mg, yield 48%) as a white solid. LC-MS(ESI): mass calcd. for C₂₄H₂₉N₃O₆, 455.51; m/z found, 456.50, (M+H)⁺.

### Compound A15. (S)-3-(6-Oxo-6,8-Dihydro-2H,7H-Spiro[Furo [2,3-e]Isoindole-3,4′-Piperidin]-7-Yl) Piperidine-2,6-Dione

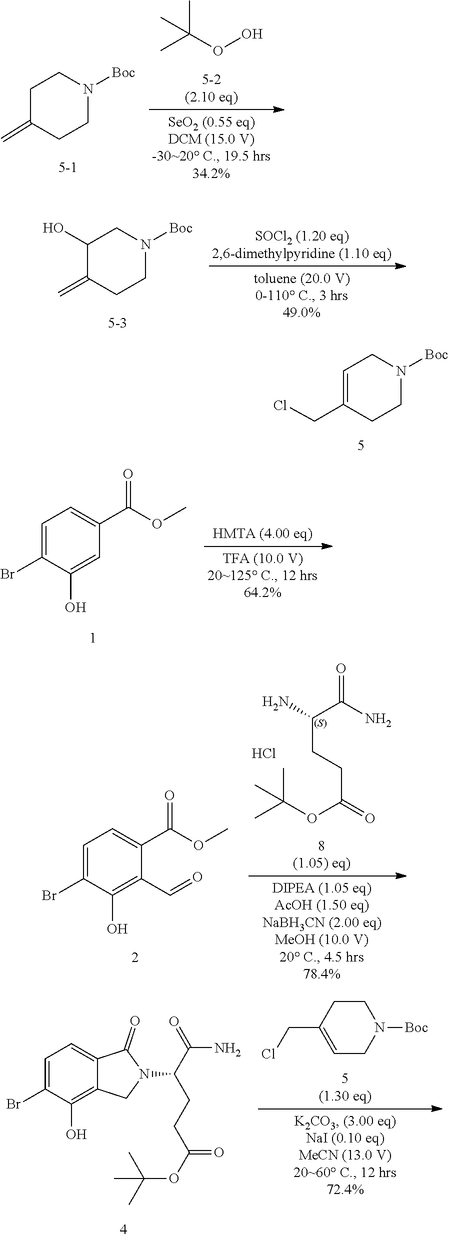

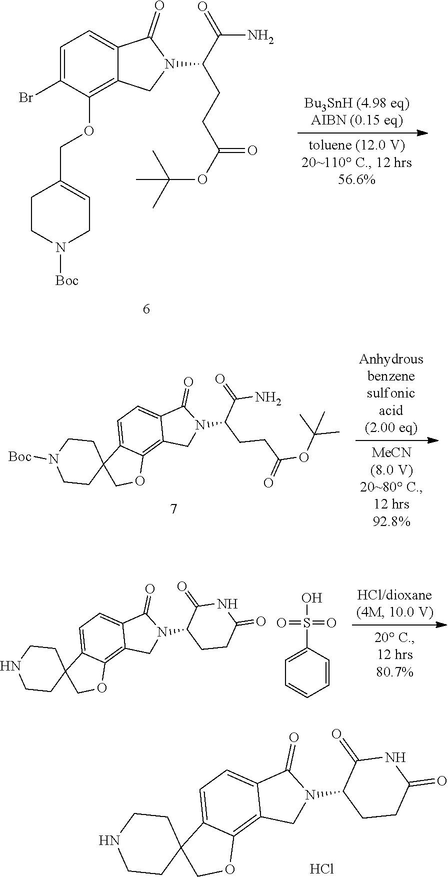

### Step 1: Synthesis of Tert-Butyl 3-Hydroxy-4-Methylene-Piperidine-1-Carboxylate

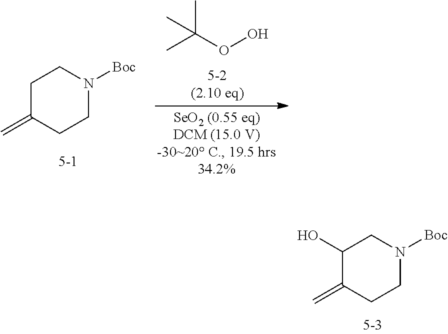

The suspension of SeO₂ (61.8 g, 558 mmol, 0.55 eq) in DCM (3000 mL) was cooled to −10° C., before 2-hydroperoxy-2-methyl-propane in H₂O (274 g, 291 mL, 2.10 eq, 70% purity) was added dropwise, and the resulting mixture was stirred for 30 min at −10° C. The reaction mixture was further cooled to −30° C., before a solution of compound 5-1 (200 g, 1.01 mol, 1 eq) in DCM (1000 mL) was added dropwise, and the resulting mixture was stirred for another 1 hr at −30° C. The reaction mixture was warmed to 20° C., and stirred for further 18 hrs, before the mixture was cooled to 0° C., and was added ice chips and water (1.0 L). The resulting mixture was stirred at 0° C. for 30 min. The organic phase was separated, and the aqueous phase was extracted with DCM (500 mL), before the combined organic phase was added 10% w/v NaHSO₃ solution (1000 mL) portion-wise at 0° C., during which period the temperature was maintained below 10° C., and the mixture was stirred for further 5 min after the addition. The organic phase was separated, and the aqueous phase was extracted with DCM (500 mL). The combined organic phase was washed with brine (1000 mL), dried over anhydrous Na₂SO₄, filtered, and concentrated in vacuo. The crude product was purified by silica gel column chromatography (Petroleum ether/Ethyl acetate=100/1 to 1/1). Compound 5-3 (370 g) was obtained as a white solid and the typical yield was 34.2%. ¹HNMR (400 MHZ, DMSO-d₆) i=5.22 (br d, J=3.9 Hz, 1H), 4.98 (s, 1H), 4.79 (s, 1H), 3.93-3.76 (m, 2H), 3.71 (td, J=4.3, 12.6 Hz, 1H), 2.92-2.77 (m, 1H), 2.76-2.53 (m, 1H), 2.30 (td, J=3.5, 13.4 Hz, 1H), 2.10-1.95 (m, 1H), 1.40 (s, 9H).

### Step 2: Synthesis of Tert-Butyl 4-(Chloromethyl)-3,6-Dihydro-2H-Pyridine-1-Carboxylate

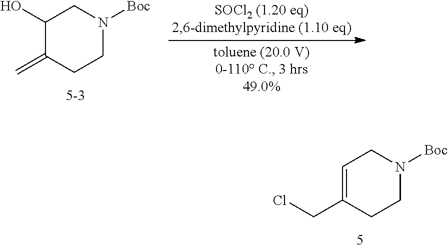

To the solution of Compound 5-3 (100 g, 469 mmol, 1.00 eq) in toluene (2000 mL) was add 2,6-dimethylpyridine (55.2 g, 60.0 mL, 516 mmol, 1.10 eq) at 15° C. The mixture was cooled to 0° C., before SOCI₂ (66.9 g, 40.8 mL, 563 mmol, 1.20 eq) was added dropwise to the mixture under N₂ atmosphere, during which period the temperature was maintained below 10° C. The mixture was stirred at 110° C. for 3 hrs, before cooled to 20° C. Brine (2×600 mL) was added and the resulting mixture was stirred at 20° C. for 30 min. The organic phase was separated, before saturated NaHCO₃ solution (600 mL) was added portion-wise at 15° C. The organic phase was separated, washed with brine (1000 mL), dried over anhydrous Na₂SO₄, filtered and concentrated in vacuo. Compound 5 (200 g) was obtained as a red oil and the typical yield was 49.0%. ¹HNMR (400 MHZ, CDCl₃-d) δ=5.72 (br s, 1H), 3.98 (s, 2H), 3.88 (br s, 2H), 3.49 (br t, J=5.6 Hz, 2H), 2.17 (br s, 2H), 1.43 (s, 9H).

### Step 3: Synthesis of Methyl 4-Bromo-2-Formyl-3-Hydroxybenzoate

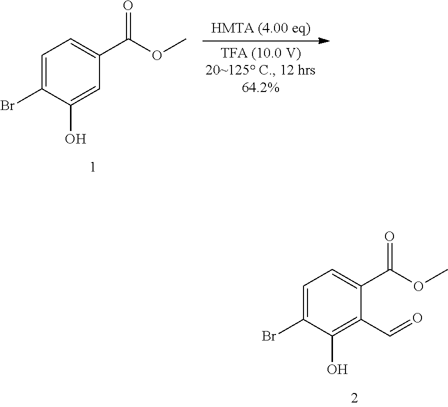

The solution of Compound 1 (200 g, 865 mmol, 1.00 eq) in TFA (2.0 L) was added HMTA (485 g, 3.46 mol, 4.00 eq) at 20° C., before the resulting mixture was stirred at 125° C. for 12 hrs. The mixture was cooled to 20° C., quenched with 2N HCl solution (5 V), and yellow precipitate was observed. The mixture was stirred for 10 min, before additional H₂O (5 V) was added, and the reaction mixture was stirred for further 1 hr. The mixture was filtered, and the filter cake was dissolved in DCM (2.0 L), filtered over celite, dried over anhydrous Na₂SO₄ and concentrated in vacuo. Compound 2 (144 g) was obtained as a gray solid, and the typical yield was 64.2%. ¹HNMR (400 MHZ, DMSO-d₆) δ=12.06 (br s, 1H), 10.38 (s, 1H), 8.00 (d, J=8.2 Hz, 1H), 7.30 (d, J=8.2 Hz, 1H), 3.87 (s, 3H).

### Step 4: Synthesis of(S)-Tert-Butyl 5-Amino-4-(5-Bromo-4-Hydroxy-1-Oxoisoindolin-2-Yl)-5-Oxopentanoate

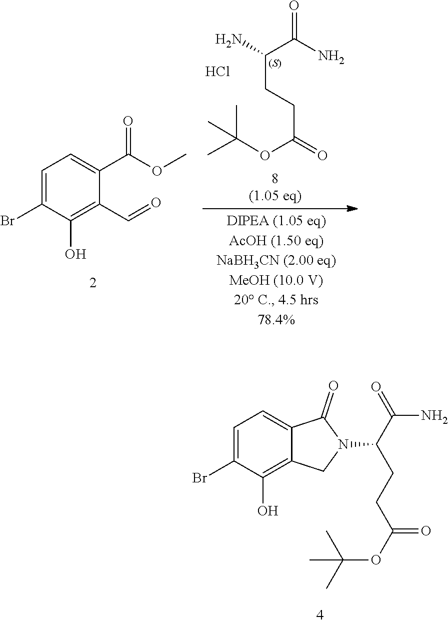

To the suspension of compound 8 (17.3 g, 72.4 mmol, 1.05 eq, HCl salt) in MeOH (300 mL) was added DIPEA (9.37 g, 72.4 mmol, 12.6 mL, 1.05 eq), compound 2, (17.8 g, 69.0 mmol, 1.00 eq) and AcOH (6.22 g, 103 mmol, 5.92 mL, 1.50 eq) at 20° C. and stirred for 1.5 hrs, before NaBH₃CN (8.67 g, 138 mmol, 2.00 eq) was added portion-wise at 20° C., and the resulting mixture was stirred at 20° C. for 3 hrs. The mixture was quenched by H₂O (200 mL) at 20° C. and concentrated under reduced pressure. The solvent residue was then extracted with EtOAc (3×150 mL), and the combined organic layer was washed with brine (2×200 mL), dried over anhydrous Na₂SO₄, filtered, and concentrated in vacuo. The crude product was purified by silica gel column chromatography (Petroleum ether/Ethyl acetate=1/1 to 100% Ethyl acetate). Compound 4 (23.0 g) was obtained as a yellow solid and the typical yield was 78.4%. ¹HNMR (400 MHZ, DMSO-d₆) δ=10.44 (s, 1H), 7.67-7.55 (m, 2H), 7.20 (s, 1H), 7.11 (d, J=7.9 Hz, 1H), 4.76-4.67 (m, 1H), 4.58 (d, J=17.9 Hz, 1H), 4.39 (d, J=17.9 Hz, 1H), 2.23-2.07 (m, 3H), 2.03-1.91 (m, 1H), 1.32 (s, 9H).

### Step 5: Synthesis of(S)-Tert-Butyl 4-(((2-(1-Amino-5-(Tert-Butoxy)-1,5-Dioxopentan-2-Yl)-5-Bromo-1-Oxoisoindolin-4-Yl)Oxy)Methyl)-5,6-Dihydropyridine-1 (2H)-Carboxylate

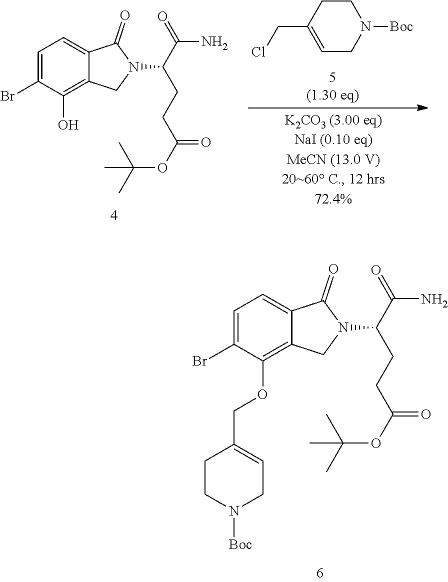 [SMILES: CC(C)(C)OC(=O)CC[C@@H](C(N)=O)N1Cc2c(ccc(Br)c2O)C1=O ; CC(C)(C)OC(=O)N1CC=C(CCl)CC1 ; CC(C)(C)OC(=O)CC[C@@H](C(N)=O)N1Cc2c(ccc(Br)c2OCC2=CCN(C(=O)OC(C)(C)C)CC2)C1=O]

To the solution of compound 4 (150 g, 363 mmol, 1.00 eq) in MeCN (2000 mL) was added K₂CO₃ (150.49 g, 1.09 mmol, 3.00 eq), NaI (5.44 g, 0.36 mmol, 0.10 eq) and compound 5 (136 g, 472 mmol, 1.30 eq, 80% purity) at 20° C. The reaction mixture was stirred at 60° C. for 12 hrs, before being cooled to 20° C. again. The resulting mixture was filtered, and filter cake was washed with DCM (2×500 mL). The filtrate was concentrated in vacuo, and the crude product was purified by silica gel column chromatography (Petroleum ether/Ethyl acetate =100/1 to 1/1). Compound 6 (337 g) was obtained as a red solid, and the typical yield was 72.4%. ¹HNMR (400 MHZ, CDCl₃-d) δ=7.67 (d, J=8.0 Hz, 1H), 7.44 (d, J=8.0 Hz, 1H), 6.33 (br s, 1H), 5.86 (br s, 1H), 5.48 (br s, 1H), 4.90 (dd, J=6.3, 8.6 Hz, 1H), 4.66-4.59 (m, 1H), 4.53 (s, 1H), 4.50 (br s, 2H), 3.98 (br s, 2H), 3.60 (br t, J=5.5 Hz, 2H), 2.43-2.10 (m, 7H), 1.49 (s, 9H), 1.41 (s, 9H).

### Step 6: Synthesis of Tert-Butyl 7-[(1S)-4-Tert-Butoxy-1-Carbamoyl-4-Oxo-Butyl]-6-Oxo-Spiro[2,8-Dihydrofuro [2,3-e]Isoindole-3,4′-Piperidine]-1′-Carboxylate

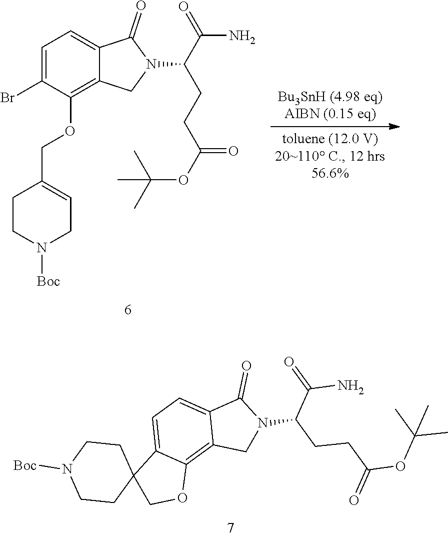 [SMILES: CC(C)(C)OC(=O)CC[C@@H](C(N)=O)N1Cc2c(ccc(Br)c2OCC2=CCN(C(=O)OC(C)(C)C)CC2)C1=O ; CC(C)(C)OC(=O)CC[C@@H](C(N)=O)N1Cc2c(ccc3c2OCC32CCN(C(=O)OC(C)(C)C)CC2)C1=O]

To the solution of compound 6 (125 g, 205 mmol, 1.00 eq) in toluene (1500 mL) was added AIBN (5.06 g, 0.03 mmol, 0.15 eq) and Bu₃SnH (270 mL, 1.02 mmol, 4.98 eq) at 20° C. The reaction mixture was stirred at 110° C. for 12 hrs, before being cooled to 20° C. Saturated KF solution (1000 mL) was added and the resulting mixture was stirred at 20° C. for further 2 hrs. The mixture was filtered and the filter cake was washed by EtOAc (2×500 mL). The organic phase was separated, and the aqueous phase was extracted with ethyl acetate (3×500 mL). The combined organic phase was washed with brine (500 mL), dried over anhydrous Na₂SO₄, filtered and concentrated in vacuo. The crude product was purified by silica gel column chromatography (Petroleum ether/Ethyl acetate=100/1 to 1/1). Compound 7 (160 g) was obtained as a white solid, and the typical yield was 56.6%. ¹HNMR (400 MHZ, CDCl₃-d) δ=7.40 (d, J=7.6 Hz, 1H), 7.20 (d, J=7.6 Hz, 1H), 6.41 (br s, 1H), 5.61 (br s, 1H), 4.92-4.85 (m, 1H), 4.55-4.49 (m, 2H), 4.12 (q, J=7.1 Hz, 3H), 2.88 (br t, J=12.0 Hz, 2H), 2.40-2.09 (m, 5H), 1.94-1.82 (m, 2H), 1.77-1.68 (m, 2H), 1.50-1.47 (m, 9H), 1.42-1.40 (m, 9H).

### Step 7: Synthesis of (3S)-3-(6-Oxospiro[2,8-Dihydrofuro [2,3-e]Isoindole-3,4′-Piperidine]-7-Yl) Piperidine-2,6-Dione Benzenesulfonate

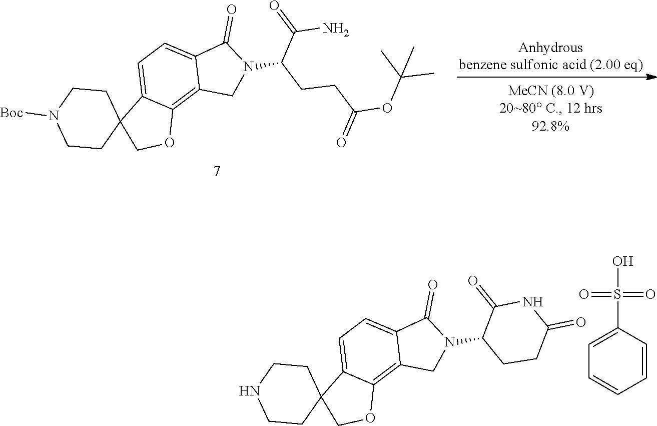

The solution of anhydrous benzene sulfonic acid (19.6 g, 124 mmol, 2.00 eq) in MeCN (400 mL) was heated to 100° C., before a solution of compound 7 (47.0 g, 62.1 mmol, 1.00 eq, 70% purity) in MeCN (100 mL) was added dropwise to the mixture. The mixture was stirred at 100° C. for 12 hrs, before being cooled to 20° C. The mixture was filtered, and the filter cake was dried under reduce pressure. The title compound (37.0 g) was obtained as a white solid, and the typical yield was 92.8%. ¹HNMR (400 MHZ, D₂O-d₂) δ=7.75 (br d, J=7.4 Hz, 2H), 7.55-7.36 (m, 5H), 5.11 (br dd, J=5.2, 13.4 Hz, 1H), 4.64 (s, 2H), 4.53-4.35 (m, 2H), 3.49 (br d, J=13.2 Hz, 2H), 3.20-3.06 (m, 2H), 2.99-2.78 (m, 2H), 2.48 (dq, J=5.3, 13.1 Hz, 1H), 2.25-2.08 (m, 3H), 2.05-1.93 (m, 5H).

### Step 8: Synthesis of (3S)-3-(6-Oxospiro[2,8-Dihydrofuro [2,3-e]Isoindole-3,4′-Piperidine]-7-Yl) Piperidine-2,6-Dione Hydrochloric Acid

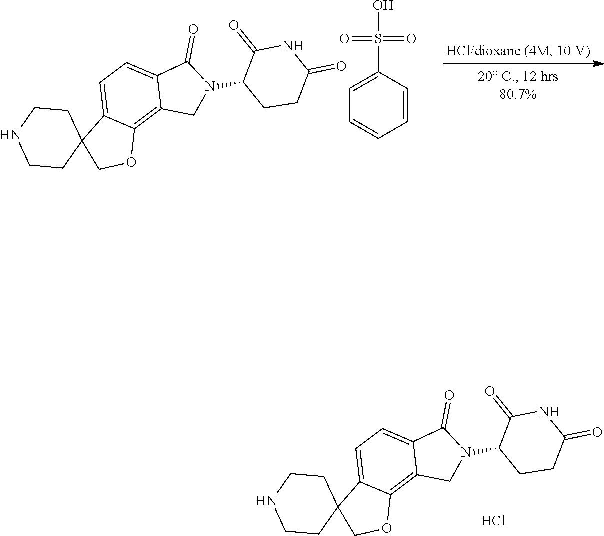

The solution of (3S)-3-(6-oxospiro[2,8-dihydrofuro [2,3-e]isoindole-3,4′-piperidine]-7-yl) piperidine-2,6-dione benzenesulfonate (37 g) in HCl/dioxane (4 M, 370 mL) was stirred at 20° C. for 12 hrs, before the mixture was filtered and the cake was washed with MeCN (2×200 mL). The filtered cake was dried under reduced pressure. The title compound (24.0 g) was obtained as a red solid, and the typical yield was 80.7%. ¹HNMR (400 MHZ, D₂O-d₂) δ=7.48-7.36 (m, 2H), 5.12 (dd, J=5.3, 13.3 Hz, 1H), 4.69-4.61 (m, 2H), 4.53-4.37 (m, 2H), 3.51 (br dd, J=3.4, 13.3 Hz, 2H), 3.22-3.06 (m, 2H), 2.98-2.80 (m, 2H), 2.56-2.43 (m, 1H), 2.29-2.08 (m, 3H), 2.05-1.90 (m, 2H).

### Compound A16. 3-(7′-Oxo-2′,3′,7′,9′-Tetrahydro-8′H-Spiro[Piperidine-4,4′-Pyrano[2,3-e]Isoindol]-8′-Yl) Piperidine-2,6-Dione

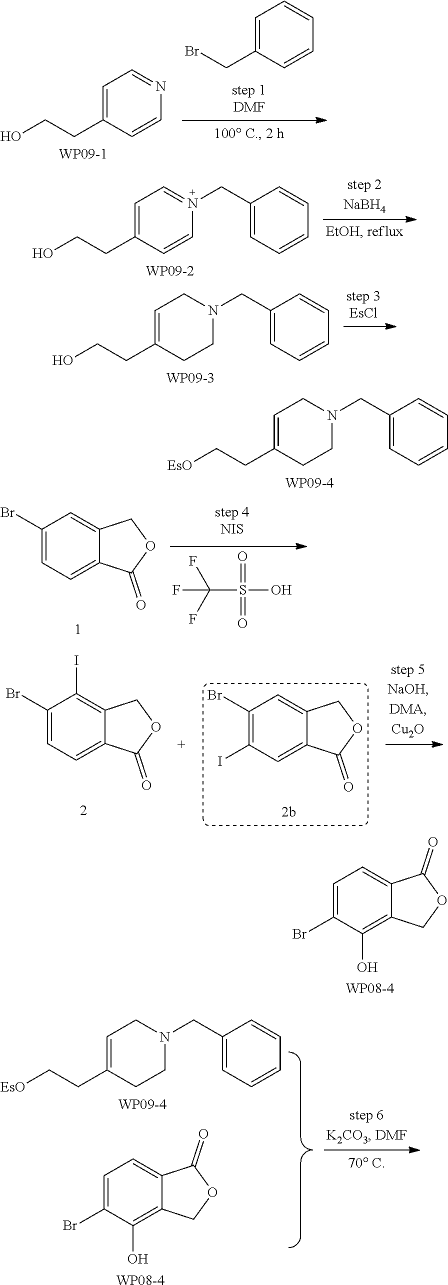

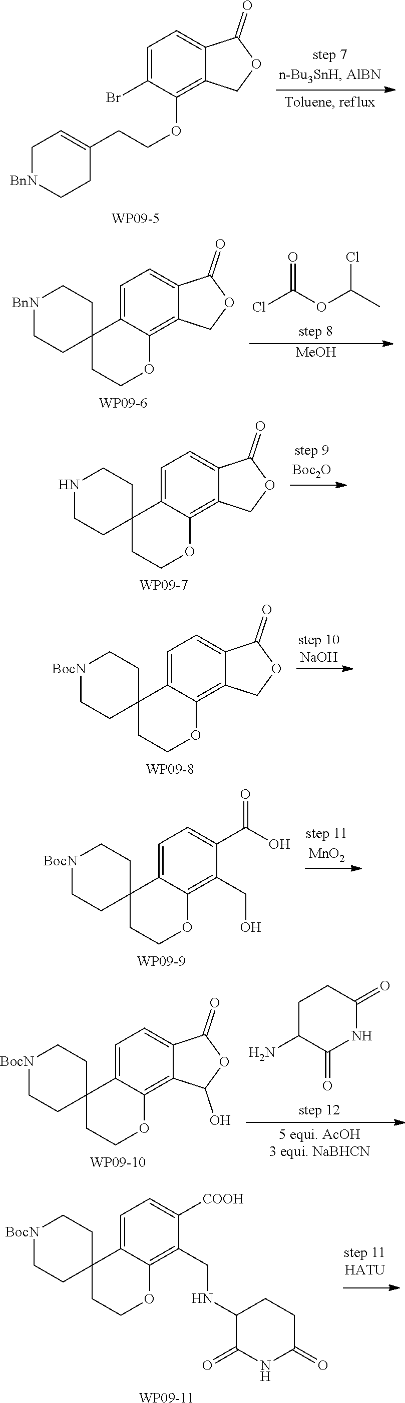

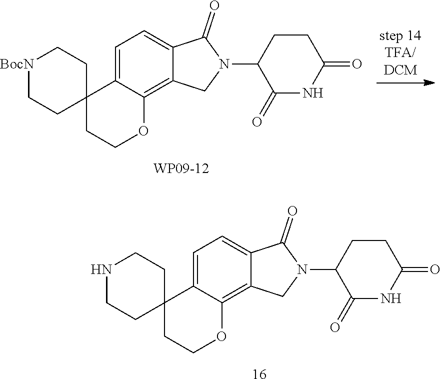

### Step 1-2:

To a solution of 2-(pyridin-4-yl) ethan-1-ol (WP09-1, 10 g, 91.6 mmol, 1.0 eq.) in DMF (40 mL) was added BnBr (15.3 g, 108 mmol, 1.1 eq.). The mixture was allowed to heat to 100° C. and stirred 3 h. TLC showed no starting material remained and a new spot formed. The residue was dissolved in EtOH (150 mL), then 4.0 g of sodium borohydride (119.1 mmol, 1.3 eq.) was added portionwise at 0° C. The mixture was continued to stir at 0° C. for 1 h and then at reflux for 2 h. The solvent was evaporated under reduced pressure, then water was added, and the mixture was extracted with EA. The combined organic phases were dried over Na₂SO₄ and evaporated. The residue was purified by flash chromatograph (DCM:MeOH=100:0-30:1) to afford 10 g of product WP09-3 (Viscous oil, 2 steps, yield 56%).

LC-MS: 218 [M+H]⁺

### Step 3:

To a solution of compound WP09-3 (10 g, 1 eq.) in DCM (200.0 mL) was added DMAP (0.1 eq.) and TEA (2 eq.) at 0° C. Then EsCl (1.5 eq.) was slowly added into and the mixture was stirred at R. T. for 1 h. The reaction was partitioned between EtOAc and water. The combined organic layers were dried over anhydrous Na₂SO₄, filtered, and concentrated in vacuo. The crude residue was purified by flash chromatograph to give compound WP09-4 as a yellow solid (10 g, yield 70%).

LC-MS: 310 [M+H]⁺

### Step 4:

To a solution of 5—Bromo-3H-isobenzofuran-1-one (1) (10 g, 1 eq.) in trifluoromethanesulfonic acid (100 g, 10 V) was added NIS(12.5 g, 1.2 eq.) at 0° C. in portions. The mixture was allowed to warm to room temperature and stirred overnight. TLC showed no starting material remained and two new spots formed. The reaction mixture was poured into ice-water and yellow solid precipitated. The mixture was filtered and the filter cake was washed with ice cold water. The filter cake was dried and obtained as a yellow solid (10 g, yield 62%), to be a mixture of product 2 (top spot on TLC) and product 2b (bottom spot on TLC, which was not further reacted in next step).

### Step 5:

To a mixture of compound 2 (10 g, 1 eq.), sodium hydroxide (5.75 g, 5 eq.) in water (100 mL, 1.5 M) and N,N-dimethylacetamide (60 mL) was added cuprous oxide (0.85 g, 0.2 eq.). The reaction mixture was heated to 80° C. and stirred for 12 h. TLC showed the compound 2 (top spot on TLC) was completely consumed. The reaction mixture was poured into water (100 mL) and treated with solid K₂CO₃ until pH 8-9, and extracted with EA. The aqueous layer neutralized using 1 N hydrochloride solution and extracted with ethyl acetate, washed with brine, and then dried over sodium sulfate and evaporated. The crude product was purified by silica gel column chromatography to give compound WP08-4 was obtained as a yellow solid (4.2 g, 39% yield).

LC-MS: 229/231 [M+H]⁺

### Step 6:

To a solution of compound WP08-4 (10 g, 1.0 eq.) in 100 mL of DMF, compound WP09-4 (16.2 g, 1.2 eq.) and K₂CO₃ (1.6 eq.) was added. The reaction mixture was heated to 70° C. and stirred overnight. The reaction mixture was poured into ice-water and extracted with ethyl acetate, washed with brine, and then dried over sodium sulfate. The solvent was evaporated at reduced pressure and the crude product was purified by silica gel column chromatography using 0-100% EtOAc/hexane. The desired product WP09-5 was obtained as a yellow foam (11 g, yield 60%).

LC-MS: 428/430 [M+H]⁺.

### Step 7:

To a solution of WP09-5 (5 g, 1.0 eq.) in toluene (50 mL) was added n-Bu₃SnH (13.6 g, 4.0 eq.) and AIBN (0.4 g, 0.1 eq.). The mixture was heated to reflux and stirred overnight. TLC (PE:EA=1:1) showed no starting material remained and new spots formed. The reaction mixture was poured into saturated aq. KF solution (100 mL) and stirred overnight. Then, the reaction mixture was extracted with ethyl acetate, washed with brine, and then dried over sodium sulfate. The crude product was purified by silica gel column chromatography (DCM: MeOH=50:1) to give compound WP09-6 was obtained as a white solid (2 g, 50% yield).

LC-MS: 350 [M+H]⁺.

### Step 8-9:

To a solution of WP09-6 (3.0 g, 1.0 eq.) in DCE (100 mL) was added α-chloroethyl chloroformate (ACE-C1,1.2 eq.) at 0° C. and then refluxing the mixture for 15 h. The intermediate ACE-piperidine formed and is usually deACEylated directly to WP09-7 by evaporating the reaction mixture in vacuo and then heating the residue in MeOH. The residue was dissolved in THF (100 mL), then trimethylamine (3.0 eq.) and Boc₂O (1.3 eq.) was added. The mixture was continued to stir for 3 h at room temperature. The solvent was evaporated under reduced pressure, then water was added, and the mixture was extracted with EA. The combined organic phases were dried over Na₂SO₄ and evaporated. The residue was purified by flash chromatograph to afford WP09-8 (1.5 g, 2 steps, yield 50%).

LC-MS: 360 [M+H]⁺. ¹H NMR (600 MHZ, Chloroform-d)δ 7.47 (d, J=7.6 Hz, 1H), 7.08 (d, J=7.6 Hz, 1H), 5.24 (s, 2H), 4.16 (t, J=6.7 Hz, 2H), 3.88 (m, 2H), 3.51 (m, 2H), 2.52 (t, J=6.8 Hz, 2H), 2.13 (m, 2H), 1.61 (m, 2H), 1.46 (s, 9H).

### Step 10:

To a solution of compound WP09-8 (2 g, 1 eq.) in tetrahydrofuran (10 mL) and water (10 mL) was added sodium hydroxide (1.2 g, 5 eq.). The mixture was stirred at 20° C. for 16 h. TLC (ethyl acetate: hexane=1:1) showed reaction was complete. The mixture was adjusted to pH=5-6 with aq. hydrochloric acid (1 M) and extracted with ethyl acetate. The organic layer was washed with brine and dried over sodium sulfate. The crude material was not further purified and used as crude for the next step.

### Step 11:

To a solution of compound WP09-9 (2 g, crude, 1 eq.) in dichloromethane (30 mL) was added manganese dioxide (20 eq.). The mixture was stirred at 20° C. for about 1 h. TLC showed reaction was complete. The mixture was diluted with dichloromethane and filtered through a pad of Celite. The filtrate was concentrated in vacuum. The crude product was purified by silica gel column chromatography (DCM:MeOH=10:1). The desired compound WP09-10 was obtained as yellow solid. (1.2 g, 2 steps, 60%).

LC-MS: 376 [M+H]⁺.

### Step 12:

To a mixture of compound WP09-10 (532 mg, 1.0 eq.) in methanol (5 mL) and dichloromethane (5 mL) was added 3-aminopiperidine-2,6-dione (698 mg, 3 eq., HCl salt), AcONa (698 mg, 6.0 eq.) and AcOH (0.85 mL, 10.0 eq.). The mixture was stirred at 25° C. for 1 h, then sodium cyanoborohydride (268 mg, 3.0 eq.) was added and the mixture was further stirred for 30 min. LCMS showed the reaction was complete. Next, the reaction mixture was quenched with water and concentrated under reduced pressure to give a residue which was purified by pre-HPLC (20% ˜50% ACN, neutral). The desired product WP09-11 as a solid (415 mg, yield 60%) after lyophilization.

### Step 13:

To a solution of compound WP09-11 (415 mg 1.0 equiv) in DMF (5 mL) was added HATU (421 mg, 1.3 equiv) and DIPEA (0.47 mL, 3.0 equiv), and the reaction was stirred at rt for 30 min. UPLC-MS indicated a new main peak with desired MS formed, then quenched with water and the mixture was extracted with ethyl acetate, washed with brine, and then dried over sodium sulfate. The desired compound WP09-12 was obtained as a brown solid (300 mg, 75% yield).

LC-MS: 470 [M+H]⁺.

### Step 14:

Compound WP09-12 was treated with TFA in DCM at room temperature to de-protect the N-Boc group to provide the cereblon ligand 16.

LC/MS(ESI) m/z: 369.2.

### Compound A17. 3-(3′,3′-Difluoro-6-Oxo-6,8-Dihydro-2H,7H-Spiro[Furo [2,3-e]Isoindole-3,4′-Piperidin]-7-Yl) Piperidine-2,6-Dione

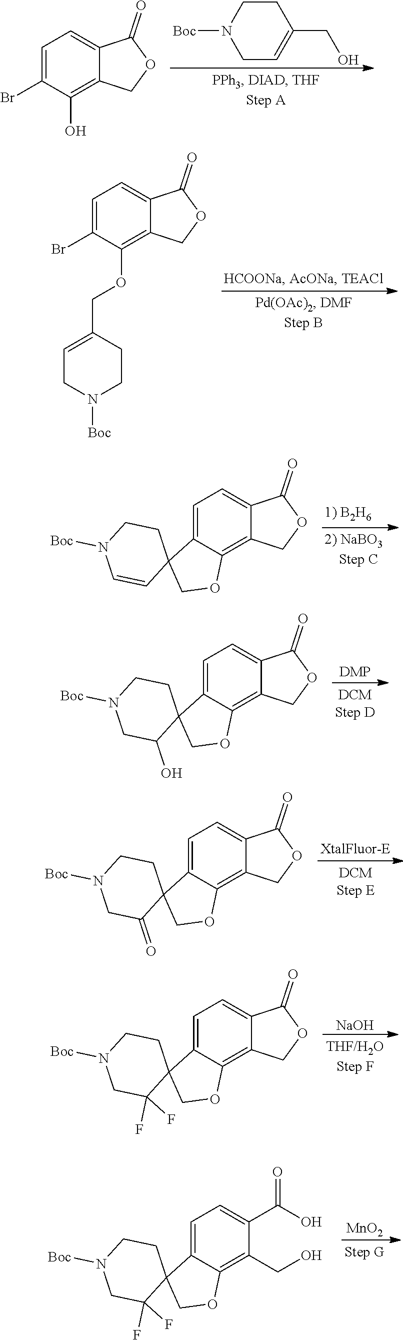

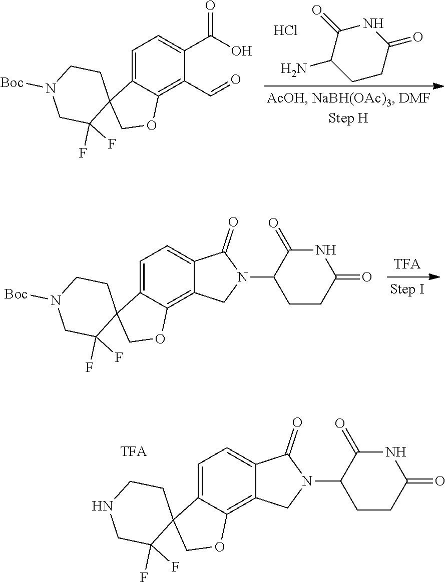 [SMILES: CC(C)(C)OC(=O)N1CCC2(COc3c2ccc(C(=O)O)c3C=O)C(F)(F)C1 ; Cl ; NC1CCC(=O)NC1=O ; CC(C)(C)OC(=O)N1CCC2(COc3c2ccc2c3CN(C3CCC(=O)NC3=O)C2=O)C(F)(F)C1 ; O=C1CCC(N2Cc3c(ccc4c3OCC43CCNCC3(F)F)C2=O)C(=O)N1]

### Step A: Tert-Butyl 4-(((5-Bromo-1-Oxo-1,3-Dihydroisobenzofuran-4-Yl)Oxy)Methyl)-3,6-Dihydropyridine-1 (2H)-Carboxylate

To a solution of 5-bromo-4-hydroxyisobenzofuran-1 (3H)-one (5.0 g, 21.8 mmol, 1.0 eq) in THF (150 mL) were added tert-butyl-4-(hydroxymethyl)-3,6-dihydropyridine-1 (2H)-carboxylate (5.59 g, 26.2 mmol, 1.2 eq) and Triphenylphosphine (8.59 g, 32.7 mmol, 1.5 eq). The mixture was stirred under N₂ at 0° C. for 20 min. Then DIAD (6.44 mL, 32.7 mmol, 1.5 eq) was dropwise added to above mixture and the mixture was stirred at room temperature overnight. After evaporation, the crude product was purified by flash column chromatography on silica gel (ethyl acetate in petroleum ether, from 0% to 20%) to afford tert-butyl 4-(((5-bromo-1-oxo-1,3-dihydroisobenzofuran-4-yl)oxy)methyl)-3,6-dihydropyridine-1 (2H)-carboxylate (9.0 g, yield 97%) as a yellow solid. LC-MS(ESI): mass calcd. for C₁₉H₂₂BrNO₅, 423.07; m/z found, 368 [M+H-56]⁺.

### Step B: Tert-Butyl 6-Oxo-2′,3′,6,8-Tetrahydro-1′H,2H-Spiro[Benzo[2,1-b: 3,4-c′]Difuran-3,4′-Pyridine]-1′-Carboxylate

To a solution of tert-butyl 4-(((5-bromo-1-oxo-1,3-dihydroisobenzofuran-4-yl)oxy)methyl)-3,6-dihydropyridine-1 (2H)-carboxylate (5.0 g, 7.9 mmol, 1.0 eq) in DMF (30 mL) were added sodium formate (591 mg, 8.69 mmol, 1.1 eq), palladium diacetate (177 mg, 790 μmol, 0.1 eq), sodium acetate (1.62 g, 19.7 mmol, 2.5 eq) and TEA (1.44 g, 8.69 mmol, 1.33 mL, 1.1 eq). The mixture was stirred under N₂ at 70° C. for 16 hours. After cooled to room temperature, the mixture was filtered and the cake was washed with EA (100 mL). The filtrate was washed with brine (60 mL×3), dried over anhydrous sodium sulfate, filtered and concentrated under reduced pressure. The residue was purified by flash column chromatography on silica gel (ethyl acetate in petroleum ether, from 0% to 30%) to afford tert-butyl 6-oxo-2′,3′,6,8-tetrahydro-1′H,2H-spiro[benzo[2,1-b: 3,4-c′]difuran-3,4′-pyridine]-1′-carboxylate (1.7 g, yield 63%) as a colorless oli. LC-MS(ESI): mass calcd. for C₁₉H₂₁NO₅, 343.14; m/z found, 344 [M+H]⁺.

### Step C: Tert-Butyl 3′-Hydroxy-6-Oxo-6,8-Dihydro-2H-Spiro[Benzo[2,1-b: 3,4-c′]Difuran-3,4′-Piperidine]-1′-Carboxylate

To a solution of tert-butyl 6-oxo-2′,3′,6,8-tetrahydro-1′H,2H-spiro[benzo[2,1-b: 3,4-c′]difuran-3,4′-pyridine]-1′-carboxylate (4.7 g, 13.7 mmol, 1.0 eq) in THF (60 mL) was added BH₃-THF (1 M) (34.2 mL, 34.2 mmol, 2.5 eq) under N₂ at −78° C. The reaction was allowed to slowly warm to 0° C. and stirred at 0° C. for 5 hours. Water (5 mL) was added to above mixture, followed by sodium perborate (5.6 g, 68.4 mmol, 5.0 eq). The resulting mixture was stirred at room temperature overnight. The mixture was diluted with DCM (100 mL), washed with brine (50 mL×2), dried over anhydrous sodium sulfate, filtered and concentrated under reduced pressure. The residue was purified by flash column chromatography on silica gel (ethyl acetate in petroleum ether, from 0% to 40%) to afford tert-butyl 3′-hydroxy-6-oxo-6,8-dihydro-2H-spiro[benzo[2,1-b: 3,4-c′]difuran-3,4′-piperidine]-1′-carboxylate (2.7 g, yield 54%) as a white powder. LC-MS(ESI): mass calcd. for C₁₉H₂₃NO₆, 361.15; m/z found, 306 [M+H-56]⁺.

### Step D: Tert-Butyl 3′,6-Dioxo-6,8-Dihydro-2H-Spiro[Benzo[2,1-b: 3,4-c′]Difuran-3,4′-Piperidine]-1′-Carboxylate

To a solution of tert-butyl 3′-hydroxy-6-oxo-6,8-dihydro-2H-spiro[benzo[2,1-b: 3,4-c′]difuran-3,4′-piperidine]-1′-carboxylate (1.8 g, 4.98 mmol, 1.0 eq) in DCM (30 mL) was added Dess-Martin periodinane (5.28 g, 12.5 mmol, 2.5 eq). The mixture was stirred at room temperature for 4 hours. The reaction was diluted with DCM (60 mL), washed with aqueous sodium thiosulfate solution (30 mL×2) and washed with brine (40 mL×2). The organic layer was dried over anhydrous sodium sulfate, filtered, and concentrated under reduced pressure. The residue was purified by flash column chromatography on silica gel (ethyl acetate in petroleum ether, from 0% to 40%) to afford tert-butyl 3′,6-dioxo-6,8-dihydro-2H-spiro[benzo[2,1-b: 3,4-c′]difuran-3,4′-piperidine]-1′-carboxylate (1.3 g, yield 73%) as a colorless oil. LC-MS(ESI): mass calcd. for C₁₉H₂₁NO₆, 359.14; m/z found, 360 [M+H]⁺.

### Step E: Tert-Butyl 3′,3′-Difluoro-6-Oxo-6,8-Dihydro-2H-Spiro[Benzo[2,1-b: 3,4-c′]Difuran-3,4′-Piperidine]-1′-Carboxylate

To a solution of tert-butyl 3′,6-dioxo-6,8-dihydro-2H-spiro[benzo[2,1-b: 3,4-c′]difuran-3,4′-piperidine]-1′-carboxylate (1.2 g, 3.34 mmol, 1.0 eq), Triethylamine trihydrofluoride (3.23 g, 20 mmol, 6.0 eq), and N,N-Diethyl-S,S-difluoro-sulfiliminium tetrafluoroborate (3.44 g, 15 mmol, 4.5 eq) in DCM (50 mL) was added TEA (1.2 mL, 8.35 mmol, 2.5 eq) at 25° C. and the mixture was stirred at this temperature for 1 hour. The reaction mixture was quenched with saturated aqueous NaHCO₃ solution (30 mL) and extracted with DCM (30 mL×3). The separated organic phase was washed with brine (30 mL), dried over anhydrous Na₂SO₄, filtered, and concentrated under reduced pressure. The residue was purified by silica gel chromatography on silica gel (ethyl acetate in petroleum ether, from 0% to 30%) to afford tert-butyl 3′,3′-difluoro-6-oxo-6,8-dihydro-2H-spiro[benzo[2,1-b: 3,4-c′]difuran-3,4′-piperidine]-1′-carboxylate (260 mg, yield 20%) as a yellow oil. LC-MS(ESI): mass calcd. for C₁₉H₂₁F₂NO₅, 381.14; m/z found, 382 [M+H]⁺.

### Step F: 1′-(Tert-Butoxycarbonyl)-3′,3′-Difluoro-7-(Hydroxymethyl)-2H-Spiro[Benzofuran-3,4′-Piperidine]-6-Carboxylic Acid

To a solution of tert-butyl 3′,3′-difluoro-6-oxo-6,8-dihydro-2H-spiro[benzo[2,1-b: 3,4-c′]difuran-3,4′-piperidine]-1′-carboxylate (280 mg, 734 μmol, 1.0 eq) in THF (9 mL), MeOH (9 mL), and H₂O (3 mL) was added NaOH (44 mg, 1.1 mmol, 1.5 eq). The mixture was stirred at 40° C. for 1 hour. After cooling to room temperature, the reaction mixture was diluted with EA (20 mL), adjusted to pH=4˜5 with aqueous HCl solution (3 N), and extracted with EA (40 mL×4). The organic layer was washed with brine (20 mL×2), dried over anhydrous sodium sulfate, filtered, and concentrated to give 1′-(tert-butoxycarbonyl)-3′,3′-difluoro-7-(hydroxymethyl)-2H-spiro[benzofuran-3,4′-piperidine]-6-carboxylic acid (290 mg, yield 99%) as a colorless oil. The crude product was directly used in next step without purification. LC-MS(ESI): mass calcd. for C₁₉H₂₃F₂NO₆, 399.15; m/z found, 398 [M−H].

### Step G: 1′-(Tert-Butoxycarbonyl)-3′,3′-Difluoro-7-Formyl-2H-Spiro[Benzofuran-3,4′-Piperidine]-6-Carboxylic Acid

A solution of 1′-(tert-butoxycarbonyl)-3′,3′-difluoro-7-(hydroxymethyl)-2H-spiro[benzofuran-3,4′-piperidine]-6-carboxylic acid (260 mg, 651 μmol, 1.0 eq) and active manganese dioxide (1.13 g, 13 mmol, 20.0 eq) in DCM (20 mL) was stirred at room temperature for 16 hours. After filtration via a short column, the filtrate is collected and concentrated under reduced pressure to give 1′-(tert-butoxycarbonyl)-3′,3′-difluoro-7-formyl-2H-spiro[benzofuran-3,4′-piperidine]-6-carboxylic acid (250 mg, yield 97%) as a colorless oil. The crude product was directly used in next step without purification. LC-MS(ESI): mass calcd. for C₁₉H₂₁F₂NO₆, 397.13; m/z found, 398 [M+H]⁺.

### Step H: Tert-Butyl 7-(2,6-Dioxopiperidin-3-Yl)-3′,3′-Difluoro-6-Oxo-7,8-Dihydro-2H,6H-Spiro[Furo [2,3-e]Isoindole-3,4′-Piperidine]-1′-Carboxylate

To a solution of 1′-(tert-butoxycarbonyl)-3′,3′-difluoro-7-formyl-2H-spiro[benzofuran-3,4′-piperidine]-6-carboxylic acid (260 mg, 654 μmol, 1.0 eq) and 3-aminopiperidine-2,6-dione hydrochloride (215 mg, 1.31 mmol, 2.0 eq) in DMF (10 mL) was added Acetic acid (0.50 mL, 8.7 mmol, 13.0 eq) and the reaction was stirred at 40° C. for 2 hours. Sodium triacetoxyborohydride (416 mg, 1.96 mmol, 3.0 eq) was added to above mixture and the resulting mixture was stirred at 40° C. for 16 hours. After cooled to room temperature, the mixture was dissolved with EA (60 mL), washed with brine (30 mL×4), dried over anhydrous sodium sulfate, filtered, and concentrated under reduced pressure. The residue was purified by flash column chromatography on silica gel (ethyl acetate in petroleum ether, from 0% to 90%) to afford tert-butyl 7-(2,6-dioxopiperidin-3-yl)-3′,3′-difluoro-6-oxo-7,8-dihydro-2H,6H-spiro[furo [2,3-e]isoindole-3,4′-piperidine]-1′-carboxylate (220 mg, yield 68%) as a grey solid. LC-MS(ESI): mass calcd. for C₂₄H₂₇F₂N₃O₆, 491.19; m/z found, 492 [M+H]⁺.

### Step I: 3-(3′,3′-Difluoro-6-Oxo-6,8-Dihydro-2H,7H-Spiro[Furo [2,3-e]Isoindole-3,4′-Piperidin]-7-Yl) Piperidine-2,6-Dione TFA Salt

To a solution of tert-butyl 7-(2,6-dioxopiperidin-3-yl)-3′,3′-difluoro-6-oxo-7,8-dihydro-2H,6H-spiro[furo [2,3-e]isoindole-3,4′-piperidine]-1′-carboxylate (200 mg, 407 μmol, 1.0 eq) in DCM (5 mL) was added trifluoroacetic acid (2 mL) and the reaction was stirred at 25° C. for 1 hour. The mixture was concentrated and dried to afford 3-(3′,3′-difluoro-6-oxo-6,8-dihydro-2H,7H-spiro[furo [2,3-e]isoindole-3,4′-piperidin]-7-yl) piperidine-2,6-dione trifluoroacetate (150 mg, yield 73%) as a grey solid. LC-MS(ESI): mass calcd. for C₁₉H₁₉F₂N₃O₄, 391.13; m/z found, 392 [M+H]⁺.

¹H NMR (400 MHZ, DMSO-d₆) δ 10.99 (d, J=4.4 Hz, 1H), 9.67 (s, 1H), 7.48-7.37 (m, 2H), 5.16-4.96 (m, 2H), 4.74-4.63 (m, 1H), 4.44 (t, J=17.0 Hz, 1H), 4.27 (t, J=16.6 Hz, 1H), 3.93-3.71 (m, 2H), 3.03 (t, J=12.0 Hz, 1H), 2.97-2.83 (m, 1H), 2.60 (d, J=17.2 Hz, 1H), 2.46-2.40 (m, 2H), 2.26-2.23 (m, 1H), 2.08 (d, J=5.0 Hz, 1H), 2.00-1.98 (m, 1H).

### Compound A18. (S)—N-(2,6-Dioxopiperidin-3-Yl)-2H-Spiro[Furo [2,3-b]Pyridine-3,4′-Piperidine]-6-Carboxamide

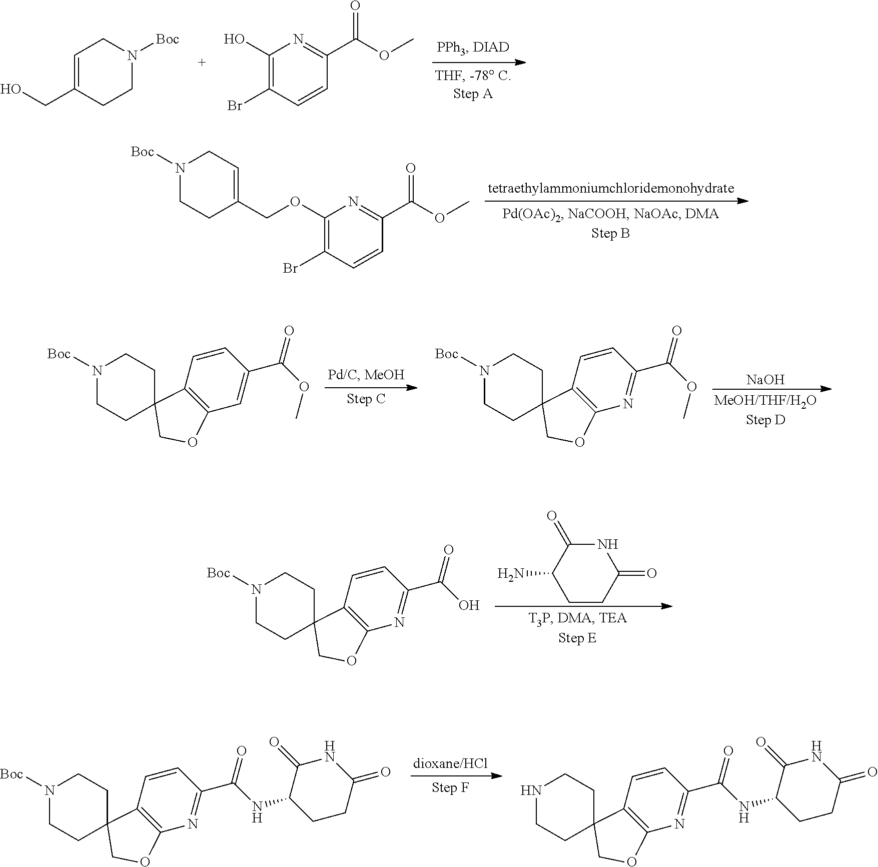

### Step A: Methyl 5-Bromo-6-((1-(Tert-Butoxycarbonyl)-1,2,3,6-Tetrahydropyridin-4-Yl) Methoxy) Picolinate

To a mixture of tert-butyl 4-(hydroxymethyl)-3,6-dihydropyridine-1 (2H)-carboxylate (5.52 g, 1.2 eq, 25.9 mmol) in THF (50.0 mL) was added methyl 5-bromo-6-hydroxypicolinate (5.00 g, 1 eq, 21.5 mmol) and Ph₃P (14.1 g, 2.5 eq, 53.9 mmol). The reaction mixture was cooled to −78° C. and DIAD (10.9 g, 10.5 mL, 2.5 eq, 53.9 mmol) was dropwise added to above mixture. The mixture was stirred at room temperature for 16 h. After evaporation, the crude product was purified by flash column chromatography on silica gel (ethyl acetate in petroleum ether, from 0% to 20%) to afford methyl 5-bromo-6-((1-(tert-butoxycarbonyl)-1,2,3,6-tetrahydropyridin-4-yl) methoxy) picolinate (7.10 g, yield 77.1%) as a white solid. LC-MS (ESI): mass calcd. for C₁₈H₂₃BrN₂O₅, 426.08; m/z found, 427.1 [M+H]⁺.

### Step B: 1′-(Tert-Butyl)6-Methyl 2′,3′-Dihydro-1′H,2H-Spiro[Furo [2,3-b]Pyridine-3,4′-Pyridine]-1′,6-Dicarboxylate

To a solution of methyl 5-bromo-6-((1-(tert-butoxycarbonyl)-1,2,3,6-tetrahydropyridin-4-yl) methoxy) picolinate (7.10 g, 1 eq, 16.6 mmol) in DMA (30.0 mL) was added HCOONa (2.3 g, 2 eq, 33.2 mmol) tetraethylammoniumchloridemonohydrate (4.58 g, 4.24 mL, 1.5 eq, 24.9 mmol), Pd(OAc)2 (746 mg, 0.2 eq, 3.32 mmol) and NaOAc (2.7 g, 2 eq, 33.2 mmol). The mixture was purged with nitrogen and heated to 100° C. overnight. The mixture was diluted with ethyl acetate (100 mL) and washed with water (200 mL). The organic layer was washed with brine (50 mL*2) and dried over sodium sulfate. The crude was purified by silica gel column chromatography using 0-30% EtOAc/hexane to give 1′-(tert-butyl)6-methyl 2′,3′-dihydro-1′H,2H-spiro[furo [2,3-b]pyridine-3,4′-pyridine]-1′,6-dicarboxylate (4.50 g, yield 78.2%) as a white solid. LC-MS(ESI): mass calcd. for C₁₈H₂₂N₂O₅, 346.15; m/z found, 347.2 [M+H]⁺.

### Step C: 1′-(Tert-Butyl)6-Methyl 2H-Spiro[Furo [2,3-b]Pyridine-3,4′-Piperidine]-1′,6-Dicarboxylate

To a solution of 1′-(tert-butyl)6-methyl 2′,3′-dihydro-1′H,2H-spiro[furo [2,3-b]pyridine-3,4′-pyridine]-1′,6-dicarboxylate (1.00 g, 1 eq, 2.89 mmol) in MeOH (10.0 mL) was added Pd/C (200 mg, 10% on Carbon, wetted with c.a.55% water). The mixture was purged with H₂ and stirred at rt overnight under H₂. The mixture was filtered, and the filtrate was concentrated. The crude product was purified by silica gel chromatography. The desired 1′-(tert-butyl)6-methyl 2H-spiro[furo [2,3-b]pyridine-3,4′-piperidine]-1′,6-dicarboxylate (900 mg, yield 89.5%) was obtained as white solid. LC-MS(ESI): mass calcd. for C₁₈H₂₄N₂O₅, 348.17; m/z found, 349.2 [M+H]⁺.

### Step D: 1′-(Tert-Butoxycarbonyl)-2H-Spiro[Furo [2,3-b]Pyridine-3,4′-Piperidine]-6-Carboxylic Acid

To a mixture of 1′-(tert-butyl)6-methyl 2H-spiro[furo [2,3-b]pyridine-3,4′-piperidine]-1′,6-dicarboxylate (900 mg, 1 eq, 2.58 mmol) in THF (5.00 mL) and H₂O (5.00 mL), MeOH (5.00 mL) was added NaOH (0.41 g, 4 eq, 10.3 mmol). The mixture was stirred at 20° C. for 2 h. The mixture was adjusted to pH=5-6 with aq. hydrochloric acid (1 M) and extracted with ethyl acetate. The organic layer was washed with brine and dried over sodium sulfate. After filtration, the filtrate was concentrated under reduced pressure to afford 1′-(tert-butoxycarbonyl)-2H-spiro[furo [2,3-b]pyridine-3,4′-piperidine]-6-carboxylic acid (600 mg, yield 69.5%) as a white solid. LC-MS(ESI): mass calcd. for C₁₇H₂₂N₂O₅, 334.15; m/z found, 335.2 [M+H]⁺.

### Step E: Tert-Butyl(S)-6-((2,6-Dioxopiperidin-3-Yl) Carbamoyl)-2H-Spiro[Furo [2,3-b]Pyridine-3,4′-Piperidine]-1′-Carboxylate

To a mixture of 1′-(tert-butoxycarbonyl)-2H-spiro[furo [2,3-b]pyridine-3,4′-piperidine]-6-carboxylic acid (600 mg, 1 eq, 1.79 mmol) in DMA (5.00 mL) was added(S)-3-aminopiperidine-2,6-dione hydrochloride (295 mg, 1 eq, 1.79 mmol), Propylphosphonic anhydride (1.14 g, 2 eq, 3.59 mmol) and TEA (363 mg, 2 eq, 3.59 mmol), and the reaction was stirred at rt for 1 h. UPLC-MS indicated a new main peak with desired MS formed, then quenched with water and purified by pre-HPLC. The desired product tert-butyl(S)-6-((2,6-dioxopiperidin-3-yl) carbamoyl)-2H-spiro[furo [2,3-b]pyridine-3,4′-piperidine]-1′-carboxylate (600 mg, yield 75.2%) was obtained as a white solid. LC-MS(ESI): mass calcd. for C₂₂H₂₈N₄O₆, 444.20; m/z found, 445.2 [M+H]⁺.

### Step F: (S)—N-(2,6-Dioxopiperidin-3-Yl)-2H-Spiro[Furo [2,3-b]Pyridine-3,4′-Piperidine]-6-Carboxamide Hydrochloride

To a mixture of tert-butyl(S)-6-((2,6-dioxopiperidin-3-yl) carbamoyl)-2H-spiro[furo [2,3-b]pyridine-3,4′-piperidine]-1′-carboxylate (200 mg, 1 eq, 450 μmol) in 1,4-dioxane/HCl (5.00 mL) The mixture was stirred at 20° C. for 2 h. The after reaction was direct concentration as to give(S)—N-(2,6-dioxopiperidin-3-yl)-2H-spiro[furo [2,3-b]pyridine-3,4′-piperidine]-6-carboxamide hydrochloride (170 mg, yield 99.2%) as a white solid. LC-MS (ESI): mass calcd. for C₁₇H₂₀N₄O₄, 344.15; m/z found, 345.1 [M+H]⁺.

¹H NMR (400 MHZ, DMSO)δ 10.84 (s, 1H), 9.27-9.16 (m, 1H), 9.14-8.97 (m, 1H), 8.86 (d, J=8.4 Hz, 1H), 7.78 (d, J=7.4 Hz, 1H), 7.67 (d, J=7.4 Hz, 1H), 4.83-4.70 (m, 1H), 4.62 (s, 2H), 3.36-3.25 (m, 2H), 3.09-2.95 (m, 2H), 2.85-2.70 (m, 1H), 2.57-2.52 (m, 1H), 2.26-2.06 (m, 3H), 2.01-1.87 (m, 3H).

### Compound A19. (S)-3-(5-Methyl-6-Oxo-6,8-Dihydro-2H,7H-Spiro[Furo [2,3-e]Isoindole-3,4′-Piperidin]-7-Yl) Piperidine-2,6-Dione

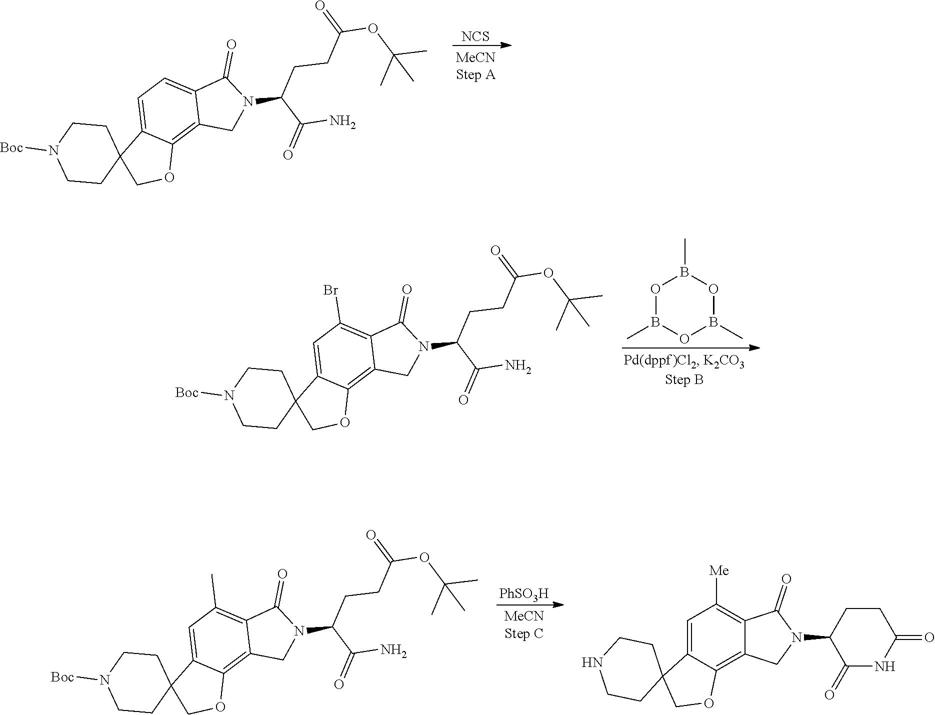

### Step A: Tert-Butyl(S)-7-(1-Amino-5-(Tert-Butoxy)-1,5-Dioxopentan-2-Yl)-5-Bromo-6-Oxo-7,8-Dihydro-2H,6H-Spiro[Furo [2,3-e]Isoindole-3,4′-Piperidine]-1′-Carboxylate

A mixture of tert-butyl(S)-7-(1-amino-5-(tert-butoxy)-1,5-dioxopentan-2-yl)-6-oxo-7,8-dihydro-2H,6H-spiro[furo [2,3-e]isoindole-3,4′-piperidine]-1′-carboxylate (600 mg, 1.13 mmol, 1 eq.) and NBS(262 mg, 1.47 mmol, 1.3 eq.) in MeCN (15 mL) was stirred at room temperature for 20 hours. Then the mixture was diluted with water (30 mL) and extracted with EA (30 mL×2). The combined organic layers were washed with brine (30 mL), dried over anhydrous Na₂SO₄ and concentrated under reduced pressure. The residue was purified by flash chromatography and eluted with 0-70% EA in PE to give tert-butyl(S)-7-(1-amino-5-(tert-butoxy)-1,5-dioxopentan-2-yl)-5-bromo-6-oxo-7,8-dihydro-2H,6H-spiro[furo [2,3-e]isoindole-3,4′-piperidine]-1′-carboxylate (300 mg, yield 43%) as a white solid. LC-MS(ESI): mass calcd. for C₂₈H₃₈BrN₃O₇, 607.19; m/z found, 608.2 [M+H]⁺.

### Step B: Tert-Butyl(S)-7-(1-Amino-5-(Tert-Butoxy)-1,5-Dioxopentan-2-Yl)-5-Methyl-6-Oxo-7,8-Dihydro-2H,6H-Spiro[Furo [2,3-e]Isoindole-3,4′-Piperidine]-1′-Carboxylate

A mixture of tert-butyl(S)-7-(1-amino-5-(tert-butoxy)-1,5-dioxopentan-2-yl)-5-bromo-6-oxo-7,8-dihydro-2H,6H-spiro[furo [2,3-e]isoindole-3,4′-piperidine]-1′-carboxylate (300 mg, 493 μmol, 1 eq.), 2,4,6-trimethyl-1,3,5,2,4,6-trioxatriborinane (124 mg, 986 μmol, 2 eq.), K₂CO₃ (204 mg, 1.48 mmol, 3 eq.) and 1,1′-bis(diphenylphosphino) ferrocene-palladium (II) dichloride (72 mg, 98.6 μmol, 0.2 eq.) in dioxane (3 mL) and H₂O (0.3 mL) was stirred at 80° C. under N₂ atmosphere for 2 hours. Then the mixture was diluted with water (10 mL) and extracted with EA (10 mL×2). The combined organic layers were washed with brine (20 mL), dried over anhydrous Na₂SO₄ and concentrated under reduced pressure. The residue was purified by flash chromatography and eluted with 0-55% EA in PE to give tert-butyl(S)-7-(1-amino-5-(tert-butoxy)-1,5-dioxopentan-2-yl)-5-methyl-6-oxo-7,8-dihydro-2H,6H-spiro[furo [2,3-e]isoindole-3,4′-piperidine]-1′-carboxylate (190 mg, yield 70%) as a yellow solid. LC-MS(ESI): mass calcd. for C₂₉H₄₁N₃O₇, 543.29; m/z found, 544.2 [M+H]⁺.

### Step C(S)-3-(5-Methyl-6-Oxo-6,8-Dihydro-2H,7H-Spiro[Furo [2,3-e]Isoindole-3,4′-Piperidin]-7-Yl) Piperidine-2,6-Dione

A mixture of tert-butyl(S)-7-(1-amino-5-(tert-butoxy)-1,5-dioxopentan-2-yl)-5-methyl-6-oxo-7,8-dihydro-2H,6H-spiro[furo [2,3-e]isoindole-3,4′-piperidine]-1′-carboxylate (160 mg, 294 μmol, 1 eq.) and anhydrous benzenesulfonic acid (140 mg, 883 μmol, 3 eq.) in MeCN (10 mL) was stirred at 80° C. under N₂ atmosphere for 3 hours. The mixture was concentrated under reduced pressure and purified by prep-HPLC to give(S)-3-(5-methyl-6-oxo-6,8-dihydro-2H,7H-spiro[furo [2,3-e]isoindole-3,4′-piperidin]-7-yl) piperidine-2,6-dione (0.3 PhSO₃H salt, 64 mg, yield 52%) as a white solid. LC-MS(ESI): mass calcd. for C₂₀H₂₃N₃O₄, 369.17; m/z found, 370.2 [M+H]⁺.

¹H NMR (400 MHZ, DMSO-d₆) δ 10.95 (s, 1H), 8.31 (bs, 1H), 7.62-7.30 (m, 1.3H, 0.3 PhSO₃H salt), 7.07 (s, 1H), 5.04 (dd, J=13.3, 5.0 Hz, 1H), 4.56 (s, 2H), 4.31 (d, J=17.2 Hz, 1H), 4.17 (d, J=17.2 Hz, 1H), 3.21-3.12 (m, 3H), 2.94-2.79 (m, 3H), 2.56 (s, 3H), 2.45-2.38 (m, 1H), 1.99-1.90 (m, 3H), 1.80-1.72 (m, 2H).

### Compound A20. (S)-3-(5-Chloro-6-Oxo-6,8-Dihydro-2H,7H-Spiro[Furo [2,3-e]Isoindole-3,4′-Piperidin]-7-Yl) Piperidine-2,6-Dione

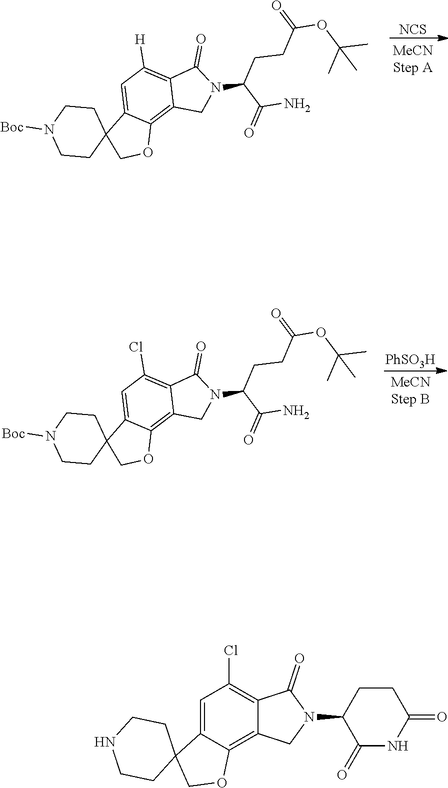 [SMILES: CC(C)(C)OC(=O)CC[C@@H](C(N)=O)N1Cc2c(ccc3c2OCC32CCN(C(=O)OC(C)(C)C)CC2)C1=O ; "PhSO3H" ; "NCS" ; CC(C)(C)OC(=O)CC[C@@H](C(N)=O)N1Cc2c3c(cc(Cl)c2C1=O)C1(CCN(C(=O)OC(C)(C)C)CC1)CO3 ; O=C1CC[C@H](N2Cc3c4c(cc(Cl)c3C2=O)C2(CCNCC2)CO4)C(=O)N1]

### Step A: Tert-Butyl(S)-7-(1-Amino-5-(Tert-Butoxy)-1,5-Dioxopentan-2-Yl)-5-Chloro-6-Oxo-7,8-Dihydro-2H,6H-Spiro[Furo [2,3-e]Isoindole-3,4′-Piperidine]-1′-Carboxylate

A mixture of tert-butyl(S)-7-(1-amino-5-(tert-butoxy)-1,5-dioxopentan-2-yl)-6-oxo-7,8-dihydro-2H,6H-spiro[furo [2,3-e]isoindole-3,4′-piperidine]-1′-carboxylate (600 mg, 1.13 mmol, 1 eq.) and NCS(197 mg, 1.47 mmol, 1.3 eq.) in MeCN (15 mL) was stirred at room temperature for 20 hours. Then the mixture was diluted with water (30 mL) and extracted with EA (30 mL×2). The combined organic layers were washed with brine (30 mL), dried over anhydrous Na₂SO₄ and concentrated under reduced pressure. The residue was purified by flash chromatography and eluted with 0-70% EA in PE to give the tert-butyl(S)-7-(1-amino-5-(tert-butoxy)-1,5-dioxopentan-2-yl)-5-chloro-6-oxo-7,8-dihydro-2H,6H-spiro[furo [2,3-e]isoindole-3,4′-piperidine]-1′-carboxylate (430 mg, yield 67%) as a white solid. LC-MS(ESI): mass calcd. for C₂₈H₃₈ClN₃O₇, 563.24; m/z found, 564.2 [M+H]⁺.

### Step B: (S)-3-(5-Chloro-6-Oxo-6,8-Dihydro-2H,7H-Spiro[Furo [2,3-e]Isoindole-3,4′-Piperidin]-7-Yl) Piperidine-2,6-Dione

A mixture of tert-butyl(S)-7-(1-amino-5-(tert-butoxy)-1,5-dioxopentan-2-yl)-5-chloro-6-oxo-7,8-dihydro-2H,6H-spiro[furo [2,3-e]isoindole-3,4′-piperidine]-1′-carboxylate (380 mg, 674 μmol, 1 eq.) and anhydrous benzenesulfonic acid (320 mg, 2.02 mmol, 3 eq.) in MeCN (2.5 mL) was stirred at 80° C. under N₂ atmosphere for 3 hours. The mixture was concentrated under reduced pressure and purified by prep-HPLC to give(S)-3-(5-chloro-6-oxo-6,8-dihydro-2H,7H-spiro[furo [2,3-e]isoindole-3,4′-piperidin]-7-yl) piperidine-2,6-dione (0.5 PhSO₃H salt, 210 mg, yield 67%) as a white solid. LC-MS(ESI): mass calcd. for C₁₉H₂₀ClN₃O₄, 389.11; m/z found, 390.2 [M+H]⁺.

¹H NMR (400 MHZ, MeOD-d₄)δ 7.86-7.40 (m, 2.5H, 0.5 PhSO₃H salt), 7.34 (s, 1H), 5.11 (dd, J=13.2, 5.2 Hz, 1H), 4.73-4.66 (m, 2H), 4.42-4.34 (m, 2H), 3.48-3.40 (m, 2H), 3.19-3.05 (m, 2H), 2.96-2.84 (m, 1H), 2.81-2.73 (m, 1H), 2.53-2.45 (m, 1H), 2.19-2.06 (m, 3H), 2.05-1.97 (m, 2H).

Compounds A²¹ and A²²: (S)-3-(5-methoxy-6-oxo-6,8-dihydro-2H,7H-spiro[furo [2,3-e]isoindole-3,4′-piperidin]-7-yl) piperidine-2,6-dione hydrochloride and (R)-3-(5-methoxy-6-oxo-6,8-dihydro-2H,7H-spiro[furo [2,3-e]isoindole-3,4′-piperidin]-7-yl) piperidine-2,6-dione hydrochloride

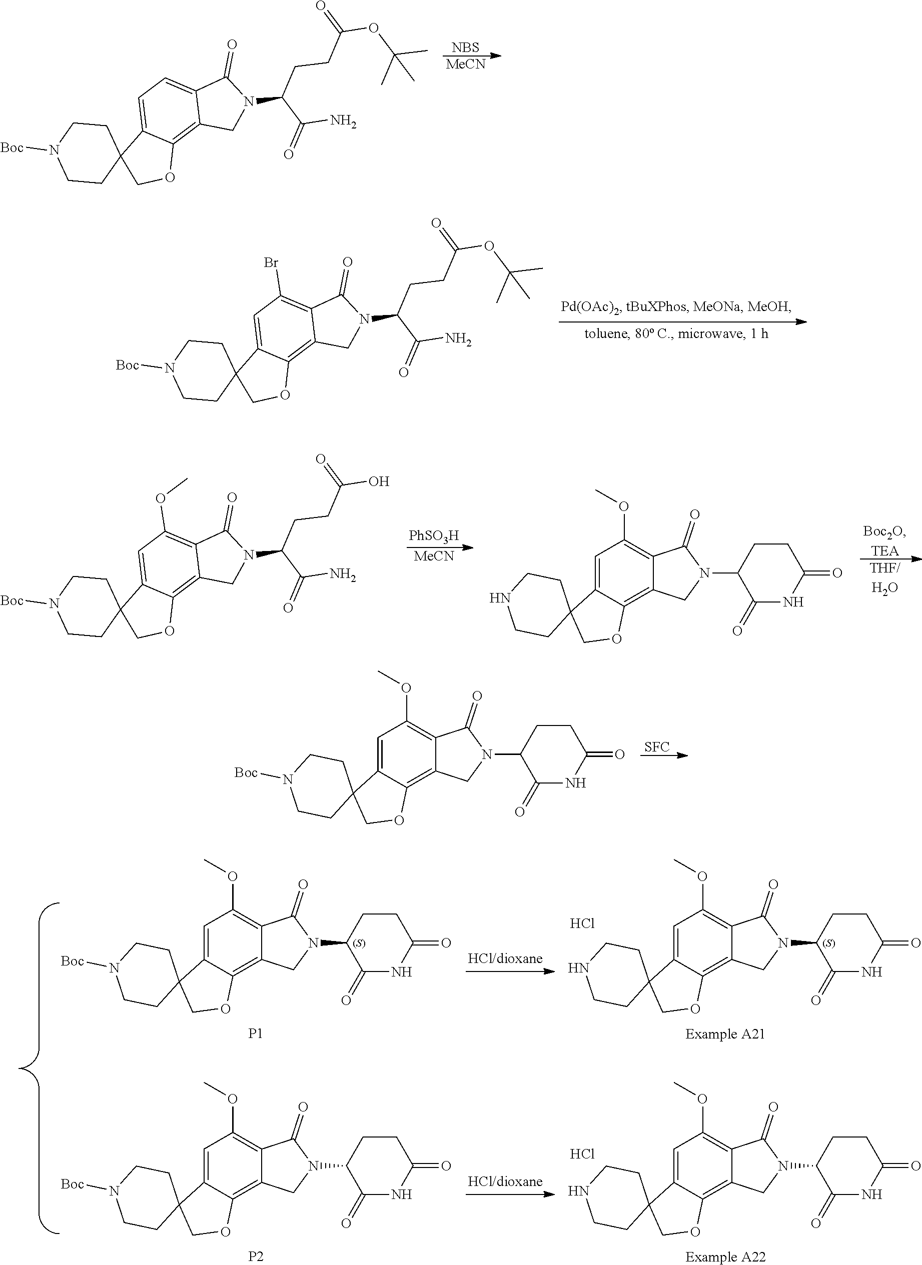

### Step 1: Tert-Butyl(S)-7-(1-Amino-5-(Tert-Butoxy)-1,5-Dioxopentan-2-Yl)-5-Bromo-6-Oxo-7,8-Dihydro-2H,6H-Spiro[Furo [2,3-e]Isoindole-3,4′-Piperidine]-1′-Carboxylate

To a mixture of tert-butyl(S)-7-(1-amino-5-(tert-butoxy)-1,5-dioxopentan-2-yl)-6-oxo-7,8-dihydro-2H,6H-spiro[furo [2,3-e]isoindole-3,4′-piperidine]-1′-carboxylate (2.00 g, 1 eq, 3.78 mmol) in MeCN (10.0 mL) was added NBS(874 mg, 1.3 eq, 4.91 mmol). The resulting mixture was then stirred at 25° C. for 3 hours. The mixture was poured into H₂O (30 mL), extracted with EtOAc (20 mL×2). The combined organic layer was washed with brine (50 mL), dried over Na₂SO₄, filtered and concentrated in vacuum. The crude product was purified by silica gel column chromatography using 0-60% EtOAc/hexane to afford tert-butyl(S)-7-(1-amino-5-(tert-butoxy)-1,5-dioxopentan-2-yl)-5-bromo-6-oxo-7,8-dihydro-2H,6H-spiro[furo [2,3-e]isoindole-3,4′-piperidine]-1′-carboxylate (1.50 g, 2.46 mmol, 65.3%) as a yellow solid. LC-MS(ESI, m/z): mass calcd. For C₂₈H₃₈BrN₃O₇, 607.2; found, 608.2 [M+H]⁺.

### Step 2:5-Amino-4-(1′-(Tert-Butoxycarbonyl)-5-Methoxy-6-Oxo-6,8-Dihydro-2H,7H-Spiro[Furo [2,3-e]Isoindole-3,4′-Piperidin]-7-Yl)-5-Oxopentanoic Acid

To a mixture of tert-butyl(S)-7-(1-amino-5-(tert-butoxy)-1,5-dioxopentan-2-yl)-5-bromo-6-oxo-7,8-dihydro-2H,6H-spiro[furo [2,3-e]isoindole-3,4′-piperidine]-1′-carboxylate (0.30 g, 1 eq, 0.49 mmol), sodium methanolate (0.13 g, 5 eq, 2.5 mmol) in Toluene (5.00 mL) and MeOH (5.00 mL) was added di-tert-butyl(2′,4′,6′-triisopropyl-[1,1′-biphenyl]-2-yl)phosphane (42 mg, 0.2 eq, 99 μmol), palladium (II) acetate (11 mg, 0.1 eq, 49 μmol). The mixture was stirred at 80° C. in sealed tube for 1 hour under Ar. The mixture was poured into 1 M HCl (50 mL), extracted with EtOAc (20 mL×2). The combined organic layer was washed with brine (50 mL), dried over Na₂SO₄, filtered and concentrated in vacuum. The crude product was purified by silica gel column chromatography using 0-20% MeOH/DCM to afford 5-amino-4-(1′-(tert-butoxycarbonyl)-5-methoxy-6-oxo-6,8-dihydro-2H,7H-spiro[furo [2,3-e]isoindole-3,4′-piperidin]-7-yl)-5-oxopentanoic acid (0.20 g, crude, 72%) as a yellow solid. LC-MS(ESI, m/z): mass calcd. For C₂₅H₃₃N₃O₈, 503.2; found, 504.1 [M+H]⁺.

### Step 3:3-(5-Methoxy-6-Oxo-6,8-Dihydro-2H,7H-Spiro[Furo [2,3-e]Isoindole-3,4′-Piperidin]-7-Yl) Piperidine-2,6-Dione

To a mixture of tert-butyl 7-(1-amino-5-(tert-butoxy)-1,5-dioxopentan-2-yl)-5-methoxy-6-oxo-7,8-dihydro-2H,6H-spiro[furo [2,3-e]isoindole-3,4′-piperidine]-1′-carboxylate (0.20 g, 1 eq, 0.36 mmol) in MeCN (5.00 mL) was added benzenesulfonic acid (0.28 g, 5 eq, 1.8 mmol). The resulting mixture was then stirred at 80° C. for 3 h. The mixture was concentrated in vacuum to afford 3-(5-methoxy-6-oxo-6,8-dihydro-2H,7H-spiro[furo [2,3-e]isoindole-3,4′-piperidin]-7-yl) piperidine-2,6-dione (0.3 g, crude) as a yellow solid. LC-MS (ESI, m/z): mass calcd. For C₂₀H₂₃N₃O₅, 385.2; found, 386.2 [M+H]⁺.

### Step 4: Tert-Butyl 7-(2,6-Dioxopiperidin-3-Yl)-5-Methoxy-6-Oxo-7,8-Dihydro-2H,6H-Spiro[Furo [2,3-e]Isoindole-3,4′-Piperidine]-1′-Carboxylate

To a mixture of 3-(5-methoxy-6-oxo-6,8-dihydro-2H,7H-spiro[furo [2,3-e]isoindole-3,4′-piperidin]-7-yl) piperidine-2,6-dione (0.70 g, crude, 1 eq, 1.8 mmol) in THF (10.0 mL) and H₂O (2.00 mL) was added TEA (0.37 g, 0.51 mL, 2 eq, 3.6 mmol) and (Boc)₂O (0.48 g, 0.50 mL, 1.2 eq, 2.2 mmol). The mixture was stirred at room temperature for 2 hours. The mixture was poured into H₂O (50 mL), extracted with EtOAc (20 mL×2). The combined organic layer was washed with brine (50 mL), dried over Na₂SO₄, filtered and concentrated in vacuum. The crude product was purified by silica gel column chromatography using 0-60% EtOAc/hexane to afford tert-butyl 7-(2,6-dioxopiperidin-3-yl)-5-methoxy-6-oxo-7,8-dihydro-2H,6H-spiro[furo [2,3-e]isoindole-3,4′-piperidine]-1′-carboxylate (0.30 g, 0.62 mmol, 34%) as a yellow solid. LC-MS(ESI, m/z): mass calcd. For C₂₅H₃₁N₃O₇, 485.2; found, 486.2 [M+H]⁺.

### Step 5: tert-butyl(S)-7-(2,6-dioxopiperidin-3-yl)-5-methoxy-6-oxo-7,8-dihydro-2H,6H-spiro[furo [2,3-e]isoindole-3,4′-piperidine]-1′-carboxylate and tert-butyl(R)-7-(2,6-dioxopiperidin-3-yl)-5-methoxy-6-oxo-7,8-dihydro-2H,6H-spiro[furo [2,3-e]isoindole-3,4′-piperidine]-1′-carboxylate

The tert-butyl 7-(2,6-dioxopiperidin-3-yl)-5-methoxy-6-oxo-7,8-dihydro-2H,6H-spiro[furo [2,3-e]isoindole-3,4′-piperidine]-1′-carboxylate (0.3 g) was purified by SFC (the conditions were described as followed:

| System: | Waters SFC 150 | |
|---|---|---|
| Column name: | DAICELCHIRALCEL ®OD | |
| Column size: | 250*25 mm 10 μm | |
| Mobile Phase A: | Supercritical CO_{2,} | |
| Mobile Phase B: | MeOH | |
| A:B: | 55:45 | |
| Wavelength: | 214 | nm |
| Flow: | 100 | ml/min |
| Column temp: | RT | |
| Back Pressure: | 100 | bar |
| Injection: | 4.5 | mL |
| Cycle time: | 5.1 | min |

| | |
|---|---|
| Solvent: | MeOH: redistilled grade |
| | Supercritical CO₂: Food grade |
| Preparation of sample | sample was dissolved |
| solution: | in about 40 mL MeOH |

to afford tert-butyl(S)-7-(2,6-dioxopiperidin-3-yl)-5-methoxy-6-oxo-7,8-dihydro-2H,6H-spiro[furo [2,3-e]isoindole-3,4′-piperidine]-1′-carboxylate (130 mg) as a yellow solid and tert-butyl(R)-7-(2,6-dioxopiperidin-3-yl)-5-methoxy-6-oxo-7,8-dihydro-2H,6H-spiro[furo [2,3-e]isoindole-3,4′-piperidine]-1′-carboxylate (100 mg) as a yellow solid. LC-MS(ESI, m/z): mass calcd. For C₂₅H₃₁N₃O₇, 485.2; found, 486.2 [M+H]⁺.

### Step 6: (S)-3-(5-Methoxy-6-Oxo-6,8-Dihydro-2H,7H-Spiro[Furo [2,3-e]Isoindole-3,4′-Piperidin]-7-Yl) Piperidine-2,6-Dione Hydrochloride (Example A²¹)

To a solution of tert-butyl(S)-7-(2,6-dioxopiperidin-3-yl)-5-methoxy-6-oxo-7,8-dihydro-2H,6H-spiro[furo [2,3-e]isoindole-3,4′-piperidine]-1′-carboxylate (130 mg, 0.27 mmol, 1.0 eq) was added HCl/dioxane (4M, 3 mL) at room temperature. The reaction mixture was stirred at room temperature for 1 h. The reaction mixture was concentrated to afford(S)-3-(5-methoxy-6-oxo-6,8-dihydro-2H,7H-spiro[furo [2,3-e]isoindole-3,4′-piperidin]-7-yl) piperidine-2,6-dione hydrochloride (134 mg) as a yellow solid. LC-MS(ESI, m/z): mass calcd. For C₂₀H₂₃N₃O₅, 385.2; found, 386.2 [M+H]⁺

¹H NMR (400 MHZ, DMSO-d₆) δ 10.95 (s, 1H), 9.11 (d, J=8.4 Hz, 1H), 8.81 (d, J=10 Hz, 1H), 6.83 (s, 1H), 5.03-4.99 (m, 1H), 4.62-4.57 (m, 2H), 4.32-4.11 (m, 2H), 3.85 (s, 3H), 3.37-3.31 (m, 2H), 3.05-2.86 (m, 3H), 2.60-2.55 (m, 1H), 2.41-2.33 (m, 1H), 2.20-2.12 (m, 2H), 1.96-1.84 (m, 3H).

### Step 7: (R)-3-(5-Methoxy-6-Oxo-6,8-Dihydro-2H,7H-Spiro[Furo [2,3-e]Isoindole-3,4′-Piperidin]-7-Yl) Piperidine-2,6-Dione Hydrochloride

To a solution of tert-butyl(R)-7-(2,6-dioxopiperidin-3-yl)-5-methoxy-6-oxo-7,8-dihydro-2H,6H-spiro[furo [2,3-e]isoindole-3,4′-piperidine]-1′-carboxylate (20 mg, 0.04 mmol, 1.0 eq) was added HCl/dioxane (4M, 3 mL) at room temperature. The reaction mixture was stirred at room temperature for 1 h. The reaction mixture was concentrated to afford (R)-3-(5-methoxy-6-oxo-6,8-dihydro-2H,7H-spiro[furo [2,3-e]isoindole-3,4′-piperidin]-7-yl) piperidine-2,6-dione hydrochloride (20 mg) as a yellow solid (Compound A22). LC-MS (ESI, m/z): mass calcd. For C₂₀H₂₃N₃O₅, 385.2; found, 386.2 [M+H]⁺

¹H NMR (400 MHZ, DMSO-d₆) δ 10.94 (s, 1H), 9.35-9.22 (m, 1H), 9.12-8.92 (m, 1 H), 6.83 (s, 1H), 5.01 (dd, J=13.2, 4.8 Hz, 1H), 4.64-4.53 (m, 2H), 4.34-4.09 (m, 2H), 3.84 (s, 3H), 3.36-3.26 (m, 2H), 3.06-2.84 (m, 3H), 2.64-2.54 (m, 1H), 2.43-2.32 (m, 1H), 2.25-2.13 (m, 2H), 1.99-1.79 (m, 3H).

### Compound B0. 3-(6-Oxo-6,8-Dihydrospiro[Furo [3,4-e]Isoindole-3,4′-Piperidin]-7 (1H)-Yl) Piperidine-2,6-Dione

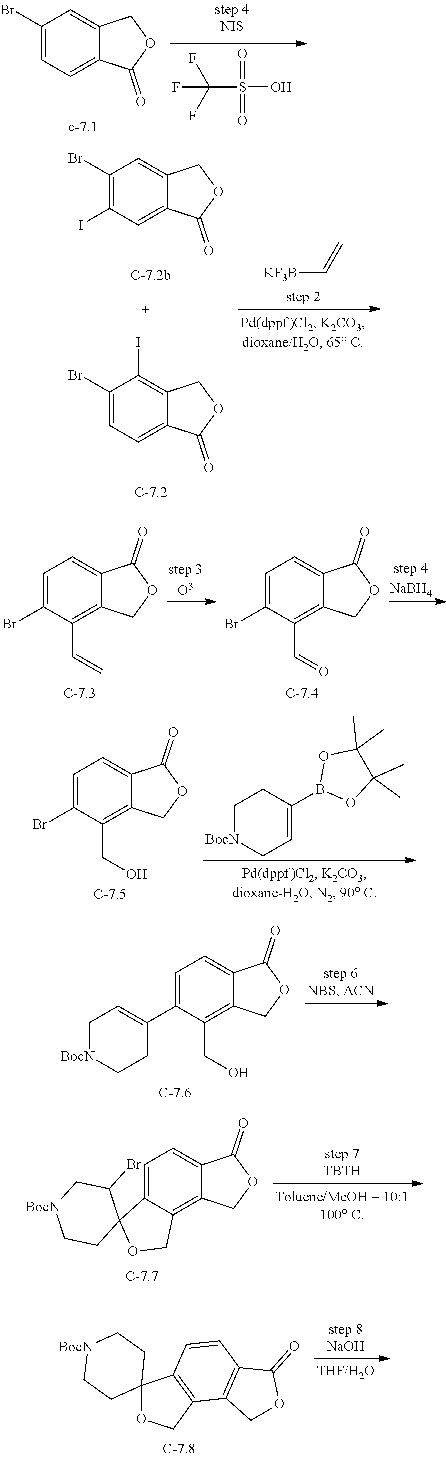

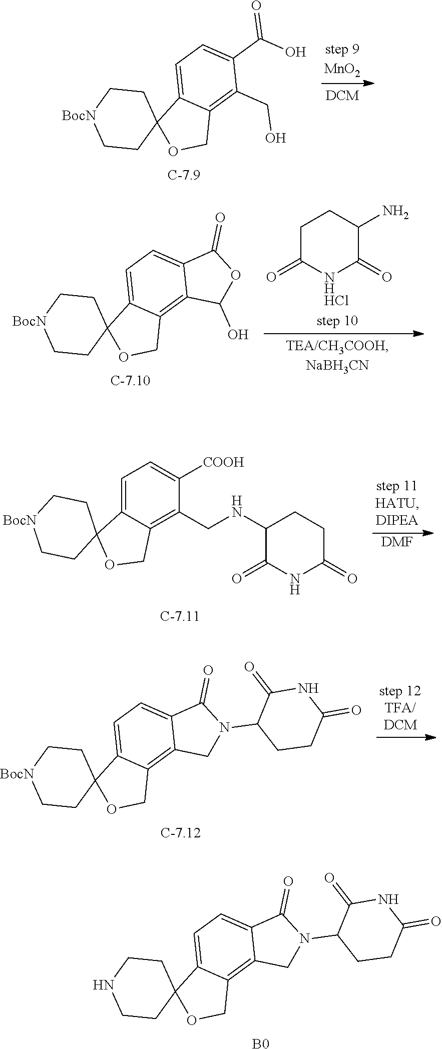

### Step 1:

To a solution of 5—Bromo-3H-isobenzofuran-1-one (C-7.1) (10 g, 1 eq.) in trifluoromethanesulfonic acid (80 mL, 20 eq.) was added NIS(12.5 g, 1.2 eq.) at 0° C. in portions. The mixture was allowed to warm to room temperature and stirred overnight. TLC showed no starting material remained and two new spots formed. The reaction mixture was poured into ice-water and yellow solid precipitated. The mixture was filtered and the filter cake was washed with ice cold water. The filter cake was dissolved in DCM and dried over sodium sulfate. The mixture was filtered and the filtrate was concentrated to afford a yellow solid. The crude product was purified by silica gel flash chromatography. The less polar product (top spot on TLC)C-7.2 was obtained as a brown solid (8 g, yield 50%).

### Step 2:

A vial was charged with compound C-7.2 (8 g, 1 eq.), Pd(dppf) Cl₂ (0.2 eq.), K₂CO₃ (3 eq.) and dioxane-H₂O (100 mL/20 mL). The mixture was purged with nitrogen and potassium vinyltrifluoroborate (2.0 eq.) was added into. The reaction was heated to 65° C. for 16 h. TLC showed reaction was complete. The mixture was diluted with ethyl acetate and washed with water. The organic layer was washed with brine and dried over sodium sulfate. The crude product was purified by silica gel column chromatography using 0-50% EtOAc/hexane to give compound C-7.3 as a yellow foam (3.2 g, yield 57%).

LC-MS: 239/241 [M+H]⁺; ¹H NMR (600 MHz, Chloroform-d) δ 7.70 (d, J=8.1 Hz, 1H), 7.58 (d, J=8.1 Hz, 1H), 6.92 (dd, J=18.0, 11.6 Hz, 1H), 5.62 (d, J=11.6 Hz, 1H), 5.42 (d, J=18.0 Hz, 1H), 5.33 (s, 2H).

### Step 3:

A solution of compound C-7.3 (5 g) in CH₂Cl₂ (100 mL) was cooled to −78° C. then O₃ was bubbled into this solution. The passage of O₃ was continued for a further 30 min until the color turned pale blue and then air was bubbled into the solution for 10 min to remove excess O₃. After dropwise addition of Me₂S(2 mL), the solution was kept stirred and warmed to room temperature. The mixture was diluted with water and extracted with DCM. The organic layer was washed with brine and dried over MgSO₄. The residue was quickly purified by chromatography to give compound C-7.4 (4 g).

### Step 4:

To a solution of compound C-7.4 (4 g, 1.0 eq.) in MeOH (40 mL, 10V) was added NaBH₄ (1.9 g, 3 eq.) at 0° C. in portions. TLC indicated compound 4 was consumed completely and LCMS indicated there was desired product. The reaction mixture was quenched by addition H₂O at 20° C., and then concentrated under reduced pressure to remove MeOH. Then the mixture was extracted with EtOAc). The combined organic layers were washed with brine, dried over Na₂SO₄, filtered and concentrated under reduced pressure. The residue was purified by column chromatography (SiO₂, Petroleum ether/Ethyl acetate=1/1 to Ethyl acetate) to give compound C-7.5 (3 g, yield 75%).

### Step 5:

A round bottomed flask equipped with a stirrer bar was charged with a mixture of compound C-7.5 (4 g, 1.0 eq.), tert-butyl 4-(4,4,5,5-tetramethyl-1,3,2-dioxaborolan-2-yl)-3,6-dihydropyridine-1 (2H)-carboxylate (7.7 g, 1.5 eq.), potassium carbonate (6.9 g, 3.0 eq.), and Pd(dppf) Cl₂ (2.4 g, 0.2 eq.). The flask was evacuated and back-filled with nitrogen (x 3). The mixture of dioxane-H₂O (100 mL/20 mL) was added and kept stirred at 90° C. for 10 hours. The cooled reaction mixture was diluted with EtOAc and filtered through Celite™ to remove insoluble material. The filtrate was washed with water, saturated aqueous sodium chloride and then dried over magnesium sulfate, filtered and the filtrate concentrated. The crude material was purified by flash silica chromatography, elution gradient MeOH in DCM. Pure fractions were combined and concentrated to afford compound C-7.6 (5 g, 89%).

### Step 6:

To a mixture of compound C-7.6 (6 g, 1 eq.) in MeCN (60 mL) was added NBS(3.7, 1.2 eq.) in one portion. The mixture was stirred at 20° C. for 16 h. The mixture was concentrated in vacuum and the crude material was purified by flash silica chromatography, elution gradient MeOH in DCM to give compound C-7.7 was obtained as a white solid (6.6 g, 90% yield).

¹H NMR (600 MHZ, Chloroform-d)δ 7.87 (d, J=7.9 Hz, 1H), 7.66 (d, J=7.9 Hz, 1H), 5.25 (s, 2H), 5.16 (d, J=13.0 Hz, 1H), 5.05 (d, J=13.0 Hz, 1H), 4.25 (m, 2H), 4.08-3.81 (m, 2H), 3.33 (m, 1H), 2.68-2.56 (m, 1H), 1.65 (d, J=13.9 Hz, 1H), 1.50 (s, 9H).

### Step 7:

To a solution of compound C-7.7 (500 mg, 1.0 eq.) in toluene (10 mL) and MeOH (1 mL) was added n-Bu₃SnH (5.0 eq.) and AIBN (0.1 eq.). The mixture was heated to reflux and stirred overnight. After cooling down, an additional n-Bu₃SnH (5.0 eq.) was added into above mixture and kept stirred at 100° C. for another 12 h. TLC showed no starting material remained and the reaction mixture was poured into saturated aq. KF solution (100 mL) and stirred for 1 h. Then, the reaction mixture was filtered and extracted with ethyl acetate, washed with brine, and then dried over sodium sulfate. The crude product was purified by silica gel column chromatography (PE:EA=4:1) to give compound C-7.8 was obtained as a white solid (60% yield). LC-MS: 346 [M+H]⁺; ¹H NMR (600 MHZ, DMSO-d₆) δ 7.78 (d, J=7.8 Hz, 1H), 7.57 (d, J=7.8 Hz, 1H), 5.39 (s, 2H), 5.09 (s, 2H), 3.98 (brs, 2H), 3.07 (brs, 2H), 1.88 (td, J=13.1, 4.9 Hz, 2H), 1.64 (dd, J=13.8, 2.4 Hz, 2H), 1.43 (s, 9H).

### Step 8:

To a solution of compound C-7.8 (1.25 g, 1 eq.) in tetrahydrofuran (10 mL) and water (10 mL) was added sodium hydroxide (720 mg, 5 eq.). The mixture was stirred at 20° C. for 16 h. TLC (ethyl acetate: hexane=1:1) showed reaction was complete. The mixture was adjusted to pH=5-6 with aq. hydrochloric acid (1 M) and extracted with ethyl acetate. The organic layer was washed with brine and dried over sodium sulfate. The crude material 9 was not further purified and used as crude for the next step.

### Step 9:

To a solution of compound C-7.9 (1 g, crude, 1 eq.) in dichloromethane (50 mL) was added manganese dioxide (20 eq.). The mixture was stirred at 20° C. for about 1 h. TLC showed reaction was complete. The mixture was diluted with dichloromethane and MeOH, then filtered through a pad of Celite. The filtrate was concentrated in vacuum and the crude product C-7.10 (0.6 g, crude) was used directly in the next step.

Step 10:

To a mixture of compound C-7.10 (300 mg, crude, 1.0 eq.) in methanol (5 mL) and dichloromethane (5 mL) was added 3-aminopiperidine-2,6-dione (162 mg, 1.5 eq., HCl salt), AcONa (204 mg, 3.0 eq.) and AcOH (150 μL, 3.0 eq.). The mixture was stirred at 20° C. for 1 h, then sodium cyanoborohydride (104 mg, 2.0 eq.) was added and the mixture was further stirred for 30 min. LCMS showed the reaction was complete. Next, the reaction mixture was concentrated under reduced pressure to give a residue which was purified by pre-HPLC (20% ˜50% ACN, neutral). The desired product C-7.11 was obtained as a white solid 120 mg after lyophilization.

### Step 11:

To a solution of compound C-7.11 (180 mg 1.0 equiv) in DMF (3 mL) was added HATU (216 mg, 1.5 equiv) and DIPEA (0.2 mL, 3.0 equiv) at 0° C., and the reaction was stirred at rt for 30 min. UPLC-MS indicated a new main peak with desired MS formed, then quenched with water and the mixture was extracted with ethyl acetate, washed with brine, and then dried over sodium sulfate. The target compound C-7.12 was obtained as a brown solid (100 mg, 60% yield).

LC-MS: 456 [M+H]⁺; ¹H NMR (400 MHZ, DMSO-d₆) δ 11.01 (s, 1H), 7.66 (d, J=7.7 Hz, 1H), 7.47 (d, J=7.7 Hz, 1H), 5.16-5.11 (m, 1H), 5.08 (m, 2H), 4.44 (d, J=17.4 Hz, 1H), 4.29 (d, J=17.4 Hz, 1H), 3.98 (m, 2H), 3.04 (m, 3H), 2.60 (d, J=17.0 Hz, 1H), 2.42-2.31 (m, 1H), 2.04-1.93 (m, 1H), 1.92-1.80 (m, 2H), 1.64 (m, 2H), 1.43 (s, 9H).

### Step 12:

Compound C-7.12 was treated with TFA in DCM at room temperature to de-protect the N-Boc group to provide the cereblon ligand BO.

LC/MS(ESI) m/z: 355.1.

### Compound B1.

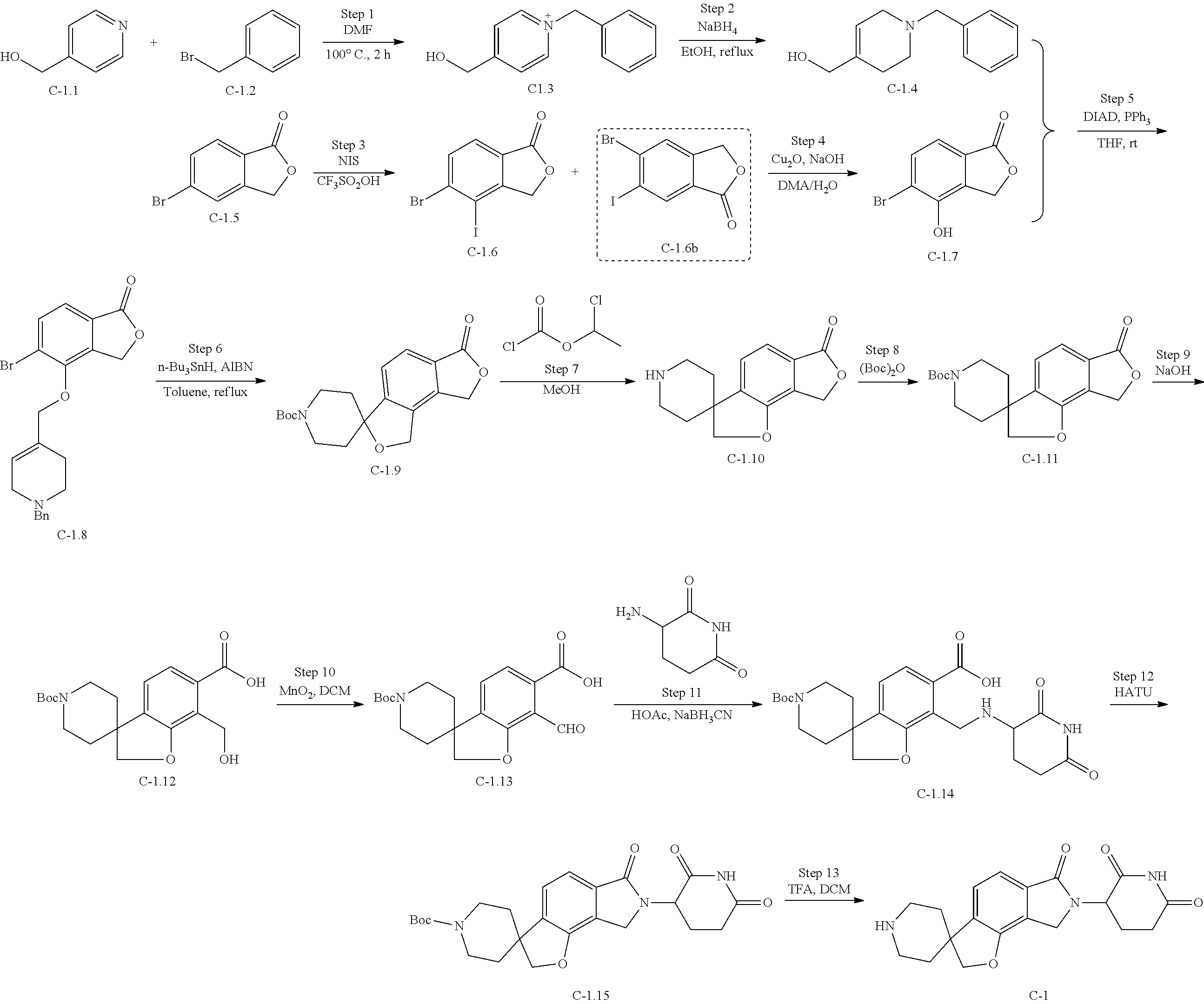

### Step 1-2:

To a solution of pyridin-4-ylmethanol (C-1.1, 100 g, 916 mmol, 1.0 eq.) in DMF (400 mL) was added BnBr (172 g, 1008 mmol, 1.1 eq.). The mixture was allowed to heat to 100° C. and stirred 3 h. TLC showed no starting material remained and a new spot formed. The residue was dissolved in EtOH (1500 mL), then 45 g of sodium borohydride (1191 mmol, 1.3 eq.) was added portionwise at 0° C. The mixture was continued to stir at 0° C. for 1 h and then at reflux for 2 h. The solvent was evaporated under reduced pressure, then water was added, and the mixture was extracted with EA. The combined organic phases were dried over Na₂SO₄ and evaporated. The residue was purified by flash chromatograph (DCM:MeOH=100:0-30:1) to afford 107 g of product C-1.4 (Viscous oil, 2 steps, yield 80%). LC-MS: 204 [M+H]⁺; ¹H NMR (400 MHZ, Chloroform-d)δ 7.41-7.28 (m, 5H), 5.65 (dt, J=3.3, 1.7 Hz, 1H), 4.03 (s, 2H), 3.62 (s, 2H), 3.07-2.92 (m, 2H), 2.63 (t, J=5.8 Hz, 2H), 2.12 (m, 2H).

### Step 3:

To a solution of 5—Bromo-3H-isobenzofuran-1-one (C-1.5) (100 g, 1 eq.) in trifluoromethanesulfonic acid (1000 g, 10 V) was added NIS(125 g, 1.2 eq.) at 0° C. in portions. The mixture was allowed to warm to room temperature and stirred overnight. TLC showed no starting material remained and two new spots formed. The reaction mixture was poured into ice-water and yellow solid precipitated. The mixture was filtered, and the filter cake was washed with ice cold water. The filter cake was dried and obtained as a yellow solid (100 g, yield 62%), to be a mixture of product C-1.6 (top spot on TLC) and product C-1.6b (bottom spot on TLC, which was not further reacted in next step).

### Step 4:

To a mixture of compound C-1.6 (100 g, 1 eq.), sodium hydroxide (57.5 g, 5 eq.) in water (1000 mL, 1.5 M) and N,N-dimethylacetamide (600 mL) was added cuprous oxide (8.5 g, 0.2 eq.). The reaction mixture was heated to 80° C. and stirred for 12 h. TLC showed the compound C-1.6 (top spot on TLC) was completely consumed. The reaction mixture was poured into water (1000 mL) and treated with solid K₂CO₃ until pH 8-9, and extracted with EA. The aqueous layer neutralized using 1 (N) hydrochloride solution and extracted with ethyl acetate, washed with brine, and then dried over sodium sulfate and evaporated. The crude product was purified by silica gel column chromatography to give compound C-1.7 was obtained as a yellow solid (42 g, 39% yield). LC/MS(ESI) m/z: 228.94; ¹H NMR (400 MHZ, DMSO-d₆) δ 10.90 (s, 1H), 7.72 (d, J=8.0 Hz, 1H), 7.23 (d, J=8.0 Hz, 1H), 5.35 (s, 2H).

### Step 5:

To a solution of compound C-1.7 (20 g, 1.0 eq.) in 200 mL of THF, compound C-1.4 (23.1 g, 1.3 eq.) and PPh₃ (34.4 g, 1.55 eq.) was added. The reaction mixture was cooled to 0° C. and DIAD (27.1 mL, 1.55 eq.) was added dropwise. The resultant mixture was then stirred overnight at room temperature. The solvent was evaporated at reduced pressure and the crude product was purified by silica gel column chromatography using 0-100% EtOAc/hexane. The desired product C-1.8 was obtained as a yellow foam (17.7 g, yield 49%). LC-MS: 414/416 [M+H]⁺; ¹H NMR (400 MHZ, Chloroform-d)δ 7.74 (d, J=8.0 Hz, 1H), 7.51 (d, J=8.0 Hz, 1H), 7.41-7.29 (m, 5H), 5.79 (dt, J=3.5, 1.8 Hz, 1H), 5.41 (s, 2H), 4.52 (s, 2H), 3.63 (s, 2H), 3.04 (dt, J=3.1, 1.5 Hz, 2H), 2.68 (t, J=5.7 Hz, 2H), 2.42-2.29 (m, 2H).

### Step 6:

To a solution of C-1.8 (14.8 g, 35.7 mmol, 1.0 eq.) in toluene (150 mL) was added n-Bu₃SnH (41.6 g, 142.9 mmol, 4.0 eq.) and AIBN (0.6 g, 3.57 mmol, 0.1 eq.). The mixture was heated to reflux and stirred overnight. TLC (PE:EA=1:1) showed no starting material remained and new spots formed. The reaction mixture was poured into saturated aq. KF solution (100 mL) and stirred overnight. Then, the reaction mixture was extracted with ethyl acetate, washed with brine, and then dried over sodium sulfate. The crude product was purified by silica gel column chromatography (DCM:MeOH=50:1) to give compound C-1.9 was obtained as a white solid (7.1 g, 60% yield). LC-MS: 336 [M+H]⁺; ¹H NMR (400 MHZ, Chloroform-d)δ 7.48 (d, J=7.7 Hz, 1H), 7.38-7.31 (m, 5H), 7.30 (d, J=7.6 Hz, 1H), 5.25 (s, 2H), 4.51 (s, 2H), 3.55 (s, 2H), 2.93 (m, 2H), 2.12-1.96 (m, 4H), 1.81-1.70 (m, 2H); 13C NMR (126 MHZ, Chloroform-d)δ 171.0, 153.45, 141.50, 138.11, 129.27, 128.47, 127.56, 127.37, 127.13, 124.24, 118.54, 81.96, 67.44, 63.51, 50.83, 44.94, 36.75.

### Step 7-8:

To a solution of C-1.9 (10 g, 29.8 mmol, 1.0 eq.) in DCE (100 mL) was added α-chloroethyl chloroformate (ACE-C1, 1.0 eq.) at 0° C. and then refluxing the mixture for 1 h. The intermediate ACE-piperidine formed and is usually deacetylated directly to C-1.10 by evaporating the reaction mixture in vacuo and then heating the residue in MeOH. The residue was dissolved in THF (100 mL), then 4.5 g of triethylamine (44.7 mmol, 1.5 eq.) and Boc₂O (38.7 mmol, 1.3 eq.) was added. The mixture was continued to stir for 3 h at room temperature. The solvent was evaporated under reduced pressure, then water was added, and the mixture was extracted with EA. The combined organic phases were dried over Na₂SO₄ and evaporated. The residue was purified by flash chromatograph to afford 6.0 g of product C-1.11 (2 steps, yield 60%). LC-MS: 346 [M+H]⁺; LC-MS: 346 [M+H]⁺; ¹H NMR (400 MHZ, Chloroform-d) δ 7.49 (d, J=7.6 Hz, 1H), 7.25 (d, J=7.6 Hz, 1H), 5.25 (s, 2H), 4.55 (s, 2H), 4.24-4.01 (m, 2H), 2.86 (m, 2H), 1.87 (dd, J=12.7, 4.6 Hz, 2H), 1.74 (d, J=13.5 Hz, 2H), 1.48 (s, 9H).

### Step 9:

To a solution of compound C-1.11 (15 g, 1 eq.) in tetrahydrofuran (100 mL) and water (100 mL) was added sodium hydroxide (8.7 g, 5 eq). The mixture was stirred at 20° C. for 16 h. TLC (ethyl acetate: hexane=1:1) showed reaction was complete. The mixture was adjusted to pH=5-6 with aq. hydrochloric acid (1 M) and extracted with ethyl acetate. The organic layer was washed with brine and dried over sodium sulfate. The crude product C-1.12 was not further purified and used as crude for the next step.

### Step 10:

To a solution of compound C-1.12 (15 g, crude, 1 eq.) in dichloromethane (300 mL) was added manganese dioxide (20 eq.). The mixture was stirred at 20° C. for about 1 h. TLC showed reaction was complete. The mixture was diluted with dichloromethane and filtered through a pad of Celite. The filtrate was concentrated in vacuum. The crude product was purified by silica gel column chromatography (DCM:MeOH=10:1). The desired compound C-1.13 was obtained as yellow solid. (8 g, 2 steps, 60%). LC-MS: 362 [M+H]⁺; ¹H NMR (400 MHz, Chloroform-d) δ 7.45 (d, J=7.6 Hz, 1H), 7.31 (d, J=7.6 Hz, 1H), 6.79 (brs, 1H), 4.60 (s, 2H), 4.11 (m, 2H), 2.86 (m, 2H), 1.97-1.70 (m, 4H), 1.47 (s, 9H).

### Step 11:

To a mixture of compound C-1.13 (3 g, 1.0 eq.) in methanol (20 mL) and dichloromethane (20 mL) was added 3-aminopiperidine-2,6-dione (4.0 g, 3 eq., TFA salt), AcONa (3.08 g, 6.0 eq.) and AcOH (5.1 mL, 10.0 eq.). The mixture was stirred at 25° C. for 2 h, then sodium cyanoborohydride (1.57 g, 3.0 eq.) was added and the mixture was further stirred for 30 min. LCMS showed the reaction was complete. Next, the reaction mixture was quenched with water and concentrated under reduced pressure to give a residue which was redissolved in acetonitrile and water (1:1, 30 mL). The solution was mixed well at beginning. After standing at 0-5° C. overnight, the mixture was filtered, and the filter cake was washed with acetonitrile and water (1:1) and vacuum dried to afford the crude product C-1.14 as a solid (900 mg, yield=60%). LC-MS: 374 [M+H]⁺.

### Step 12:

To a solution of compound C-1.14 (900 mg 1.0 equiv) in DMF (15 mL) was added HATU (795 mg, 1.1 equiv) and DIPEA (0.72 mL, 3.0 equiv), and the reaction was stirred at rt for 30 min. UPLC-MS indicated a new main peak with desired MS formed, then quenched with water and the mixture was extracted with ethyl acetate, washed with brine, and then dried over sodium sulfate. Compounds C-1.15 was obtained as a brown solid (675 mg, 75% yield). LC/MS(ESI) m/z: 456.21. ¹H NMR (400 MHZ, Chloroform-d)δ 8.00 (s, 1H), 7.50 (d, J=7.7 Hz, 1H), 7.28 (s, 1H), 5.23 (dd, J=13.3, 5.1 Hz, 1H), 4.55 (d, J=1.4 Hz, 2H), 4.46 (d, J=16.0 Hz, 1H), 4.32 (d, J=16.0 Hz, 1H), 4.15 (s, 2H), 3.01-2.77 (m, 4H), 2.38 (dd, J=13.1, 5.0 Hz, 1H), 2.29-2.17 (m, 1H), 1.92 (t, J=12.5 Hz, 2H), 1.83-1.72 (m, 2H), 1.52 (s, 9H).

### Step 13:

Compound C-1.15 was treated with TFA in DCM at room temperature to de-protect the N-Boc group to provide the cereblon ligand C-1. LC/MS(ESI) m/z: 356.15. ¹H NMR (400 MHz, CDCl₃) δ 8.00 (s, 1H), 7.50 (d, J=7.7 Hz, 1H), 7.28 (s, 1H), 5.23 (dd, J=13.3, 5.1 Hz, 1H), 4.55 (d, J=1.4 Hz, 2H), 4.46 (d, J=16.0 Hz, 1H), 4.32 (d, J=16.0 Hz, 1H), 4.15 (s, 2H), 3.01-2.77 (m, 4H), 2.38 (dd, J=13.1, 5.0 Hz, 1H), 2.29-2.17 (m, 1H), 1.92 (t, J=12.5 Hz, 2H), 1.83-1.72 (m, 2H).

Compound B2: (S)-3-(6′-oxo-1′,2′,6′,8′-tetrahydro-7′H-spiro[piperidine-4,3′-pyrrolo[3,4-g[indol]-7′-yl) piperidine-2,6-dione (C-3);

Compound B3: (S)-3-(1′-methyl-6′-oxo-1′,2′,6′,8′-tetrahydro-7′H-spiro[piperidine-4,3′-pyrrolo[3,4-g]indol]-7′-yl) piperidine-2,6-dione (C-5).

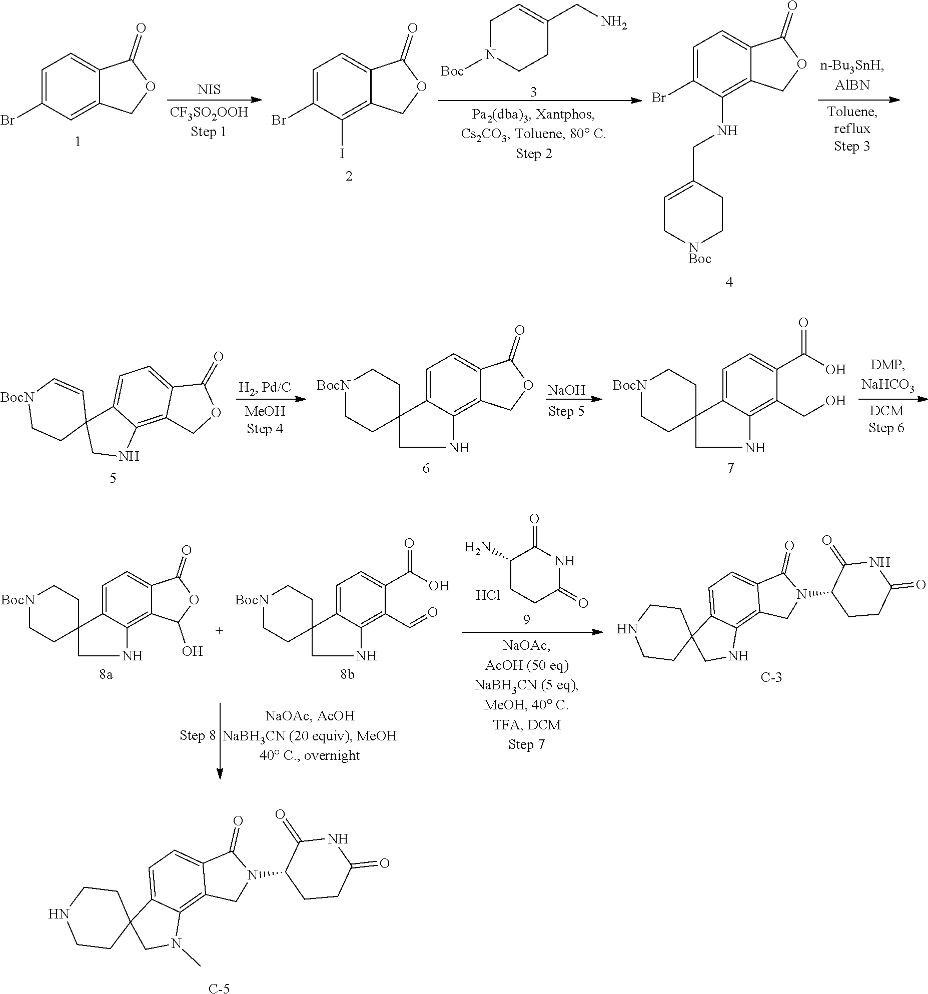

### Step 1:

To a solution of 5—Bromo-3H-isobenzofuran-1-one (1) (10 g, 1 eq.) in trifluoromethanesulfonic acid (100 g, 10 V) was added NIS(12.5 g, 1.2 eq.) at 0° C. in portions. The mixture was allowed to warm to room temperature and stirred overnight. TLC showed no starting material remained and two new spots formed. The reaction mixture was poured into ice-water and yellow solid precipitated. The mixture was filtered and the filter cake was washed with ice cold water. The filter cake was dried and obtained as a yellow solid (10 g, yield 62%), to be a mixture of product 2 (top spot on TLC) and product 2b (bottom spot on TLC, which was not further reacted in next step).

### Step 2:

Compound 3 was made according to the procedure reported (Bioorg. Med. Chem. Lett. 2016, 26, 228-234). To a flask containing compound 2 (500 mg, 1.0 eq.), compound 3 (377 mg, 1.2 eq.), Pd₂ (dba)3 (136 mg, 0.1 eq.), Xantphos (257 mg, 0.3 eq.) and Cs₂CO₃ (1447 mg, 3.0 eq.) was added Toluene (15 mL). The reaction was evacuated and backfilled with N₂ three times. The reaction was stirred at 80° C. for 6 h and then was allowed to cool to room temperature and filtered. The filtrate was evaporated, and the residue was purified by silica gel chromatography (0-25% ethyl acetate in hexane) to afford product 4 as a light yellow powder 316 mg (yield=51%).

LC-MS: 323.14 [M+H]⁺. ¹H NMR (400 MHZ, Chloroform-d)δ 7.61 (d, J=8.0 Hz, 1H), 7.17 (d, J=7.9 Hz, 1H), 5.64-5.53 (m, 1H), 5.36 (s, 2H), 3.93-3.79 (m, 4H), 3.53 (t, J=5.7 Hz, 2H), 2.17-2.05 (m, 2H), 1.46 (s, 9H).

Step 3 and step 4:

To a solution of compound 4 (300 mg, 1.0 eq.) and AIBN (35 mg, 0.3 eq.) in Toluene (10 mL) was added Bu₃SnH (954 μL, 5.0 eq.). The reaction was stirred at 110° C. in a sealed tube for 24 h. Then cooled to room temperature, quenched with saturated aq. KF solution (20 mL) and kept the mixture stirring overnight. The result mixture was extracted with ethyl acetate (3 times). The combined organic layers were washed with brine (3 times), dried over Na₂SO₄, filtered, and concentrated under reduced pressure to give a crude mixture. The mixture was purified by silica gel chromatography (0-30% ethyl acetate in hexane) to afford the crude product 5 as a light-yellow oil (90 mg).

LC-MS: 343.37 [M+H]⁺.

To a solution of compound 5 (90 mg) in MeOH (5 mL) was added Pd/C (90 mg). The reaction was evacuated and backfilled with H₂ and stirred at room temperature under H₂ atmosphere for 6 h. Then filtered through celite and the filtration was concentrated under reduced pressure to give the cude product, which is purified by silica gel chromatography (0-50% ethyl acetate in hexane) to afford the compound 6 as a white solid (50 mg, 20% yield for steps 3 and 4).

LC-MS: 345.22 [M+H]⁺. ¹H NMR (400 MHZ, Chloroform-d)δ 7.37 (d, J=7.6 Hz, 1H), 7.19 (d, J=7.6 Hz, 1H), 5.20 (s, 2H), 4.19-4.04 (m, 2H), 3.67 (s, 2H), 3.00-2.77 (m, 2H), 1.91-1.68 (m, 4H), 1.48 (s, 9H). 13C NMR (101 MHZ, CDCl₃)δ 171.42, 154.87, 143.02, 142.81, 128.22, 126.10, 123.66, 117.49, 79.98, 67.97, 56.18, 44.96, 40.85, 35.64, 28.54.

### Step 5:

To a solution of 6 (48 mg, 1.0 equiv) in THF/MeOH/H₂O (2 mL/2 mL/1 mL) was added NaOH (111 mg, 20 equiv). The reaction was stirred at rt overnight, then concentrated to remove most of the THF/MeOH. The residue was diluted with 1 mL water, followed by neutralization with 2 N aq HCl to PH 4-6, then extracted with EA (5 mL, 6 times). The combined organic layer was washed with brine, filtered, dried with Na₂SO₄, and concentrated under reduced pressure to give the crude product 7 as a light-yellow oil 50 mg, which was directly used in the next step.

LC-MS: 363.28 [M+H]⁺.

### Step 6:

To a solution of 7 (40 mg, 1.0 equiv) in DCM (5 mL) was added NaHCO₃ (28 mg, 3.0 eq.), followed by add DMP (47 mg, 1.0 equiv) potionwise. 10 min Later, the reaction mixture was diluted with DCM and washed with brine, dried over Na₂SO₄, filtered, and concentrated to afford the crude product 8 as a yellow oil 40 mg, which was directly used in the next step. LC-MS: 361.27 [M+H]⁺.

### Step 7:

To a solution of 9 (73 mg, 4.0 equiv) and NaOAc (28 mg, 4 equiv) in MeOH (4 mL) was added 8 (40 mg, 1.0 equiv) and AcOH (317 μL, 50 eq.). 15 min Later, NaBH₃CN (34.5 mg, 5.0 eq.) was added, and the resulted mixture was stirred at 40° C. for 3 h. The reaction mixture was concentrated to remove some MeOH, and then purified by pre-HPLC to give the Boc-protected C-3, which is further treated with TFA and concentrated to remove TFA. The final compound C-3 was obtained as a white solid 10 mg.

LC-MS: 423.16 [M+H]⁺. ¹H NMR (400 MHZ, Methanol-d₄)δ 7.28-7.17 (m, 2H), 5.14 (dd, J=13.3, 5.2 Hz, 1H), 4.39-4.23 (m, 2H), 3.67 (s, 2H), 3.51-3.39 (m, 2H), 3.25-3.12 (m, 2H), 2.98-2.84 (m, 1H), 2.84-2.72 (m, 1H), 2.56-2.39 (m, 1H), 2.23-1.96 (m, 5H).

### Step 8:

To a solution of 9 (18 mg, 4.0 equiv) and NaOAc (6.9 mg, 4 equiv) in MeOH (3 mL) was added 8 (40 mg, 1.0 equiv) and AcOH (0.5 mL). 15 min Later, NaBH₃CN (34.5 mg, 20 eq.) was added in potionwise, and the resulted mixture was stirred at 40° C. for overnight. The reaction mixture was concentrated to remove some MeOH, and then purified by pre-HPLC to give the Boc-protected C-5, which is further treated with TFA and concentrated to remove TFA. The final compound C-5 was obtained as a gray solid 4.7 mg.

LC-MS: 469.26 [M+H]⁺. ¹H NMR (400 MHZ, Methanol-d₄)δ 7.21 (s, 2H), 5.14 (dd, J=13.3, 5.2 Hz, 1H), 4.72-4.55 (m, 2H), 3.51-3.39 (m, 4H), 3.24-3.13 (m, 2H), 2.99 (s, 3H), 2.96-2.83 (m, 1H), 2.83-2.74 (m, 1H), 2.60-2.46 (m, 1H), 2.22-2.03 (m, 3H), 2.03-1.94 (m, 2H).
Biological Assay

The biological activities of the compounds of the present application can be assessed with methods and assays known in the art.

In vitro Assay: IC₅₀ Measurements for binding to CRBN/DDB1

The binding potency was determined using HTRF assay technology (Perkin Elmer). Compounds were serially diluted in DMSO and 0.2 μL volume was transferred to white 384-well plate. The reaction was conducted in total volume of 20 μL with addition of 2 nM His tagged CRBN+DDB-DLS7+CXU4 (Wuxi, catalogue #RP210521GA) to compounds followed by addition of 60 nM Fluorescent probe Cy⁵-labeled Thalidomide (Tenova Pharma, catalogue #T52461), and 0.4 nM of MAb Anti-6HIS Tb cryptate Gold (Cisbio, catalogue #61HI2TLA in the assay buffer (50 mM HEPES pH 7.5, 1 mM TCEP, 0.01% Brij-35, 50 mM NaCl, and 0.1% BSA). After one hour incubation at room temperature, the HTRF signals were read on Envision reader (Perkin Elemer). Data were analyzed using XLfit using four parameters dose response curve to determine IC₅₀s and shown in Table E1.

TABLE E1
CRBN binding IC₅₀

| | CRBN Binding |
|---|---|
| Example | IC₅₀ (μM) |
| A1 | 0.94 |
| A2 | NT |
| A3 | 0.27 |
| A4 | 0.096 |
| A5 | 3.8 |
| A6 | 2.2 |
| A7 | 3.0 |
| A8 | 0.75 |
| A9 | 0.21 |
| A10 | 1.1 |
| A11 | 0.62 |
| A12 | 1.3 |
| A13 | 5.2 |
| A14 | 0.29 |
| A15 | 0.055 |
| A17 | 0.23 |
| A18 | >10,000 |
| A19 | 0.18 |
| A20 | 0.038 |
| A21 | 0.17 |
| A22 | 3.90 |

The binding to cereblon (CRBN) was determined using the Cereblon Binding Kit (Cisbio, #64BDCRBNPEG) following the manufacturer's instruction. Briefly, serially diluted compounds were incubated with GST-tagged wild-type human CRBN protein, XL665-labelled Thalidomide and Europium Cryptate labelled GST antibody at room temperature for about 3 hours. Time Resolved Fluorescence Resonance Energy Transfer (TR-FRET) measurements were acquired on a CALRIOstar plate reader with MARS data analysis software (BMG Labtech), with the following settings: 665/10 nm and 620/10 nm emission, 60 us delay and 400 us integration. The TR-FRET ratio was taken as the 665/620 nm intensity ratio. The readings were normalized to the control (0.5%) and the IC₅₀ was calculated by nonlinear regression (four parameters sigmoid fitted with variable slope) analysis using the GraphPad Prism 8 software.

TABLE E2
CRBN binding IC₅₀

| | CRBN Binding |
|---|---|
| Example | IC₅₀ (μM) |
| A16 | 0.42 |
| B0 | 14 |
| B1 | 1.0 |
| B2 | 2.8 |
| B3 | 3.1 |

### II. Bifunctional Degraders

### 1. ER Degraders

### Chroman Series

### Compound CHR-A71:3-(1′-((7-(4-((3S,4R)-7-Hydroxy-3-Phenylchroman-4-Yl)Phenyl)-7-Azaspiro[3.5]Nonan-2-Yl)Methyl)-7-Oxo-5,7-Dihydro-2H,6H-Spiro[Furo [2,3-f]Isoindole-3,4′-Piperidin]-6-Yl) Piperidine-2,6-Dione

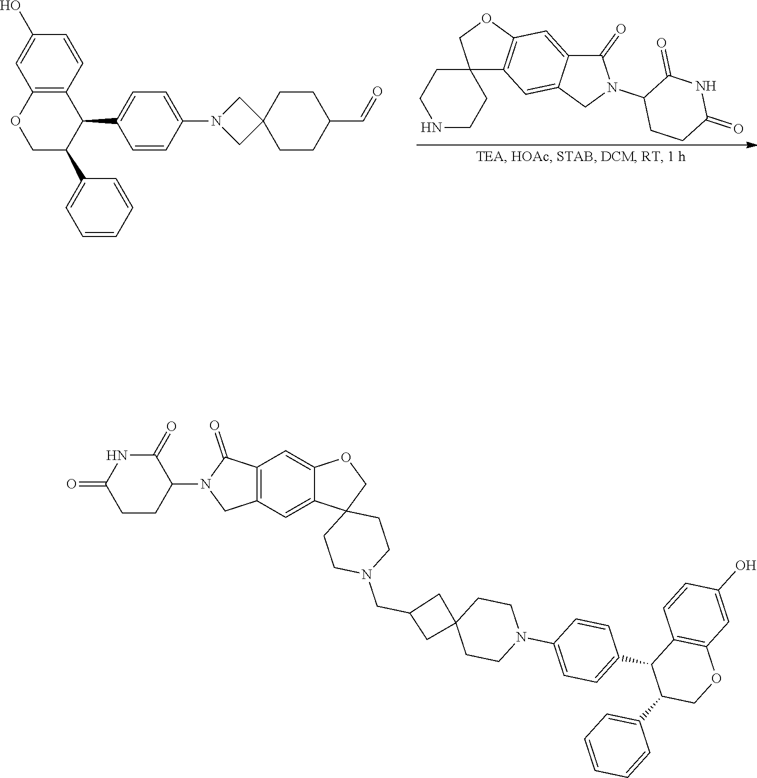

To a mixture of 7-(4-((3S,4R)-7-hydroxy-3-phenylchroman-4-yl)phenyl)-7-azaspiro[3.5]nonane-2-carbaldehyde (30 mg, 0.066 mmol, 1 eq.), (S)-3-(7-oxo-5,7-dihydro-2H,6H-spiro[furo [2,3-f]isoindole-3,4′-piperidin]-6-yl) piperidine-2,6-dione hydrochloride (31 mg, 0.079 mmol, 1.2 eq.), TEA (10 mg, 0.099 mmol, 1.5 eq.) in DCM (2.0 mL) was added acetic acid (6.7 mg, 0.11 mmol, 1.7 eq.) followed by sodium triacetoxyborohydride (28 mg, 0.132 mmol, 2 eq.). The mixture was stirred at room temperature for 30 minutes and concentrated. The residue was purified by reverse-phase chromatography (0-50% Acetonitrile/0.05% Formic acid)) to afford 3-(1′-((7-(4-((3S,4R)-7-hydroxy-3-phenylchroman-4-yl)phenyl)-7-azaspiro[3.5]nonan-2-yl)methyl)-7-oxo-5,7-dihydro-2H,6H-spiro[furo [2,3-f]isoindole-3,4′-piperidin]-6-yl) piperidine-2,6-dione (9.84 mg, 19% yield) as white solid. LC-MS purity: 100% (UV at 254 nm), 793.5 [M+H]⁺

¹H NMR (400 MHZ, DMSO)δ 10.96 (s, 1H), 8.20 (s, 1H), 7.44 (s, 1H), 7.21-7.08 (m, 3H), 7.00 (s, 1H), 6.82-6.54 (m, 5H), 6.44-6.20 (m, 4H), 5.12-5.02 (m, 1H), 4.50-4.40 (m, 2H), 4.38-4.27 (m, 2H), 4.25-4.14 (m, 3H), 3.53-3.50 (m, 1H), 3.01-2.95 (m, 2H), 2.93-2.85 (m, 3H), 2.79 (d, J=9.4 Hz, 2H), 2.64-2.54 (m, 1H), 2.45-2.30 (m, 4H), 2.02-1.84 (m, 7H), 1.71-1.57 (m, 4H), 1.55-1.46 (m, 2H), 1.41 (t, J=9.8 Hz, 2H).

### Compound CHR-A99:3-(1′-((2-(4-((3R,4S)-7-hydroxy-3-phenylchroman-4-yl)phenyl)-2-azaspiro[3.5]nonan-7-yl)methyl)-7-oxo-5,7-dihydro-2H,6H-spiro[furo [2,3-f]isoindole-3,4′-piperidin]-6-yl) piperidine-2,6-dione

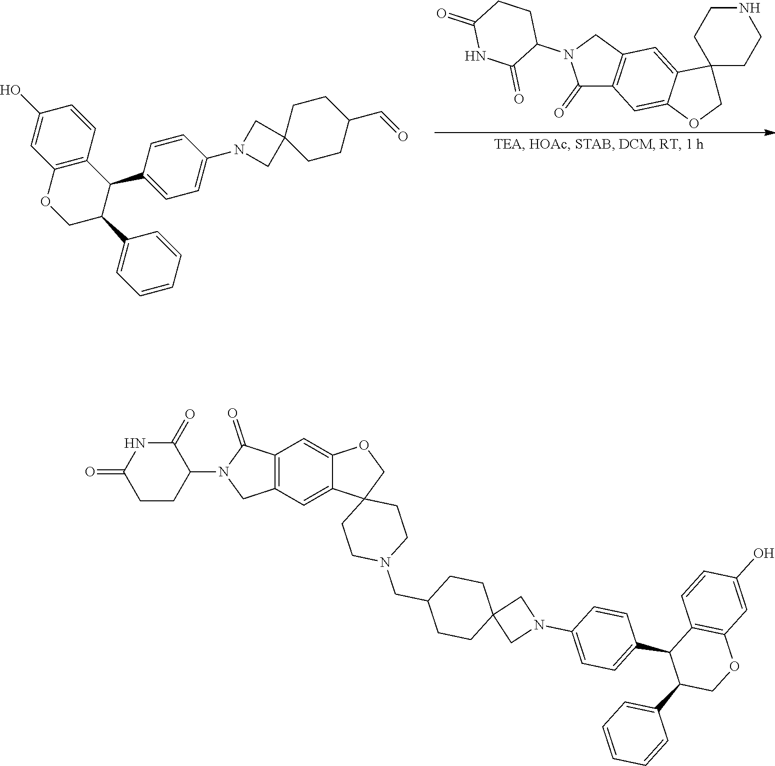 [SMILES: O=CC1CCC2(CC1)CN(c1ccc([C@@H]3c4ccc(O)cc4OC[C@@H]3c3ccccc3)cc1)C2 ; O=C1CCC(N2Cc3cc4c(cc3C2=O)OCC42CCNCC2)C(=O)N1 ; O=C1CCC(N2Cc3cc4c(cc3C2=O)OCC42CCN(CC3CCC4(CC3)CN(c3ccc([C@H]5c6ccc(O)cc6OC[C@H]5c5ccccc5)cc3)C4)CC2)C(=O)N1]

To a mixture of 2-(4-((3S,4R)-7-hydroxy-3-phenylchroman-4-yl)phenyl)-2-azaspiro[3.5]nonane-7-carbaldehyde (30 mg, 0.066 mmol, 1 eq.), 3-(7-oxo-5,7-dihydro-2H,6H-1′12-spiro[furo [2,3-f]isoindole-3,4′-piperidin]-6-yl) piperidine-2,6-dione (23 mg, 0.072 mmol, 1.2 eq.), TEA (6.7 mg, 0.066 mmol, 1.0 eq.) in DCM (2.0 mL) was added acetic acid (7.9 mg, 0.132 mmol, 2.0 eq.) followed by sodium triacetoxyborohydride (28 mg, 0.132 mmol, 2 eq.). The mixture was stirred at room temperature for 30 minutes and concentrated. The residue was purified by reverse-phase chromatography (0-50% acetonitrile/0.05% formic acid)) to afford rac-3-(1′-((2-(4-((3R,4S)-7-hydroxy-3-phenylchroman-4-yl)phenyl)-2-azaspiro[3.5]nonan-7-yl)methyl)-7-oxo-5,7-dihydro-2H,6H-spiro[furo [2,3-f]isoindole-3,4′-piperidin]-6-yl) piperidine-2,6-dione (8.5 mg, 16.3% yield) as white solid. LC-MS purity: 99.7% (UV at 254 nm), 793.4 [M+H]⁺

¹H NMR (400 MHZ, DMSO)δ 10.95 (s, 1H), 7.45 (s, 1H), 7.24-7.07 (m, 3H), 7.00 (s, 1H), 6.82-6.72 (m, 2H), 6.64 (d, J=8.4 Hz, 1H), 6.39-6.22 (m, 4H), 6.09 (d, J=8.4 Hz, 2H), 5.07 (dd, J=13.2, 5.2 Hz, 1H), 4.49-4.40 (m, 2H), 4.37-4.27 (m, 2H), 4.25-4.11 (m, 3H), 3.54-3.46 (m, 2H), 3.42-3.39 (m, 2H), 3.37-3.33 (m, 2H), 2.95-2.78 (m, 3H), 2.62-2.55 (m, 1H), 2.41-2.30 (m, 1H), 2.12 (d, J=6.8 Hz, 2H), 2.02-1.79 (m, 7H), 1.73-1.60 (m, 4H), 1.55-1.37 (m, 3H), 0.98-0.83 (m, 2H).

Compounds shown in the following table were prepared in a manner analogous to Compounds CHR-A71 and CHR-A99 by reductive amination.

TABLE E4

| | | Calcd. | Found. |
|---|---|---|---|
| Compound | | (M + | (M + |
| No. | ¹H NMR | H)⁺ | H)⁺ |
| CHR-A86 | DMSO-d6: δ 10.96 (s, 1H), 8.30 (s, | 753.4 | 753.4 |
| | 1H), 7.46 (s, 1H), 7.21-7.10 (m, | | |
| | 3H), 7.00 (s, 1H), 6.83-6.73 (m, | | |
| | 2H), 6.63 (dd, 3H), 6.38 (d, J = 8.4 | | |
| | Hz, 2H), 6.33-6.22 (m, 2H), 5.07 | | |
| | (dd, 1H), 4.46 (s, 2H), 4.37-4.29 | | |
| | (m, 2H), 4.24-4.10 (m, 3H), 3.59- | | |
| | 3.50 (m, 3H), 2.98-2.76 (m, 3H), | | |
| | 2.69-2.55 (m, 2H), 2.39-2.32 (m, | | |
| | 2H), 2.22-2.14 (m, 2H), 2.00- | | |
| | 1.86 (m, 5H), 1.79-1.63 (m, 5H), | | |
| | 1.22-1.09 (m, 2H). | | |
| CHR-A113 | DMSO-d6: δ 10.97 (s, 1H), 9.49- | 792.4 | 794.5 |
| | 9.02 (m, 1H), 7.39 (d, J = 7.6 Hz, | | |
| | 1H), 7.26 (d, J = 7.6 Hz, 1H), 7.21- | | |
| | 7.07 (m, 3H), 6.79-6.71 (m, 2H), | | |
| | 6.63 (dd, 3H), 6.37 (d, J = 8.6 Hz, | | |
| | 2H), 6.31-6.24 (m, 2H), 5.08 (dd, | | |
| | 1H), 4.51 (s, 2H), 4.42-4.28 (m, | | |
| | 2H), 4.25-4.11 (m, 3H), 3.52- | | |
| | 3.48 (m, 1H), 3.02-2.85 (m, 5H), | | |
| | 2.83-2.76 (m, 2H), 2.70-2.53 (m, | | |
| | 2H), 2.43-2.31 (m, 3H), 2.02- | | |
| | 1.83 (m, 7H), 1.70-1.59 (m, 4H), | | |
| | 1.54-1.47 (m, 2H), 1.45-1.35 (m, | | |
| | 2H). | | |
| CHR-A114 | DMSO-d6: δ 10.97 (s, 1H), 9.27 (s, | 792.4 | 794.5 |
| | 1H), 7.39 (d, J = 7.6 Hz, 1H), 7.26 | | |
| | (d, J = 7.6 Hz, 1H), 7.19-7.09 (m, | | |
| | 3H), 6.80-6.72 (m, 2H), 6.65 (d, J = | | |
| | 8.2 Hz, 1H), 6.42-6.23 (m, 4H), | | |
| | 6.09 (d, J = 8.4 Hz, 2H), 5.08 (dd, | | |
| | 1H), 4.51 (s, 2H), 4.41-4.28 (m, | | |
| | 2H), 4.25-4.11 (m, 3H), 3.54- | | |
| | 3.37 (m, 5H), 2.97-2.77 (m, 3H), | | |
| | 2.68-2.55 (m, 1H), 2.43-2.30 (m, | | |
| | 1H), 2.12 (d, J = 6.8 Hz, 2H), 2.01- | | |
| | 1.81 (m, 7H), 1.75-1.62 (m, 4H), | | |
| | 1.55-1.38 (m, 3H), 0.99-0.84 (m, | | |
| | 2H). | | |

### Indazole Series

### Compound IDZ-B126: (3S)-3-(1′-((9-(5-((6S,8R)-8-methyl-7-(2,2,2-trifluoroethyl)-6,7,8,9-tetrahydro-3H-pyrazolo[4,3-f]isoquinolin-6-yl)pyrimidin-2-yl)-1-oxa-9-azaspiro[5.5]undecan-3-yl)methyl)-6-oxo-6,8-dihydro-2H,7H-spiro[furo [2,3-e]isoindole-3,4′-piperidin]-7-yl) piperidine-2,6-dione

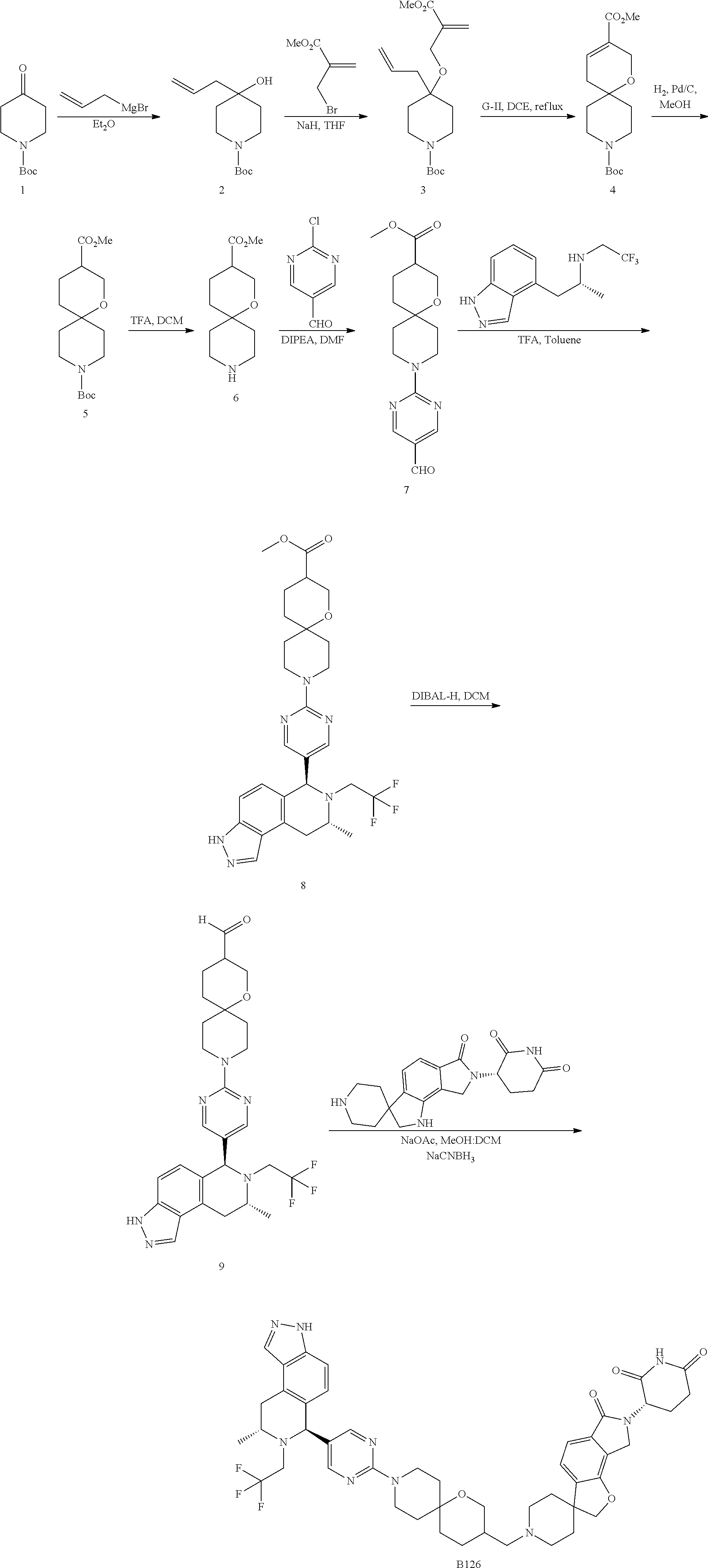

### Step 1: Tert-Butyl 4-Allyl-4-Hydroxypiperidine-1-Carboxylate

Allyl magnesium bromide (1M sol. in Et₂O, 26 mL) was added at 0° C. to a solution of N-Boc-4-piperidone (1, 4.03 g, 20 mmol) in Et₂O (80 mL). It was stirred for 10 min. The reaction mixture was warmed to room temperature and stir for 4 h. Followed by quenching by addition of sat. aq. NH₄Cl. It was then extract with EtOAc. The organic phase was separated and washed twice with water then brine, then dried over sodium sulfate, filtered and concentrated in vacuo. The crude mixture was purified using column chromatography on silica gel (0% to 100% ethyl acetate in hexanes. The desired compound 2 (4.84 g, ˜90% yield) was obtained as a colorless oil. ¹H NMR: (400 MHZ, CDCl₃) δ 5.77-5,94 (m, 1H), 5.19 (dd, J=10.4, 1.8 Hz, 1H), 5.14 (dd, J=17.1, 1.9 Hz, 1H), 3.81 (dt, J=13.4, 3.3 Hz, 2H), 3.08-3.24 (m, 2H), 2.23 (d, J=7.6 Hz, 2H), 1.53 (dd, J=10.4, 4.8 Hz, 4H), 1.46 (s, 9H).

### Step 2: Tert-Butyl 4-Allyl-4-((2-(Methoxycarbonyl) Allyl)Oxy) Piperidine-1-Carboxylate

A 60% oil dispersion of sodium hydride (0.438 g, 1.2 eq) was added to a solution of tert-butyl 4-allyl-4-hydroxypiperidine-1-carboxylate (2, 2.2 g, 1 eq) in anhydrous DMF (10 mL/mmol) and the mixture cooled to 0° C. The mixture was warmed to room temperature over 1 hour and methyl 2-(bromomethyl) acrylate (1.63 g, 1 eq) in DMF was added dropwise to the solution over 5 minutes. The mixture was stirred for 12 h. The reaction mixture cooled down to 0° C., a saturated solution of ammonium chloride was added to the reaction mixture and the mixture was diluted with ethyl acetate. The organic phase was separated and washed twice with water then brine, then dried over sodium sulfate, filtered and concentrated in vacuo. The crude mixture was purified using column chromatography on silica gel (0% to 100% ethyl acetate in hexanes, Rf: 0.3; 30% EA/Hx). The desired compound 3 was obtained as a colorless oil. Yield: 60-70%

### Step 3:9-(Tert-Butyl)3-Methyl 1-Oxa-9-Azaspiro[5.5]Undec-3-Ene-3,9-Dicarboxylate

tert-butyl 4-{[2-(methoxycarbonyl) prop-2-en-1-yl]oxy}-4-(prop-2-en-1-yl) piperidine-1-carboxylate (3, 340 mg, 1 eq) in anhydrous 1,2-dichloroethane (20 mL/mmol) was combined with G-II (0.05 eq) and the mixture was heated at 50° C. for 4 h. The mixture was cooled to room temperature and quenched by passing air. It was then filtered and evaporated and purified by flash. tert-butyl 3-oxo-1-oxa-9-azaspiro[5.5]undecane-9-carboxylate (4) was obtained as an oil. Yield: ˜80%

### Step 4:9-(Tert-Butyl)3-Methyl 1-Oxa-9-Azaspiro[5.5]Undecane-3,9-Dicarboxylate

Pd/C (100 mg, 10% wt.) was added to a solution of compound 4 (1 gm, 3.31 mmol) in MeOH (33 mL, 10 mL/mmol). The reaction mixture was degassed with H₂ and stirred under a H₂ atmosphere for 12 h at room temperature. The mixture was then filtered through celite and washed with MeOH. Concentration under reduced pressure followed by purification by flash chromatography (0% to 100% ethyl acetate in hexanes) gave the desired compound 5 in 60% yield.

### Step 5: Methyl 1-Oxa-9-Azaspiro[5.5]Undecane-3-Carboxylate

To a solution of 5 (300 mg) in DCM (5 mL) was added TFA (2.5 mL). The reaction mixture was stirred overnight, then concentrated under reduced pressure and used for the next steps without further purification.

### Step 6: Methyl 9-(5-Formylpyrimidin-2-Yl)-1-Oxa-9-Azaspiro[5.5]Undecane-3-Carboxylate

methyl 1-oxa-9-azaspiro[5.5]undecane-3-carboxylate (1 gm, 4.4 mmol) was added to a solution of 2-chloropyrimidine-5-carbaldehyde (600 mg, 4.4 mmol) in acetonitrile (20 mL). To it 2 mL of DIPEA was added and the reaction mixture was stirred at 90° C. for 4 h. The mixture was cooled to room temperature and evaporated and purified by flash. methyl 9-(5-formylpyrimidin-2-yl)-1-oxa-9-azaspiro[5.5]undecane-3-carboxylate (7) was obtained as an yellow solid. Yield: ˜80%

### Steps 7: Methyl 9-(5-((6S,8R)-8-Methyl-7-(2,2,2-Trifluoroethyl)-6,7,8,9-Tetrahydro-3H-Pyrazolo[4,3-f]Isoquinolin-6-Yl)Pyrimidin-2-Yl)-1-Oxa-9-Azaspiro[5.5]Undecane-3-Carboxylate

To a mixture of (R)-1-(1H-indazol-4-yl)-N-(2,2,2-trifluoroethyl) propan-2-amine (286 mg, 1.0 mmol, 1 eq) in TFA (1 mL) and toluene (10 mL) was added methyl 9-(5-formylpyrimidin-2-yl)-1-oxa-9-azaspiro[5.5]undecane-3-carboxylate (370 mg, 1.0 mmol, 1 eq). The mixture was stirred at 100° C. for overnight. LCMS showed the reaction was completed. The reaction was concentrated and purified by flash. The product methyl 9-(5-((6S,8R)-8-methyl-7-(2,2,2-trifluoroethyl)-6,7,8,9-tetrahydro-3H-pyrazolo[4,3-f]isoquinolin-6-yl)pyrimidin-2-yl)-1-oxa-9-azaspiro[5.5]undecane-3-carboxylate (8) was obtained as an yellow solid. LC/MS(ESI) m/z: 573.37.

### Steps 8:9-(5-((6S,8R)-8-Methyl-7-(2,2,2-Trifluoroethyl)-6,7,8,9-Tetrahydro-3H-Pyrazolo[4,3-Flisoquinolin-6-Yl)Pyrimidin-2-Yl)-1-Oxa-9-Azaspiro[5.5]Undecane-3-Carbaldehyde

To a solution of 8 (558 mg, 1.0 mmol, 10 mL/mmol) in DCM at −78° C., 1.5 mL of DIBAL-H (1.0 M in DCM) was added dropwise. Then, the temperature was slowly increased to −20° C. and stirred for 6 h. After that, the reaction was slowly quenched with satd. Na₂SO₄ at 0° C. and was then filtered and washed several times with EtOAc. Purification by flash chromatography to obtain the desired product (9) LCMS(ESI) m/z: 546.20 [M+18].

### Steps 9: (3S)-3-(1'-((9-(5-((6S,8R)-8-methyl-7-(2,2,2-trifluoroethyl)-6,7,8,9-tetrahydro-3H-pyrazolo[4,3-f]isoquinolin-6-yl)pyrimidin-2-yl)-1-oxa-9-azaspiro[5.5]undecan-3-yl)methyl)-6-oxo-6,8-dihydro-2H,7H-spiro[furo [2,3-e]isoindole-3,4′-piperidin]-7-yl) piperidine-2,6-dione

To a mixture of compound 9 (53 mg, 0.1 eq.) in methanol (5 mL) and dichloromethane (5 mL) was added(S)-3-(6-oxo-6,8-dihydro-2H,7H-spiro[furo [2,3-e]isoindole-3,4′-piperidin]-7-yl) piperidine-2,6-dione (35 mg, 0.1 eq.), and AcONa (24 mg, 0.3 eq.). The mixture was stirred at 25° C. for 20 mins, then sodium cyanoborohydride (0.2 mL, 0.2 eq., 1M in THF) was added and the mixture was further stirred for 10 mins. LCMS showed the reaction was complete. It was purified by pre-HPLC, and the desired product (IDZ-B126) was obtained as white solid. LCMS(ESI) m/z: 868.40 [M+1].

Compounds shown in the following table were prepared in a manner analogous to Compound IDZ-B126 by reductive amination.

TABLE E4

| Compound | | |
|---|---|---|
| No. | Structure | [M + H]+ |
| IDZ-B47 | 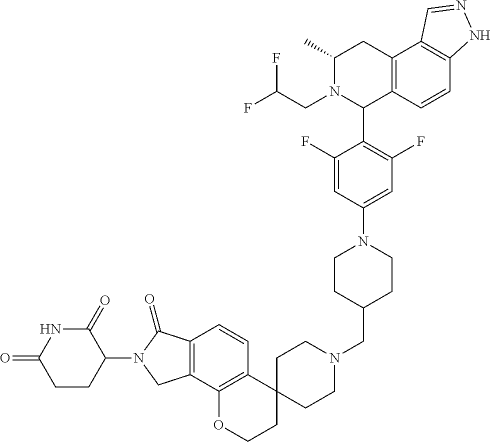 [SMILES: C[C@@H]1Cc2c(ccc3[nH]ncc23)C(c2c(F)cc(N3CCC(CN4CCC5(CCOc6c5ccc5c6CN(C6CCC(=O)NC6=O)C5=O)CC4)CC3)cc2F)N1CC(F)F] | 828.30 |
| IDZ-B56 | 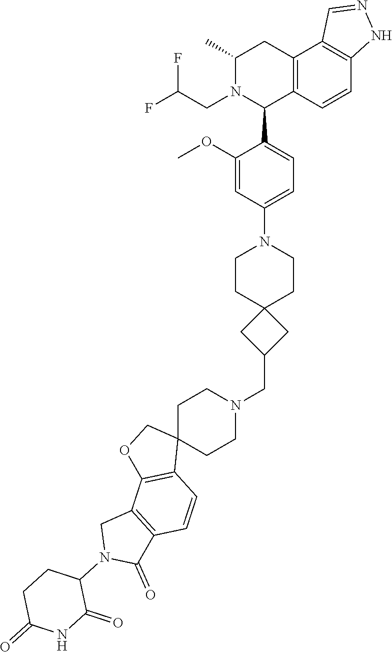 [SMILES: COc1cc(N2CCC3(CC2)CC(CN2CCC4(CC2)COc2c4ccc4c2CN(C2CCC(=O)NC2=O)C4=O)C3)ccc1[C@@H]1c2ccc3[nH]ncc3c2C[C@@H](C)N1CC(F)F] | 848.40 |
| IDZ-B58 | 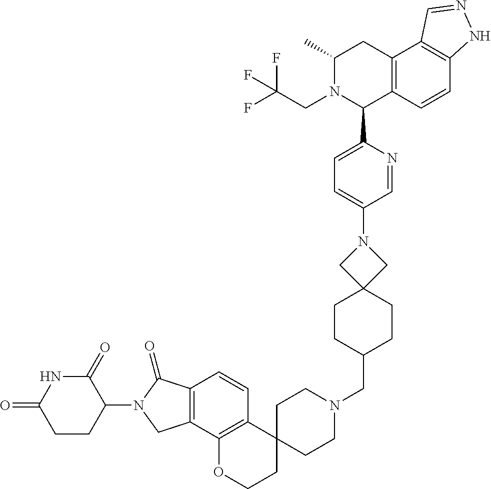 [SMILES: C[C@@H]1Cc2c(ccc3[nH]ncc23)[C@@H](c2ccc(N3CC4(CCC(CN5CCC6(CCOc7c6ccc6c7CN(C7CCC(=O)NC7=O)C6=O)CC5)CC4)C3)cn2)N1CC(F)(F)F] | 851.41 |
| IDZ-B60 | 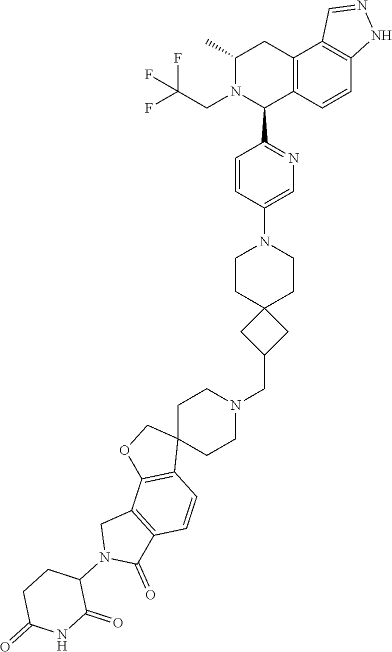 [SMILES: C[C@@H]1Cc2c(ccc3[nH]ncc23)[C@@H](c2ccc(N3CCC4(CC3)CC(CN3CCC5(CC3)COc3c5ccc5c3CN(C3CCC(=O)NC3=O)C5=O)C4)cn2)N1CC(F)(F)F] | 837.31 |

### Biological Assay for Degraders

In-cell Western (ICW) assays in MCF-7 and T47D cell lines.

### Reagents and Consumables for ICW

1) MCF-7 from HDB
2) T47D from HDB
3)CS-FBS, BI, Cat #04-201-1
4) phenol red-free RPMI1640, Thermo, Cat #11835
5) P/S, Biosera Liquid, Cat #XC-A⁴¹²²
6)384-well cell plate (black), Corning, Cat #3764
7 PFA, Electron Microscopy Sciences, Cat #15710
8) Intercept (PBS) Blocking Buffer, Licor, Cat #927-70001
9) Triton X-100, Sigma, Cat #X-100
10) ER antibody, CST, Cat #13258
11) IRDye 800CW Goat anti-Rabbit IgG, LiCor, Cat #926-32211
12) CellTag 700 Stain, Licor, Cat #926-41090
13) Odyssey® DLx Imaging System, LiCor
14) EnVision, PerkinElmer
   · Procedures for ICW assays
   In vitro Assay: MCF-7 and T47D ICW assay

Day 1: MCF-7 and T47D cell (From HDB) were seeded in 384-well black plate with phenol red-free RPMI1640+10% CS-FBS+1% P/S medium (1*104 for MCF-7 and 1.5*104 for T47D cells/well, 30 ul medium) for overnight at 37° C., 5% CO₂ incubator.

Day 2: Cells were treated at desired compound concentrations (0.02 to 300 nM) and DMSO as vehicle control for 16 hrs at 37° C., 5% CO₂ incubator.

Day 3: After 16 hrs of compounds treatment, cells were fixed by 4% PFA and permeabilized with elution buffer (0.1% Triton X-100 in 1% PBS Solution). Subsequently, cells were blocked with Intercept (PBS) Blocking Buffer (Li—COR, Odyssey Blocking Buffer), and were stained with ER (1:500, Cell signaling) primary antibody for overnight at cold room.

Day 4: Remove the buffer, add IRDye 800CW Goat anti-Rabbit IgG Secondary Antibody (1:2000) and CellTag 700 Stain (1:500) in Intercept (PBS) Blocking Buffer. Finally, cell plate is placed in incubator to dry. Image and signal were captured on Odyssey® DLx Imaging System. Data was further analyzed using XLfit using four parameters dose response curve to determine DC₅₀ and Dₘₐₓ.

### Data Analysis

### Data are Analyzed by Image Studio V5.2 and XLfit.

In-cell western blot analysis. a. seed cells in black-sided/clear bottom 96- or 384-well plates at 40,000 or 10,000 cells/well, overnight; b. add diluted compounds (final 0.5% DMSO), 16 hours. 16 h later, remove medium, add 100 μL or 25 μL of 3.7-4.0% formaldehyde (PBS: FA=9:1), RT 20 min, no shaking; c. wash with PBS, and permeabilized with 100 μL or 25 L/well of 1×PBS+0.1% Triton X-100 10 minutes; d. block with 100 μL or 25 μL Licor blocking buffer (Li-Cor), RT 1 h, moderate shaking; d. Add 100 μL or 25 μL of anti-ER (cs-8644, 1:500-1,000)+GAPDH (Millipore MAB374, 1:1000) in Block+0.05% Tween 20. RT 2 h, gentle shaking. Negative control: cells plus secondary antibodies (no primary antibodies); e. wash x 4 with PBS+0.05-0.1% Tween 20, gentel shaking; f. anti-rabbit-680 and anti-mouse-800 (both 1:1000 in LiCor block+0.05% Tween20, RT 1 h, gentle shaking, no light. LI-COR: 0.2% to reduce background; g. wash x 4 with PBS+0.05% Tween 20, gental shaking; h. add 100 μL or 25 μL of PBS to each well and read on CLX plate reader. The relative ER percentage in treated cells were obtained by comparing the values of treated wells to those in untreated and DMSO-treated wells as 100%.

Western Blot Analysis. Western blot analysis was performed essentially as described previously. The cells treated with indicated compounds were lysed in Radioimmunoprecipitation Assay Protein Lysis and Extraction Buffer (25 mmol/L Tris.HCl, pH 7.6, 150 mmol/L NaCl, 1% Nonidet P-40, 1% sodium deoxycholate, and 0.1% sodium dodecyl sulfate) containing proteinase inhibitor cocktail (Roche Diagnostics, Mannheim, Germany). Equal amounts of total protein were electrophoresed through 10% SDS-polyacrylamide gels after determination of protein concentration by BCA assay (Fisher Scientific, Pittsburgh, PA). The separated protein bands were transferred onto PVDF membranes (GE Healthcare Life Sciences, Marlborough, MA) and blotted against different antibodies, as indicated. The blots were scanned, and the band intensities were quantified using GelQuant.NET software provided by biochemlabsolutions.com. The relative mean intensity of target proteins was expressed after normalization to the intensity of glyceraldehyde-3-phosphate dehydrogenase bands.

TABLE E5

| Compound | MCF7 DC₅₀ | MCF7 Dₘₐₓ | T47D DC₅₀ | T47D Dₘₐₓ |
|---|---|---|---|---|
| No. | (nM) | (%) | (nM) | (%) |
| CHR-A71 | A | B | A | B |
| CHR-A86 | | | A | C |
| CHR-A99 | A | B | A | B |
| CHR-A113 | A | B | A | B |
| CHR-A114 | A | A | A | A |

| Compound | | Cell Growth Inhibition in |
|---|---|---|
| No. | ICW DC50 (nM) | T47D cell line IC50 (nM) |
| IDZ-B47 | | A |
| IDZ-B56 | C | |
| IDZ-B58 | C | A |
| IDZ-B60 | A | A |
| IDZ-B126 | A | A |

### 2. STAT3 DEGRADERS

TABLE E8

| | Structure | Chemical Name |
|---|---|---|
| STD- 22 | 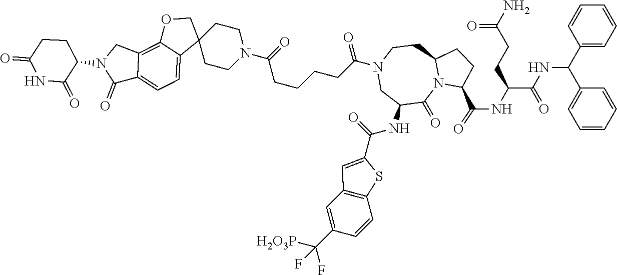 | ((2-(((5S,8S,10aR)-8-(((S)-5- amino-1-(benzhydrylamino)-1,5- dioxopentan-2-yl)carbamoyl)- 3-(6-(7-((S)-2,6-dioxopiperidin- 3-yl)-6-oxo-7,8-dihydro-2H,6H- spiro[furo[2,3-e]isoindole-3,4′- piperidin]-1′-yl)-6-oxohexanoyl)- 6-oxodecahydropyrrolo[1,2- a][1,5]diazocin-5- yl)carbamoyl)benzo[b]thiophen- 5-yl)difluoromethyl)phosphonic acid |
| STD 69 | 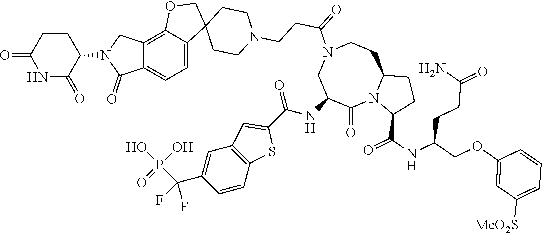 [SMILES: CS(=O)(=O)c1cccc(OC[C@H](CCC(N)=O)NC(=O)[C@@H]2CC[C@@H]3CCN(C(=O)CCN4CCC5(CC4)COc4c5ccc5c4CN([C@H]4CCC(=O)NC4=O)C5=O)C[C@H](NC(=O)c4cc5cc(C(F)(F)P(=O)(O)O)ccc5s4)C(=O)N32)c1] | ((2-(((5S,8S,10aR)-8-(((S)-5- amino-1-(3- (methylsulfonyl)phenoxy)-5- oxopentan-2-yl)carbamoyl)-3-(3- (7-((S)-2,6-dioxopiperidin-3-yl)- 6-oxo-7,8-dihydro-2H,6H- spiro[furo[2,3-e]isoindole-3,4′- piperidin]-1′-yl)propanoyl)-6- oxodecahydropyrrolo[1,2- a][1,5]diazocin-5- yl)carbamoyl)benzo[b]thiophen- 5-yl)difluoromethyl)phosphonic acid |
| STD 70 | 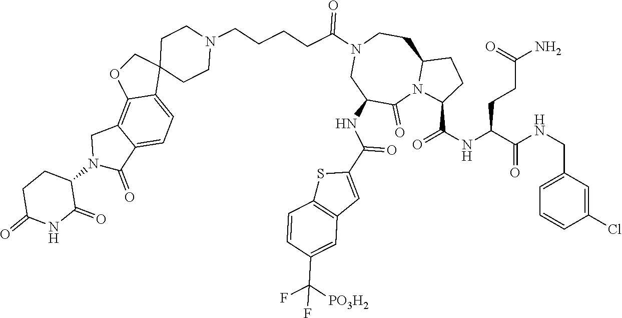 | ((2-(((5S,8S,10aR)-8-(((S)-5- amino-1-((3- chlorobenzyl)amino)-1,5- dioxopentan-2-yl)carbamoyl)-3- (5-(7-((S)-2,6-dioxopiperidin-3- yl)-6-oxo-7,8-dihydro-2H,6H- spiro[furo[2,3-e]isoindole-3,4′- piperidin]-1′-yl)pentanoyl)-6- oxodecahydropyrrolo[1,2- a][1,5]diazocin-5- yl)carbamoyl)benzo[b]thiophen- 5-yl)difluoromethyl)phosphonic acid |
| STD 71 | 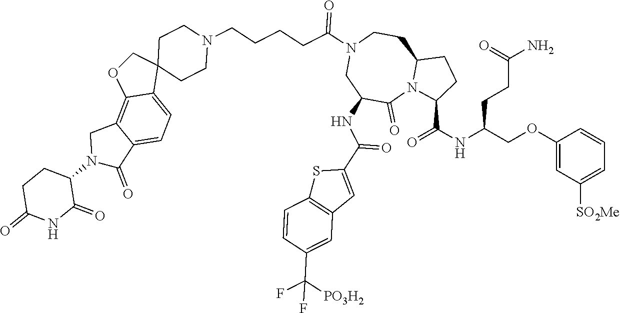 | ((2-(((5S,8S,10aR)-8-(((S)-5- amino-1-(3- (methylsulfonyl)phenoxy)-5- oxopentan-2-yl)carbamoyl)- 3-(5-(7-((S)-2,6-dioxopiperidin- 3-yl)-6-oxo-7,8-dihydro-2H,6H- spiro[furo[2,3-e]isoindole-3,4′- piperidin]-1′-yl)pentanoyl)-6- oxodecahydropyrrolo[1,2- a][1,5]diazocin-5- yl)carbamoyl)benzo[b]thiophen- 5-yl)difluoromethyl)phosphonic acid |
| STD 80 | 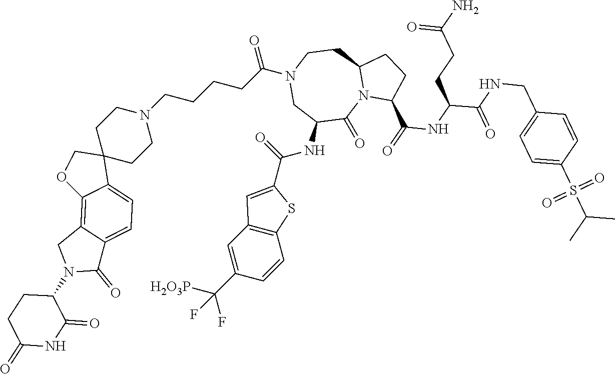 | ((2-(((5S,8S,10aR)-8-(((S)-5- amino-1-((4- (isopropylsulfonyl)benzyl) amino)- 1,5-dioxopentan-2- yl)carbamoyl)-3-(5-(7-((S)-2,6- dioxopiperidin-3-yl)-6-oxo-7,8- dihydro-2H,6H-spiro[furo[2,3- e]isoindole-3,4′-piperidin]-1′- yl)pentanoyl)-6- oxodecahydropyrrolo[1,2- a][1,5]diazocin-5- yl)carbamoyl)benzo[b]thiophen- 5-yl)difluoromethyl)phosphonic acid |
| STD 84 | 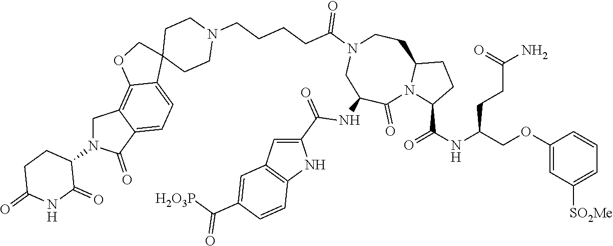 | (2-(((5S,8S,10aR)-8-(((S)-5- amino-1-(3- (methylsulfonyl)phenoxy)-5- oxopentan-2-yl)carbamoyl)-3- (5-(7-((S)-2,6-dioxopiperidin- 3-yl)-6-oxo-7,8-dihydro-2H,6H- spiro[furo[2,3-e]isoindole-3,4′- piperidin]-1′-yl)pentanoyl)-6- oxodecahydropyrrolo[1,2-a] [1,5]diazocin-5-yl)carbamoyl)- 1H-indole-5- carbonyl)phosphonic acid |
| STD 85 | 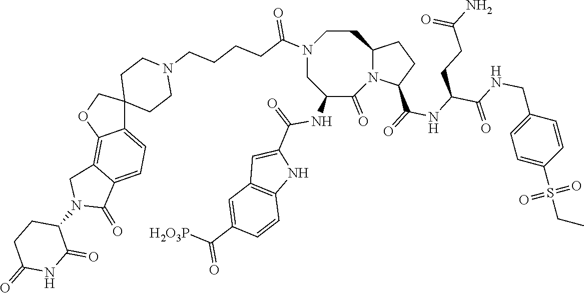 | (2-(((5S,8S,10aR)-8-(((S)-5- amino-1-((4- (ethylsulfonyl)benzyl)amino)- 1,5-dioxopentan-2- yl)carbamoyl)-3-(5-(7-((S)-2,6- dioxopiperidin-3-yl)-6-oxo-7,8- dihydro-2H,6H-spiro[furo[2,3- e]isoindole-3,4′-piperidin]-1′- yl)pentanoyl)-6- oxodecahydropyrrolo[1,2-a] [1,5]diazocin-5-yl)carbamoyl)- 1H-indole-5- carbonyl)phosphonic acid |
| STD 86 | 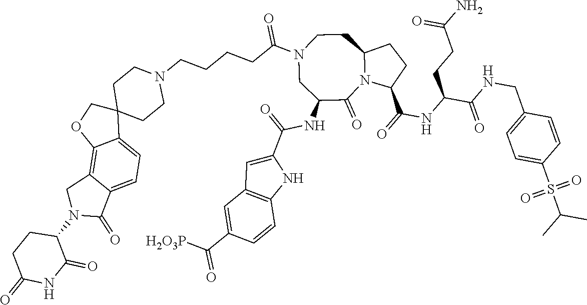 | (2-(((5S,8S,10aR)-8-(((S)-5- amino-1-((4- (isopropylsulfonyl)benzyl) amino)- 1,5-dioxopentan-2- yl)carbamoyl)-3-(5-(7-((S)-2,6- dioxopiperidin-3-yl)-6-oxo-7,8- dihydro-2H,6H-spiro[furo[2,3- e]isoindole-3,4′-piperidin]-1′- yl)pentanoyl)-6- oxodecahydropyrrolo[1,2-a] [1,5]diazocin-5-yl)carbamoyl)- 1H-indole-5- carbonyl)phosphonic acid |

### · Synthetic Schemes and Procedures

### STD84. (2-(((5S,8S,10aR)-8-(((S)-5-amino-1-(3-(methylsulfonyl) phenoxy)-5-oxopentan-2-yl) carbamoyl)-3-(5-(7-((S)-2,6-dioxopiperidin-3-yl)-6-oxo-7,8-dihydro-2H,6H-spiro[furo [2,3-e]isoindole-3,4′-piperidin]-1′-yl) pentanoyl)-6-oxodecahydropyrrolo[1,2-a][1,5]diazocin-5-yl) carbamoyl)-1H-indole-5-carbonyl)phosphonic acid

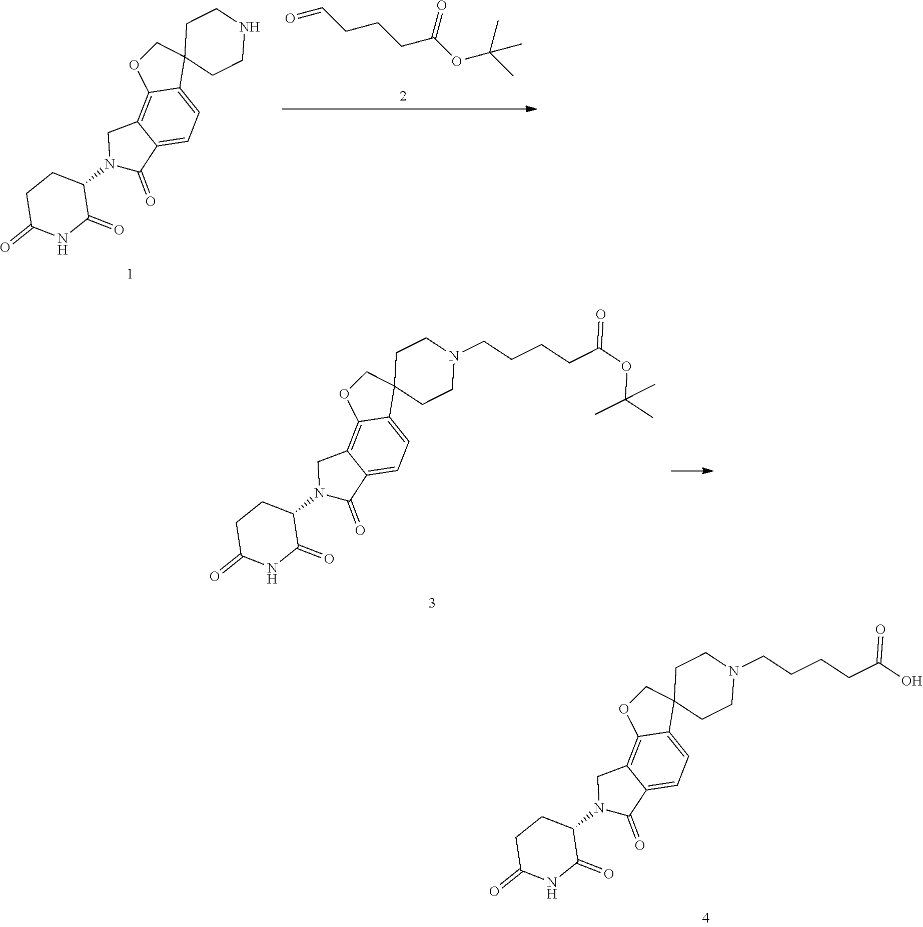 [SMILES: O=C1CC[C@H](N2Cc3c(ccc4c3OCC43CCNCC3)C2=O)C(=O)N1 ; CC(C)(C)OC(=O)CCCC=O ; CC(C)(C)OC(=O)CCCCN1CCC2(CC1)COc1c2ccc2c1CN([C@H]1CCC(=O)NC1=O)C2=O ; O=C(O)CCCCN1CCC2(CC1)COc1c2ccc2c1CN([C@H]1CCC(=O)NC1=O)C2=O]

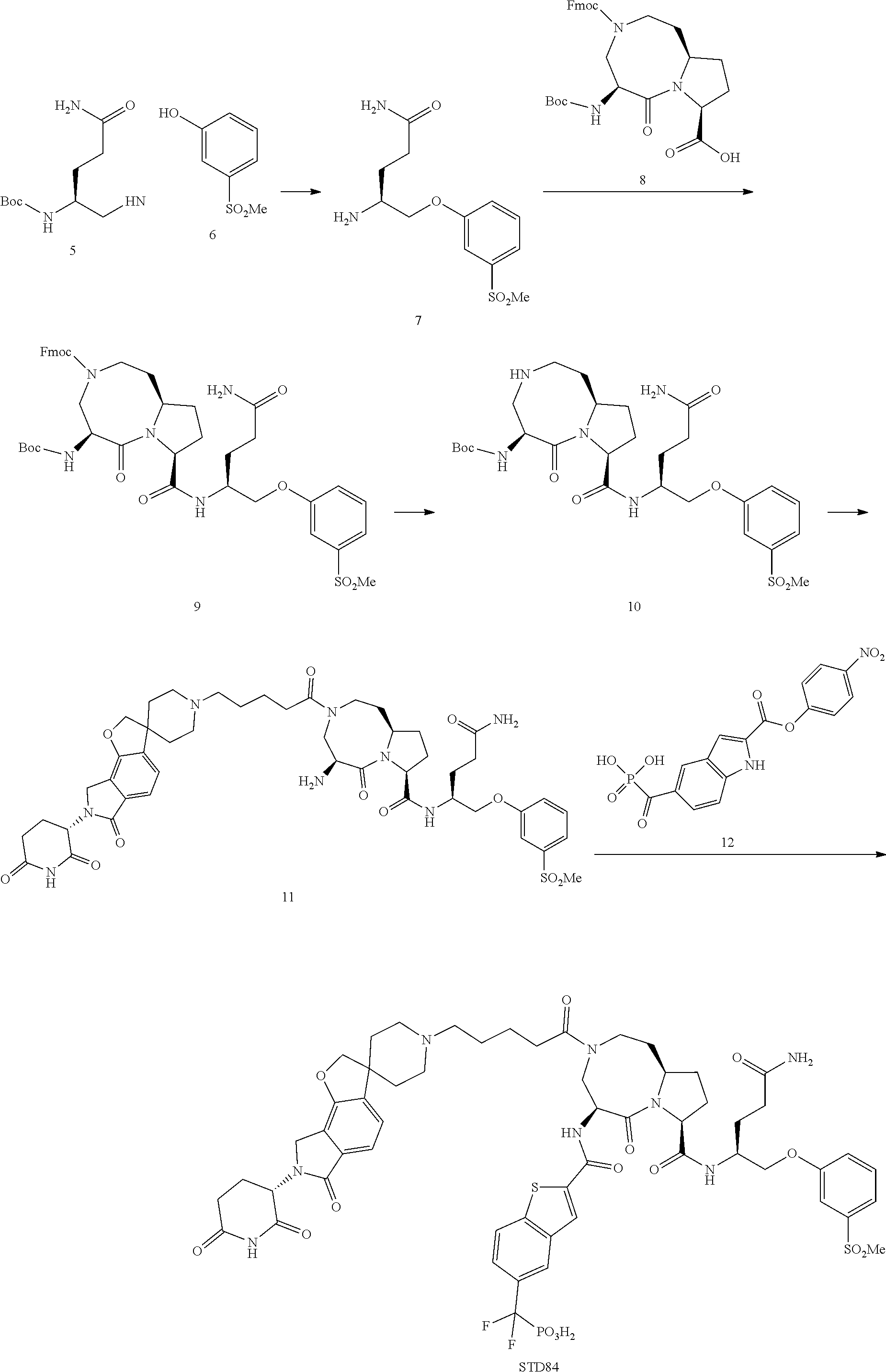

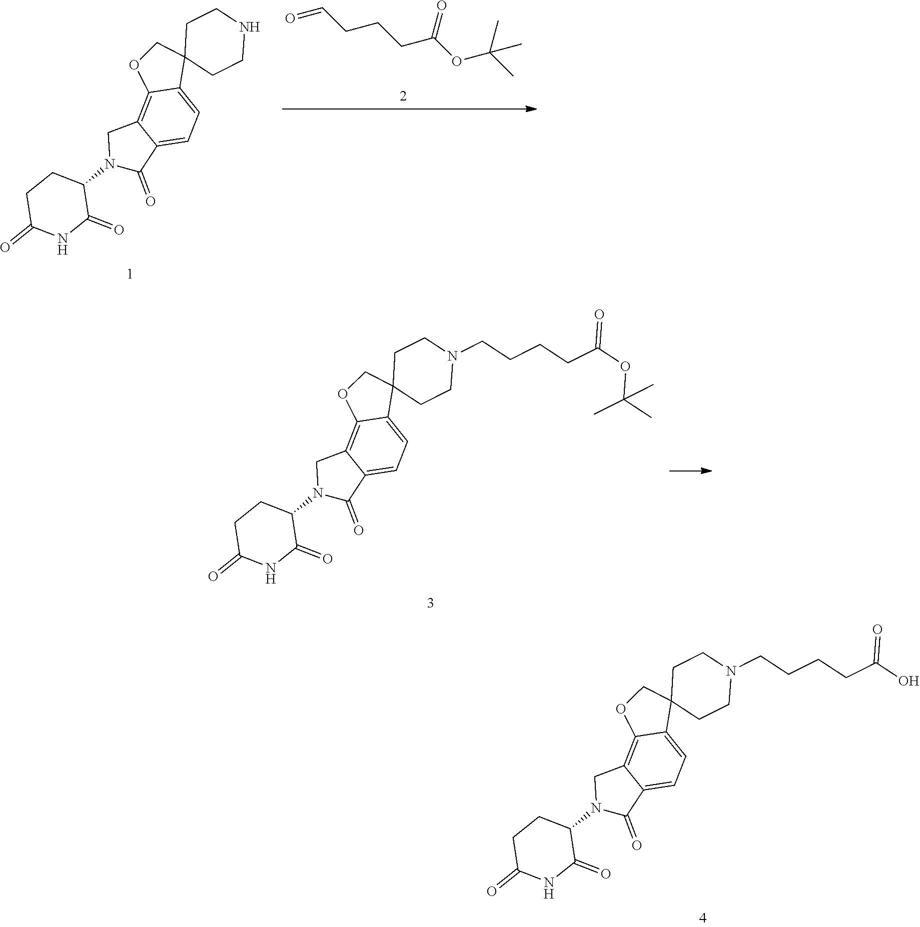 [SMILES: O=C1CC[C@H](N2Cc3c(ccc4c3OCC43CCNCC3)C2=O)C(=O)N1 ; CC(C)(C)OC(=O)CCCC=O ; CC(C)(C)OC(=O)CCCCN1CCC2(CC1)COc1c2ccc2c1CN([C@H]1CCC(=O)NC1=O)C2=O ; O=C(O)CCCCN1CCC2(CC1)COc1c2ccc2c1CN([C@H]1CCC(=O)NC1=O)C2=O]

Compound 4: To a solution of 1 (C1, 1 mmol) in DCE (20 mL) was added AcOH (3 mmol) and 2 (1 mmol) subsequently. After 0.5 h, NaBH(OAc)3 (1.5 mmol) was added in three portions in 1 h. The mixture was stirred 6 h, quenched with water and concentrated under vacuum. The residue was purified by reverse phase preparative HPLC to give the compound 3. UPLC-MS calculated[M+H]⁺: 512.3, found: 512.5. The obtained 3 was dissolved in DCM (10 mL), then trifluoroacetic acid (3 mL) was added slowly. After stirring for 3 h at rt, the reaction mixture was evaporated to give the crude 4 without further purification. UPLC-MS calculated[M+H]⁺: 456.2, found: 456.7.

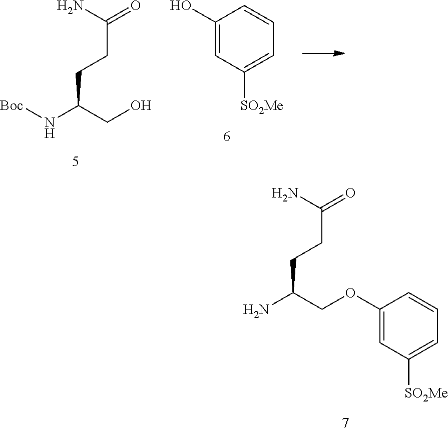 [SMILES: CC(C)(C)OC(=O)N[C@H](CO)CCC(N)=O ; CS(=O)(=O)c1cccc(O)c1 ; CS(=O)(=O)c1cccc(OC[C@@H](N)CCC(N)=O)c1]

Compound 7: DIAD (1.5 mmol) was added dropwise at 0° C. to the mixture of 5 (1 mmol), 6 (1 mmol) and PPh₃P (1.5 mmol) in THF (10 mL), and the mixture was stirred for 3 h at rt. The reaction mixture was concentrated and purified by reverse phase preparative HPLC. The obtained product was dissolved in DCM (20 mL), then trifluoroacetic acid (2 mL) was added slowly. After stirring for 5 h at rt, the reaction mixture was evaporated to give the 11 without further purification. UPLC-MS calculated[M+H]⁺: 287.1, found: 287.6.

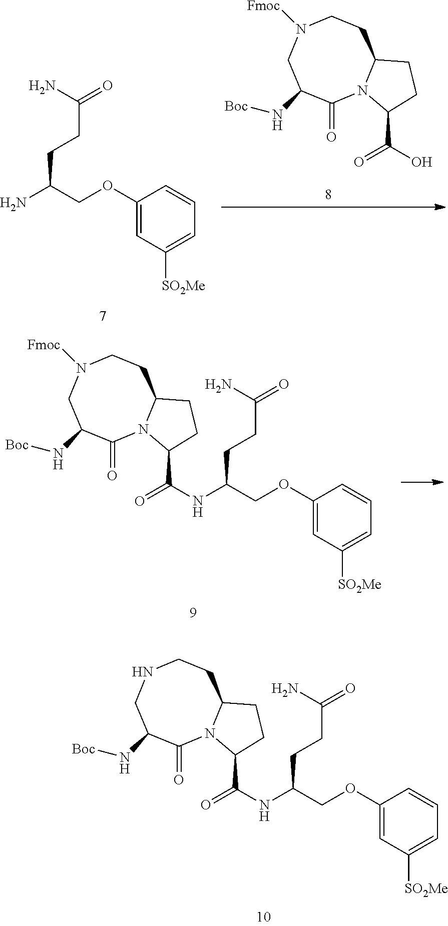

Compound 10: HATU (0.55 mmol, 1.1 equiv.) was added to a solution of the 7 (0.5 mmol, 1 equiv.), 8 (0.5 mmol, 1 equiv.) and DIEA (1.5 mmol, 3 equiv.) in DMF (60 mL) and the resulting mixture was stirred at rt for 1 h. The solution was diluted with EtOAc and washed with H₂O, saturated sodium bicarbonate aqueous solution and brine, and dried over sodium sulfate. After removal of the solvent in vacuo, the residue was purified by flash chromatography on silica gel to afford 9. The obtained product was dissolved in Acetonitrile (10 mL) and Diethylamine (0.5 mL) was added to the solution. The resulting mixture was stirred at rt for 20 min and the solvent was removed under vacuum. The residue was purified by HPLC to yield 10. UPLC-MS calculated[M+H]⁺: 596.3, found: 596.5.

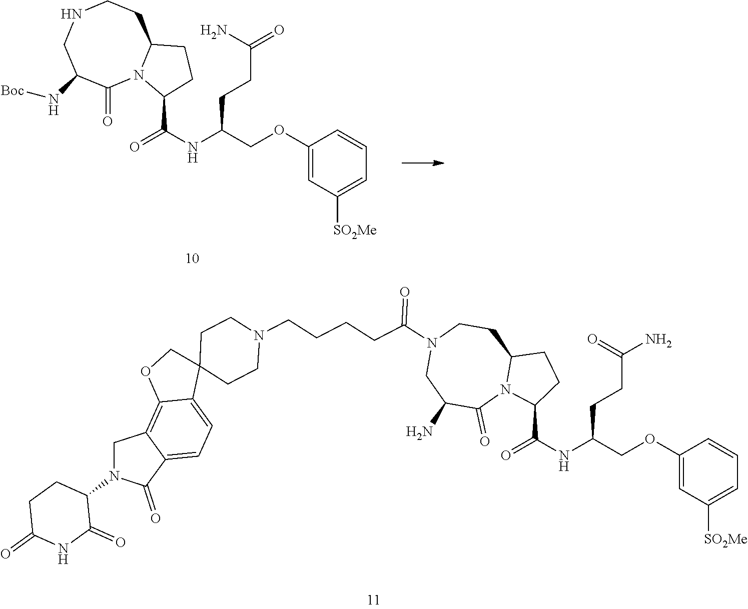 [SMILES: CC(C)(C)OC(=O)N[C@H]1CNCC[C@H]2CC[C@@H](C(=O)N[C@@H](CCC(N)=O)COc3cccc(S(C)(=O)=O)c3)N2C1=O ; CS(=O)(=O)c1cccc(OC[C@H](CCC(N)=O)NC(=O)[C@@H]2CC[C@@H]3CCN(C(=O)CCCCN4CCC5(CC4)COc4c5ccc5c4CN([C@H]4CCC(=O)NC4=O)C5=O)C[C@H](N)C(=O)N32)c1]

Compound 11: HATU (0.22 mmol, 1.1 equiv.) was added to a solution of 10 (0.2 mmol, 1 equiv.), 4 (0.2 mmol, 1 equiv.) and DIEA (0.6 mmol, 3 equiv.) in DMF (5 mL) and the resulting mixture was stirred at rt for 1 h. This mixture was purified by reverse phase preparative HPLC. The obtained product was dissolved in DCM (10 mL), then trifluoroacetic acid (1 mL) was added slowly. After stirring for 5 h at rt, the reaction mixture was evaporated to give the 11 without further purification. UPLC-MS calculated[M+H]⁺: 933.4, found: 933.8.

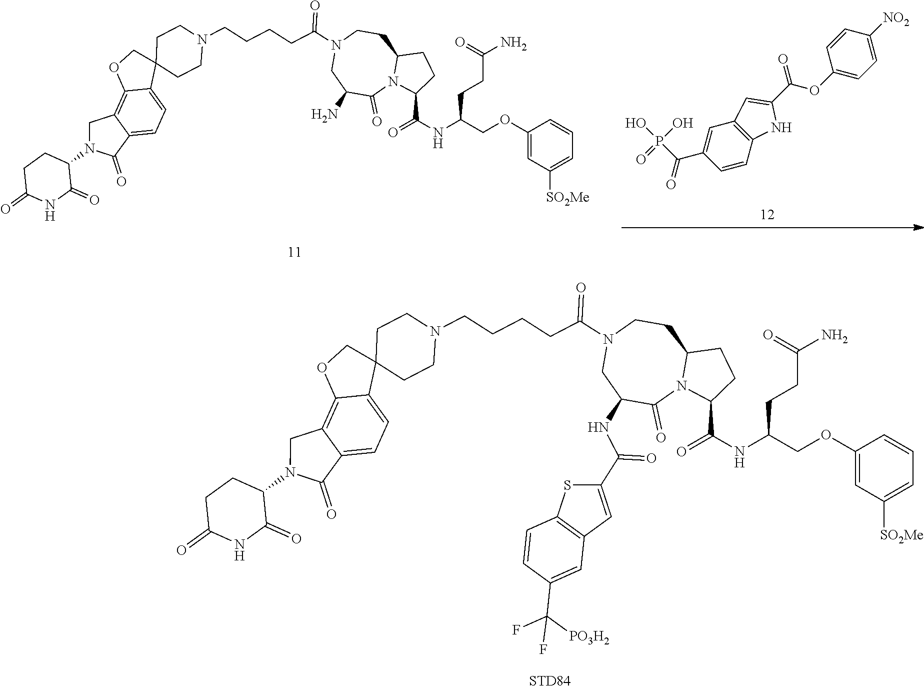

SD-987: DIEA (12 μL, 0.068 mmol, 3 equiv.) was added to the mixture of compound 12 (32 mg, 0.081 mmol, 1.2 equiv.), compound 11 (65 mg, 0.069 mmol, 1 equiv.) and HOBt (18 mg, 0.14 mmol, 2 equiv.) in DMF (3 mL). The resulted mixture was stirred at room temperature for 1 hour. Purification of this reaction mixture by HPLC gave the compound STD84. UPLC-MS calculated[M+2H]²⁺: 592.7, found: 592.8.

### STD85. (2-(((5S,8S,10aR)-8-(((S)-5-amino-1-((4-(ethylsulfonyl)benzyl)amino)-1,5-dioxopentan-2-yl) carbamoyl)-3-(5-(7-((S)-2,6-dioxopiperidin-3-yl)-6-oxo-7,8-dihydro-2H,6H-spiro[furo [2,3-e]isoindole-3,4′-piperidin]-1′-yl) pentanoyl)-6-oxodecahydropyrrolo[1,2-a][1,5]diazocin-5-yl) carbamoyl)-1H-indole-5-carbonyl)phosphonic acid

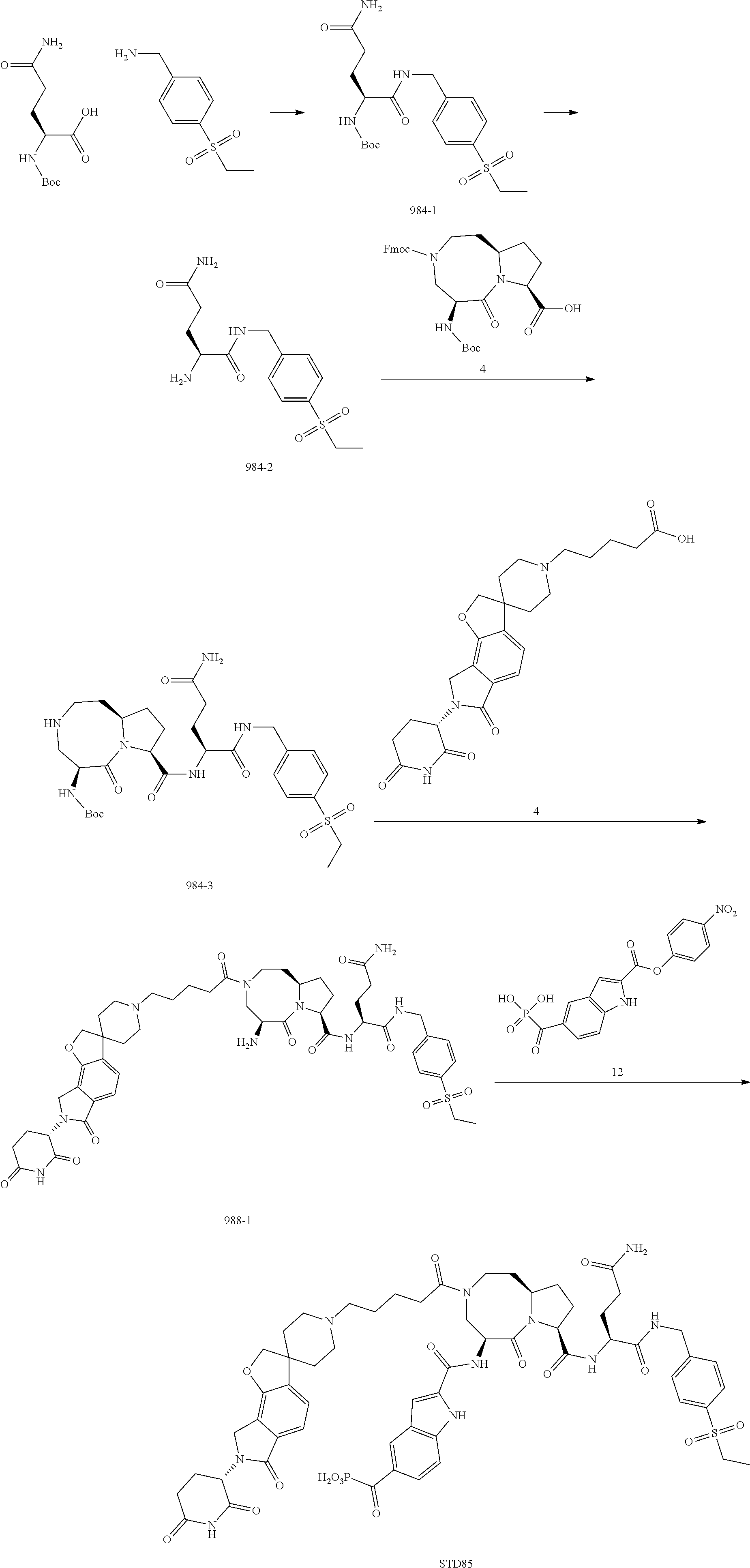

984-1: HATU (8.5 g, 22.3 mmol, 1.1 equiv.) was added to a solution of the Boc-Gln-OH (20.3 mmol, 1 equiv.), (4-(ethylsulfonyl)phenyl) methanamine (20.3 mmol, 1 equiv.) and DIEA (10.6 mL, 60.9 mmol, 3 equiv.) in DMF (60 mL) and the resulting mixture was stirred at rt for 1 h. The solution was diluted with EtOAc and washed with H₂O, saturated sodium bicarbonate aqueous solution and brine, and dried over sodium sulfate. After removal of the solvent in vacuo, the residue was purified by flash chromatography on silica gel to afford 984-1. UPLC-MS calculated[M+H]⁺: 428.2, found: 428.7.

984-2: TFA (5 mL) was added slowly to a solution of 984-1 (3 g) in DCM (50 mL) and the resulting reaction solution was stirred at rt for 6 h and then evaporated. The residue was used directly in the next step without further purification. UPLC-MS calculated[M+H]⁺: 328.1, found: 328.5.

984-3: HATU (0.55 mmol, 1.1 equiv.) was added to a solution of the 984-2 (0.5 mmol, 1 equiv.), 8 (0.5 mmol, 1 equiv.) and DIEA (1.5 mmol, 3 equiv.) in DMF (60 mL) and the resulting mixture was stirred at rt for 1 h. The solution was diluted with EtOAc and washed with H₂O, saturated sodium bicarbonate aqueous solution and brine, and dried over sodium sulfate. After removal of the solvent in vacuo, the residue was purified by flash chromatography on silica gel to afford 9. The obtained product was dissolved in Acetonitrile (10 mL) and Diethylamine (0.5 mL) was added to the solution. The resulting mixture was stirred at rt for 20 min and the solvent was removed under vacuum. The residue was purified by HPLC to yield 984-3. UPLC-MS calculated[M+H]⁺: 637.3, found: 637.5.

988-1: HATU (0.22 mmol, 1.1 equiv.) was added to a solution of 984-3 (0.2 mmol, 1 equiv.), 4 (0.2 mmol, 1 equiv.) and DIEA (0.6 mmol, 3 equiv.) in DMF (5 mL) and the resulting mixture was stirred at rt for 1 h. This mixture was purified by reverse phase preparative HPLC. The obtained product was dissolved in DCM (10 mL), then trifluoroacetic acid (1 mL) was added slowly. After stirring for 5 h at rt, the reaction mixture was evaporated to give the 988-1 without further purification. UPLC-MS calculated[M+H]⁺: 974.4, found: 974.6.

STD85: DIEA (12 μL, 0.068 mmol, 3 equiv.) was added to the mixture of compound 12 (0.081 mmol, 1.2 equiv.), compound 988-1 (0.069 mmol, 1 equiv.) and HOBt (0.14 mmol, 2 equiv.) in DMF (3 mL). The resulted mixture was stirred at room temperature for 1 hour. Purification of this reaction mixture by HPLC gave the compound STD85. UPLC-MS calculated[M+2H]²⁺: 613.2, found: 613.7.

### Biological Assessment

### Cell Viability Assay

SU-DHL-1 and SUP-M2 cell viability was determined by CellTiter-Glo 2.0 Cell Viability Assay (Promega). SU-DHL-1 cell line was purchased from ATCC (Manassas, VA) and SUP-M2 (ACC-509) was purchased from DSMZ (Germany). RPMI 1640 medium and fetal bovine serum (FBS) were purchased from Gibco/Thermo Fisher Scientific. In a typical procedure, cells seeded at 1000 cells per well in 384-well white plates (Corning) were incubated with serially diluted compounds for 4 days at 37° C. with 5% CO₂. At the end of treatment, CellTiter-Glo 2.0 was added to the wells and luminescence was acquired on TECAN PSARK plate reader. Untreated cells were used as control. Data points were fit with a four-parameter equation to generate a concentration-response curve. IC₅₀ values were calculated using a nonlinear regression analysis of the mean±SD from triplicate.

### HiBiT Assay

STAT3-HIBIT KI HeLa cell line was purchased from Promega. This cell line is a clone created by using CRISPR-Cas9 to fuse HiBIT to the 3′ end of STAT3 in HeLa cells. DMEM medium and FBS were purchased from Gibco/Thermo Fisher Scientific. STAT3 degradation was determined based on quantification of luminescent signal using Nano-Glo HiBIT Lytic Detection System. In a typical procedure, cells seeded at 20000 cells per well in 96-well white plates (Corning) were incubated with serially diluted compounds for 24 hours at 37° C. with 5% CO₂. At the end of treatment, Nano-Glo HiBIT Lytic Detection reagents was added to the wells and luminescence was acquired on TECAN PSARK plate reader. Untreated cells were used as control. Data points were fit with a four-parameter equation to generate a concentration-response curve. IC₅₀ values were calculated using a nonlinear regression analysis of the mean±SD from triplicate.

TABLE E9

| | Degradation (HiBit) | | Cell Viability IC50 (uM) | | |
|---|---|---|---|---|---|
| | DC50 | | | SU- | |
| Compound No. | (uM) | Dmax | MOLM-16 | DHL-1 | SUP-M2: |

| | | | | |
|---|---|---|---|---|
| STD 22 | C | D | | |
| STD 69 | C | B | C | C |
| STD 70 | B | A | C | C |
| STD 71 | B | A | B | C |
| STD 80 | B | A | B | C |
| STD 84 | B | A | B | C |
| STD 85 | B | A | B | C |
| STD 86 | B | A | B | C |

STAT3 DC₅₀: A (<0.1 μM), B (0.1-1.0 μM), C (1.0-10 μM), and D (>10 μM).
IC₅₀: A (<0.1 μM), B (0.1-1.0 μM), C (1.0-10 μM), and D (>10 μM).
Dₘₐₓ: A(>=80%), B(>=60% and <80%), C (>=40% and <60%), and D(<40%)

### 3. CBP/p300 DEGRADERS

TABLE E10

| Cpd. No. | Structures | Chemical Name |
|---|---|---|
| CPD-001 | 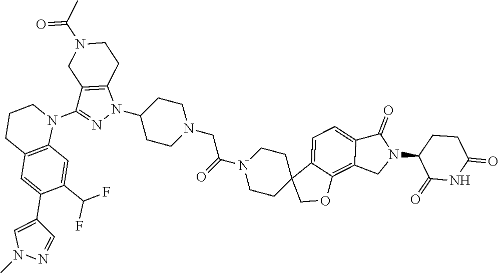 [SMILES: CC(=O)N1CCc2c(c(N3CCCc4cc(-c5cnn(C)c5)c(C(F)F)cc43)nn2C2CCN(CC(=O)N3CCC4(CC3)COc3c4ccc4c3CN([C@H]3CCC(=O)NC3=O)C4=O)CC2)C1] | (S)-3-(1′-(2-(4-(5-acetyl-3-(7- (difluoromethyl)-6-(1-methyl-1H-pyrazol- 4-yl)-3,4-dihydroquinolin-1(2H)-yl)- 4,5,6,7-tetrahydro-1H-pyrazolo[4,3- c]pyridin-1-yl)piperidin-1-yl)acetyl)-6- oxo-6,8-dihydro-2H,7H-spiro[furo[2,3- e]isoindole-3,4′-piperidin]-7-yl)piperidine- 2,6-dione |
| CPD-002 | 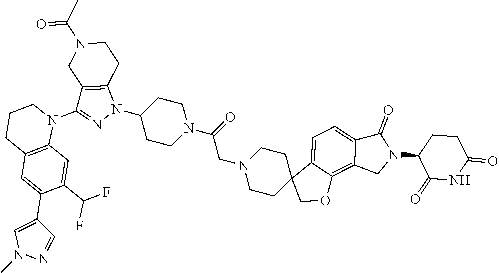 [SMILES: CC(=O)N1CCc2c(c(N3CCCc4cc(-c5cnn(C)c5)c(C(F)F)cc43)nn2C2CCN(C(=O)CN3CCC4(CC3)COc3c4ccc4c3CN([C@H]3CCC(=O)NC3=O)C4=O)CC2)C1] | (S)-3-(1′-(2-(4-(5-acetyl-3-(7- (difluoromethyl)-6-(1-methyl-1H-pyrazol- 4-yl)-3,4-dihydroquinolin-1(2H)-yl)- 4,5,6,7-tetrahydro-1H-pyrazolo[4,3- c]pyridin-1-yl)piperidin-1-yl)-2-oxoethyl)- 6-oxo-6,8-dihydro-2H,7H-spiro[furo[2,3- e]isoindole-3,4′-piperidin]-7-yl)piperidine- 2,6-dione |
| CPD-003 | 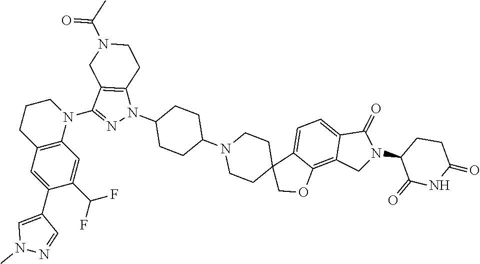 [SMILES: CC(=O)N1CCc2c(c(N3CCCc4cc(-c5cnn(C)c5)c(C(F)F)cc43)nn2C2CCC(N3CCC4(CC3)COc3c4ccc4c3CN([C@H]3CCC(=O)NC3=O)C4=O)CC2)C1] | (S)-3-(1′-(4-(5-acetyl-3-(7- (difluoromethyl)-6-(1-methyl-1H-pyrazol- 4-yl)-3,4-dihydroquinolin-1(2H)-yl)- 4,5,6,7-tetrahydro-1H-pyrazolo[4,3- c]pyridin-1-yl)cyclohexyl)-6-oxo-6,8- dihydro-2H,7H-spiro[furo[2,3-e]isoindole- 3,4′-piperidin]-7-yl)piperidine-2,6-dione |
| CPD-004 | 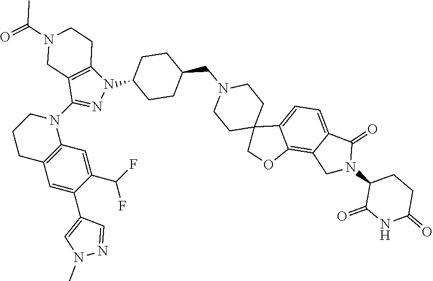 [SMILES: CC(=O)N1CCc2c(c(N3CCCc4cc(-c5cnn(C)c5)c(C(F)F)cc43)nn2[C@H]2CC[C@H](CN3CCC4(CC3)COc3c4ccc4c3CN([C@H]3CCC(=O)NC3=O)C4=O)CC2)C1] | (S)-3-(1′-(((1r,4S)-4-(5-acetyl-3-(7- (difluoromethyl)-6-(1-methyl-1H-pyrazol- 4-yl)-3,4-dihydroquinolin-1(2H)-yl)- 4,5,6,7-tetrahydro-1H-pyrazolo[4,3- c]pyridin-1-yl)cyclohexyl)methyl)-6-oxo- 6,8-dihydro-2H,7H-spiro[furo[2,3- e]isoindole-3,4′-piperidin]-7-yl)piperidine- 2,6-dione |
| CPD-005 | 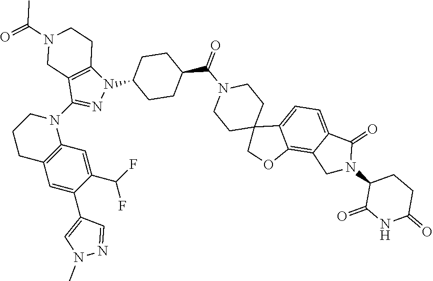 [SMILES: CC(=O)N1CCc2c(c(N3CCCc4cc(-c5cnn(C)c5)c(C(F)F)cc43)nn2[C@H]2CC[C@H](C(=O)N3CCC4(CC3)COc3c4ccc4c3CN([C@H]3CCC(=O)NC3=O)C4=O)CC2)C1] | (S)-3-(1′-((1r,4S)-4-(5-acetyl-3-(7- (difluoromethyl)-6-(1-methyl-1H-pyrazol- 4-yl)-3,4-dihydroquinolin-1(2H)-yl)- 4,5,6,7-tetrahydro-1H-pyrazolo[4,3- c]pyridin-1-yl)cyclohexane-1-carbonyl)-6- oxo-6,8-dihydro-2H,7H-spiro[furo[2,3- e]isoindole-3,4′-piperidin]-7-yl)piperidine- 2,6-dione |
| CPD-006 | 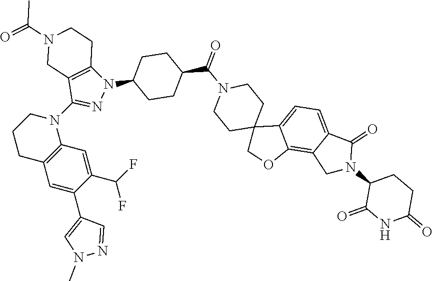 [SMILES: CC(=O)N1CCc2c(c(N3CCCc4cc(-c5cnn(C)c5)c(C(F)F)cc43)nn2[C@H]2CC[C@@H](C(=O)N3CCC4(CC3)COc3c4ccc4c3CN([C@H]3CCC(=O)NC3=O)C4=O)CC2)C1] | (S)-3-(1′-((1s,4R)-4-(5-acetyl-3-(7- (difluoromethyl)-6-(1-methyl-1H-pyrazol- 4-yl)-3,4-dihydroquinolin-1(2H)-yl)- 4,5,6,7-tetrahydro-1H-pyrazolo[4,3- c]pyridin-1-yl)cyclohexane-1-carbonyl)-6- oxo-6,8-dihydro-2H,7H-spiro[furo[2,3- e]isoindole-3,4′-piperidin]-7-yl)piperidine- 2,6-dione |

### ·Synthetic Schemes and Procedures

### CPD-004. (S)-3-(1′-(((1r,4S)-4-(5-acetyl-3-(7-(difluoromethyl)-6-(1-methyl-1H-pyrazol-4-yl)-3,4-dihydroquinolin-1 (2H)-yl)-4,5,6,7-tetrahydro-1H-pyrazolo[4,3-c]pyridin-1-yl)cyclohexyl)methyl)-6-oxo-6,8-dihydro-2H,7H-spiro[furo [2,3-e]isoindole-3,4′-piperidin]-7-yl) piperidine-2,6-dione

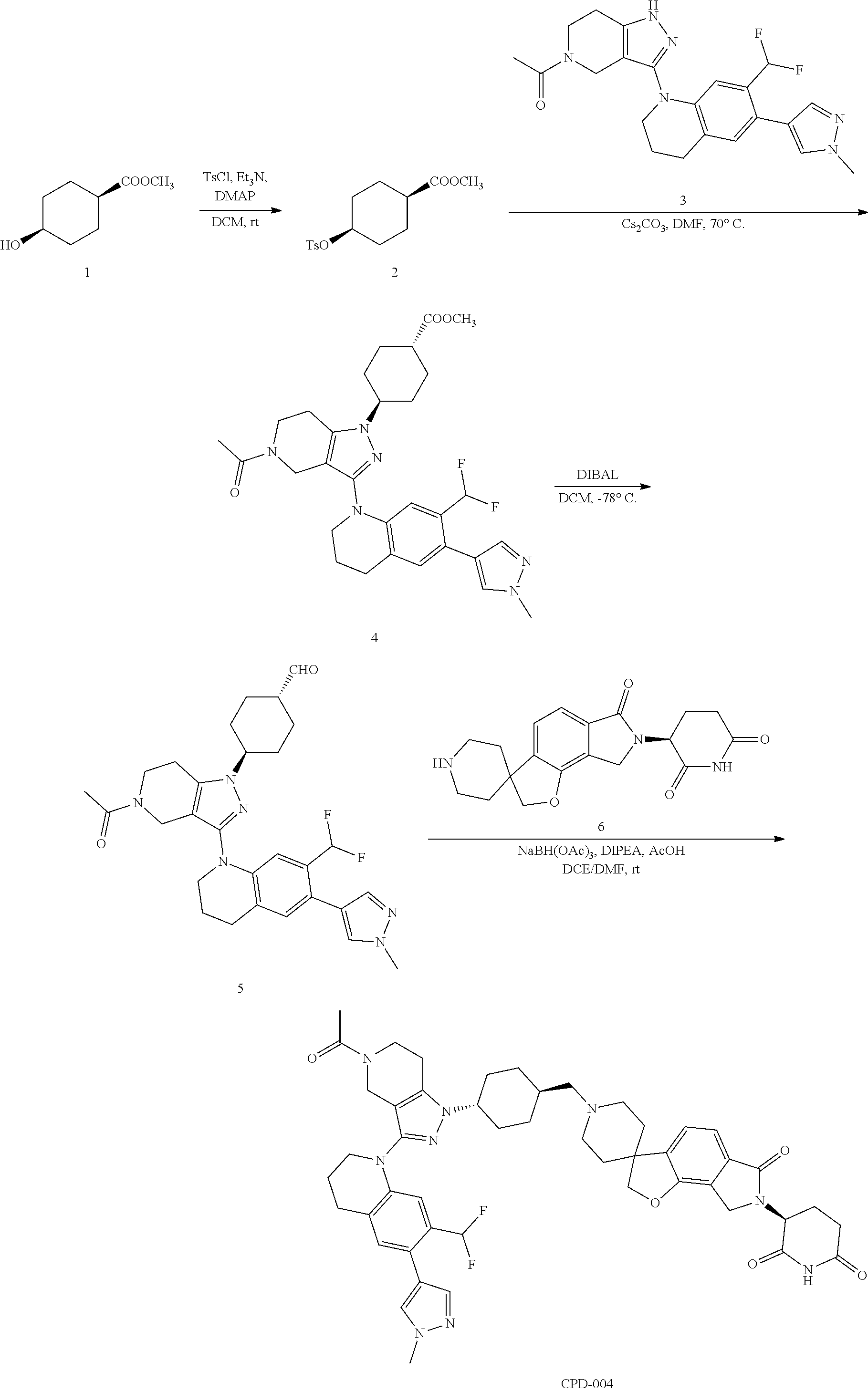

### Step 1: Synthesis of Methyl Cis-4-(Tosyloxy)Cyclohexane-1-Carboxylate (2)

To a solution of methyl cis-4-hydroxycyclohexane-1-carboxylate (1, 5 g, 31.6 mmol), 4-methylbenzenesulfonyl chloride (9.0 g, 47.2 mmol) and DMAP (0.77 g, 6.3 mmol) in DCM (50 mL) was added Et₃N(13.2 mL, 94.7 mmol). The mixture was stirred at room temperature for 16 h. Water was added, and the mixture was extracted with DCM. The combined organic layers were washed with brine, dried over anhydrous Na₂SO₄, filtered, and concentrated under reduced pressure. The crude residue was purified by silica gel flash chromatography (n-Hexane/EtOAc from 100:0 to 80:20) to give the title compound 2 as a yellow oil (9.3 g, yield=94%). LC-MS: m/z [M+H]⁺=334.85. ¹H NMR (400 MHZ, Chloroform-d) δ 7.81-7.75 (m, 2H), 7.35-7.29 (m, 2H), 4.70 (tt, J=5.0, 2.9 Hz, 1H), 3.66 (s, 3H), 2.44 (s, 3H), 2.37-2.26 (m, 1H), 1.92-1.78 (m, 4H), 1.75-1.65 (m, 2H), 1.59-1.47 (m, 2H).

Step 2: synthesis of methyl trans-4-(5-acetyl-3-(7-(difluoromethyl)-6-(1-methyl-1H-pyrazol-4-yl)-3,4-dihydroquinolin-1 (2H)-yl)-4,5,6,7-tetrahydro-1H-pyrazolo[4,3-c]pyridin-1-yl)cyclohexane-1-carboxylate (4)

Intermediate 3 was made as the reported literature (Romero, F. A., et al. J. Med. Chem. 2017, 60, 9162-9183). LC-MS: m/z [M+H]⁺=427.23. ¹H NMR (400 MHZ, DMSO-d₆) δ 12.46-12.26 (m, 1H), 7.75 (s, 1H), 7.50 (s, 1H), 7.10 (s, 1H), 6.96-6.60 (m, 2H), 4.19-4.07 (m, 2H), 3.86 (s, 3H), 3.75-3.54 (m, 4H), 2.88-2.62 (m, 4H), 2.07 (s, 2H), 2.01-1.88 (m, 3H).

To a solution of 1-(3-(7-(difluoromethyl)-6-(1-methyl-1H-pyrazol-4-yl)-3,4-dihydroquinolin-1 (2H)-yl)-1,4,6,7-tetrahydro-5H-pyrazolo[4,3-c]pyridin-5-yl) ethan-1-one (3, 300 mg, 0.70 mmol) and 2 (658 mg, 2.1 mmol) in DMF (4 mL) was added Cs₂CO₃ (917 mg, 2.8 mmol). The mixture was stirred at 70° C. for 7 h, and then directly purified by pre-HPLC: acetonitrile/H₂O from 45% to 100% in 55 min, flow rate (60 ml/min). The desired product started coming out when acetonitrile/H₂O=52%. The title compound 4 was obtained as a white solid (169 mg, yield=42%). LC-MS: m/z [M+H]⁺=567.25. ¹H NMR (400 MHZ, Chloroform-d) § 7.64 (s, 1H), 7.46 (s, 1H), 7.08-6.97 (m, 1H), 6.83 (s, 1H), 6.47 (td, J=55.5, 11.6 Hz, 1H), 4.25 (s, 1H), 4.13 (s, 1H), 4.05-3.99 (m, 3H), 3.97-3.89 (m, 2H), 3.82-3.75 (m, 1H), 3.74-3.66 (m, 5H), 2.92-2.81 (m, 3H), 2.80-2.73 (m, 1H), 2.47-2.35 (m, 1H), 2.24-2.01 (m, 11H), 1.69-1.52 (m, 2H).

### Step 3: Synthesis of Trans-4-(5-Acetyl-3-(7-(Difluoromethyl)-6-(1-Methyl-1H-Pyrazol-4-Yl)-3,4-Dihydroquinolin-1 (2H)-Yl)-4,5,6,7-Tetrahydro-1H-Pyrazolo[4,3-c]Pyridin-1-Yl)Cyclohexane-1-Carbaldehyde (5)

4 (223 mg, 0.39 mmol) was dissolved in anhydrous DCM (15 mL) and the solution was degassed and charged with N₂ 3 time. DIBAL (25% in toluene, 1.06 mL, 1.56 mmol) was added dropwise at −78° C. over 1 h and the reaction mixture was stirred at −78° C. for additional 2 h. Then the reaction was quenched with aqueous ammonium chloride. The result mixture was extracted with DCM, and the organic layers were washed with brine, dried over anhydrous Na₂SO₄, filtered, and concentrated under reduce pressure. The crude residue was purified by pre-HPLC: acetonitrile/H₂O from 35% to 100% in 65 min, flow rate (60 ml/min). The desired product started coming out when acetonitrile/H₂O=52%. The title compound 5 was obtained as a white solid (146 mg, yield=69%). LC-MS: m/z [M+H]⁺=537.24. ¹H NMR (400 MHZ, Chloroform-d) δ 7.56-7.51 (m, 1H), 7.43-7.37 (m, 1H), 7.07-6.96 (m, 1H), 6.89-6.83 (m, 1H), 6.51 (td, J=55.6, 10.9 Hz, 1H), 4.25 (s, 1H), 4.12 (s, 1H), 3.98-3.93 (m, 3H), 3.93-3.82 (m, 2H), 3.78-3.64 (m, 3H), 2.91-2.82 (m, 2H), 2.81-2.69 (m, 2H), 2.40-2.28 (m, 1H), 2.24-2.00 (m, 11H), 1.51-1.36 (m, 2H).

### Step 4: synthesis of 6-((trans-4-(5-acetyl-3-(7-(difluoromethyl)-6-(1-methyl-1H-pyrazol-4-yl)-3,4-dihydroquinolin-1 (2H)-yl)-4,5,6,7-tetrahydro-1H-pyrazolo[4,3-c]pyridin-1-yl)cyclohexyl)methyl)-2-(2,6-dioxopiperidin-3-yl)-6,7-dihydropyrrolo[3,4-f]isoindole-1,3 (2H,5H)-dione (CPD-004)

To a solution of 5 (25 mg, 0.047 mmol), 6 (27 mg, 0.069 mmol), DIPEA (24 μL, 0.140 mmol) and AcOH (27 μL, 0.47 mmol) in DCE/DMF=4 mL/2 mL was added NaBH(OAc)3 (29.6 mg, 0.140 mmol) into 3 portions over 2 h, and the mixture was stirred at rt overnight. The organic solvent DCE was removed under reduce pressure. The resulting residue was purified by pre-HPLC: acetonitrile/H₂O from 25% to 100% in 75 min, flow rate (60 ml/min). The desired product started coming out when acetonitrile/H₂O=34%. The title compound CPD-004 was obtained as a white solid (16 mg, yield=40%). UPLC-MS: m/z [M+H]⁺=876.62, purity >95%.

### CPD-001. (S)-3-(1′-(2-(4-(5-acetyl-3-(7-(difluoromethyl)-6-(1-methyl-1H-pyrazol-4-yl)-3,4-dihydroquinolin-1 (2H)-yl)-4,5,6,7-tetrahydro-1H-pyrazolo[4,3-c]pyridin-1-yl) piperidin-1-yl) acetyl)-6-oxo-6,8-dihydro-2H,7H-spiro[furo [2,3-e]isoindole-3,4′-piperidin]-7-yl) piperidine-2,6-dione

### CPD-002. (S)-3-(1′-(2-(4-(5-acetyl-3-(7-(difluoromethyl)-6-(1-methyl-1H-pyrazol-4-yl)-3,4-dihydroquinolin-1 (2H)-yl)-4,5,6,7-tetrahydro-1H-pyrazolo[4,3-c]pyridin-1-yl) piperidin-1-yl)-2-oxoethyl)-6-oxo-6,8-dihydro-2H,7H-spiro[furo [2,3-e]isoindole-3,4′-piperidin]-7-yl) piperidine-2,6-dione

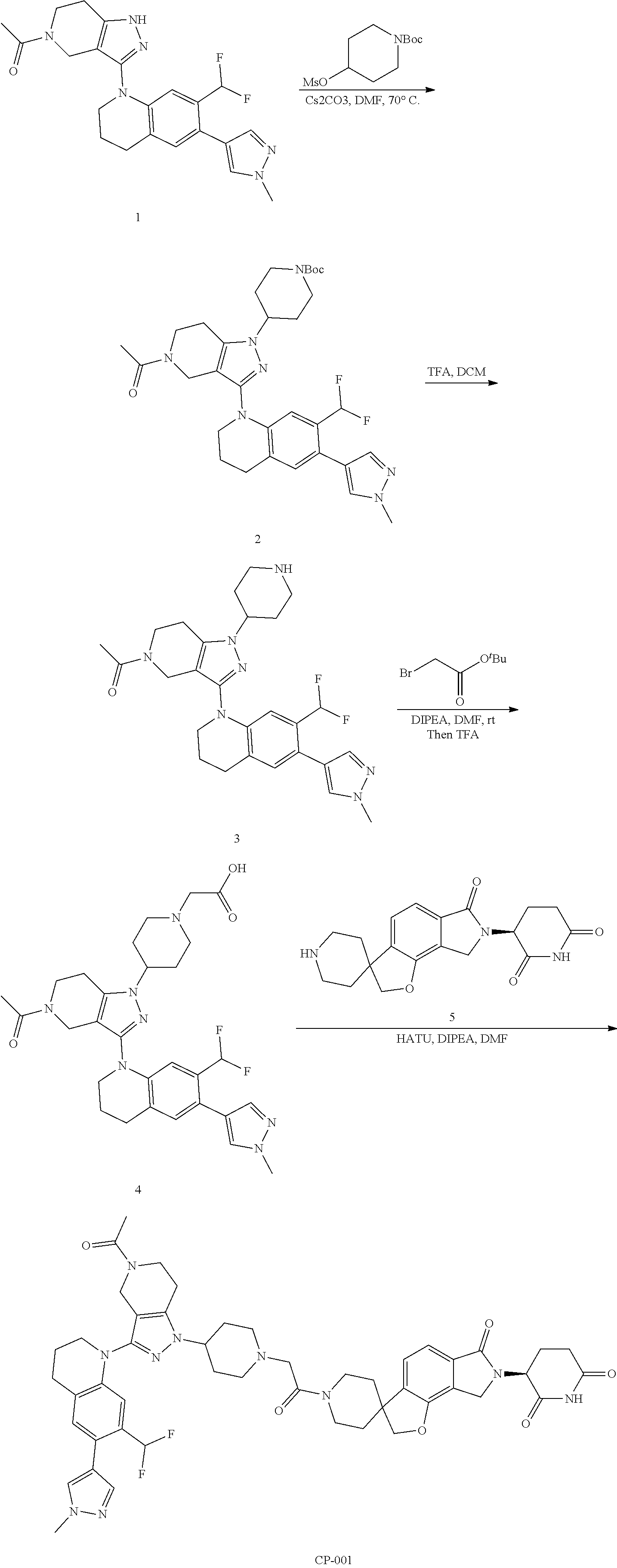

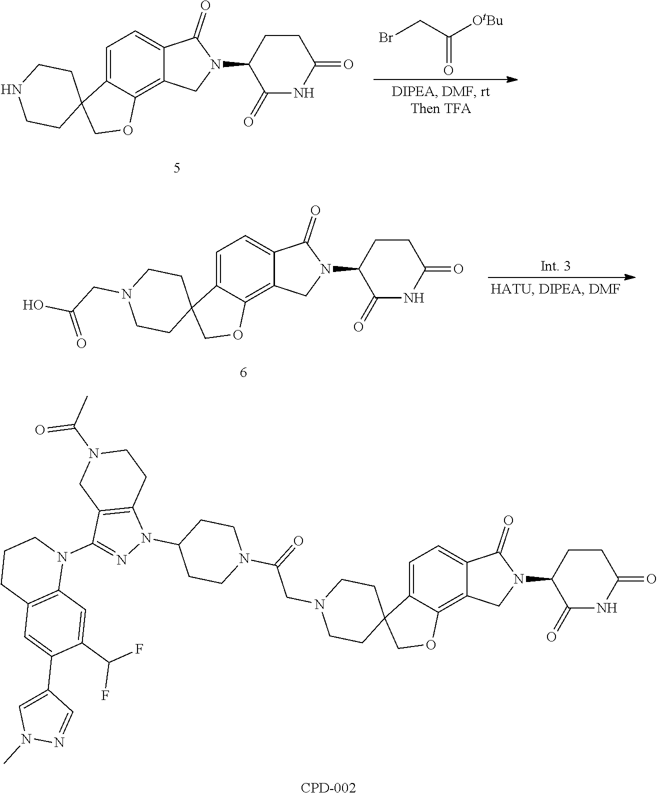 [SMILES: O=C1CC[C@H](N2Cc3c(ccc4c3OCC43CCNCC3)C2=O)C(=O)N1 ; CC(C)(C)OC(=O)CBr ; O=C(O)CN1CCC2(CC1)COc1c2ccc2c1CN([C@H]1CCC(=O)NC1=O)C2=O ; CC(=O)N1CCc2c(c(N3CCCc4cc(-c5cnn(C)c5)c(C(F)F)cc43)nn2C2CCN(C(=O)CN3CCC4(CC3)COc3c4ccc4c3CN([C@H]3CCC(=O)NC3=O)C4=O)CC2)C1]

### CPD-003. (S)-3-(1′-(4-(5-Acetyl-3-(7-(Difluoromethyl)-6-(1-Methyl-1H-Pyrazol-4-Yl)-3,4-Dihydroquinolin-1 (2H)-Yl)-4,5,6,7-Tetrahydro-1H-Pyrazolo[4,3-c]Pyridin-1-Yl)Cyclohexyl)-6-Oxo-6,8-Dihydro-2H,7H-Spiro[Furo [2,3-Flisoindole-3,4′-Piperidin]-7-Yl) Piperidine-2,6-Dione

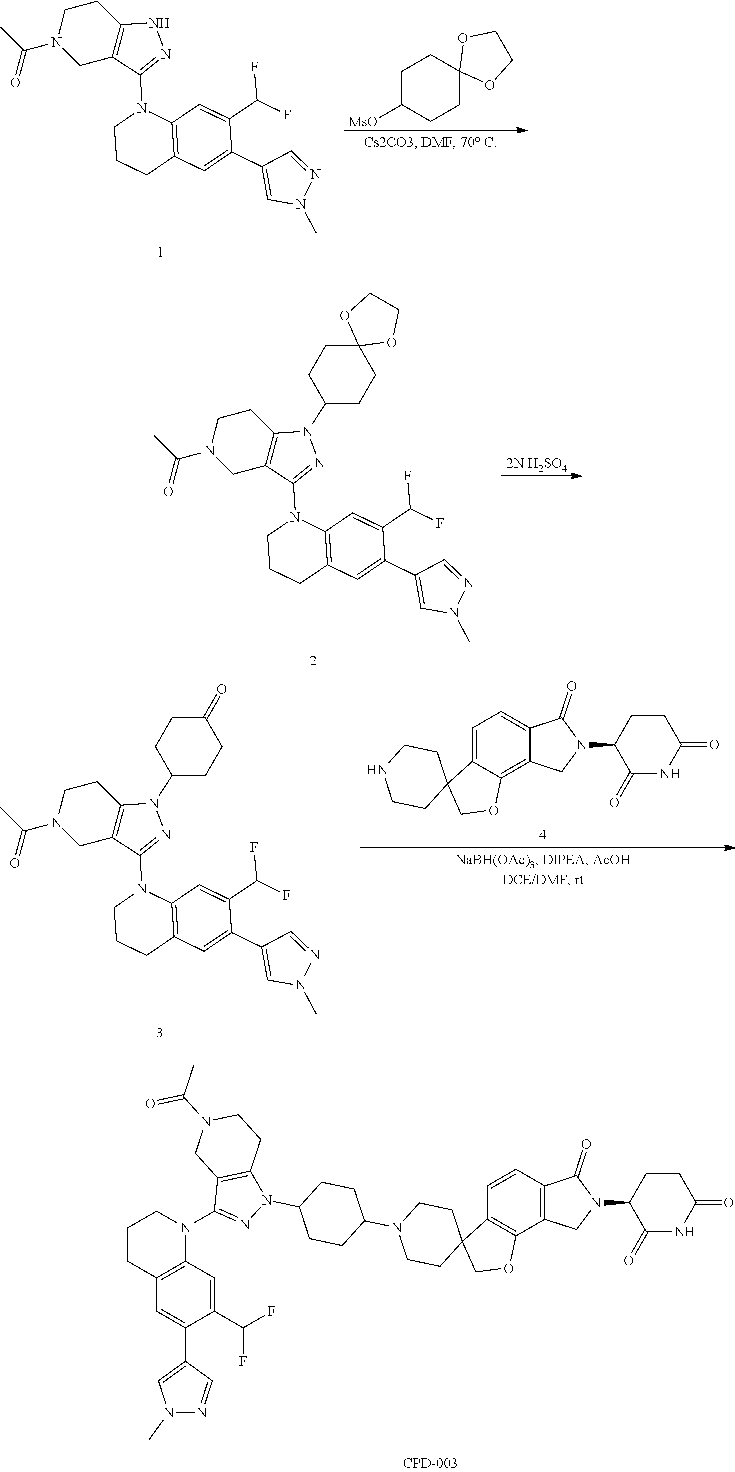

### CPD-005. (S)-3-(1′-((1r,4S)-4-(5-acetyl-3-(7-(difluoromethyl)-6-(1-methyl-1H-pyrazol-4-yl)-3,4-dihydroquinolin-1 (2H)-yl)-4,5,6,7-tetrahydro-1H-pyrazolo[4,3-c]pyridin-1-yl)cyclohexane-1-carbonyl)-6-oxo-6,8-dihydro-2H,7H-spiro[furo [2,3-e]isoindole-3,4′-piperidin]-7-yl) piperidine-2,6-dione

### CPD-006. (S)-3-(1′-((1s,4R)-4-(5-acetyl-3-(7-(difluoromethyl)-6-(1-methyl-1H-pyrazol-4-yl)-3,4-dihydroquinolin-1 (2H)-yl)-4,5,6,7-tetrahydro-1H-pyrazolo[4,3-c]pyridin-1-yl)cyclohexane-1-carbonyl)-6-oxo-6,8-dihydro-2H,7H-spiro[furo [2,3-e]isoindole-3,4′-piperidin]-7-yl) piperidine-2,6-dione

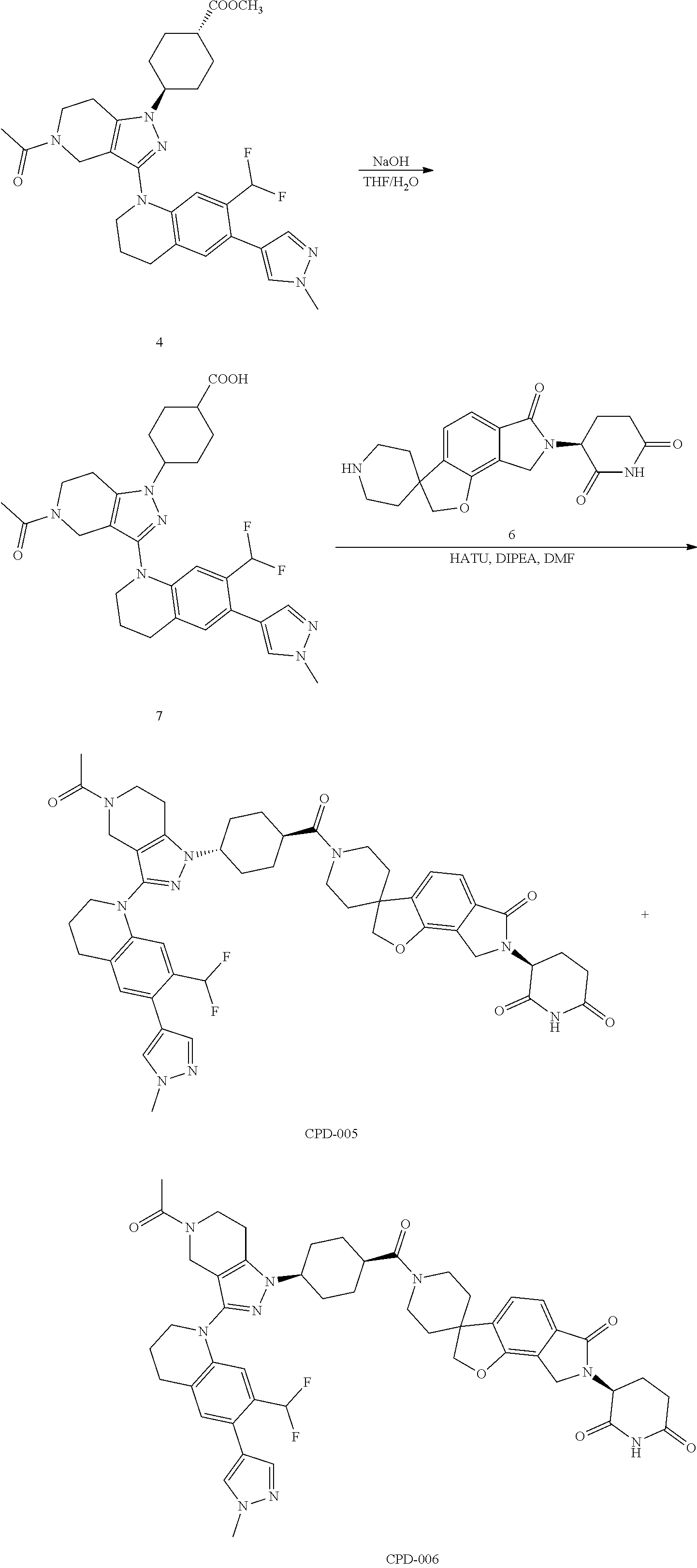 [SMILES: COC(=O)[C@H]1CC[C@H](n2nc(N3CCCc4cc(-c5cnn(C)c5)c(C(F)F)cc43)c3c2CCN(C(C)=O)C3)CC1 ; "NaOH" ; CC(=O)N1CCc2c(c(N3CCCc4cc(-c5cnn(C)c5)c(C(F)F)cc43)nn2C2CCC(C(=O)O)CC2)C1 ; O=C1CC[C@H](N2Cc3c(ccc4c3OCC43CCNCC3)C2=O)C(=O)N1 ; CC(=O)N1CCc2c(c(N3CCCc4cc(-c5cnn(C)c5)c(C(F)F)cc43)nn2[C@H]2CC[C@H](C(=O)N3CCC4(CC3)COc3c4ccc4c3CN([C@H]3CCC(=O)NC3=O)C4=O)CC2)C1 ; CC(=O)N1CCc2c(c(N3CCCc4cc(-c5cnn(C)c5)c(C(F)F)cc43)nn2[C@H]2CC[C@@H](C(=O)N3CCC4(CC3)COc3c4ccc4c3CN([C@H]3CCC(=O)NC3=O)C4=O)CC2)C1]

TABLE E11

UPLC-MS

| Cpd. No | [M + H]+ | Purity |
|---|---|---|
| CPD-001 | 905.53 | >95% |
| CPD-002 | 905.65 | >95% |
| CPD-003 | 862.57 | >95% |
| CPD-004 | 876.62 | >95% |
| CPD-005 | 890.61 | >95% |
| CPD-006 | 890.61 | >95% |

### Degradation Potency Data

WesternT Blot Analysis. Western blot analysis was performed essentially as described previously. The cells treated with indicated compounds were lysed in Radioimmunoprecipitation Assay Protein Lysis and Extraction Buffer (25 mmol/L Tris.HCl, pH 7.6, 150 mmol/L NaCl, 1% Nonidet P-40, 1% sodium deoxycholate, and 0.1% sodium dodecyl sulfate) containing proteinase inhibitor cocktail (Roche Diagnostics, Mannheim, Germany). Equal amounts of total protein were electrophoresed through 10% SDS-polyacrylamide gels after determination of protein concentration by BCA assay (Fisher Scientific, Pittsburgh, PA). The separated protein bands were transferred onto PVDF membranes (GE Healthcare Life Sciences, Marlborough, MA) and blotted against different antibodies, as indicated. The blots were scanned, and the band intensities were quantified using GelQuant.NET software provided by biochemlabsolutions.com. The relative mean intensity of target proteins was expressed after normalization to the intensity of glyceraldehyde-3-phosphate dehydrogenase bands. The activity of representative Compounds of the Disclosure are provided in Table E12.

TABLE E12

| | |
|---|---|
| CBP Degradation | P300 Degradation |
| (22Rv1, 5 h) | (22Rv1, 5 h) |

| Cpd. No | DC₅₀ | DCₘₐₓ | DC₅₀ | DCₘₐₓ |
|---|---|---|---|---|
| CPD-001 | A | A | B | A |
| CPD-002 | A | A | A | A |
| CPD-003 | A | A | A | A |
| CPD-004 | A | A | A | A |
| CPD-005 | A | A | A | A |
| CPD-006 | A | A | B | A |

CBP/p300 degradation activity DC₅₀: A (≤0.1 nM), B (0.1-1 nM), C (≥1 nM);
Dₘₐₓ: A (≥90%), B (80-90%), C (≤80%).

### 4. ANDROGEN RECEPTOR (AR) DEGRADERS

TABLE E13

| Compound | | |
|---|---|---|
| No. | Structure | Chemical Name |
| ARD-1 | 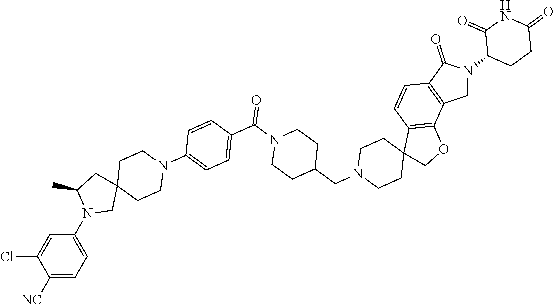 [SMILES: C[C@H]1CC2(CCN(c3ccc(C(=O)N4CCC(CN5CCC6(CC5)COc5c6ccc6c5CN([C@H]5CCC(=O)NC5=O)C6=O)CC4)cc3)CC2)CN1c1ccc(C#N)c(Cl)c1] | 2-chloro-4-((S)-8-(4-(4-((7- ((S)-2,6-dioxopiperidin-3-yl)- 6-oxo-7,8-dihydro-2H,6H- spiro[furo[2,3-e]isoindole- 3,4′-piperidin]-1′- yl)methyl)piperidine-1- carbonyl)phenyl)-3-methyl- 2,8-diazaspiro[4.5]decan-2- yl)benzonitrile |
| ARD-2 | 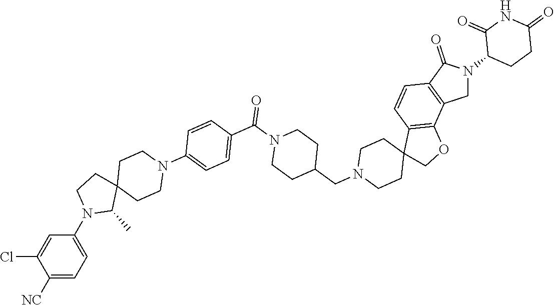 [SMILES: C[C@@H]1N(c2ccc(C#N)c(Cl)c2)CCC12CCN(c1ccc(C(=O)N3CCC(CN4CCC5(CC4)COc4c5ccc5c4CN([C@H]4CCC(=O)NC4=O)C5=O)CC3)cc1)CC2] | 2-chloro-4-((S)-8-(4-(4-((7- ((S)-2,6-dioxopiperidin-3-yl)- 6-oxo-7,8-dihydro-2H,6H- spiro[furo[2,3-e]isoindole- 3,4′-piperidin]-1′- yl)methyl)piperidine-1- carbonyl)phenyl)-3-methyl- 2,8-diazaspiro[4.5]decan-2- yl)benzonitrile |
| ARD-3 | 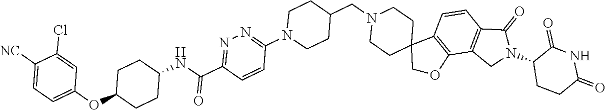 [SMILES: N#Cc1ccc(O[C@H]2CC[C@H](NC(=O)c3ccc(N4CCC(CN5CCC6(CC5)COc5c6ccc6c5CN([C@H]5CCC(=O)NC5=O)C6=O)CC4)nn3)CC2)cc1Cl] | N-((1r,4r)-4-(3-chloro-4- cyanophenoxy)cyclohexyl)-6- (4-((7-((S)-2,6- dioxopiperidin-3-yl)-6-oxo- 7,8-dihydro-2H,6H- spiro[furo[2,3-e]isoindole- 3,4′-piperidin]-1′- yl)methyl)piperidin-1- yl)pyridazine-3-carboxamide |
| ARD-4 | 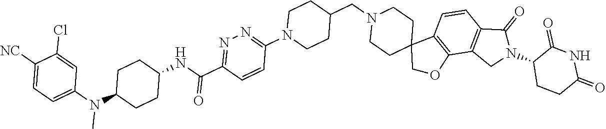 [SMILES: CN(c1ccc(C#N)c(Cl)c1)[C@H]1CC[C@H](NC(=O)c2ccc(N3CCC(CN4CCC5(CC4)COc4c5ccc5c4CN([C@H]4CCC(=O)NC4=O)C5=O)CC3)nn2)CC1] | N-((1r,4r)-4-((3-chloro-4- cyanophenyl)(methyl)amino)c yclohexyl)-6-(4-((7-((S)-2,6- dioxopiperidin-3-yl)-6-oxo- 7,8-dihydro-2H,6H- spiro[furo[2,3-e]isoindole- 3,4′-piperidin]-1′- yl)methyl)piperidin-1- yl)pyridazine-3-carboxamide |

### Synthetic Schemes and Procedures

### ARD-1. 2-Chloro-4-((S)-8-(4-(4-((7-((S)-2,6-Dioxopiperidin-3-Yl)-6-Oxo-7,8-Dihydro-2H,6H-Spiro[Furo [2,3-e]Isoindole-3,4′-Piperidin]-1′-Yl)Methyl) Piperidine-1-Carbonyl)Phenyl)-3-Methyl-2,8-Diazaspiro[4.5]Decan-2-Yl)Benzonitrile

### ARD-2. 2-Chloro-4-((S)-8-(4-(4-((7-((S)-2,6-Dioxopiperidin-3-Yl)-6-Oxo-7,8-Dihydro-2H,6H-Spiro[Furo [2,3-e]Isoindole-3,4′-Piperidin]-1′-Yl)Methyl) Piperidine-1-Carbonyl)Phenyl)-3-Methyl-2,8-Diazaspiro[4.5]Decan-2-Yl)Benzonitrile

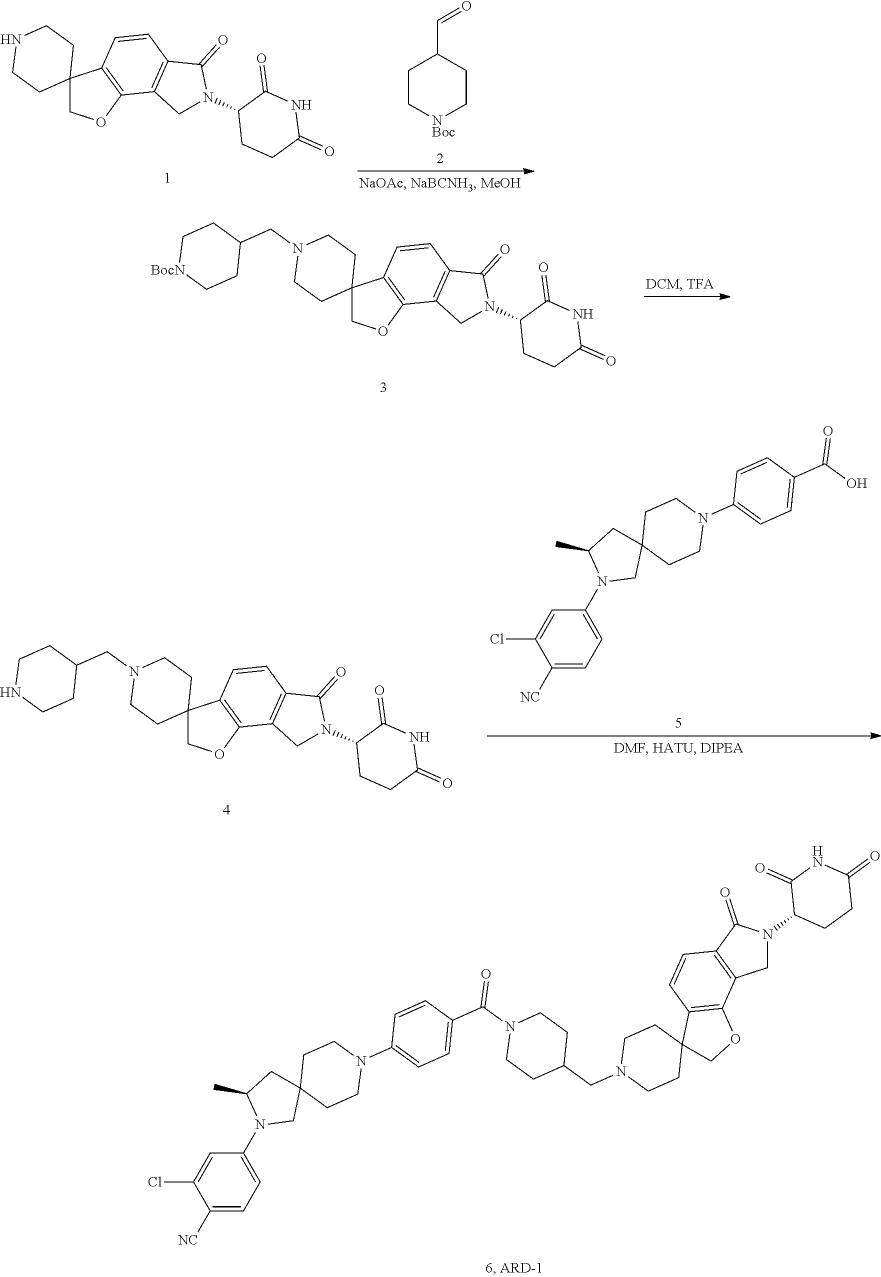 [SMILES: O=C1CC[C@H](N2Cc3c(ccc4c3OCC43CCNCC3)C2=O)C(=O)N1 ; CC(C)(C)OC(=O)N1CCC(C=O)CC1 ; CC(C)(C)OC(=O)N1CCC(CN2CCC3(CC2)COc2c3ccc3c2CN([C@H]2CCC(=O)NC2=O)C3=O)CC1 ; O=C1CC[C@H](N2Cc3c(ccc4c3OCC43CCN(CC4CCNCC4)CC3)C2=O)C(=O)N1 ; C[C@H]1CC2(CCN(c3ccc(C(=O)O)cc3)CC2)CN1c1ccc(C#N)c(Cl)c1 ; C[C@H]1CC2(CCN(c3ccc(C(=O)N4CCC(CN5CCC6(CC5)COc5c6ccc6c5CN([C@H]5CCC(=O)NC5=O)C6=O)CC4)cc3)CC2)CN1c1ccc(C#N)c(Cl)c1]

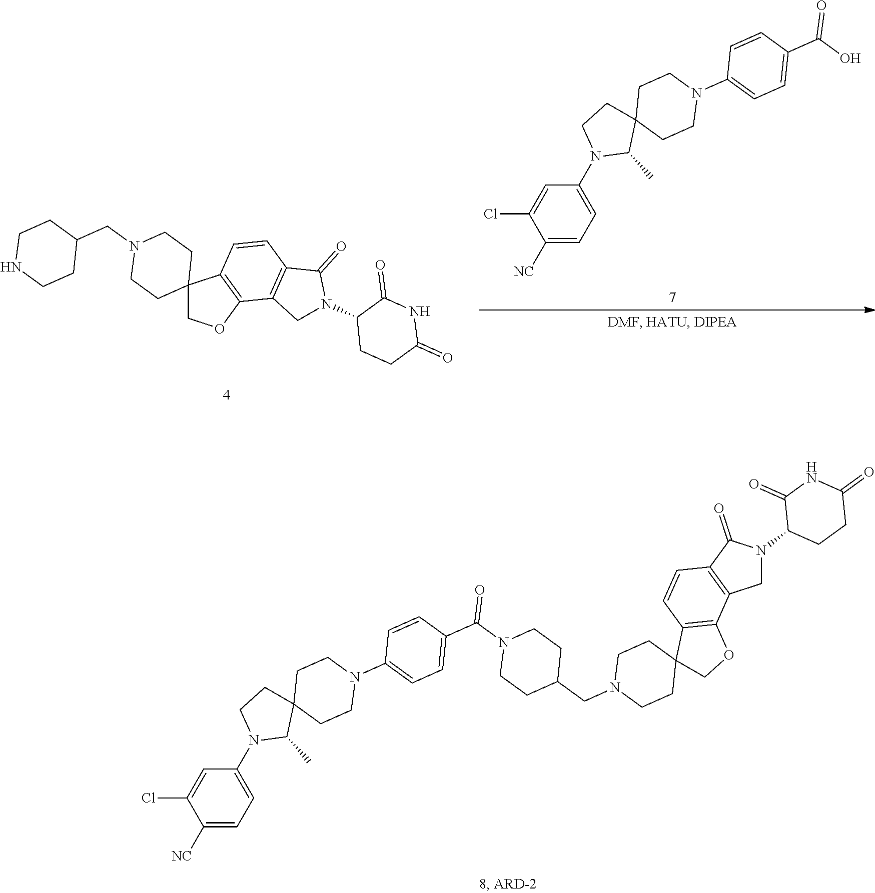 [SMILES: O=C1CC[C@H](N2Cc3c(ccc4c3OCC43CCN(CC4CCNCC4)CC3)C2=O)C(=O)N1 ; C[C@@H]1N(c2ccc(C#N)c(Cl)c2)CCC12CCN(c1ccc(C(=O)O)cc1)CC2 ; C[C@@H]1N(c2ccc(C#N)c(Cl)c2)CCC12CCN(c1ccc(C(=O)N3CCC(CN4CCC5(CC4)COc4c5ccc5c4CN([C@H]4CCC(=O)NC4=O)C5=O)CC3)cc1)CC2]

### Step 1

Compound 1 (1.0 eq) and NaOAc (4.0 eq) were dissolved in MeOH (10X). After 5 mins, 2 (1.2 eq) was added, and NaBCNH3 (3.0 eq) was followed after another 10 mins. The reaction was completed in 15 mins. All the volatile was removed under vacuum, and the residue was purified by Combiflash with DCM and MeOH to give 3 as white solid in 85% yield.

### Step 2

Compound 3 was dissolved in DCM (10X) and TFA (3X) was added. The deprotection was finished in 0.5 h. All the volatile was removed under vacuum to give 4 as white solid.

### Step 3

Compound 5 (1.0 eq), DIPEA (3.0 eq) were dissolved in DMF (10X), and HATU (1.3 eq) was added. After 15 min, compound 4 (1.0 eq) and DIPEA (2.0 eq) in DMF (3X) was added to the above solution. The reaction was finished in 0.5 h. The mixture was acidified and purified with pren-HPLC using 36% acetonitrile in H₂O to give the titled compound 6 (ARD-1). UPLS-MS: 4.1 min, 844.30.

### Step 4

Compound 8 (ARD-2) was synthesized following the procedure of ARD-2980. Pren-HPLC: 38% acetonitrile in H₂O. UPLS-MS: 4.2 min, 844.29.

### ARD-3. N-((1r,4r)-4-(3-Chloro-4-Cyanophenoxy)Cyclohexyl)-6-(4-((7-((S)-2,6-Dioxopiperidin-3-Yl)-6-Oxo-7,8-Dihydro-2H,6H-Spiro[Furo [2,3-e]Isoindole-3,4′-Piperidin]-1′-Yl)Methyl) Piperidin-1-Yl)Pyridazine-3-Carboxamide

### ARD-4. N-((1r,4r)-4-((3-Chloro-4-Cyanophenyl)(Methyl)Amino)Cyclohexyl)-6-(4-((7-((S)-2,6-Dioxopiperidin-3-Yl)-6-Oxo-7,8-Dihydro-2H,6H-Spiro[Furo [2,3-e]Isoindole-3,4′-Piperidin]-1′-Yl)Methyl) Piperidin-1-Yl)Pyridazine-3-Carboxamide

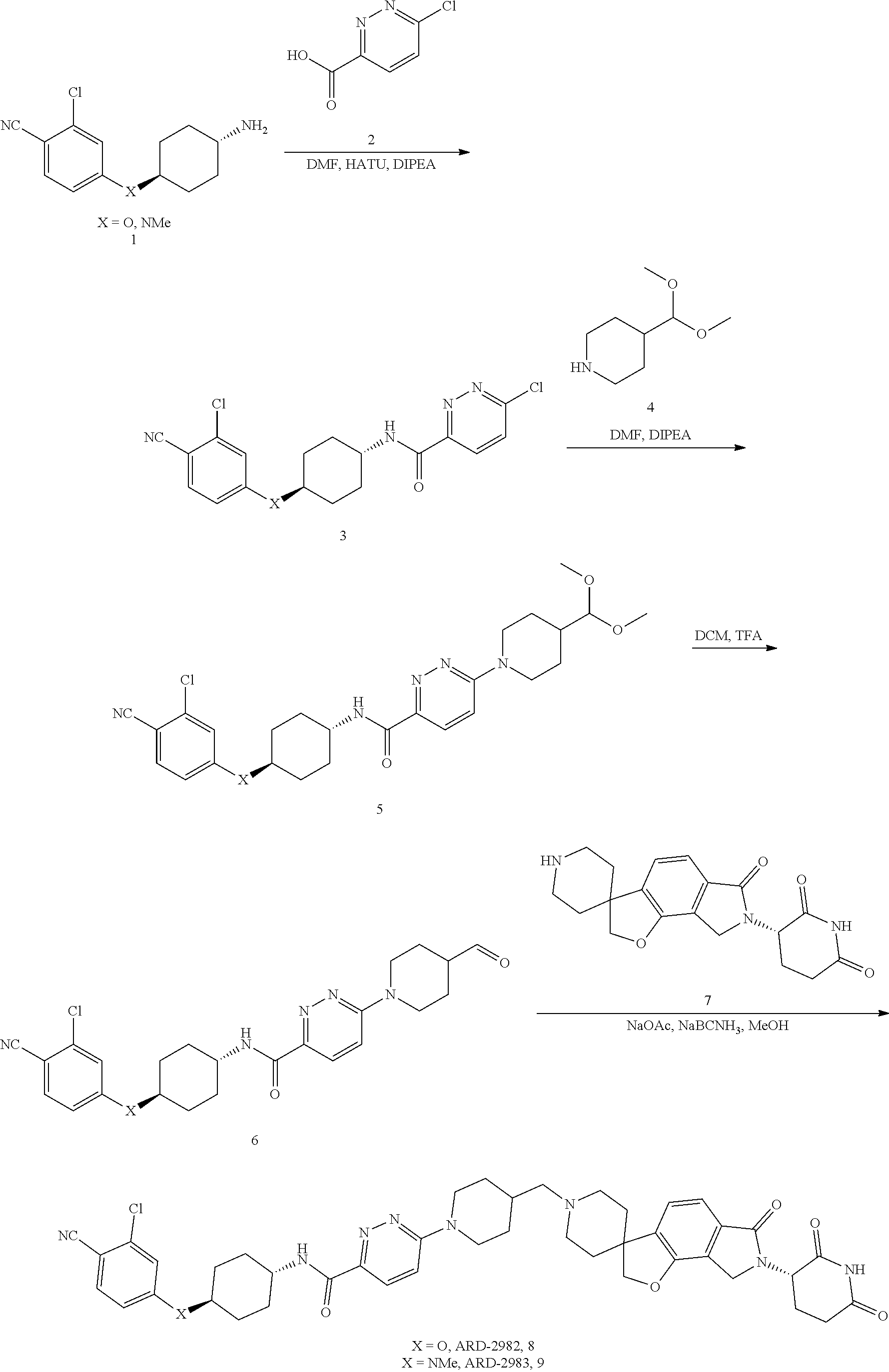

### Step 1

Compound 2 (1.0 eq), DIPEA (3.0 eq) were dissolved in DMF (10X), and HATU (1.3 eq) was added. After 15 min, compound 1 (1.0 eq) and DIPEA (2.0 eq) in DMF (3X) was added to the above solution. The reaction was finished in 0.5 h. The reaction mixture was partitioned between EtOAc and H₂O. The organic layer was separated, concentrated, purified with Combiflash using EtOAc and Hexane to give 3 in 80%.

### Step 2

Compound 3 (1.0 eq), DIPEA (4.0 eq) and 4 (1.3 eq) were dissolved in DMF (10X). The reaction was stirring at 110° C. for 2 h. The mixture was cooled down to rt and partitioned between EtOAc and H₂O. The organic layer was separated, concentrated, purified with Combiflash using EtOAc and Hexane to give 5 in 65%.

### Step 3

Compound 5 was dissolved in DCM (10X) and TFA (5X) was added. The deprotection was finished in around 4 h. All the volatile was removed under vacuum to give 6 as white solid.

### Step 4

Compound 7 (1.0 eq) and NaOAc (4.0 eq) were dissolved in MeOH (10X). After 5 mins, 6 (1.2 eq) was added, and NaBCNH3 (3.0 eq) was followed after another 10 mins. The reaction was completed in 15 mins. All the volatile was removed under vacuum, and the residue was purified by pren-HPLC to give target compound.

ARD-3: Pren-HPLC: 41% acetonitrile in H₂O. UPLS-MS: 4.3 min, 807.29. ARD-4: Pren-HPLC: 42% acetonitrile in H₂O. UPLS-MS: 4.3 min, 820.42.

### Degradation Potency Data

### Western Blot Analysis.

Western blot analysis was performed essentially as described previously. The cells treated with indicated compounds were lysed in Radioimmunoprecipitation Assay Protein Lysis and Extraction Buffer (25 mmol/L Tris.HCl, pH 7.6, 150 mmol/L NaCl, 1% Nonidet P-40, 1% sodium deoxycholate, and 0.1% sodium dodecyl sulfate) containing proteinase inhibitor cocktail (Roche Diagnostics, Mannheim, Germany). Equal amounts of total protein were electrophoresed through 10% SDS-polyacrylamide gels after determination of protein concentration by BCA assay (Fisher Scientific, Pittsburgh, PA). The separated protein bands were transferred onto PVDF membranes (GE Healthcare Life Sciences, Marlborough, MA) and blotted against different antibodies, as indicated. The blots were scanned, and the band intensities were quantified using GelQuant.NET software provided by biochemlabsolutions.com. The relative mean intensity of target proteins was expressed after normalization to the intensity of glyceraldehyde-3-phosphate dehydrogenase bands.

TABLE E14

| Compound No. | Dₘₐₓ | IC₅₀ |
|---|---|---|
| ARD-1 | >75% | <10 nM |
| ARD-2 | >75% | <10 nM |
| ARD-3 | >75% | <10 nM |
| ARD-4 | >75% | <10 nM |

### 5. BRD9 DEGRADERS

TABLE E15

| Compound | | |
|---|---|---|
| No. | Structure | Chemical Name |
| BRD-1 | 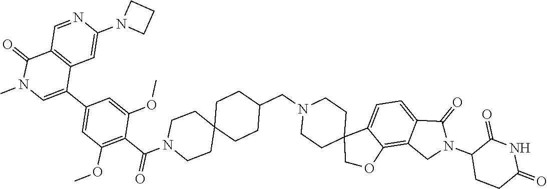 [SMILES: COc1cc(-c2cn(C)c(=O)c3cnc(N4CCC4)cc23)cc(OC)c1C(=O)N1CCC2(CCC(CN3CCC4(CC3)COc3c4ccc4c3CN(C3CCC(=O)NC3=O)C4=O)CC2)CC1] | 3-(1′-((3-(4-(6-(azetidin-1-yl)- 2-methyl-1-oxo-1,2-dihydro- 2,7-naphthyridin-4-yl)-2,6- dimethoxybenzoyl)-3- azaspiro[5.5]undecan-9- yl)methyl)-6-oxo-6,8-dihydro- 2H,7H-spiro[furo[2,3- e]isoindole-3,4′-piperidin]-7- yl)piperidine-2,6-dione |
| BRD-2 | 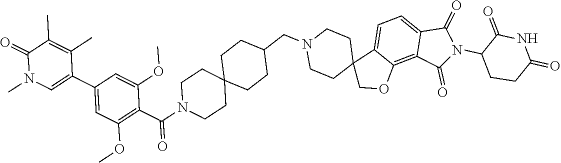 [SMILES: COc1cc(-c2cn(C)c(=O)c(C)c2C)cc(OC)c1C(=O)N1CCC2(CCC(CN3CCC4(CC3)COc3c4ccc4c3C(=O)N(C3CCC(=O)NC3=O)C4=O)CC2)CC1] | 1′-((3-(2,6-dimethoxy-4- (1,4,5-trimethyl-6-oxo-1,6- dihydropyridin-3-yl)benzoyl)- 3-azaspiro[5.5]undecan-9- yl)methyl)-7-(2,6- dioxopiperidin-3-yl)-7-hydro- 2H,6H-spiro[furo[2,3- e]isoindole-3,4′-piperidine]- 6,8-dione |

### Synthetic Schemes and Procedures

### BRD9-1. 3-(1′-((3-(4-(6-(Azetidin-1-Yl)-2-Methyl-1-Oxo-1,2-Dihydro-2,7-Naphthyridin-4-Yl)-2,6-Dimethoxybenzoyl)-3-Azaspiro[5.5]Undecan-9-Yl)Methyl)-6-Oxo-6,8-Dihydro-2H,7H-Spiro[Furo [2,3-e]Isoindole-3,4′-Piperidin]-7-Yl) Piperidine-2,6-Dione

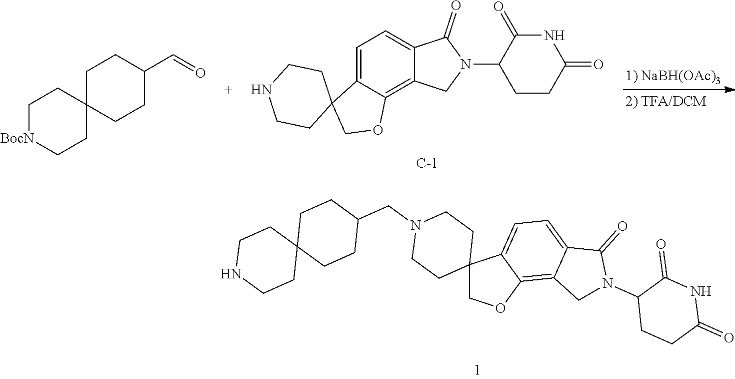

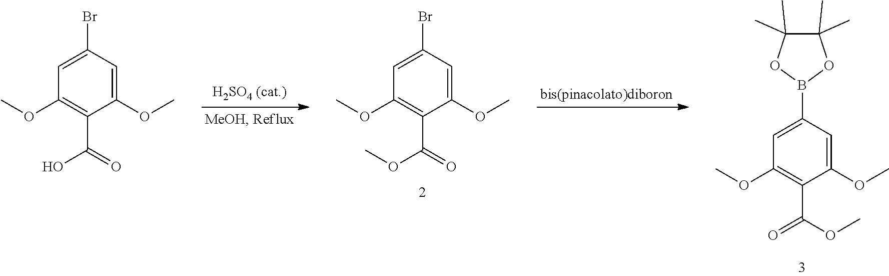

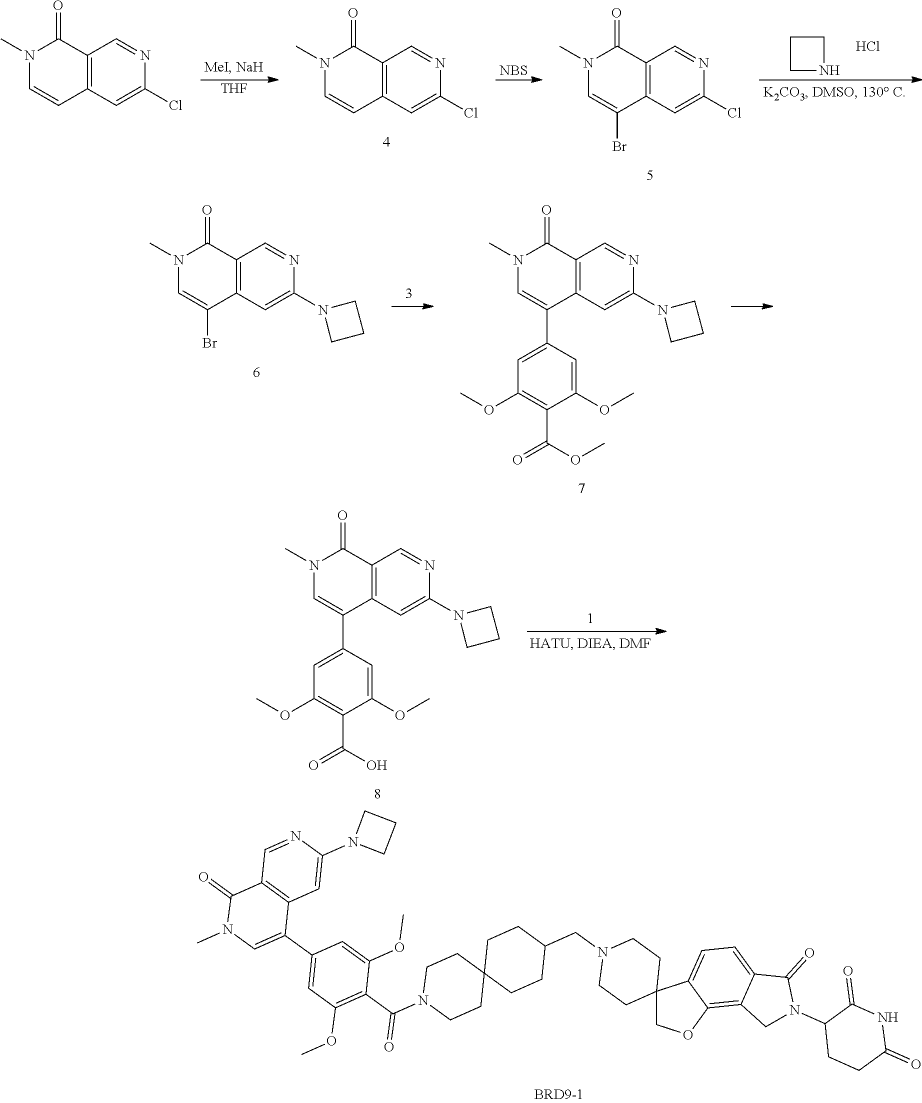

### Step 1:3-(1′-((3-Azaspiro[5.5]Undecan-9-Yl)Methyl)-6-Oxo-6,8-Dihydro-2H,7H-Spiro[Furo [2,3-e]Isoindole-3,4′-Piperidin]-7-Yl) Piperidine-2,6-Dione (1)

A mixture of tert-butyl 9-formyl-3-azaspiro[5.5]undecane-3-carboxylate (1.5 eq), 3-(6-oxo-6,8-dihydro-2H,7H-spiro[furo [2,3-e]isoindole-3,4′-piperidin]-7-yl) piperidine-2,6-dione (1 eq), and TEA (1.1 eq) in DMF (0.2 M) was stirred at rt. After 1 hour, NaBH(OAc)3 (2 eq) was added, and the reaction was stirred overnight at rt under nitrogen. The crude was purified by combi-flush RP-HPLC afforded desired product as a white solid in medium to good yield LC-MS: [M+H]⁺=621.46 (retention time: 1.56 min). Subsequent de-protection with TFA in DCM gave 3-(1′-((3-azaspiro[5.5]undecan-9-yl)methyl)-6-oxo-6,8-dihydro-2H,7H-spiro[furo [2,3-e]isoindole-3,4′-piperidin]-7-yl) piperidine-2,6-dione (1) as a white solid powder. ¹H NMR (400 MHZ, Methanol-d₄)δ 7.43 (d, J=7.7 Hz, 1H), 7.35 (d, J=7.7 Hz, 1H), 5.13 (dd, J=13.3, 5.1 Hz, 1H), 4.69 (s, 2H), 4.48 (d, J=17.1 Hz, 1H), 4.42 (d, J=17.1 Hz, 1H), 3.67 (d, J=12.8 Hz, 2H), 3.21-3.05 (m, 8H), 2.91 (ddd, J=17.7, 13.4, 5.3 Hz, 1H), 2.78 (ddd, J=17.6, 4.7, 2.4 Hz, 1H), 2.50 (qd, J=13.2, 4.7 Hz, 1H), 2.41-2.30 (m, 2H), 2.17 (dtd, J=12.8, 5.3, 2.4 Hz, 1H), 2.10-2.01 (m, 2H), 1.89-1.70 (m, 7H), 1.61 (t, J=6.0 Hz, 2H), 1.40-1.22 (m, 5H). Chemical Formula: C₃₀H₄₀N₄O₄. LC-MS: [M+H]⁺=521.4 (retention time: 0.26 min).

### Step 2: Methyl 4-Bromo-2,6-Dimethoxybenzoate (2)

To an aliquot of 4-bromo-2,6-dimethoxybenzoic acid dissolved in methanol (˜0.2 M) was added catalytic amount of concentrated sulfuric acid. The mixture is allowed to heat at 80° C. with reflux. Upon completion the mixture is concentrated via rotvap. The crude was purified by flash column chromatography using a gradient from 0% to 20% of MeOH in DCM to afford methyl 4-bromo-2,6-dimethoxybenzoate as slight yellow solid in quantitative yield. LC/MS: [M+1]⁺=276.7 (retention time: 3.98 min).

### Step 3: Methyl 2,6-Dimethoxy-4-(4,4,5,5-Tetramethyl-1,3,2-Dioxaborolan-2-Yl)Benzoate (3)

A mixture of methyl 4-bromo-2,6-dimethoxybenzoate (2) (960 mg, 3.48 mmol; 1 eq), bis(pinacolato)diboron (1.06 g, 4.18 mmol; 1.2 eq), KOAc (1.02 g, 10.44; 3.0 eq), and Pd(dppf) C₁₂ (284 mg, 0.35 mmol; 0.1 eq) in 17 mL dioxane (0.2 M) was stirred overnight at 100° C. under nitrogen atmosphere. After cooled down to rt, the resulting mixture was filtered through Celite and washed several times with EtOAc. The organic phase was washed with H₂O and brine, dried over Na₂SO₄, filtered, and evaporated in vacuum. The crude was purified by flash column chromatography using a gradient from 0% to 100% of EtOAc in hexanes to obtain the desired compound with decent yield (1.04 g, 3.24 mmol; 93%). ¹H NMR (400 MHZ, DMSO-d₆) δ 6.91 (s, 2H), 3.91 (s, 6H), 3.77 (s, 3H), 1.31 (s, 12H); LC-MS: [M+H]⁺=322.9 (retention time: 4.93 min).

### Step 4:6-Chloro-2-Methyl-2,7-Naphthyridin-1 (2H)-One (4)

To a stirred mixture of 6-chloro-2H-2,7-naphthyridin-1-one (1.41 g, 7.81 mmol, 1.0 eq) in anhydrous THF (28 mL) was added NaH (937 mg, 23.43 mmol, 3.0 eq, 60%) in portions at 0° C. After 10 min, to above mixture was added MeI (3.33 g, 23.43 mmol, 3.0 eq) at 0° C. The mixture was allowed to stir for 10 min at 0° C. Then the mixture was allowed to stir for 12 h at room temperature. The resulting mixture was concentrated by rotvap. The crude solid was slurried with water (10 mL), and the solid was filtered and collected to give the 6-chloro-2-methyl-2,7-naphthyridin-1-one as a yellow solid, that was used directly without further purification.

### Step 5:4-Bromo-6-Chloro-2-Methyl-2,7-Naphthyridin-1 (2H)-One (5)

To a stirred mixture of 6-chloro-2-methyl-2,7-naphthyridin-1-one (1.0 eq) in DMF was added NBS(1.1 eq), the resulting mixture was stirred for 2 h at 90° C. The reaction mixture was cooled and diluted with DCM, and washed with water (3×100 mL), the organic layers were combined, dried and concentrated. Then the residue was slurried with EtOAc, the slurry was filtered, washed with EtOAc to afford 4-bromo-6-chloro-2-methyl-2,7-naphthyridin-1-one (1.14 g, 4.15 mmol, 53% for 2 steps) as a white solid. LCMS(ESI) m/z: [M+H]⁺=274.8 (retention time: 1.44 min).

### Step 6:6-(Azetidin-1-Yl)-4-Bromo-2-Methyl-2,7-Naphthyridin-1 (2H)-One (6)

To a solution of 4-bromo-6-chloro-2-methyl-2,7-naphthyridin-1-one (1.14 g, 4.15 mmol, 1.0 eq) and azetidine HCl salt (736 mg, 12.45 mmol, 3 eq) in DMSO (13 mL) was added K₂CO₃ (2.86 g, 20.73 mmol, 5 eq). The resulting solution was stirred at 130° C. for 2 hours. The resulting mixture was cooled and diluted with water (25 mL), and then extracted with EtOAc (3×30 mL). The combined organic layers were washed with brine, dried over anhydrous Na₂SO₄, concentrated under reduced pressure to afford 6-(azetidin-1-yl)-4-bromo-2-methyl-2,7-naphthyridin-1 (2H)-one as a grey solid, that was used directly without further purification. LCMS(ESI) m/z: [M+H]⁺=295.9 (retention time: 1.23 min).

### Step 7: Methyl 4-(6-(Azetidin-1-Yl)-2-Methyl-1-Oxo-1,2-Dihydro-2,7-Naphthyridin-4-Yl)-2,6-Dimethoxybenzoate (7)

A mixture of 6-(azetidin-1-yl)-4-bromo-2-methyl-2,7-naphthyridin-1 (2H)-one (150 mg, 0.506 mmol; 1 eq), methyl 2,6-dimethoxy-4-(4,4,5,5-tetramethyl-1,3,2-dioxaborolan-2-yl)benzoate (163 mg, 0.506 mmol; 1 eq), Tetrakis(triphenylphosphine) palladium (0) (29 mg, 0.025 mmol; 0.05 eq), and K₂CO₃ (140 mg, 1.02 mmol; 2.0 eq) in dioxane (0.2 M): H₂O (4:1, v/v) was stirred overnight at 90° C. under nitrogen atmosphere. After cooled down to rt, the resulting mixture was filtered through Celite and washed several times with EtOAc. The organic phase was washed with H₂O and brine, dried over Na₂SO₄, filtered, and evaporated in vacuum. The crude was purified by preparative HPLC to afford methyl 4-(6-(azetidin-1-yl)-2-methyl-1-oxo-1,2-dihydro-2,7-naphthyridin-4-yl)-2,6-dimethoxybenzoate. LC/MS: [M+1]+=410.3 (retention time: 1.40 min).

### Step 8:4-(6-(Azetidin-1-Yl)-2-Methyl-1-Oxo-1,2-Dihydro-2,7-Naphthyridin-4-Yl)-2,6-Dimethoxybenzoic Acid (8)

An aliquot of methyl 4-(6-(azetidin-1-yl)-2-methyl-1-oxo-1,2-dihydro-2,7-naphthyridin-4-yl)-2,6-dimethoxybenzoate dissolved in THF: MeOH (2 mL: 1 mL; 2:1=v/v). To this mixture 2 mL of 6N NaOH (aq.) was added followed by reflux at 80° C. for overnight. Upon completion the reaction mixture was cooled down to 0° C. with ice bath, then neutralized with aq. HCl slowly. Subsequent purification via reverse phase HPLC afforded 4-(6-(azetidin-1-yl)-2-methyl-1-oxo-1,2-dihydro-2,7-naphthyridin-4-yl)-2,6-dimethoxybenzoic acid (80 mg, 0.202 mmol, 40% for 2 steps). ¹H NMR (400 MHZ, Methanol-d₄)δ 8.91 (d, J=2.6 Hz, 1H), 7.58 (d, J=2.2 Hz, 1H), 6.76 (s, 2H), 6.35 (d, J=2.9 Hz, 1H), 4.23 (t, J=7.0 Hz, 4H), 3.87 (s, 6H), 3.57 (s, 3H), 2.59-2.47 (m, 2H). LC/MS: [M+1]⁺=396.4 (retention time: 0.98 min).

### Step 9:3-(1′-((3-(4-(6-(Azetidin-1-Yl)-2-Methyl-1-Oxo-1,2-Dihydro-2,7-Naphthyridin-4-Yl)-2,6-Dimethoxybenzoyl)-3-Azaspiro[5.5]Undecan-9-Yl)Methyl)-6-Oxo-6,8-Dihydro-2H,7H-Spiro[Furo [2,3-e]Isoindole-3,4′-Piperidin]-7-Yl) Piperidine-2,6-Dione (BRD9-1)

To a solution of 4-(6-(azetidin-1-yl)-2-methyl-1-oxo-1,2-dihydro-2,7-naphthyridin-4-yl)-2,6-dimethoxybenzoic acid (27 mg, 0.068 mmol; 1 eq) in DMF (2 mL), HATU (31 mg, 0.082 mmol, 1.2 eq), 3-(1′-((3-azaspiro[5.5]undecan-9-yl)methyl)-6-oxo-6,8-dihydro-2H,7H-spiro[furo [2,3-e]isoindole-3,4′-piperidin]-7-yl) piperidine-2,6-dione (71 mg, 0.136 mmol; 2 eq), and DIEA (5 eq) were added. The reaction mixture was stirred at rt for 2 h. Upon completion 1/2 volume of H₂O was added, followed by RP-HPLC to afford desired compound BRD9-1 (37 mg, 0.041 mmol; 61%). ¹H NMR (400 MHZ, Methanol-d₄)δ 8.85 (dd, J=2.1, 0.7 Hz, 1H), 7.62 (d, J=1.6 Hz, 1H), 7.41 (dd, J=9.0, 7.7 Hz, 1H), 7.32 (dd, J=22.8, 7.7 Hz, 1H), 6.80 (d, J=3.6 Hz, 2H), 6.46-6.37 (m, 1H), 5.13 (dd, J=13.3, 5.1 Hz, 1H), 4.90 (s, 2H), 4.80 (s, 2H), 4.69 (d, J=2.9 Hz, 2H), 4.47 (d, J=17.2 Hz, 1H), 4.40 (d, J=17.0 Hz, 1H), 4.27 (q, J=9.2, 8.5 Hz, 4H), 3.86 (s, 6H), 3.76 (d, J=5.6 Hz, 2H), 3.66 (d, J=11.3 Hz, 2H), 3.57 (d, J=2.3 Hz, 3H), 3.18-3.04 (m, 4H), 2.90 (ddd, J=17.6, 13.4, 5.4 Hz, 1H), 2.78 (ddd, J=17.6, 4.9, 2.6 Hz, 1H), 2.52 (ddt, J=26.7, 13.3, 7.2 Hz, 3H), 2.33 (q, J=9.2, 6.5 Hz, 2H), 2.17 (ddd, J=9.9, 5.3, 2.5 Hz, 1H), 2.10-2.01 (m, 2H), 1.85 (d, J=9.8 Hz, 2H), 1.74-1.69 (m, 2H), 1.69 (s, 1H), 1.56 (s, 1H), 1.49 (t, J=5.8 Hz, 1H), 1.39 (d, J=6.8 Hz, 1H), 1.31 (d, J=10.0 Hz, 3H); Chemical Formula: C₅₁H₅₉N₇O₈; LC-MS: [M+H]⁺=899.85 (rt: 1.32 min).

### BRD9-2. 1′-((3-(2,6-dimethoxy-4-(1,4,5-trimethyl-6-oxo-1,6-dihydropyridin-3-yl)benzoyl)-3-azaspiro[5.5]undecan-9-yl)methyl)-7-(2,6-dioxopiperidin-3-yl)-7-hydro-2H,6H-spiro[furo [2,3-e]isoindole-3,4′-piperidine]-6,8-dione

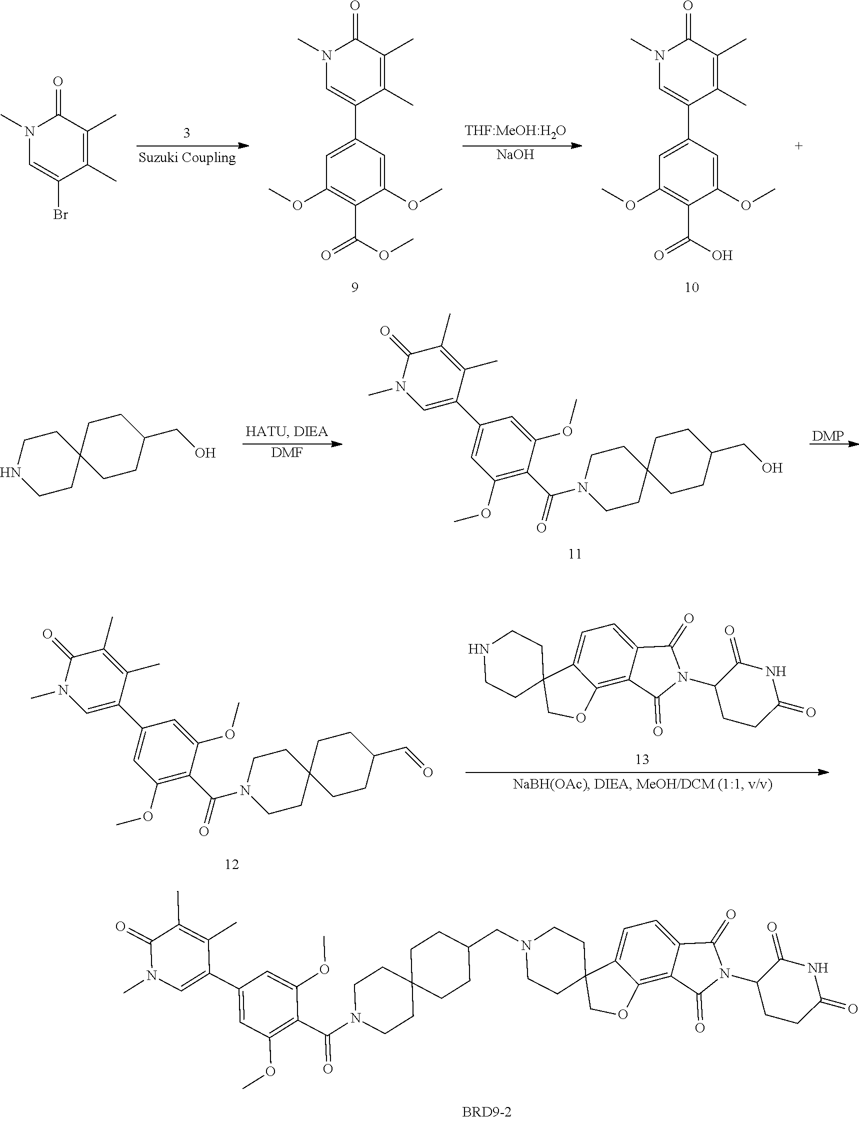

### Step 1: Methyl 2,6-Dimethoxy-4-(1,4,5-Trimethyl-6-Oxo-1,6-Dihydropyridin-3-Yl)Benzoate (9)

A mixture of 5-bromo-1,3,4-trimethylpyridin-2 (1H)-one (646 mg, 3.18 mmol; 1 eq), methyl 2,6-dimethoxy-4-(4,4,5,5-tetramethyl-1,3,2-dioxaborolan-2-yl)benzoate (1.024 g, 3.18 mmol; 1 eq), Tetrakis(triphenylphosphine) palladium (0) (184 mg, 0.16 mmol; 0.05 eq), and K₂CO₃ (878 mg, 6.36 mmol; 2.0 eq) in dioxane (0.2 M): H₂O (4:1, v/v) was stirred overnight at 90° C. under nitrogen atmosphere. After cooled down to rt, the resulting mixture was filtered through Celite and washed several times with EtOAc. The organic phase was washed with H₂O and brine, dried over Na₂SO₄, filtered, and evaporated in vacuum. The crude was purified by preparative HPLC to afford methyl 2,6-dimethoxy-4-(1,4,5-trimethyl-6-oxo-1,6-dihydropyridin-3-yl)benzoate (9) with decent yield. LC/MS: [M+1]⁺=332.1 (retention time: 2.92 min).

### Step 2:2,6-Dimethoxy-4-(1,4,5-Trimethyl-6-Oxo-1,6-Dihydropyridin-3-Yl)Benzoic Acid (10)

An aliquot of methyl 2,6-dimethoxy-4-(1,4,5-trimethyl-6-oxo-1,6-dihydropyridin-3-yl)benzoate dissolved in THF: MeOH (2 mL: 1 mL; 2:1=v/v). To this mixture 2 mL of 6N NaOH (aq.) was added followed by reflux at 80° C. for overnight. Upon completion the reaction mixture was cooled down to 0° C. with ice bath, then neutralized with aq. HCl slowly. Subsequent purification via reverse phase HPLC afforded 2,6-dimethoxy-4-(1,4,5-trimethyl-6-oxo-1,6-dihydropyridin-3-yl)benzoic acid (10) (585 mg, 1.84 mmol, 58% for 2 steps). ¹H NMR (400 MHZ, Methanol-d₄)δ 7.45 (s, 1H), 6.59 (s, 2H), 3.84 (s, 6H), 3.60 (s, 3H), 2.16 (d, J=12.0 Hz, 6H). LC/MS: [M+1]⁺=318.1 (retention time: 1.99 min).

### Step 3:5-(4-(9-(Hydroxymethyl)-3-Azaspiro[5.5]Undecane-3-Carbonyl)-3,5-Dimethoxyphenyl)-1,3,4-Trimethylpyridin-2 (1H)-One (11)

To a solution of 2,6-dimethoxy-4-(1,4,5-trimethyl-6-oxo-1,6-dihydropyridin-3-yl)benzoic acid (63 mg, 0.197 mmol, 1 eq) in DMF (2 mL), HATU (1.2 eq), (3-azaspiro[5.5]undecan-9-yl) methanol (54 mg, 0.296 mmol, 1.5 eq), and DIEA (10 eq) were added. The reaction mixture was stirred at rt for 2 h. Upon completion 1/2 volume of H₂O was added, followed by RP-HPLC to afford 5-(4-(9-(hydroxymethyl)-3-azaspiro[5.5]undecane-3-carbonyl)-3,5-dimethoxyphenyl)-1,3,4-trimethylpyridin-2 (1H)-one (11) (72 mg, 0.149 mmol; 76%). LC-MS: [M+H]⁺=283.3 (retention time: 3.44 min).

### Step 4:3-(2,6-Dimethoxy-4-(1,4,5-Trimethyl-6-Oxo-1,6-Dihydropyridin-3-Yl)Benzoyl)-3-Azaspiro[5.5]Undecane-9-Carbaldehyde (12)

To a solution of 5-(4-(9-(hydroxymethyl)-3-azaspiro[5.5]undecane-3-carbonyl)-3,5-dimethoxyphenyl)-1,3,4-trimethylpyridin-2 (1H)-one (72 mg, 0.149 mmol) in dichloromethane (3 mL) was added 1.3 eq DMP. The mixture was stirred at room temperature for 30 mins. TLC (ethyl acetate: hexane=1:1) showed reaction was complete. The mixture was diluted with dichloromethane (3 ml) and filtered through a pad of Celite. The filtrate was concentrated in vacuum. The crude product was purified by silica gel column chromatography to yield 3-(2,6-dimethoxy-4-(1,4,5-trimethyl-6-oxo-1,6-dihydropyridin-3-yl)benzoyl)-3-azaspiro[5.5]undecane-9-carbaldehyde (30 mg, 0.063 mmol; 42%).

### Step 5: 1′-((3-(2,6-Dimethoxy-4-(1,4,5-Trimethyl-6-Oxo-1,6-Dihydropyridin-3-Yl)Benzoyl)-3-Azaspiro[5.5]Undecan-9-Yl)Methyl)-7-(2,6-Dioxopiperidin-3-Yl)-7-Hydro-2H,6H-Spiro[Furo [2,3-e]Isoindole-3,4′-Piperidine]-6,8-Dione (dBRD9-1)

A mixture of 3-(2,6-dimethoxy-4-(1,4,5-trimethyl-6-oxo-1,6-dihydropyridin-3-yl)benzoyl)-3-azaspiro[5.5]undecane-9-carbaldehyde (30 mg, 0.063 mmol, 1.5 eq), 7-(2,6-dioxopiperidin-3-yl)-7-hydro-2H,6H-spiro[furo [2,3-e]isoindole-3,4′-piperidine]-6,8-dione (16 mg, 0.042 mmol, 1 eq), and TEA (1.1 eq) in DMF (0.2 M) was stirred at rt. After 1 hour, NaBH(OAc)3 (2 eq) was added, and the reaction was stirred overnight at rt under nitrogen. The crude was purified by Pre-HPLC afforded desired product as a white solid (20 mg, 0.024 mmol, 58%). ¹H NMR (400 MHZ, Methanol-d₄)δ 7.59 (dd, J=7.4, 2.6 Hz, 1H), 7.51-7.43 (m, 2H), 6.61 (d, J=1.6 Hz, 2H), 5.15-5.06 (m, 1H), 4.82 (d, J=2.9 Hz, 3H), 3.83 (d, J=1.6 Hz, 6H), 3.80-3.65 (m, 4H), 3.60 (d, J=2.3 Hz, 3H), 3.27 (d, J=7.4 Hz, 3H), 3.09 (dd, J=14.9, 7.1 Hz, 3H), 2.94-2.80 (m, 1H), 2.80-2.64 (m, 2H), 2.38-2.26 (m, 2H), 2.20-2.09 (m, 9H), 1.86 (d, J=13.3 Hz, 3H), 1.69 (d, J=17.3 Hz, 3H), 1.55 (d, J=5.7 Hz, 1H), 1.49 (t, J=6.0 Hz, 1H), 1.37 (d, J=5.6 Hz, 2H), 1.30 (m, 3H); Chemical Formula: C₄₇H₅₅N₅O₉; LC-MS: [M+H]⁺=834.52 (retention time: 1.31 min).

### Biological Activity

HiBiT Assay for protein degradation:

The HiBit assay was performed using engineered HEK293 CRISPR knock-in cell utilizing the Nano-Glo HiBiT Lytic Detection System from Promega, Cat #N3040. Compounds were transferred, 25 nL DMSO or test compounds (final DMSO @ 0.1%) to intermediate plates (Corning3570) using ECHO550. Cells were seeded onto compounds at 2000 cells/25 μL/well and incubated for 6 hrs in tissue culture incubator at 37° C. and 5% CO₂. The LgBiT Protein was diluted at 1:100 and the Nano-Glo® HiBiT Lytic Substrate 1:50 into an appropriate volume of room temperature using the Nano-Glo® HiBiT Lytic Buffer. At end of compound incubation time 15 ul of the detection reagent was added to each well (without LgBiT for negative control wells) using the Thermo Scientific multidrop combi. The plate was shaken for 10 mins at RT using Combi. After brief centrifugation (2000 rpm 1 mins), plate was read on Envision (Ultrasensitive luminescence model). The results of the HiBiT Assay are summarized in Table E16 below.

TABLE E16

BRD9
HEK293 HiBit

| Compound No. | DC₅₀ (nM) | Dₘₐₓ (%) |
|---|---|---|
| BRD-1 | A | A |
| BRD-2 | B | A |

Note:
DC₅₀: A (<10 nM), B (10-100 nM), C (101-500 nM), and D (>500 nM).
Dₘₐₓ: A (>90% degradation), B (70-90% degradation), C (50-69% degradation), and D (<50% degradation).

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. A compound of Formula II: 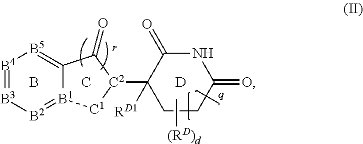
or a pharmaceutically acceptable salt, solvate, or stereoisomer thereof, wherein
B² is N or CR^{B2};
B³ is N or CR^{B3};
B⁴ is N or CR^{B4};
B⁵ is N or CR^{B5};
R^{B2}, R^{B3}, R^{B4}, and R^{B5} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{C}S(═O)R^{a}, —NR^{C}S(═O)₂OR^{b}, —NR^{C}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
wherein one of R^{B2} and R^{B3}, R^{B3} and R^{B4}, or R^{B4} and R^{B5}, together with the carbon atoms to which they are bonded, form Ring A, wherein Ring A is optionally substituted 7- to 16-membered spiro heterocycle;
--denotes an optional covalent bond between B¹ and C₁;
i) when the bond between B¹ and C¹ is present:
r is 1;
B¹ is C;
C¹ is —C(R^{C1})₂—, —C(═O)—, —(C═O)—N(R^{C1}′)—*, or —N═C(R^{C1}′)—*;
each R^{C1} is independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}; or
two R^{C1}, together with the carbon atom to which they are attached, form C₃₋₆ carbocycle or 3- to 6-membered heterocycle, wherein the carbocycle or heterocycle is optionally substituted with one or more R^{u};
R^{C1}′ is H or C₁₋₆ alkyl optionally substituted with one or more R^{u}, and * denotes attachment to Ring B; and
C² is N;
ii) when the bond between B¹ and C¹ is absent:
r is 0 or 1;
B¹ is N or CR^{B1};
R^{B1} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
C¹ is absent; or
C¹ is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
C₂ is N or O;
wherein i) when C² is N, then C¹ is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}; ii) when C₂ is O, then C¹ is absent;
R^{D1} is hydrogen, deuterium, or C₁₋₆ alkyl optionally substituted with one or more R^{u};
q is an integer from 0 to 2,
each RD is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}; and
d is an integer selected from 0 to 5,
wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{C}S(═O)R^{a}, —NR^{C}S(═O)₂OR^{b}, —NR^{b}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₃₋₆ carbocyclyl, 3- to 6-membered heterocyclyl, C₆ aryl, and 5- or 6-membered heteroaryl; or
two R^{u}, together with the one or more intervening atoms, form C₆₋₁₀ aryl, 5- to 10-membered heteroaryl, C₃₋₁₂ carbocyclyl, or 3- to 12-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each R^{c} and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
or
R^{c} and R^{d}, together with the nitrogen atom to which they are attached, form 3- to 12-membered heterocyclyl or 5- to 10-membered heteroaryl, wherein the heterocyclyl or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₃₋₆ carbocyclyl, and 3- to 6-membered heterocyclyl;
wherein each of R^{a}, Rb, R^{c}, and R^{d} is independently and optionally substituted with one or more R^{z};
each R^{z} is independently oxo, halogen, —CN, —NO₂, —OH, —NH₂, C₁₋₆ alkyl, C₁₋₆ alkoxy, C₁₋₆ alkylamino, C₃₋₆ carbocyclyl, 3- to 6-membered heterocyclyl, C₆ aryl, or 5- or 6-membered heteroaryl.

2. A conjugate of Formula II: 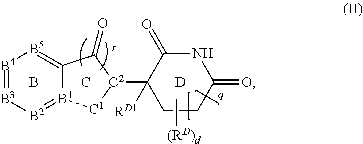
wherein:
B² is N or CR^{B2};
B³ is N or CR^{B3};
B⁴ is N or CR^{B4};
B⁵ is N or CR^{B5};
R^{B2}, R^{B3}, R^{B4}, and R^{B5} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{C}S(═O)R^{a}, —NR^{C}S(═O)₂OR^{b}, —NR^{C}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
wherein one of R^{B2} and R^{B3}, R^{B3} and R^{B4}, or R^{B4} and R^{B5}, together with the carbon atoms to which they are bonded, form Ring A attached to -L-T, wherein Ring A is optionally substituted 7- to 16-membered spiro heterocycle;
-denotes an optional covalent bond between B¹ and C₁;
i) when the bond between B¹ and C¹ is present:
r is 1;
B¹ is C;
C¹ is —C(R^{C1})₂—, —C(═O)—, —(C═O)—N(R^{C1}′)—*, or —N═C(R^{C1}′)—*;
each R^{C1} is independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}; or
two R^{C1}, together with the carbon atom to which they are attached, form C₃₋₆ carbocycle or 3- to 6-membered heterocycle, wherein the carbocycle or heterocycle is optionally substituted with one or more R^{u};
R^{C1}′ is H or C₁₋₆ alkyl optionally substituted with one or more R^{u}, and * denotes attachment to Ring B; and
C² is N;
ii) when the bond between B¹ and C¹ is absent:
r is 0 or 1;
B¹ is N or CR^{B1};
R^{B1} is hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
C¹ is absent; or
C¹ is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u};
C₂ is Nor O;
wherein i) when C² is N, then C¹ is hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}; ii) when C₂ is O, then C¹ is absent;
R^{D1} is hydrogen, deuterium, or C₁₋₆ alkyl optionally substituted with one or more R^{u};
q is an integer from 0 to 2,
each RD is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u};
d is an integer selected from 0 to 5,
L is linker; and
T is a ligand for a protein,
wherein:
each R^{u} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₆₋₁₀ aryl, 5- to 10-membered heteroaryl, C₃₋₁₂ carbocyclyl, 3- to 12-membered heterocyclyl, —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{C}S(═O)R^{a}, —NR^{C}S(═O)₂OR^{b}, —NR^{b}s (═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}; wherein the alkyl, alkoxy, alkylamino, alkenyl, alkynyl, carbocyclyl, heterocyclyl, aryl, or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₃₋₆ carbocyclyl, 3- to 6-membered heterocyclyl, C₆ aryl, and 5- or 6-membered heteroaryl; or
two R^{u}, together with the one or more intervening atoms, form C₆₋₁₀ aryl, 5- to 10-membered heteroaryl, C₃₋₁₂ carbocyclyl, or 3- to 12-membered heterocyclyl;
each R^{a} is independently C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
each R^{b} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl; and
each Re and R^{d} is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl;
or
R^{c} and R^{d}, together with the nitrogen atom to which they are attached, form 3- to 12-membered heterocyclyl or 5- to 10-membered heteroaryl, wherein the heterocyclyl or heteroaryl is optionally substituted with one or more substituents selected from oxo, halogen, —CN, —NO₂, —OH, —NH₂, C₁₋₆ alkyl, C₁₋₆ alkoxy, C₁₋₆ alkylamino, C₃₋₆ carbocyclyl, and 3- to 6-membered heterocyclyl;
wherein each of R^{a}, Rb, R^{c}, and R^{d} is independently and optionally substituted with one or more R^{z};
each R^{z} is independently oxo, halogen, —CN, —NO₂, —OH, —NH₂, C₁₋₆ alkyl, C₁₋₆ alkoxy, C₁₋₆ alkylamino, C₃₋₆ carbocyclyl, 3- to 6-membered heterocyclyl, C₆ aryl, or 5- or 6-membered heteroaryl.

3. The compound or conjugate of claim 1 or 2, wherein the compound or conjugate of Formula II is a compound or conjugate of Formula II-1 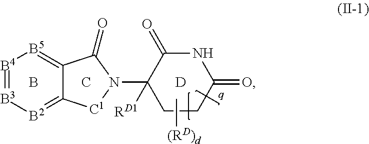
or a pharmaceutically acceptable salt, solvate, or stereoisomer thereof.

4. The compound or conjugate of claim 3, wherein C¹ is —CH₂—.

5. The compound or conjugate of claim 1 or 2, wherein the compound or conjugate of Formula II is a compound or conjugate of Formula II-2 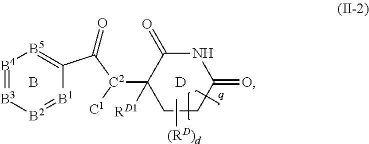
or a pharmaceutically acceptable salt, solvate, or stereoisomer thereof.

6. The compound or conjugate of any one of claims 1-5, wherein R^{B2} and R^{B3}, together with the carbon atoms to which they are bonded, form Ring A or Ring A attached to -L-T.

7. The compound or conjugate of any one of claims 1-5, wherein R^{B3} and R^{B4}, together with the carbon atoms to which they are bonded, form Ring A or Ring A attached to -L-T.

8. The compound or conjugate of any one of claims 1-7, wherein
Ring A is: 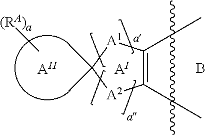
or
Ring A attached to -L-T is 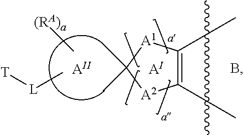
wherein:
** denotes attachment to C;
Ring A¹ is C₃₋₈ carbocycle or 3- to 8-membered heterocycle;
each A¹ is independently —C(R^{A1})₂—, —NR^{A1}′—, —O—, —S—, —S(═O)—, or —S(═O)₂—;
each A² is independently —C(R^{A2})₂—, —NR^{A2}′—, —O—, —S—, —S(═O)—, or —S(═O)₂—;
each occurrence of RAI and R^{A2} is independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{C}S(═O)R^{a}, —NR^{C}S(═O)₂OR^{b}, —NR^{C}S(═O)₂NR^{c}R^{d}, —NR^{o}C(═O)NR^{c}R^{d}, —NR^{o}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u};
two geminal R^{A1} or two geminal R^{A2} together form an oxo; or
two geminal R^{A1} or two geminal R^{A2}, together with the carbon atom to which they are attached, form C₃₋₆ carbocycle or 3- to 6-membered heterocycle, wherein the carbocycle or heterocycle is optionally substituted with one or more R^{u};
each occurrence of R^{A1}′ and R^{A2}′ is independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u};
a′ and a″ are independently an integer selected from 0-3, wherein one of a′ and a″ is 0, and a′ and a″ are not both 0;
each R^{A} is independently oxo, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —SR^{b}, —S(═O)R^{a}, —S(═O)₂R^{a}, —S(═O)₂OR^{b}, —S(═O)₂NR^{c}R^{d}, —NR^{C}S(═O)₂R^{a}, —NR^{C}S(═O)R^{a}, —NR^{C}S(═O)₂OR^{b}, —NR^{C}S(═O)₂NR^{c}R^{d}, —NR^{b}C(═O)NR^{c}R^{d}, —NR^{b}C(═O)R^{a}, —NR^{b}C(═O)OR^{b}, —OS(═O)₂R^{a}, —OS(═O)₂OR^{b}, —OS(═O)₂NR^{c}R^{d}, —OC(═O)R^{a}, —OC(═O)OR^{b}, —OC(═O)NR^{c}R^{d}, —C(═O)R^{a}, —C(═O)OR^{b}, or —C(═O)NR^{c}R^{d}, wherein the alkyl, alkoxy, alkylamino, alkenyl, alkynyl, carbocyclyl, heterocyclyl, aryl, or heteroaryl is optionally substituted with one or more R^{u}; and
a is an integer selected from 0 to 8, as valency permits.

9. The compound or conjugate of claim 8, wherein Ring A¹ is heterocyclyl.

10. The compound or conjugate of claim 8 or 9, wherein
Ring A is: 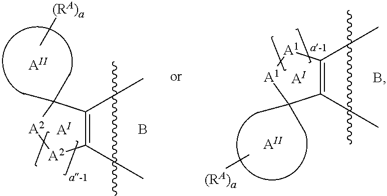
or
Ring A attached to -L-T is

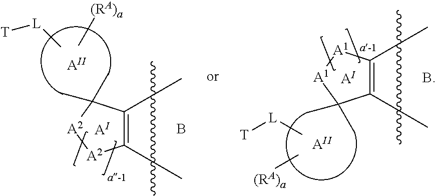

11. The compound or conjugate of claim 8 or 9, wherein each A¹ is independently —C(R^{A1})₂—, —NR^{A1}′—, or —O—, or each A² is independently —C(R^{A2})₂—, —NR^{A2}′—, or —O—.

12. The compound or conjugate of any one of claims 8-10, wherein
each occurrence of RAI and R^{A2} is independently hydrogen, halogen, —CN, —NO₂, —OH, —NH₂, C₁₋₆ alkyl, C₁₋₆ alkoxy, C₁₋₆ alkylamino, C₃₋₆ carbocyclyl, or 3- to 6-membered heterocyclyl, wherein the alkyl, alkoxy, alkylamino, carbocyclyl, or heterocyclyl is optionally substituted with one or more R^{u}; and
each occurrence of R^{A1}′ and R^{A2}′ is independently hydrogen, C₁₋₆ alkyl, C₃₋₆ carbocyclyl, 3- to 6-membered heterocyclyl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, carbocyclyl, or heterocyclyl is optionally substituted with one or more R^{u}.

13. The compound or conjugate of claim 11, wherein each A¹ is independently —CH₂—, —NH—, or —O—, or each A² is independently —CH₂—, —NH—, or —O—.

14. The compound or conjugate of claim 10, wherein
Ring A is 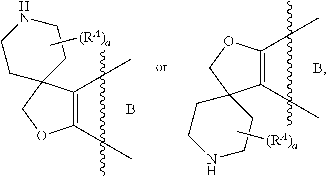
or
Ring A attached to -L-T is

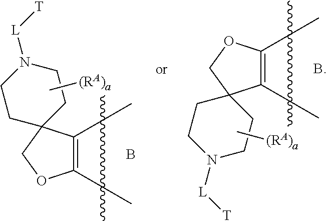

15. The compound or conjugate of claim 10, wherein the compound or conjugate of Formula II is
1) a compound of Formula II-1-a-ii, II-1-a-iii, II-1-a-iv, II-1-a-v, II-1-a-vi, II-1-a-vii, II-1-a-ix, II-1-a-x, II-1-a-xi, II-1-a-xii, or II-1-a-xiii: 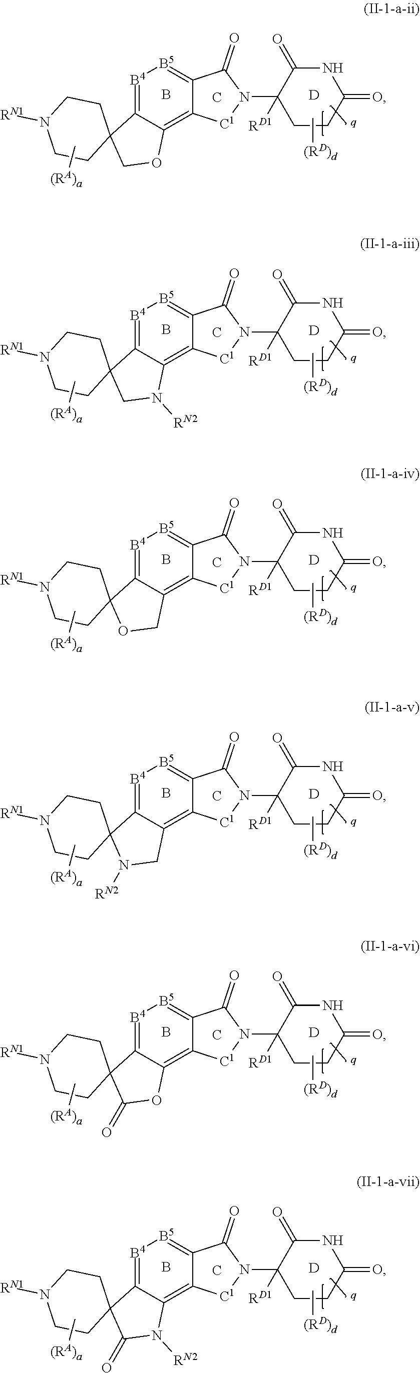 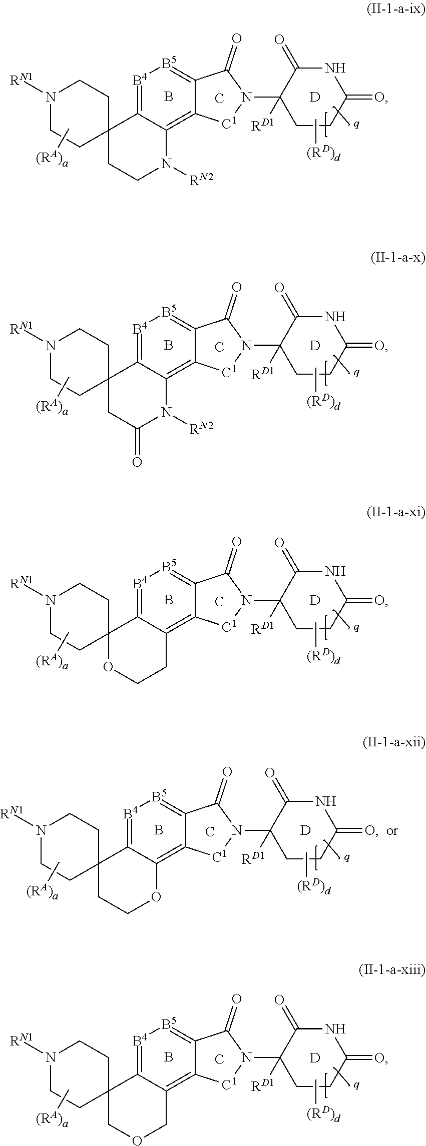
or a pharmaceutically acceptable salt, solvate, or stereoisomer thereof, or
2) a conjugate of Formula II′-1-a-ii, II′-1-a-iii, II′-1-a-iv, II′-1-a-v, II′-1-a-vi, II′-1-a-vii, II′-1-a-ix, II′-1-a-x, II′-1-a-xi, II′-1-a-xii, or II-1-a-xiii 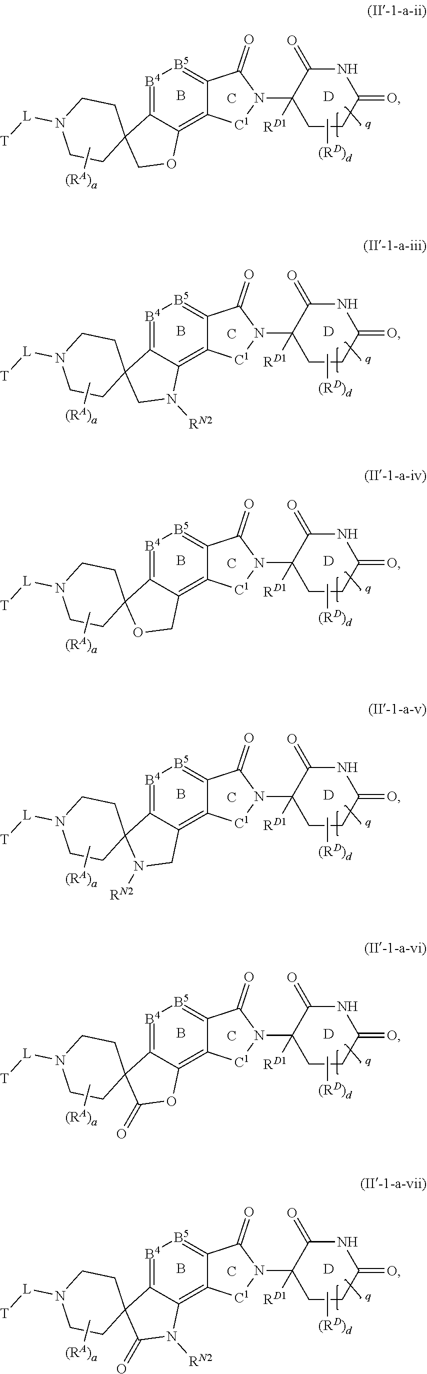 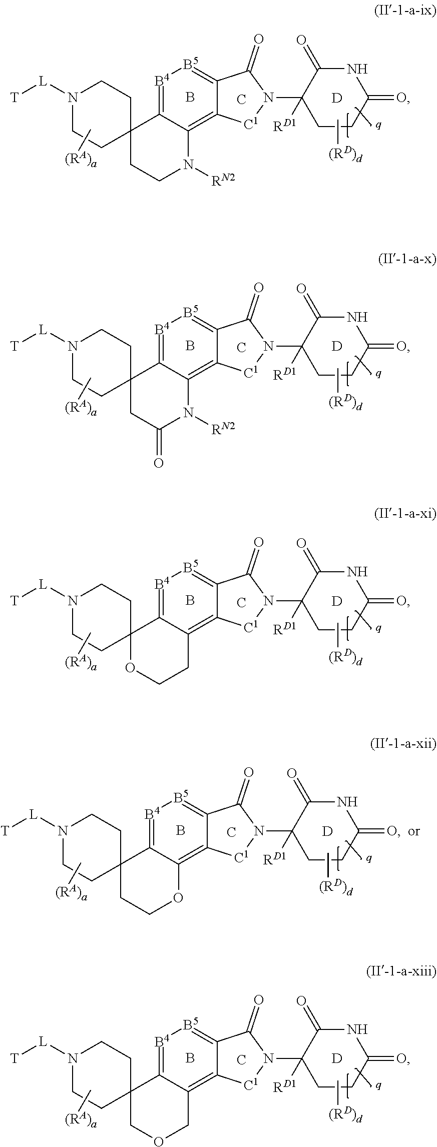
or a pharmaceutically acceptable salt, solvate, or stereoisomer thereof,
wherein:
R^{N1} and R^{N2} are independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}; or
R^{N1} and R^{N2} are independently an amino-protecting group.

16. The compound or conjugate of claim 15, wherein B⁴ is CR^{B4} and B⁵ is CR^{B5}, wherein R^{B4} and R^{B5} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

17. The compound or conjugate of claim 16, wherein R^{B4} and R^{B5} are both hydrogen.

18. The compound or conjugate of claim 10, wherein the compound or conjugate of Formula II is
1) a compound of Formula II-1-b-ii, II-1-b-iii, II-1-b-iv, II-1-b-v, II-1-b-vi, II-1-b-vii, II-1-b-ix, II-1-b-x, II-1-b-xi, II-1-b-xii, or II-1-b-xiii: 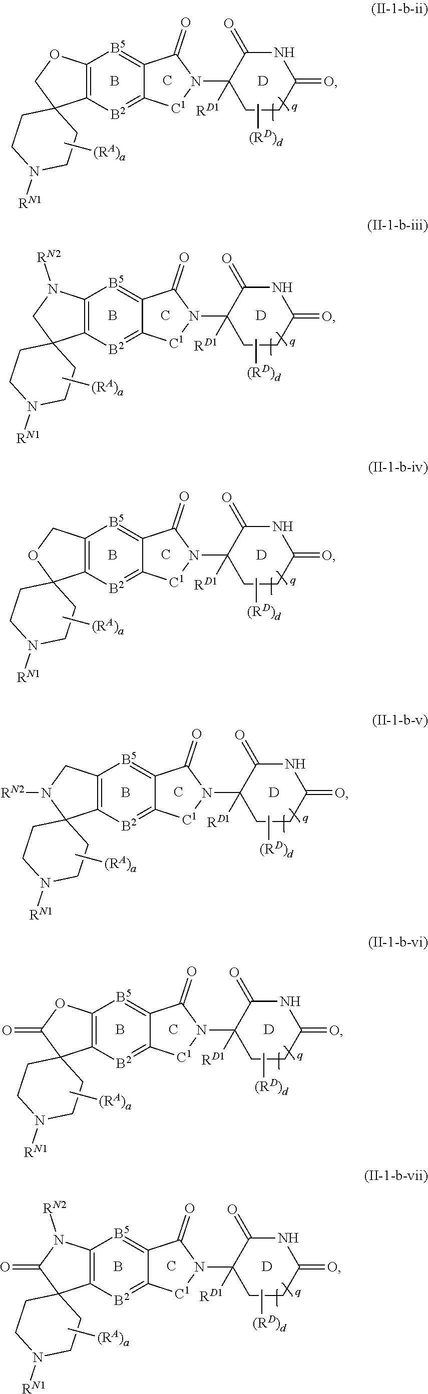 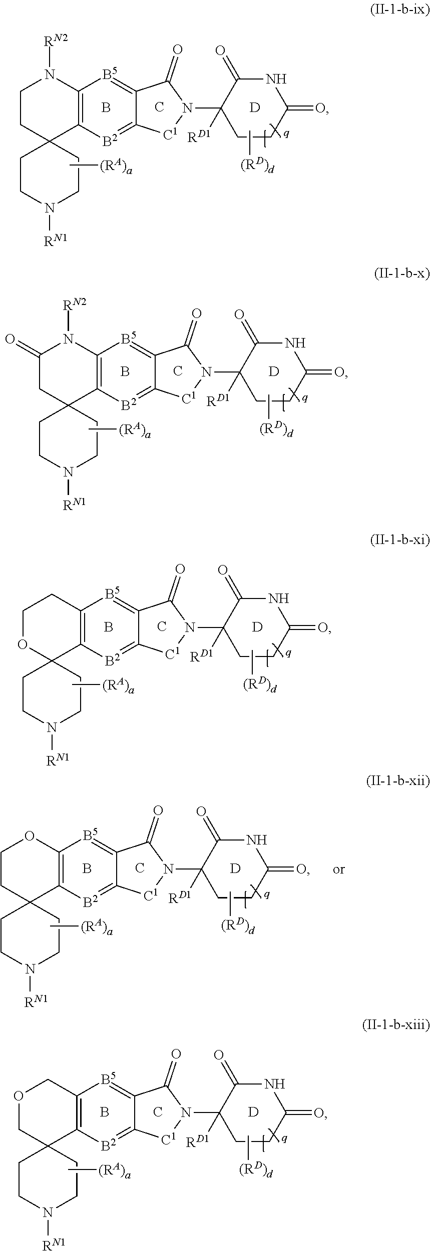
or a pharmaceutically acceptable salt, solvate, or stereoisomer thereof, or
2) a conjugate of Formula II′-1-b-ii, II′-1-b-iii, II′-1-b-iv, II′-1-b-v, II′-1-b-vi, II′-1-b-vii, II′-1-b-ix, II′-1-b-x, II′-1-b-xi, II′-1-b-xii, or II′-1-b-xiii: 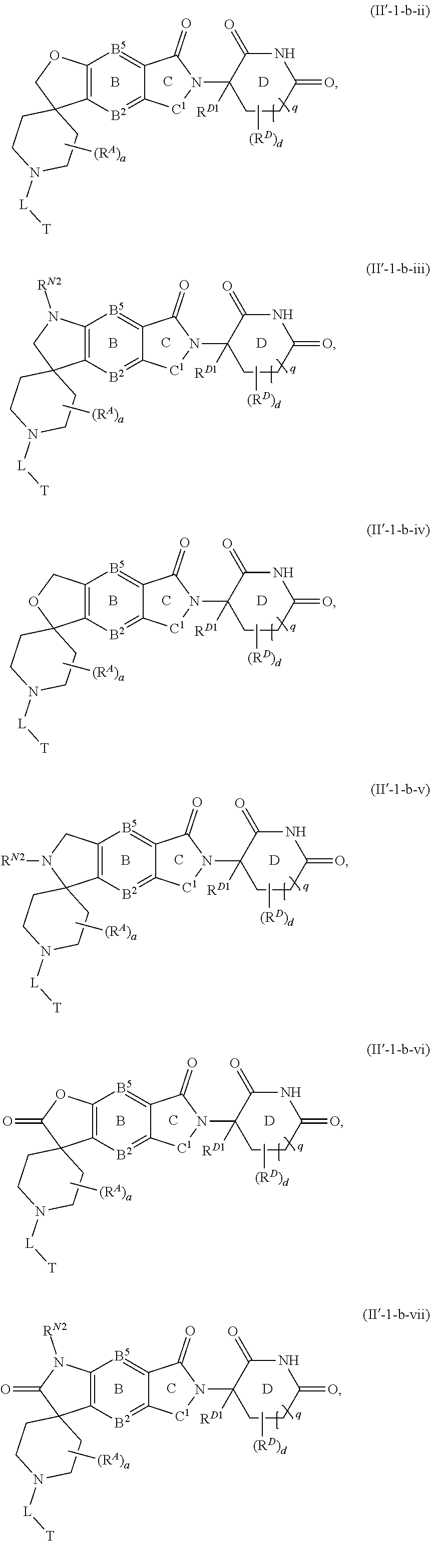 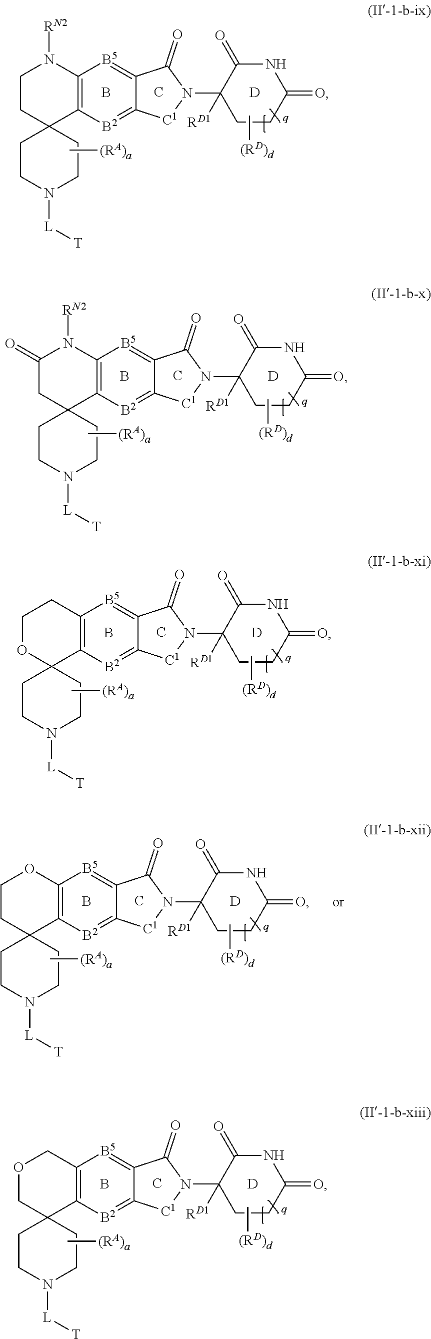
or a pharmaceutically acceptable salt, solvate, or stereoisomer thereof,
wherein:
R^{N1} and R^{N2} are independently hydrogen, C₁₋₆ alkyl, C₂₋₆ alkenyl, C₂₋₆ alkynyl, C₃₋₁₂ carbocyclyl, 3- to 12-membered heterocyclyl, C₆₋₁₀ aryl, 5- to 10-membered heteroaryl, —S(═O)₂R^{a}, —S(═O)₂OR^{b}, —S(═O)₂NR^{c}R^{d}, —C(═O)R^{a}, —C(═O)OR^{b}, or —C(═O)NR^{c}R^{d}, wherein the alkyl, alkenyl, alkynyl, carbocyclyl, heterocyclyl, aryl, or heteroaryl is optionally substituted with one or more R^{u}; or
R^{N1} and R^{N2} are independently an amino-protecting group.

19. The compound or conjugate of claim 18, wherein B² is CR^{B2} and B⁵ is CR^{B5}, wherein R^{B2} and R^{B5} are independently hydrogen, halogen, —CN, —NO₂, —OH, —NH₂, C₁₋₆ alkyl, C₁₋₆ alkoxy, C₁₋₆ alkylamino, C₂₋₆ alkenyl, C₂₋₆ alkynyl, C₃₋₁₂ carbocyclyl, 3- to 12-membered heterocyclyl, C₆₋₁₀ aryl, or 5- to 10-membered heteroaryl, wherein the alkyl, alkoxy, alkylamino, alkenyl, alkynyl, carbocyclyl, heterocyclyl, aryl, or heteroaryl is optionally substituted with one or more R^{u}.

20. The compound or conjugate of claim 19, wherein R^{B2} and R^{B5} are both hydrogen.

21. The compound or conjugate of any one of claims 1-20, wherein R^{D1} is hydrogen.

22. The compound or conjugate of any one of claims 1-21, wherein d is 0.

23. The compound or conjugate of any one of claims 1-22, wherein q is 1.

24. A compound selected from the compounds in Tables 1 and 2 or a pharmaceutically acceptable salt thereof.

25. A pharmaceutical composition comprising the compound or conjugates of any one of claims 1-23, and a pharmaceutically acceptable excipient.

26. A method of binding cereblon E3 ubiquitin ligase protein complex in a subject or biological sample comprising administering the compound of any one of claims 1-23 to the subject or contacting the biological sample with the compound of any one of claims 1-23.

27. Use of the compound of any one of claims 1-23 in the manufacture of a medicament for binding cereblon E3 ubiquitin ligase protein complex in a subject or biological sample.

29. A compound of any one of claims 1-23 for use in binding cereblon E3 ubiquitin ligase protein complex in a subject or biological sample.

30. A method of degrading a protein in a subject or biological sample comprising administering the compound or conjugate of any one of claims 1-23 to the subject or contacting the biological sample with the compound or conjugate of any one of claims 1-23.

31. Use of the compound or conjugate of any one of claims 1-23 in the manufacture of a medicament for degrading a protein in a subject or biological sample.

32. A compound or conjugate of any one of claims 1-23 for use in degrading a protein in a subject or biological sample.

33. The method, use, or compound for use of any one of claims 30-33, wherein the protein is an estrogen receptor, a STAT3 protein, an androgen receptor, a SMARCA2 protein, a SMARCA4 protein, a BRD9 protein, or a CBP/p300 protein.
